(19) United States
(12) Patent Application Publication
(10) Pub. No.: US 2022/0331028 A1
(43) Pub. Date: Oct. 20, 2022

(54) System for Capturing Movement Patterns and/or Vital Signs of a Person

(71) Applicant: MetraLabs GmbH Neue Technologien und Systeme, Imenau (DE)

(72) Inventors: Christian Sternitzke, Leipzig (DE); Anke Mayfarth, Ilmenau (DE)

(21) Appl. No.: 17/639,254

(22) PCT Filed: Aug. 31, 2020
(86) PCT No.: PCT/EP2020/074240

§ 371 Date: Feb. 28, 2022

(30) Foreign Application Priority Data
Aug. 30, 2019 (DE) 10 2019 123 304.6
Jan. 30, 2020 (DE) 10 2020 102 315.4
May 12, 2020 (DE) 10 2020 112 853.3

(51) Int. Cl. A61B 34/32; A61B 5/11; A61B 5/00; B25J 11/00; G06V 40/20; G06V 40/10

(52) U.S. Cl. CPC A61B 34/32; A61B 5/1113; A61B 5/6887; A61B 5/0077; B25J 11/008; G06V 40/20; G06V 40/10

(57) ABSTRACT

System and method for capturing a movement sequence of a person. The method comprises capturing a plurality of images of the person executing a movement sequence by means of a contactless sensor, the plurality of images representing the movements of the body elements of the person, generating at least one skeleton model having limb positions for at least some of the plurality of images, and calculating the movement pattern from the movements of the body elements of the person by comparing changes in the limb positions in the at least one skeleton model generated. In addition, vital signs and/or signal processing parameters of the person can be acquired and evaluated.

### CROSS-REFERENCE TO RELATED APPLICATIONS

This application claims priority over German patent applications DE 10 2019 123 304.6, filed on Aug. 30, 2019; DE 10 2020 102 315.4, filed on Jan. 30, 2020; and DE 10 2020 112 853.3, filed on May 12, 2020. The contents of all of the above-mentioned German patent applications are hereby incorporated by reference herein in their entirety.

### BACKGROUND OF THE INVENTION

### Field of the Invention

The invention comprises a service robot for the automated performance of geriatric tests.

### Brief Description of the Related Art

The health system is currently suffering from a major shortage of skilled workers. As a result of this personnel shortage, there is less and less time available to treat patients. This lack of time not only results in dissatisfaction on the part of both patients and medical staff, but potentially also in the inadequate treatment of illnesses, which in turn not only causes suffering in the patients, but also reduces the value creation of an economy. These effects are accompanied by an increasing need to document the patient's condition as a recourse to defend against claims for damages that can be attributed to inadequate therapy from a medical point of view. In some cases, this documentation obligation can bring about a self-reinforcing effect.

The service robot described in this document addresses this issue by having the service robot independently perform geriatric tests that are currently carried out by medical staff though the use of multiple sensors. This service robot is also capable of accurately documenting the completed exercises, which enables the healthcare facility of the service robot to meet the documentation obligation and other compliance obligations in this respect without having to separately assign staff for the purpose. Another effect is that the use of the service robot standardizes the assessment of tests. This is because, at present, the assessment of a patient is subject to the experience of the assessing medical staff, whose experience differs from that of other medical staff. Therefore, where medical staff may make varying assessments for a single exercise, the use of the service robot results in a uniform assessment.

In addition to the field of geriatrics, in which the service robot is able, for example, to determine the Barthel Index, perform the “Timed Up and Go” test and/or the mini-mental state exam with varying characteristics, the service robot is also configured in one aspect in such a way that the service robot can alternatively and additionally address further tasks in a clinic. These include, for example, spectrometric examinations, which can be used to analyze various substances in or on the skin of a person. These analyses can be used, for example, to determine the Delirium Detection Score.

In one aspect, the service robot is also configured to perform delirium detection and/or delirium monitoring. In this scope, the service robot, in one aspect, can determine possible attentiveness disorders of the patient based on the recognition of a sequence of acoustic signals. In an alternative and/or additional aspect, the service robot can assess cognitive abilities based on image recognition, and/or cognitive abilities via implementation in motor functions, e.g., by counting fingers pointed by the patient in response to a primarily visual prompt made by the service robot. Alternatively and/or additionally, the service robot is able to determine the pain status of a person. This can be done by way of emotion recognition, capturing the movements of the upper extremities, and/or the vocalization of pain by ventilated and/or non-ventilated patients. The service robot may, in one aspect, determine a patient's blood pressure, their respiratory rate, and also use this information, apart from original diagnostic and or therapeutic purposes, to control its own hardware and software components.

Independently of this, the service robot may be configured to detect manipulation attempts, for example, during data acquisition. Furthermore, the service robot may check whether users suffer from mental and/or physical impairments that may impact the quality of the tests to be performed or the results thereof. Moreover, in one aspect, the service robot can adapt its signal processing quality, as well as signal output, to environmental conditions. This includes adjustments to input and output, user dialogs, etc.

In addition, the use of the service robot provides considerable relief to medical staff, as this medical staff must perform this work that can be time-consuming and sometimes also monotonous and that has no direct impact on a patient's health, thereby preventing the staff from implementing measures that directly improve a person's health.

### STATE OF THE ART

Experts are familiar with various service robots in healthcare and geriatrics. CN108422427, for example, describes a rehabilitation robot capable of serving food on trays. In a similar vein, CN206833244 features a service robot that distributes materials in a hospital. Chinese patent applications CN107518989 and CN101862245 are also based on a hospital setting. These both refer to a service robot that transports patients in a way similar to a wheelchair. CN205950753 describes a robot that recognizes patients using sensors and guides them through a hospital. CN203338133 details a robot designed to assist nursing staff by helping patients in their daily tasks in a hospital. In contrast, CN203527474 refers to a robot that uses its arm to assist elderly people.

CN108073104 describes a care robot that provides care to infected patients by providing or administering medication to the patients, massaging them, feeding them, communicating with them, etc. Here, the care robot reduces the risk of infection for medical staff by reducing the number of patient contacts. A robot for accompanying elderly people can be found in CN107598943. This robot has some monitoring functions, but in particular a floor cleaning function.

CN106671105 is a mobile service robot for the care of elderly people. The service robot uses sensor technology to monitor physical parameters such as temperature, but also facial expressions. The service robot also detects whether the person has fallen and can alert help accordingly via a network.

Similarly, CN104889994 and CN204772554 feature a service robot from the medical field that detects patients' heart rate and supplies them with oxygen, and also includes speech recognition and a multimedia module for entertainment purposes. Blood oxygen detection is also included in the scope of CN105082149. CN105078445 refers to a service robot that allows the recording of an electrocardiogram and the measurement of blood oxygen content, particularly for elderly people. CN105078450 includes an electroencephalogram measurement and therefore follows a similar direction.

Some of the health robots explicitly relate to the performance of exercises with patients or also tests. In relatively abstract terms, CN108053889 describes a system that performs exercises with a patient based on stored information, while CN108039193 outlines a system for the automatic generation of health reports for use in a robot. The capture of movements/fitness exercises by means of a robot, the recording and storage of data of thereof for purposes of analysis, and the transmission of this data to external systems are described in CN107544266. At the same time, this robot is capable of monitoring the intake of medication by means of various sensors.

CN106709254 describes a robot employed for the medical diagnosis of a patient which can simultaneously use the diagnosis to generate a treatment plan. For this purpose, the robot evaluates speech and image information and compares it with information stored in its memory. A neural network is used for this purpose.

CN106407715 describes a service robot that uses speech processing and image recognition to record a patient's medical history. In addition to querying via speech input and output devices employing a touchpad, the robot also features a camera which takes a photo of the tongue for further documentation of the patient's medical history.

CN105078449 presents a service robot with a tablet computer as a communication device used, among other functions, for cognitive function training or cognitive/psychological test for the detection of Alzheimer's disease in patients. For this purpose, the tablet records a telephone conversation between the patient and a child that following a specific procedure, on the basis of which it deduces whether the patient is suffering from Alzheimer's disease.

One aspect of the service robot analyzes gestures of the hand for folding a sheet. Hand gesture recognition is established per se in the state of the art. Recognizing and tracking the fingers represents a particular challenge, however. For example, U.S. Ser. No. 10/268,277 describes a general system of hand gesture recognition, as does U.S. Pat. No. 9,372,546 or 9,189,068. U.S. Pat. No. 9,690,984, for example, describes a camera-based hand recognition system based on a skeleton model employed with the aid of machine learning algorithms. These approaches are primarily related to empty hands. In contrast, U.S. Pat. No. 9,423,879 is devoted to recognizing and tracking objects in hands and proposes the use of a thermal sensor to differentiate the hands and fingers (through the heat discharged) from other objects (which tend to be cooler).

Only two documents of the prior art were identified that relate to the recognition of sheets or sheet-like objects in the hands of users. For example, U.S. Pat. No. 9,117,274 describes how a depth camera is used to detect a paper document that a user is holding in his or her hand, while in a next step this sheet of paper, which exemplifies a flat surface, is used as a surface for projecting an image with which the user can interact. The sheet is identified by means of its corners, which are compared with quadrilaterals stored in a memory, which have been spatially rotated in space. In contrast, U.S. Ser. No. 10/242,527 describes how gaming tables (in a casino) are monitored by automatically recognizing hand gestures, including playing chips or even playing cards that bear some resemblance to a sheet. However, there is no description of how this recognition is achieved, but instead primarily for what purpose such evaluations are done. In addition, playing cards have rounded corners, which is generally not the case with a sheet.

With regard to the evaluation of the cognitive state of a person, approaches are also described in the prior art, which in turn have an influence on the control of a robot. For example, US20170011258 describes how a robot is manipulated based on a person's emotional state, where this state is evaluated primarily by the person's facial expression, which is captured using a histogram-of-gradients analysis. The emotional state of a person can generally be assessed by means of classification methods based on clustering or with the aid of neural networks. For example, US2019012599 describes quite generally how to use a multilayer convolutional neural network to generate weights based on video recordings of a face, which exhibits at least one convolutional layer and at least one hidden layer, the last layer of which describes emotions of a person, which furthermore determines weights for input variables of at least one layer, calculates the weights in at least one feed-forward process, and updates them in the scope of back-propagation.

With respect to the detection of the mental state of a person, various works can be found in the prior art. For example, U.S. Pat. No. 9,619,613 uses a special device that employs vibrations, among other things, to evaluate the mental state of a person. U.S. Pat. No. 9,659,150, for example, uses acceleration sensors to perform the Timed Up and Go test. In U.S. Pat. No. 9,307,940, stimuli are triggered to test mental abilities by outputting a sequence of stimuli of defined length and capturing the patient's response. U.S. Pat. No. 8,475,171, for example, uses virtual reality to show a patient various images and to diagnose Alzheimer's disease, for example, through the patient's recognition of these images. U.S. Ser. No. 10/111,593, for example, uses movement analysis to detect delirium. In contrast, CN103956171 tries to draw conclusions about a test score of the mini-mental state exam on the basis of a patient's pronunciation.

The service robot is configured in such a way that the service robot can collect other medical parameters by means of its sensors, including blood pressure by contactless means, for example by means of a camera. The state of the art for determining blood pressure via camera-based evaluation is for the most part in the research stage. Zaunseder et al. (2018) provides an overview primarily of methods that perform a color evaluation of blood flow. The review article by Rouast et al. (2018) goes somewhat further. Specifically, Karylyak et al. (2013) or Wang et al. (2014) deals with evaluation algorithms for the determination of blood pressure based on available signal data, for example, while McDuff et al. (2014) is dedicated to the determination of the times of systolic and diastolic pressure, for example, while Bai et al. (2018) evaluates the effectiveness of a new signal filter, for example. General approaches for determining blood pressure from recorded measured values can be found, for example, in Parati et al. (1995). Liu et al. (2018) deals with specific implementations of color-based evaluation and also compares different subregions of the face, similarly to e.g. Verkruysse et al. (2008), while Lee et al. (2019) describes a specific implementation based on movements of the face. Unakafov (2018), on the other hand, compares different methods based on a freely available data set. A step towards practical application is taken, for example, by the approach of Pasquadibisceglie et al. (2018), which integrates a color-based evaluation method into a mirror. In contrast, Luo et al. (2019) uses a smartphone to record the color data. A more concrete step towards implementation is taken by the approach of Wei et al. (2018) which uses the recording of color data and already exhibits the character of a clinical study. In contrast, the approach of Ghijssen et al. (2018) takes a different direction. Here, light is transmitted through a finger by means of a laser and detected on the opposite side by a sensor, whereby the emitted light exhibits speckle patterns, which, on the one hand, allow the detection of the rhythmic vascular blood flow as well as, as in the previously described approaches, the recording of the rhythmic vascular expansion of the vessels.

### SOURCES

Zaunseder et al. Cardiovascular assessment by imaging photoplethysmography—a review. Biomed. Eng.-Biomed. Tech. 2018; 63(5): 617-634, DOI: 0.1515/bmt-2017-01.
Kurylyak et al. Blood Pressure Estimation from a PPG Signal, 2013 IEEE International Instrumentation and Measurement Technology Conference (I2MTC). DOI: 10.1109/I2MTC.2013.6555424.
McDuff et al. Remote Detection of Photoplethysmographic Systolic and Diastolic Peaks Using a Digital Camera. IEEE TRANSACTIONS ON BIOMEDICAL ENGINEERING, VOL. 61, NO. 12, DECEMBER 2014, DOI: 10.1109/TBME.2014.2340991.
Bai et al. Real-Time Robust Noncontact Heart Rate Monitoring With a Camera, IEEE Access VOLUME 6, 2018. DOI: 10.1109/ACCESS.2018.2837086.
Pasquadibisceglie et al. A personal healthcare system for contact-less estimation of cardiovascular parameters. 2018 AEIT International Annual Conference. DOI: 10.23919/AEIT.2018.8577458.
Wang et al. Cuff-Free Blood Pressure Estimation Using Pulse Transit Time and Heart Rate. 2014 12th International Conference on Signal Processing (ICSP). DOI: 10.1109/ICOSP.2014.7014980.
Luo et al. Smartphone-Based Blood Pressure Measurement Using Transdermal Optical Imaging Technology. Circular Cardiovascular Imaging. 2019; 12:e008857. DOI: 10.1161/CIRCIMAGING.119.008857.
Wei et al. Transdermal Optical Imaging Reveal Basal Stress via Heart Rate Variability Analysis: A Novel Methodology Comparable to Electrocardiography. Frontiers in Psychology 9:98. DOI: 10.3389/fpsyg.2018.00098.
Parati et al. Spectral Analysis of Blood Pressure and Heart Rate Variability in Evaluating Cardiovascular Regulation. Hypertension. 1995; 25:1276-1286. DOI: 10.1161/01.HYP.25.6.1276.
Rouast et al. Remote heart rate measurement using low-cost RGB face video: a technical literature review. Front. Comput. Sci., 2018, 12(5): 858-872. DOI: 10.1007/s11704-016-6243-6.
Lee et al. Vision-Based Measurement of Heart Rate from Ballistocardiographic Head Movements Using Unsupervised Clustering. Sensors 2019, 19, 3263. DOI: 10.3390/s19153263.
Liu et al., Transdermal optical imaging revealed different spatiotemporal patterns of facial cardiovascular activities. Scientific Reports, (2018) 8:10588. DOI: 10.1038/s41598-018-28804-0.
Unakafov. Pulse rate estimation using imaging photoplethysmography: generic framework and comparison of methods on a publicly available dataset. Biomed. Phys. Eng. Express 4 (2018) 045001. DOI: 10.1088/2057-1976/aabd09.
Verkruysse et al. Remote plethysmographic imaging using ambient light. 22 Dec. 2008/Vol. 16, No. 26/OPTICS EXPRESS 21434. DOI: 10.1364/OE.16.021434.
Ghijssen et al. Biomedical Optics Express Vol. 9, Issue 8, pp. 3937-3952 (2018). DOI: 10.1364/BOE.9.003937.
Yamada et al. 2001 (DOI: 10.1109/6979.911083)
Roser and Mossmann (DOI: 10.1109/IVS.2008.4621205)
US20150363651A1
McGunnicle 2010 (DOI: 10.1364/JOSAA.27.001137)
Espy et al. (2010) (DOI: 10.1016/j.gaitpost.2010.06.013)
Senden et al. (DOI: 10.1016/j.gaitpost.2012.03.015)
Van Schooten et al. (2015) (DOI: 10.1093/gerona/glu225)
Kasser et al. (2011) (DOI: 10.1016/j.apmr.2011.06.004)

In addition, the service robot can detect substances on or within the skin, in part by skin contact and in part contactlessly. Spectrometric approaches are primarily applied here. Approaches using spectrometers or similar technology are described for example, in U.S. Pat. Nos. 6,172,743, 6,008,889, 6,088,605, 5,372,135, US20190216322, US2017146455, U.S. Pat. Nos. 5,533,509, 5,460,177, 6,069,689, 6,240,306, 5,222,495, and 8,552,359.

### SUMMARY OF THE INVENTION

System and method for capturing a movement sequence of a person. The method comprises capturing a plurality of images of the person executing a movement sequence by means of a contactless sensor, the plurality of images representing the movements of the body elements of the person, generating at least one skeleton model having limb positions for at least some of the plurality of images, and calculating the movement pattern from the movements of the body elements of the person by comparing changes in the limb positions in the at least one skeleton model generated. In addition, vital signs and/or signal processing parameters of the person can be acquired and evaluated.

### BRIEF DESCRIPTION OF THE FIGURES

The figures show the following:

FIG. 1 is a schematic diagram of a structure of a service robot in accordance with an embodiment of the present invention.

FIG. 2 is a top view of the wheels of the service robot in accordance with an embodiment of the present invention.

FIG. 3 is a diagram of a management system for the service robot in accordance with an embodiment of the present invention.

FIG. 4 is a flow diagram of a method for recognition of a chair using 2D LIDAR in accordance with an embodiment of the present invention.

FIG. 5 is a flow diagram of a method for recognition of a person on a chair using 2D LIDAR in accordance with an embodiment of the present invention.

FIG. 6 is a flow diagram of a method for persuading a person to sit down in accordance with an embodiment of the present invention.

FIG. 7 is a flow diagram of a method for navigation of a person to a chair that meets a certain criterion in accordance with an embodiment of the present invention.

FIG. 8 is a flow diagram of a method for recognition of doors in particular by means of LIDAR in accordance with an embodiment of the present invention.

FIG. 9 is a flow diagram of a method for recognition of a fixed marker in front of an object in accordance with an embodiment of the present invention

FIG. 10 is a flow diagram of a method for labeling of movement data from the Get Up and Go test in accordance with an embodiment of the present invention.

FIG. 11 is a flow diagram of a method for detection of repeated speech sequences in accordance with an embodiment of the present invention.

FIGS. 12A and 12B are a flow chart of a method for recording and evaluation of the folding of a sheet in accordance with an embodiment of the present invention.

FIG. 13 is a flow diagram of a method for evaluation of a written sentence by the service robot in accordance with an embodiment of the present invention.

FIG. 14 is a flow diagram of a method for detection of possible manipulation of the service robot by third parties in accordance with an embodiment of the present invention.

FIG. 15 is a flow diagram of a method for manipulation vs. assistance by third parties in accordance with an embodiment of the present invention.

FIGS. 16A and 16B are flow charts of a method for calibration of the service robot taking user interference into account in accordance with an embodiment of the present invention.

FIG. 17 is a flow diagram of a method for a service robot moving in the direction of the patient in accordance with an embodiment of the present invention.

FIG. 18 is a flow diagram of a method for passing a door in accordance with an embodiment of the present invention.

FIG. 19 is a flow diagram of tests to determine the risk of dementia of surgical patients and postoperative monitoring by a service robot in accordance with an embodiment of the present invention.

FIG. 20 is a flow diagram of data from the service robot processed for therapy suggestions in accordance with an embodiment of the present invention.

FIGS. 21A and 21B are flow diagrams of a determination of measurement regions on the patient in accordance with an embodiment of the present invention.

FIG. 21C is a flow diagram of measurement and evaluation of the spectrometric examination in accordance with an embodiment of the present invention.

FIG. 22 Output and evaluation of patient responses to a tone sequence

FIG. 23 Evaluation of image recognition of a patient for diagnostic purposes in accordance with an embodiment of the present invention.

FIG. 24 Ensuring sufficient visibility of the service robot display in accordance with an embodiment of the present invention.

FIG. 25 Pose recognition of the hand with view of displayed numbers in accordance with an embodiment of the present invention.

FIG. 26 Display of two fingers and detection of patient response in accordance with an embodiment of the present invention.

FIG. 27 Evaluation of emotions by service robot in accordance with an embodiment of the present invention.

FIG. 28 Evaluation of the activity of the upper extremities of a patient in accordance with an embodiment of the present invention.

FIGS. 29A, 29B and 29C are flow charts of a method for recording of coughing of a patient in accordance with an embodiment of the present invention.

FIG. 30 Blood pressure determination in accordance with an embodiment of the present invention.

FIG. 31 Self-learning moisture recognition on surfaces in accordance with an embodiment of the present invention.

FIG. 32 Navigation during moisture detection on surfaces in accordance with an embodiment of the present invention.

FIG. 33 Evaluation of fall events in accordance with an embodiment of the present invention.

FIG. 34 Monitoring of vital signs during an exercise/test in accordance with an embodiment of the present invention.

FIG. 35 Evaluation of a person's gait sequence with regard to their risk of falling in accordance with an embodiment of the present invention.

FIG. 36 Sequence of a mobility test in accordance with an embodiment of the present invention.

FIG. 37 Determination of sitting balance in accordance with an embodiment of the present invention.

FIG. 38 Determination of standing up in accordance with an embodiment of the present invention.

FIG. 39 Determination of stand-up attempt in accordance with an embodiment of the present invention.

FIG. 40 Determination of standing balance in accordance with an embodiment of the present invention.

FIG. 41 is a flow diagram of a method for determination of standing balance and distance between feet in accordance with an embodiment of the present invention.

FIG. 42 Determination of standing balance/impact in accordance with an embodiment of the present invention.

FIG. 43 Classification of gait initiation in accordance with an embodiment of the present invention.

FIG. 44 Determination of step position in accordance with an embodiment of the present invention.

FIG. 45 Determination of step height in accordance with an embodiment of the present invention.

FIG. 46 Determination of gait symmetry in accordance with an embodiment of the present invention.

FIG. 47 Determination of step continuity in accordance with an embodiment of the present invention.

FIGS. 48A, 48B and 48C are flow diagrams of a method for determination of path deviation accordance with an embodiment of the present invention.

FIG. 49 Determination of trunk stability in accordance with an embodiment of the present invention.

FIG. 50 Determination of track width in accordance with an embodiment of the present invention.

FIG. 51 Determination of turning in accordance with an embodiment of the present invention.

FIG. 52 Determination of sitting down in accordance with an embodiment of the present invention.

FIG. 53 Improvement of the signal-to-noise ratio for skeleton model evaluation in accordance with an embodiment of the present invention.

FIG. 54 Adjustment of the image section during detection of sensor movements in accordance with an embodiment of the present invention.

FIG. 55 Navigation for lateral recognition of a person in accordance with an embodiment of the present invention.

FIG. 56 Determination of training plan configuration in accordance with an embodiment of the present invention.

FIG. 57 Architectural view in accordance with an embodiment of the present invention.

FIG. 58 Manipulation detection based on audio signals in accordance with an embodiment of the present invention.

FIG. 59 System for score determination for rising from/sitting down on a chair in accordance with an embodiment of the present invention.

FIG. 60 System for synchronizing movements between a person and a service robot in accordance with an embodiment of the present invention.

FIG. 61 System for the recording and evaluation of a folding exercise in accordance with an embodiment of the present invention.

FIG. 62 System for manipulation detection in accordance with an embodiment of the present invention.

FIG. 63 Spectrometry system in accordance with an embodiment of the present invention.

FIG. 64 Attention analysis system in accordance with an embodiment of the present invention.

FIG. 65 Cognitive analysis system in accordance with an embodiment of the present invention.

FIG. 66 System for determining pain status in accordance with an embodiment of the present invention.

FIG. 67 System for determining blood pressure in accordance with an embodiment of the present invention.

FIG. 68 System for measuring substances in accordance with an embodiment of the present invention.

FIG. 69 System for moisture assessment in accordance with an embodiment of the present invention.

FIG. 70 System for fall detection in accordance with an embodiment of the present invention.

FIG. 71 System for recording vital signs in accordance with an embodiment of the present invention.

FIG. 72 System for determining a fall risk score in accordance with an embodiment of the present invention.

FIG. 73 System for determining the balance of a person in accordance with an embodiment of the present invention.

FIG. 74 System for determining the position of a foot in accordance with an embodiment of the present invention.

FIG. 75 System for classifying a turning movement in accordance with an embodiment of the present invention.

FIG. 76 System for gait classification in accordance with an embodiment of the present invention.

FIG. 77 System for modifying optical signals of a sensor in accordance with an embodiment of the present invention.

FIG. 78 System for adjusting an image section in accordance with an embodiment of the present invention.

FIG. 79 System for capturing lateral images in accordance with an embodiment of the present invention.

FIG. 80 Iterative classifier generation for a large number of skeleton points in accordance with an embodiment of the present invention.

FIG. 81 Sequence for the evaluation of moisture on surfaces in accordance with an embodiment of the present invention.

FIG. 82 Path planning for moisture detection on the floor in accordance with an embodiment of the present invention.

FIGS. 83A and 83 are flow diagrams of a method for the determination of foot position in accordance with an embodiment of the present invention.

FIG. 84 Method for the determination of turning movements in accordance with an embodiment of the present invention.

FIG. 85 Method for recording the movement pattern of a person along a line in accordance with an embodiment of the present invention.

### DETAILED DESCRIPTION OF THE INVENTION

The term “user” is understood to mean a person who uses the service robot 17, in this case primarily evaluated sensorially by the service robot 17 through the described apparatus. User may be elderly people with whom the service robot 17 performs a geriatric test, but also relatives or third parties who, for example, assist elderly people in their interaction with the service robot 17, or who perform the test for elderly people.

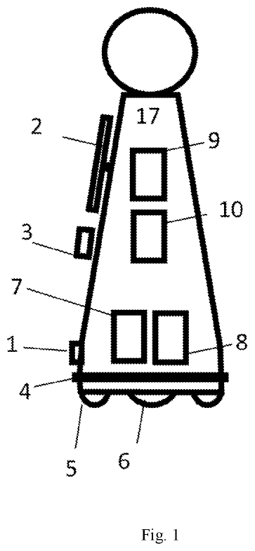

FIG. 1 illustrates the mobile service robot 17. The service robot 17 has a laser scanner (LIDAR) 1 for scanning the environment of the service robot 17. Other sensors are also possible here alternatively and/or additionally, for example a camera (2D and/or 3D) 185 and an ultrasonic and/or radar sensor 194.

The service robot 17 has at least one display 2, which in one aspect is a touchpad. In the aspect illustrated in FIG. 1, the service robot 17 has two of these touchpads. The touchpads in turn have, for example, a microphone 193 and a loudspeaker 192 that allow acoustic communication with the service robot 17. Furthermore, the service robot 17 has at least one sensor 3 for the contactless three-dimensional recording of the movement data of a patient. In a non-limiting example, the sensor is a Microsoft Kinect device. Alternatively, an Orbbec Astra 3D camera may be used. Such 3D cameras feature a stereo camera system for depth detection, which allows the evaluation of a skeleton model of a patient, and in most cases also have an RGB camera for color detection. In an alternative aspect, a conventional mono camera can be used. Technologies that can be used in 3D cameras in this regard are Time-of-Flight (ToF) sensors or speckle sensors.

At a distance of, for example, 5 cm above the ground, a pressure-sensitive bumper 4 is located around the outer shell of the service robot 17, at least in the areas that lie in a possible direction of travel of the service robot 17. The processing unit 9 is connected to the pressure-sensitive bumper 4 and recognizes collisions of the service robot 17 with an object. In the event of a collision, the drive unit 7 is stopped immediately.

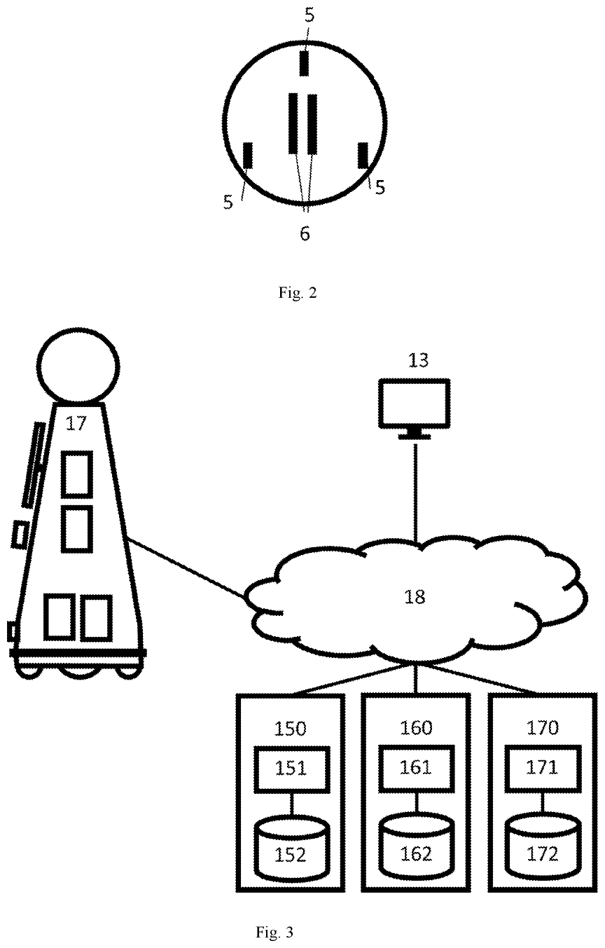

In one aspect, the service robot 17 has two drive wheels 6 that are centered and arranged parallel to each other (see FIG. 2). Around them, for example on a circular path, are two or three more support wheels 5. This arrangement of the support wheels 5 allows the service robot 17 to rotate on the spot by driving the drive wheels 6 in opposite directions. For this purpose, the axis of the two or three support wheels 5 is mounted in such a way that the axis can rotate 360 degrees about the vertical axis. When using two support wheels 5, the distance between the drive wheels is greater than shown in FIG. 2, which prevents the service robot 17 from tipping too easily.

The service robot 17 also has an energy source 8 to supply energy to the drive and processing unit 9, the sensors (laser scanner 1, sensor 3, and bumper 4), and the input and output units 2. The energy source 8 is a battery or a rechargeable battery. Alternative energy sources, such as a fuel cell, which includes a direct methanol or solid oxide fuel cell, are also conceivable.

The processing unit 9 has at least one memory 10 and at least one interface 188 (such as WLAN) for data exchange. In an optional aspect, these include (not shown) a device for reading a mobile memory (for example, a transponder/RFID token). In another aspect, this mobile memory is also writable. In one aspect, this or another interface 188 (such as WLAN) allows wireless communication with a network. The service robot 17 has rules described later in this document for performing evaluations stored in the memory 10. Alternatively and/or additionally, these rules may also be stored in the memory of a cloud 18 accessed by the service robot 17 via the at least one interface 188 (such as WLAN). This must not be mentioned thus explicitly elsewhere but will be included upon disclosure.

The sensor 3 recognizes a person and the person's actions and creates a skeleton model based on the person's movements. In one aspect, the sensor 3 is also capable of recognizing walking/forearm crutches (UAGS). Furthermore, the service robot 17 optionally has one or more microphones 193, which may be implemented independently of the touch pads in order to record the person's speech and evaluate it in a processing unit.

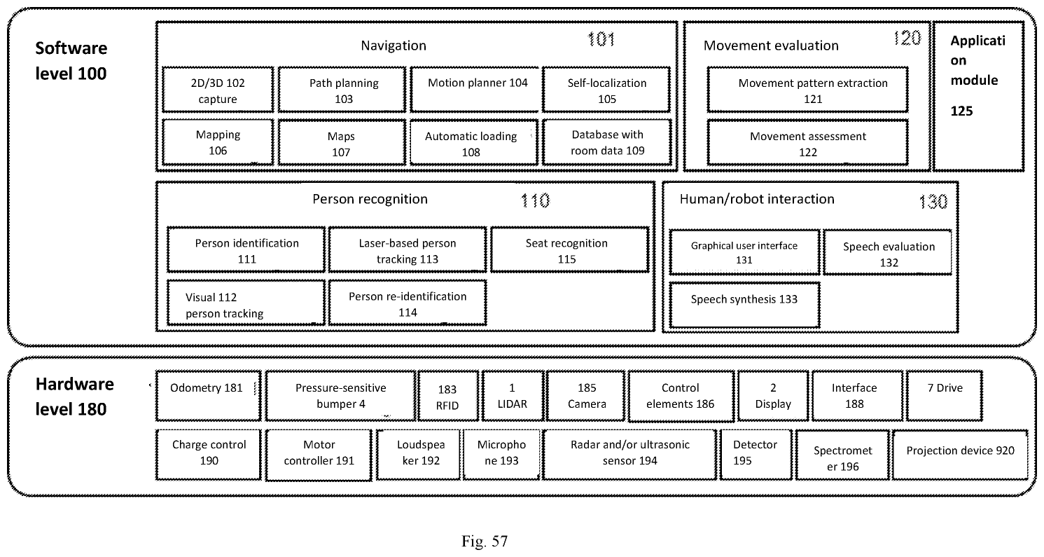

FIG. 57 illustrates the architectural view, which however hides the applications described in this document. On the software level, there are various modules with basic functions of the service robot 17. For example, various modules are included in the navigation module 101. Among them is a 2D or 3D environment detection module 102, which, for example, evaluates environment information based on various sensor data. The path planning module 103 allows the service robot 17 to determine its own path that it travels. The movement planner 104 uses, for example, the path planning results from the path planning module 103 and calculates an optimal path for the service robot while accounting for or optimizing various cost functions. In addition to the data from path planning, the cost functions include data from obstacle avoidance, a preferred direction of travel, etc., which may also be an expected direction of movement of a monitored person, for example. Aspects of movement dynamics also play a role, such as speed adaptation, for example, in bends, etc. The self-localization module 105 allows the service robot 17 to determine its own position on a map, for example by means of odometry data, the comparison of acquired environment parameters from 2D/3D environment detection with environment parameters stored in a map from the map module 107, etc. The mapping module 106 allows the service robot 17 to map its environment. Created maps are stored, for example, in the map module 107, which may, however, also contain maps other than just self-created maps. The loading module 108 is for automatic loading. In addition, there may be a database of room data 109 that includes, for example, information on which room to perform an evaluation with a person, etc. A movement evaluation module 120 includes, for example, a movement extraction module 121 and a movement assessment module 122. Each of these includes movement evaluation rules, which are described in more detail later in this document. The person recognition module 110 includes, for example, a person identification module 111, which includes, for example, rules for determining from acquired sensor data whether the person is a person or something else. A visual person tracking module 112 for visual person tracking is based, for example, primarily on camera data as input variables, and the laser-based person tracking module 113 uses LIDAR 1 accordingly. A person reidentification module 114 makes it possible, for example, in the event of an interruption of the tracking process, to classify a person detected thereafter as a person who was previously tracked or not. A seat recognition module 115 makes it possible, for example, to detect a chair. The service robot 17 further has a human/robot interaction module 130 comprising a graphical user interface 131, a speech synthesis unit 133, and a speech evaluation module 132. In addition, there is an application module 125, which can include a variety of applications, such as exercises and tests with persons, which will be described in more detail subsequently.

On the hardware level 180, there is an odometry unit 181, for example an interface for communication with RFID transponders, a camera 185, control elements 186, an interface 188 such as WLAN, a charge control 190 for the energy supply, a motor controller 191, loudspeakers 192, at least one microphone 193, for example a radar sensor and/or an ultrasonic sensor 194, a detector 195, which is described in more detail elsewhere, and also, for example, a spectrometer 196 and, for example, a projection device 920. The LIDAR 1, display 2, and drive 7 were already described above.

FIG. 3 illustrates that the service robot 17 is connected to the cloud 18 via an interface 188. A therapist has the ability to access a patient administration module 160 stored in the cloud 18 via a terminal 13 with a processing unit 161, which in turn is connected to a memory 162.

Medical staff can store patient data in the patient administration module 160 or, in one aspect, import such patient data from other systems via an interface 188 (such as WLAN). Such other systems primarily include hospital information systems (HIS) and/or patient data management system as commonly used in hospitals or medical practices. Patient data includes the patient's name and, if applicable, room number, as well as information on the patient's general health, etc. Here, the processing unit 161 in the patient administration module 160 generates an ID for each person, which is stored with the personal data in the memory 162. The medical staff can define the tests to be performed. The management system is connected to a rule set 150 via the cloud 18 comprising a processing unit 151 and a memory 152. The rule set 150 contains rules for performing and evaluating the exercises, which may match those of the service robot 17 and are, for example, maintained centrally in the rule set and then distributed to multiple service robots 178.

The rule set 150 is used to store the classification of objects and movements, but also the combination thereof in order to evaluate the observations made for the purpose of the test. For example, the positions of the legs, upper body, arms, hands, etc. are stored on the basis of a skeleton model. In addition, objects to be evaluated as part of the test may be recognized. The rule set 150 may be initially created on the basis of a template with the assistance of experts, i.e., limit values for individual limbs may be defined. Fuzzy algorithms may also be used for the limit values. Alternatively, individual images or image sequences that can again be translated into a skeleton model, for example, with regard to images of a person, can be labeled by medical staff and machine learning algorithms including neural networks are used to determine classifications that map the threshold values.

In one aspect, a cloud-based navigation module 170 including a navigation processing unit 171 and a navigation memory 172 is also available.

The service robot 17 may be connected to a cloud application in the cloud 18. The therapist can assign a mobile memory unit, such as a token, to the person who is to perform the test. The token contains the patient ID and/or another token ID assigned to the person/his or her ID. The person can use this token and/or the serial number and/or the ID to identify him- or herself to the service robot 17. Identification is also possible by other means, for example by entering login data in a screen-guided menu, but also by means of biometric features such as, for example, a facial scan or software on a mobile device that makes a code available to be entered or read into the service robot 17. The service robot 17 now downloads the test stored by the medical staff from the cloud 18, but without the personal data, via an interface 188 (such as WLAN)—the assignment is made via the personal ID. After completing the test, the service robot 17 loads the test data in encrypted form into the patient administration module 160—the assignment is made via the personal ID. The data is only decrypted in the patient administration module 160 (see below). The medical staff can subsequently evaluate the data, as explained in more detail through relevant examples provided below.

In another aspect, the medical staff transfers the instructions for performing a test or a subcomponent thereof to a storage medium (e.g., a transponder in the form of an RFID tag), which the person receives in order to identify him- or herself to the service robot 17, for which purpose the service robot has an RFID interface 183. In the process, the data from the storage medium is transmitted to the service robot 17, including the personal ID that was specified by the patient administration module 160. After completing the test, the service robot 17 transfers the data back to the storage medium so that medical staff can transfer the data to the patient administration module 160 when reading the storage medium. In an additional and/or alternative aspect, the data may also be transmitted to the patient administration module 160 in encrypted form via a wireless or wired interface 188 (such as WLAN).

Combinations of the approach described above and data exchange via storage medium (e.g., transponder) are also possible.

The service robot has sensors in the form of the camera 185, a LIDAR 1, a radar sensor, and/or an ultrasonic sensor 194 that can be used not only for navigation purposes but also, for example, for person detection and tracking, which is why these sensors, together with corresponding software modules, form a person detection and tracking unit 4605 on the hardware side, whereby further sensors can also be used here, for example in interaction with an inertial sensor 5620, which is located on the person to be detected and/or tracked. With regard to person detection and person tracking, a person recognition module 110 can be used in a first step, which recognizes a person based on sensor data and can have various submodules. This includes, for example, a person identification module 111 that allows a person to be identified. In addition, characteristic features of the person can be stored, for example. The person reidentification module 114 makes it possible to recognize the person again, for example after an interruption of person tracking, which can be performed by a visual person tracking module 112 (evaluating data from a camera 185, for example) or a laser-based person tracking module 113 (evaluating data from a LIDAR 1, for example). The person may be recognized in the person reidentification module 114 by means of pattern matching, the patterns resulting, for example, from the stored personal features. A movement evaluation module 120 allows the evaluation of various movements. Captured movements can first be pre-processed in the movement extraction module 121, i.e., features of the movements are extracted, which are classified and assessed in the movement assessment module 122, for example, to identify a specific movement. In this regard, with respect to the detection and evaluation of movements of a person, a skeleton model can be created in the skeleton creation module 5635, which determines skeleton points at the joints of the person and direction vectors between the skeleton points. Feature extraction based on skeleton points takes place, for example, in the skeleton model-based feature extraction module 5460. A number of specific feature extraction modules are listed in this document, as well as several feature classification modules that can in turn be based on these feature extraction modules, for example. In one aspect, these include a gait feature extraction module 5605, which also uses data from the skeleton creation module 5635, a gait feature classification module 5610, and a gait process classification module 5615.

With respect to the terminology used, some clarifications are necessary: In one aspect, for example, hand skeleton points are mentioned, which can be used as a proxy for the position of a hand, e.g., when evaluating a person's grip on objects. Depending on the evaluation, this can also include finger skeleton points, insofar as fingers can be evaluated over the detection distance. In the following, we will refer to persons and users, for example. A person can be understood relatively broadly, while a user is generally a person who has identified him- or herself to the service robot 17. However, the terms can be used synonymously in many instances, though the differentiation is particularly relevant when it comes to manipulation detection.

With regard to threshold value comparisons, this document refers at times to exceeding a threshold value, which then results in a certain evaluation of a situation. Moreover, different calculations can be used, which could in part lead to a contrary interpretation of the evaluation results. An example of this is the comparison of two patterns used for person recognition. If, for example, a similarity coefficient is calculated for this purpose, e.g., a correlation, a high correlation that lies above a certain threshold value means that, for example, the two persons are identical. However, if there is a difference between the individual values, a high difference value means the opposite, i.e., a high dissimilarity. However, such alternative calculations are considered synonymous with, for example, the first calculation via the correlation.

The use of machine learning methods may, for example, eliminate the need for defining explicit threshold values, e.g., for movement patterns, in favor of pattern evaluation. That is, instead of threshold value comparisons, e.g., for dedicated distances of a skeleton point, pattern matching is carried out that evaluates multiple skeleton points simultaneously. To the extent that the following refers to a threshold comparison, especially with regard to a movement pattern, a method for a pattern matching can also be devised in case machine learning algorithms are used. As a basis for such a pattern matching, body poses of a movement pattern, for example, whether correct or incorrect, can be recorded over time and evaluated coherently. On the basis of extracted features, such as skeleton points, it is possible to create a classifier that carries out matching on the basis of other recorded body poses that have been specified as correct or incorrect and the courses of the skeleton points derived from them.

### Determination of the Barthel Index

One of the tests that the service robot 17 can perform is to determine Barthel index/carry out the Barthel test. This Barthel test is used to assess basic abilities with respect to independence or the need for care, such as eating and drinking, personal hygiene, mobility and, stool/urine control, on the basis of a behavioral observation. For this purpose, the service robot 17 is configured in such a way that a user is asked questions on these topics by means of the communication devices. The user may be the person to be assessed. Alternatively and/or additionally, further persons, e.g., relatives, can also be asked questions on these topics by means of the communication device. In this case, the questions are asked either via menu navigation of a display 2 of the service robot 17 or via a speech interface. Alternatively or in addition to the display 2 and/or microphones 193 built into the service robot 17, a separate display 2 connected to the service robot 17 via an interface 188 (such as WLAN), e.g. a tablet computer, can also be used, which the person can take in hand or place on a table, making it easier to answer and complete the exercises. The question dialog is used to differentiate between the person to be assessed and, for example, relatives. Additional and/or alternative differentiations are also possible according to the approaches that are described in more detail below, e.g., in the section on manipulation detection.

Recognition of a Chair with a Patient Who is to Perform a Timed Up and go Test

One of the tests that can be performed using the service robot 17 is the “Timed Up and Go” test. In this test, a person being evaluated sits in an armchair, stands up, walks three meters, then turns around and sits back down. The time used for this is recorded and, on the basis of a table, converted into a score.

The service robot 17 uses a laser scanner 1 to scan the room in which the service robot 17 is located, calculates the distances to the walls, and creates a virtual map in the scope of the mapping process carried out by the mapping module 106. This map reproduces the outlines of the room, but also notes objects located between the laser scanner 1 and the walls, likewise in the XY plane. The map thereby created is stored in the map module 107. If the laser scanner 1 does not have a 360-degree view, the service robot 17 performs travel movements allowing the service robot 17 to scan its surroundings approximately 360°. The service robot 17 performs this scanning, for example, from different positions in the room, in order to recognize obstacles standing in isolation, for example. Once the service robot 17 has scanned the room and created a virtual map, the service robot 17 is able to recognize the room by scanning a part of the room again. This further scanning succeeds with greater precision the more the room is scanned. In the process, the service robot 17 records, for example, the distance it has covered and measures the distances in such a way that the service robot 17 can determine its position in the room. In addition, the distance covered can also be measured by evaluating the rotational movement of the wheels in connection with their circumference. If a camera 185 is used to create the maps instead of a laser scanner, it is easier to determine the position, since characteristic dimensions are recognized not only in the XY plane, but also in the Z plane, making it possible to identify unique dimensions within the room more quickly this identification can be carried out using two-dimensional representation only.

More than one sensor can also be used in the room mapping by the mapping module 106, for example the combination of the LIDAR 1 and the sensor 3, where the sensor 3 is an RGB camera that, for example, detects the coloring in the room and assigns a color value to each point in the XY plane that the LIDAR 1 records. For this purpose, the processing unit of the service robot 17 performs image processing in such a way that first a Z coordinate is assigned to each point in the XY plane, with the Z coordinate resulting from the inclination of the LIDAR and its height relative to the ground. The RGB camera, in turn, has a known relative position to the LIDAR, as well as a known orientation angle and a known recording angle, making it possible to determine the distance in the image of, for example, a horizontal straight line that is 2 m away and 50 cm above the ground. These parameters can be used to assign each spatial coordinate determined by LIDAR 1 to a pixel in the RGB image and therefore also the color values of the pixel.

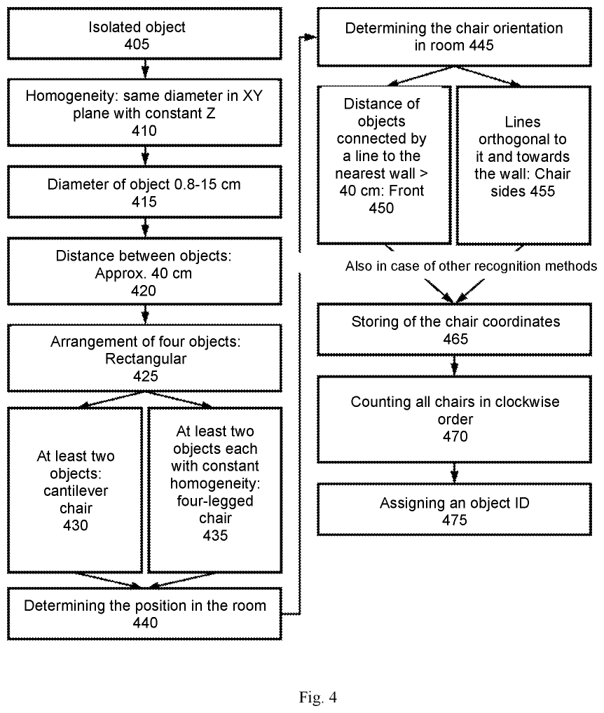

The LIDAR 1 makes it possible to determine the position in the room where a chair is presumably located. The recognition method is described in FIG. 4. Chairs typically have one to four legs, with single-legged chairs being office swivel chairs, which are less suitable for persons of advanced age with possible walking difficulties due to their potential rotation about the Z-axis. Much more likely are chairs with two or four legs, whereby the two-legged chairs are most likely cantilever chairs in most cases. A further characteristic of chair legs is that they stand in isolation in the XY plane, with the LIDAR 1 recognizing objects standing in isolation in step 405. Furthermore, chair legs primarily exhibit a homogeneous cross-section relative to each other in the XY-plane with a constant Z (step 410). The diameter of the objects (i.e., the potential chair legs) is within a range of 0.8 cm and 15 cm, e.g. between 1 cm and 4 cm, and is determined in step 415. The mutual distance between the objects that potentially turn out to be chair legs in the XY plane is typically approx. 40 cm 420. Moreover, in the case of four-legged chairs, the legs are primarily arranged in the form of a rectangle (step 425). This means that two objects with the same diameter indicate the presence of a cantilever chair with two legs (step 430). If the cross-sections of the front legs of the chair relative to each other and the rear legs of the chair relative to each other are identical, the chair is presumably a four-legged chair (step 435).

On the basis of these characteristics (two or four objects standing in isolation that have an approximately symmetrical cross-section, have a mutual distance of approx. 40 cm, and are approximately rectangular in arrangement, if applicable), the service robot 17 is now able to assign the attribute “chair” to such objects and, in the virtual map created by means of LIDAR 1 and/or one or more further sensor(s), to determine such positions of chairs in step 440 at which there is a high probability that one or more chairs are located. Each identified chair is also assigned a spatial orientation in step 445. In most cases, the chairs are located approximately parallel to a wall and typically exhibit a distance to this wall between 2 cm and 20 cm, with this distance applying to the back of the chair. Accordingly, the line between two of the legs of the chair that is situated parallel to the wall and is typically 40-70 cm away from the wall is assigned the property “front side chair” 450, the two areas which are orthogonal thereto are labeled as the “back sides” of the chair in step 455. Additionally and/or alternatively, in the case of four-legged chairs, the side further away from the nearest wall may also be recognized as the front side.

Instead of LIDAR 1, a 2D or 3D camera 185 may also be used to recognize the chairs. In this case, the processing unit sends the images via an interface 188 (such as WLAN) and an API, if necessary to a web service in the cloud 18 that is set up to perform image classifications, or the processing unit makes use of image classification algorithms stored in the memory 10 of the service robot 17 that are able to recognize a chair, including a chair with armrests, in the images created by the 2D or 3D camera 185. There are a variety of algorithms that perform such classifications initially and create a model that can then be applied either in the web service in the cloud 18 or in the memory 10 of the service robot 17 to the images created by the 2D or 3D camera 185 of the service robot 17, including neural networks such as convolutional neural networks.

Regardless of the method of chair identification, the service robot 17 is capable of storing the location of a chair, for example, in its own memory 10, which interacts with the navigation module 101 of the service robot 17 465. In addition, the service robot 17 detects the number of chairs in the room 470 and counts these chairs in a clockwise sequence. Alternatively, a different sequence is also possible. Based on this sequence, the chairs are assigned a number that is stored as an object ID 475.

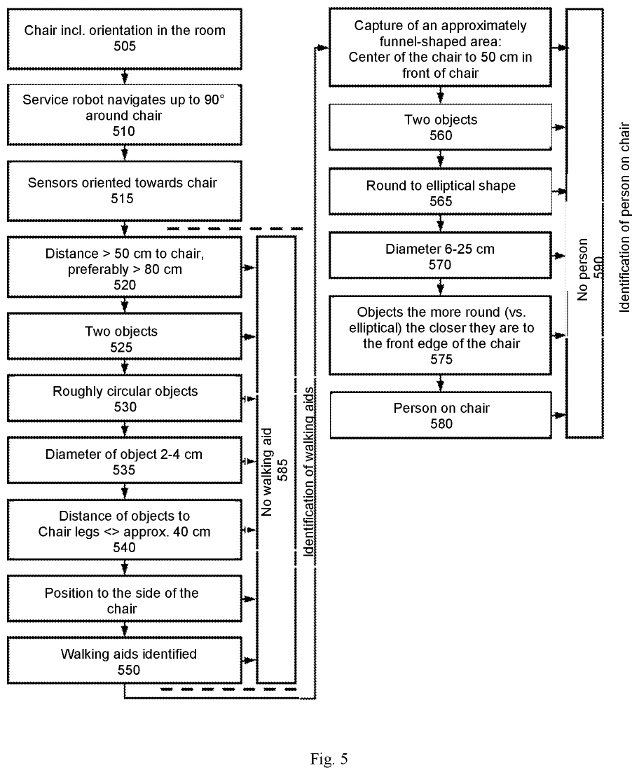

Based on the described procedure, the service robot 17 is capable of mapping a room including any exiting chairs, i.e., it can determine the position of the chairs in the room including their orientation. However, in order to perform the Timed Up and Go test, it is necessary for a person to be seated on one of the chairs, with the person possibly also having walking aids that are located in the vicinity of the chair. In the case where the service robot 17 is configured to use the LIDAR 1 of the service robot 17 to identify people, the following procedure is used: The position and orientation of the chair in the room is identified via the previous procedures illustrated in FIG. 5 in step 505. In order to identify legs and any walkers and to distinguish them from objects shaped in cross-section in the XY direction, the service robot 17 navigates in step 510 a minimum of approximately 20°, e.g. at least 45°, but ideally at least 90°, around the location where a chair is located 510, with the LIDAR 1 and/or one or more other sensors oriented in the direction of the chair (step 515). In the process, the service robot 17 maintains a distance of more than 50 cm, e.g., more than 80 cm 520. This increases a forecasting accuracy that the chair legs are recognized by the service robot 17, thus allowing the conclusion that a person is sitting on the chair.

Provided that a person is sitting on the chair, there are optionally two other objects near the chair 525 that are approximately circular 530 and have a diameter of less than 4 cm, for example less than 3 cm 535. In all likelihood, these objects have distances to each other and/or the chair legs that deviate significantly from the approx. 40 cm 540 that the chair legs have to each other. Moreover, with high probability, these distances are situated laterally relative to the chair legs 545. Taking this information into account, the processing unit 9 in the service robot 17 is capable of identifying the identified objects as walking aids 550. If more than one these features are not detected, no walking aids are identified 585. Naive Bayes estimations can be used for this purpose, for example. Since not every person needs to have walking aids to complete the test, steps 525-550 are optional and/or unnecessary for identifying the person on a chair by means of LIDAR 1. In spatial terms, the legs of the person sitting on the chair are presumably located within the perimeter of the front legs of the chair.

One or both legs may be positioned in front of, between, or behind the front legs of the chair. This yields a roughly funnel-shaped area extending forward in a roughly radial pattern from roughly the center of the chair that extends to a maximum of approx. 50 cm above the line connecting the two chair legs 555. The data collected by LIDAR 1 is evaluated in order to identify two 560 approximately round to elliptical objects 565 that are 6-30 cm in diameter, e.g., 7-20 cm 570 within this area. The legs can also be located between the front chair legs or even behind these chair legs. The closer the objects are to the line between the two front chair legs, the more the shape of the objects approximates a circular shape 575. Provided these criteria are largely met, the rule set 150 stored in the service robot 17 recognizes a person on the chair 580 based on the LIDAR data. Alternatively, no person is recognized on the chair 590.

Alternatively to the LIDAR 1, the person and walking aids can also be recognized via a general image classification, as described, for example, somewhat earlier in this document. In this respect, the service robot 17 likewise improves the forecasting accuracy if the service robot 17 targets the chair from multiple positions, allowing the 2D or 3D camera 185 to record the chair, as similarly described in the previous paragraph, for example. Instead of a general image classification, the methods (SDKs) adequately described in the prior art for person recognition via 2D or 3D cameras 185 that function based on skeleton recognition, for example the Kinect SDK, Astra Orbbec SDK, Open Pose, PoseNet by Tensorflow, etc. can also be used.

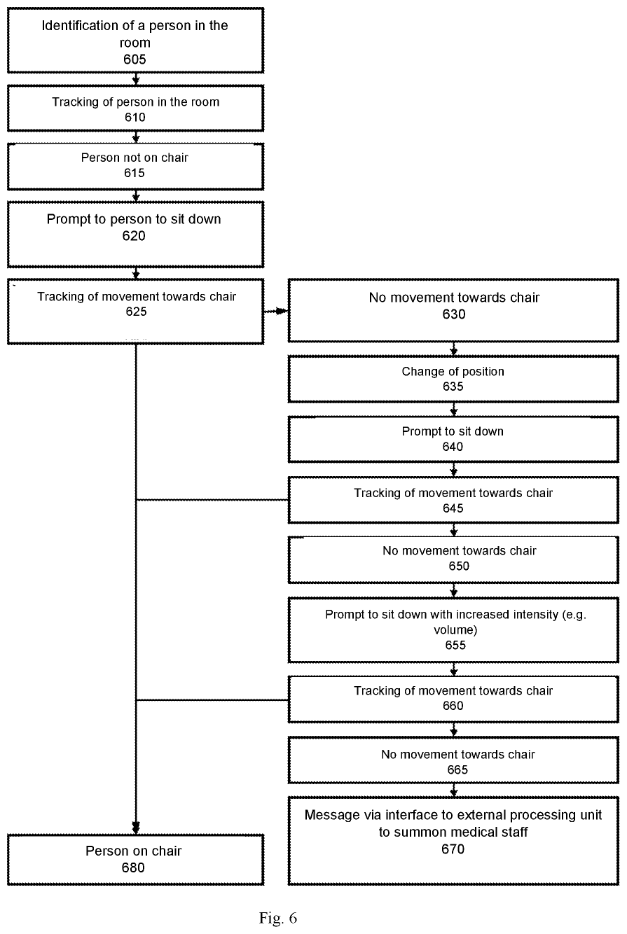

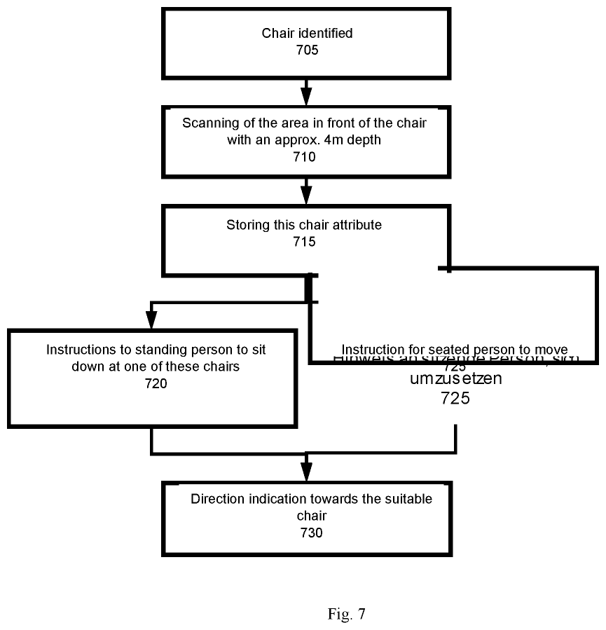

Furthermore, as illustrated in FIG. 6, the service robot 17 is capable of identifying a person in the room 605, for which various alternative and/or additional approaches may be employed: For this purpose, on the one hand, the LIDAR 1 can be used to identify two cross-sections from different angles, which have a diameter of at least 5 cm, e.g. at least 7 cm, which are not exactly round and have a static distance of at least 3 cm, e.g. at least 5 cm. Alternatively or additionally, a person can be identified based on an image classification using the 2D or 3D cameras 185, for example using the SDKs already mentioned in the previous section. In one aspect, classification as a person is more likely to occur if the position in the room changes over time. Furthermore, the service robot 17 uses algorithms in the prior art that, using the SDKs of the sensors (such as cameras 185) and/or third-party software, enable a skeleton model of the person to be created and tracked over time 610, for example, using the visual person tracking module 112 and/or the laser-based person tracking module 113. If a person is identified who is not located on the chair 615, the service robot 17 prompts that person to sit on the chair, which is carried out, for example, using acoustic and/or visible means 620. In the process, the service robot 17 tracks the person's movement toward the chair 625. If the service robot 17 does not detect any movement towards the chair 630, the service robot 17 changes its position 635. The reason for this measure is that the service robot 17 may be in the way of the person or the sensors may not have detected the person correctly. If the detection erroneously assumed the presence of a person, this process is interrupted, or it is alternatively continued (not shown in FIG. 6). In step 640, the service robot 17 then again prompts the person to sit down. In the process, the service robot 17 again tracks the movement 645, for example by means of the visual person tracking module 112 and/or the laser-based person tracking module 113. If the service robot 17 detects no movement toward the chair in step 650, the service robot 17 again prompts the person to sit down, but with an increased prompting intensity, achieved for example by utilizing a higher speech output volume, an alternative speech output, visual signals, etc. 655. Once again, the person is tracked with respect to his or her movement towards a chair 660. If no movement of the assumed person towards the chair is detected 665, the service robot 17 sends information via an interface 188 (such as WLAN) to a processing unit that interacts with medical staff via a display 2 and prompts the medical staff to move towards the service robot 17 and assist this service robot 17 670. In an additional and/or alternative aspect, the service robot 17 detects the degree of person detection, i.e., the service robot 17 uses internal rules to determine the detection quality, e.g. deviations from detection threshold values, and, based on this, determines a number of prompts that the service robot 17 directs to the person.

Since there may be more than one chair in the room that the person can sit on, and there may not be sufficient space in front of each chair to cover the required distance of 3 m, the service robot 17 features a correction mechanism. This correction mechanism enables the service robot 17 to identify, from the set of identified chairs 705, those chairs in front of which, in orthogonal direction towards the front of the chair, there is a free area without obstacles which has a minimum length of 3.5 m, for example at least 4 m 710. Provided that there is the required free space in front of a chair necessary to perform the test, this property is stored as an attribute in the memory 10 of the service robot 17 715. This information is used when a user navigates towards the chair, or this information is used to ensure that the person is seated on a chair that is suitable for performing the test in virtue of having sufficient space in front of the chair. Additionally and/or alternatively, the chair can also be identified by means of a floor marker, which is identified, for example, using the method described a few paragraphs further below.

For this purpose, when prompting a (standing) person to sit down, the service robot 17 can indicate suitable chairs to this standing person in step 720. The service robot 17 may also prompt the person to stand up again and to sit down in another chair 725. In one aspect, this chair is further identified in step 730. For this purpose, the service robot 17 uses the object IDs and the sequence in which the chairs are positioned. In addition, information is available within the memory 10 of the service robot 17 that, for example, the person is sitting on Chair No. 6, but only Chair No. 4 and No. 7 are eligible for performing the test because there is sufficient space in front of them. The service robot 17 can then integrate the information in the prompt to the person to switch chairs that the person can position him- or herself on a chair, for example, two seats to the left of the service robot 17 or one seat to the right of the service robot 17. In this case, the service robot 17 is able to correct such information based on the orientation in the room occupied by the person and the service robot 17 in such a way that the information output relates to the orientation or perspective of the person. In the example provided, this would be two seats to the right of the person or one seat to the left. Analogously, the service robot 17 can also use the coordinates of a standing person and of a suitable chair in order to refer this standing person to this chair, for example by means of the request “please sit on the chair diagonally to your left”, if appropriate specifying a distance. In an additional and/or alternative aspect, color information about the chair can also be included that, for example, was previously collected via an RGB camera.

If the chair is empty and the service robot 17 does not identify a person in the room, but the service robot 17 has been instructed to perform a test with a person in connection with the chair, the service robot 17 positions itself, for example, at a distance of more than one meter away from the chair. In one aspect, the service robot 17 also has information via its navigation module 101 indicating the direction from which a patient may approach. In one aspect, this information may have been explicitly stored in the system.

In an additional or alternative aspect, the service robot 17 is capable of recognizing doors or passages. In the case of closed doors, there is an offset within a wall of at least 1 cm oriented perpendicular to the wall surface 805, while orthogonally thereto, the offset is a distance greater than 77 cm, e.g., greater than 97 cm but less than 120 cm 810. Alternatively or additionally, the offset is a double offset with a distance of several cm, the inner distance being the approx. 77 cm mentioned above, or better approx. 97 cm. With the aid of this information, it is possible to detect a closed door, especially with the use of the LIDAR 1. In the case of an open door, the LIDAR 1 allows the service robot 17 to recognize either a plane which is adjacent to one of the edges in the XY direction at a length of approx. 77-97 cm 815 and has an angle to the edge of 1-178° 820, an angle which is variable over time 825, and/or a distance of at least 90 cm behind the identified opening with no further boundary of the XY plane in the map 830, which the service robot 17 records, for example, via the LIDAR 1.

In the case of a 2D or 3D camera 185, on the one hand, algorithms based on learning typical door characteristics may again be applied. On the other hand, information from the Z-direction can also be processed in this way and, if necessary, combined with data from the XY-direction, which helps to identify a doorway with a higher probability if the area identified as a possible door or passage in the XY-plane has a height limit of 1.95-2.25 m. Additional and/or alternative object information may also be included relating to a door handle.

In the event that the chair is unoccupied, the service robot 17 uses its navigation module 101 to establish the direct path between that chair and the door or passageway that is not blocked by any obstacle, based on the determined position of the door or passage and the chair, for example, by determining the Euclidean distance. The service robot 17 positions itself outside this path, for example with a spatial orientation that allows its sensors to target the chair and/or the door/passage.

If the service robot 17 identifies a person entering the room, the service robot 17 prompts him or her to sit on the chair as described above.

If a person is now seated on the chair, the service robot 17 signals the person via an output unit, for example a loudspeaker 192, alternatively and/or additionally also via a display 2, to stand up and walk straight ahead for three meters and then return to the chair.

The service robot 17 is able to recognize a distance marker on the floor by means of a 2D or 3D camera 185, for which standard pattern recognition methods are used. In the first step, the service robot 17 uses the position information of the identified chair. To ensure that it is a distance marker and not, for example, a normal floor pattern, the service robot 17 first uses its navigation module 101 to determine a position in the room situated approximately orthogonally relative to the front of the chair at a distance of 3 m. It then scans the area on the floor at this approximate position in order to identify such a marker. Other areas of the floor are scanned to determine whether the pattern is unique or repeating. If it is unique or, if applicable, stored in the memory 10 of the service robot 17, this pattern is used as a marker for the 3 m point.

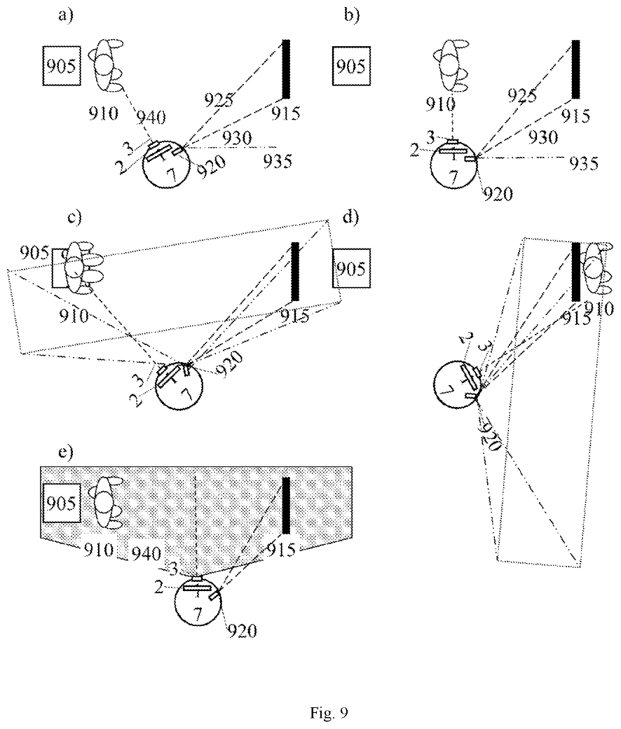

One disadvantage of a fixed floor marker is that a chair may possibly be moved, be it due to cleaning work in the room, people sitting on that chair, or other reasons. Therefore, in an additional and/or alternative aspect, the service robot 17 is provided with a projection device 920 to project a marker at a distance of 3 m to the front of the chair in an orthogonal direction. The XY coordinates of both the chair and the 3 m point are in turn determined, for example, via the navigation module 101, with this navigation module 101 having been previously updated by the service robot 17 with respect to the position of the chair. A light source, e.g., a laser or an LED, which in turn can be focused using lenses or similar means, is used for this purpose. This projection device 920 is capable of projecting an area onto the floor in the form of a bar, if appropriate with lettering prompting the person to move up to it. In one aspect (see FIGS. 9 a) to b) for a top view), the projection device 920 is movably mounted independently of the service robot 17, allowing the service robot 17 to position itself frontally towards the person 910 (as shown by the line 940), for example through its own rotational movements, while the projected marker 915 always remains in the same position orthogonally to the front of the chair 905. In addition, in one aspect, the light source of the projection device 920 may itself be movably mounted, and in another aspect, mirrors, e.g., micromirrors or micro-structured elements, direct the light in such a way that this light remains at the same location when a movement is executed, such as a rotational movement of the service robot 17. The angles thereby change between the times a) and b) during which the person 910 moves towards the marker 915, including the angle between the lines 925 and 935 and between 930 and 935. In an alternative and/or additional aspect, the service robot 17 may also move parallel to person's walking direction at all times.

In another aspect (FIGS. 9 c) to d)), the light source is capable of projecting onto an area on the floor having a width of more than 3 m from the perspective of the service robot 17. This projection device 920 thereby covers the distance that the person, starting from the chair, is supposed to cover. In the process, the central axis of the projection device 920 is rotated by an angle between 10° and 60°, e.g., 20-50°, from the central axis of the camera 185 facing the Z axis of rotation of the service robot 17, i.e. in the direction in which the person is supposed to move from the perspective of the service robot 17. For example, if the person is located on the chair to the left of the service robot 17 while the 3 m point is to the right of the service robot 17, the projection marker (such as the bar) at the 3 m point is in the right edge of the projected area from the perspective of the service robot 17. If the service robot 17 rotates in the direction of the fixed 3 m point, the projection marker moves to the left edge of the projected area. Here, for example, a projection device 920 can be used, such as is found in conventional (LCD) beamers, in which a matrix is controlled by software in such a way that different areas of the projected surface are illuminated with different brightness. If the chair with the person is to the right of the service robot 17 and the 3 m point is to the left, the orientations are mirror-inverted correspondingly. In FIG. 9 c), the person 915 is sitting on the chair 905. The projection device 920 can illuminate an area resulting from the dotted rectangle. The 3 m marker 915 is located in its right-hand area. If the service robot 17 rotates, the projected area moves clockwise together with the service robot 17, keeping the 3 m marker at the fixed XY coordinate, which means the 3 m marker moves to the left-hand area of the projected area (FIG. 9 d)). The embodiment described here assumes a fixed projection direction 920. In an alternative and/or additional aspect, the projection device 920 is movably mounted (effect not shown in detail).

In an alternative aspect, the service robot 17 does not rotate and uses the LIDAR 1 and/or the 2D or 3D camera 185 to detect more than the complete distance that the person has to cover (see FIG. 9 e), where the minimum area detected by the sensors is dotted). In another aspect, the 2D or 3D camera 185 is adjustably mounted, while the projection device 920 or light source is rigidly or also adjustably mounted (not shown separately).

A processor in the service robot 17 calculates the projected area based on the coordinates of the navigation module 101 of the service robot 17, which previously determines the position of the chair, the 3 m point and the service robot 17, the inclination of the projection device 920 and its height in order to project a bar that has the least possible distortion from the point of view of the person completing the exercise and that is roughly parallel to the front of the chair. The marker may have a different shape depending on the design.

The person is tracked by means of a procedure adequately described in the prior art, for example using the visual person tracking module 112 and/or the laser-based person tracking module 113. For this purpose, the service robot 17 also detects the posture of the upper body in order to detect when the person starts to sit up. From this point in time onwards, the time the person takes to complete the test is also recorded. Timekeeping ends when the person has turned around, returned to the chair, and sat down on it again. The algorithms allow the turning movement to be identified, for example, as a pattern by generating a skeleton model with skeleton points of the person in such a way that, during the turning movement, the arms are approximately parallel to the plane coinciding with the distance to be covered. In one aspect, skeleton points of the arms are evaluated over time and a determination is made of an angular change of over 160° of symmetrical skeleton points to the line connecting the start and turning positions.

In an alternative and/or additional aspect, the service robot 17 is configured in such a way that the service robot 17 determines the distance the patient covers while traversing the path. Since the start position and the turning position are 3 m apart, the length of the distance to be covered is 6 m, starting and ending at the chair at the start position, which is also the end position, with the turning position at a distance of 3 m away from the chair. In this case, the service robot 17 does not necessarily have to detect the marker on the floor. The distance covered can be determined in various ways, including by adding up the step lengths. This can be based on the distance of the ankle joints or ankles, which are recognized by a 2D or 3D camera 185 in conjunction with the evaluation frameworks used for the purpose and to which points in three-dimensional space are assigned, the distances of which can be determined by the service robot 17, e.g., in the form of vectors. Alternatively and/or additionally, the distance covered by the patient can be determined by adding up Euclidean distances between coordinate points which the patient passes and which can be determined from a map of the surroundings in which the patient and the service robot 17 are located, whereby the coordinates of the patient can be determined using reference positions. These include distances to recognized spatial boundaries or the position of the service robot 17, which can be determined using self-localization (self-localization module 105).

As the patient is being tracked, e.g. by means of the visual person tracking module 112 and/or the laser-based person tracking module 113, the service robot 17 calculates the distance covered and sets this distance covered in relation to the total distance that the patient must cover. An output device such as the display 2 and/or the speech synthesis unit 133 allows the service robot 17 to provide feedback to the patient via about how far the patient still has to walk, how many steps that is, when the patient can turn around, etc.

Based on the recorded time, the service robot 17 transmits a score based on reference data of the rule set 150 stored in the memory 10. The service robot 17 is capable of transmitting the score and/or the recorded time to the patient administration module 160 in the cloud 18 via an interface 188 (such as WLAN).

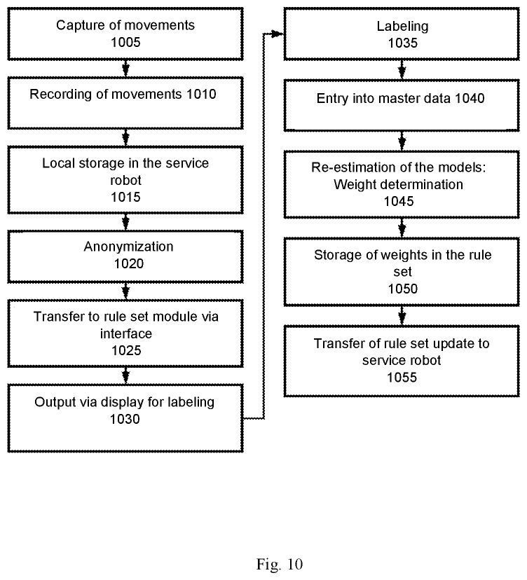

As shown in FIG. 10, in one aspect, the service robot 17 is able to use its sensor 3 to detect the movements of the person in step 1005, capture these movements as video in step 1010, store them in step 1015, and, in step 1030, transmit them via an interface 188 (such as WLAN) to a cloud memory in the cloud 18 located in the rule set 150. The data is transmitted in encrypted form. The facial features of the person to be assessed are rendered unrecognizable beforehand in order to preserve his or her anonymity 1025. The video material is available within a rule set 150 for labeling purposes in order to further improve the reference data of the rule set 150 by means of self-learning algorithms. For these purposes, among others, access to the stored data is possible via a terminal 1030, so that medical staff can evaluate and label the video recordings 1035. Here, labeling refers to the manual classification of the postures of a person, e.g. a person is sitting in a chair, standing up, moving forward or backward, turning around, etc. Labels can also be assigned to times for events identified in a video sequence. For this purpose, for example, individual start or end points of movements are marked in time while the movements, e.g. body poses, describing an orientation of the limbs, e.g. over the course of time, are simultaneously classified. The data labeled in this manner, for example, is subsequently stored in the database in which the master data is also located 1040. The rule set 150 can subsequently perform, for example, an independent improvement of the classification rules by means of algorithms, e.g. of neural networks. This improvement is achieved primarily in two ways: a) by capturing situations that have not been described before because they may be rare, and b) by increasing the number of cases. Both result in an ability to make more precise weight estimates 1045 when making a classification. In the process, appropriate assignments are made to the vector spaces resulting from the patient's posture, movements, etc., that allow a better estimation of the poses of the person to be assessed. This includes rising from the chair, walking, turning around, and sitting down again. The new weights are stored 1050 in the rule set 150 for the purpose of improved classification and transmitted to the service robot 17 via an interface 188 (such as WLAN) in the form of an update.

FIG. 59 summarizes the system for detecting and evaluating movements around standing up and sitting down on a chair as follows: The system comprises a processing unit 9, a memory 10, and at least one sensor for the contactless detection of the movement of a person, with the system having in its memory 10 a chair detection module 4540, an output device such as a loudspeaker 192 and/or a display 2 for transmitting instructions, a time-distance module for determining the time required to cover the distance 4510 and/or a speed-distance module 4515 for determining the speed of the captured person on a path, and a time-distance assessment module 4520 for assessing the speed of the person on a path and/or the time required to cover the distance. In addition, the system may have a hearing test unit 4525 for performing a hearing ability test, an eye test unit 4530, and/or a mental ability test unit 4535. The system may be a service robot 17. In one aspect, the system includes a projection device (920), e.g. to project the marker representing the turning point and/or the starting point. In one aspect, the system has a person recognition module 110, a person identification module 111, a tracking module (112, 113), a movement evaluation module 120, a skeleton creation module 5635, and/or a skeleton model-based feature extraction module 5640.

### Mini-Mental State Exam

### Mini-Mental State Exam: Speech Exercises

The service robot 17 is further configured to perform the mini-mental state exam. The purpose of the mini-mental state exam is to identify cognitive impairments, such as dementia. As part of the test, the communication devices of the service robot 17 (speech input and output, display 2) pose questions to the patient, who can answer them via the communication device of the service robot 17 (for example, as a speech input, as an answer to be selected on a screen, as a freehand input, e.g. of a date, place of residence, etc.). For the performance of the exam, the display 2 of the service robot 17 can be used on the one hand, and, on the other hand, a separate display 2 connected to the service robot 17 via an interface 188 (such as WLAN), e.g. a tablet computer, can also be used, which the person can take in hand or place on a table, making it easier to answer and complete the exercises.

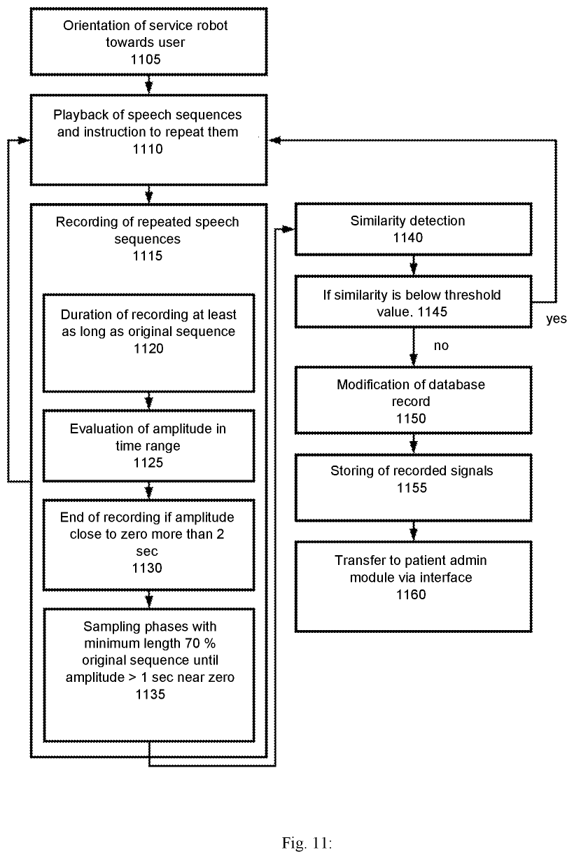

The service robot 17 is configured to enable the service robot 17 to communicate with a person, as shown by the method described in FIG. 11. For this purpose, in one aspect, the service robot 17 orients itself in the room in such a way that the display 2 of the service robot 17 is approximately parallel to the axis that passes through the user's two shoulders, hip, and/or knees, which are recognized via the skeleton model obtained using the 2D or 3D camera 185 and their SDKs. The service robot 17 thereby orients itself so that it faces the user 1105. As part of the interaction with a user, at least one speech sequence stored in the memory 10 is played back via a loudspeaker 192 and a user is prompted via a display 2 and/or via a speech output to repeat the played-back sequence 1110. Following the prompt, the service robot 17 records acoustic signals emitted from the user via a microphone 193 1115 over the same duration, for example, as required to output the speech sequence to be repeated by the user 1120. This step and the subsequent steps are performed by the speech evaluation module 132. The service robot 17 analyzes the amplitudes of the signal within the time range 1125. If the amplitude drops to zero or near zero (e.g. <90% of the maximum of the amplitudes) for more than 1 second, e.g. more than 2 seconds, the recording is terminated 1130. In addition, sampling is carried out, with the sample width defined over phases of near zero amplitudes that are greater than 1 second and have a length of at least 70% of the sequence that the user is supposed to repeat and that is stored in the service robot 17 1135.

This ensures that multiple speech attempts by the user are recorded and evaluated individually. The service robot 17 compares the samples either in the time range or frequency range and calculates similarity values 1140 taking common methods used in audio technology into account, especially cross-correlation. In an alternative or additional aspect, if the similarity value is below a threshold value 1145, for example, the service robot 17 again prompts the user to repeat the sequence (connection 1145=>1110). If the similarity value is above a certain threshold value, then the service robot 17 modifies a value in a database within the memory 10 of the service robot 17 that relates to the user 1150. The recorded speech signals emitted by the user are stored 1155 and, along with the modified value from the database, are transmitted 1160 to the patient administration module 160 via an interface 188 (such as WLAN). In an additional or alternative aspect, only the sequence recorded by the service robot 17 with the highest similarity value compared to the model sequence is stored. In addition, the system counts the number of attempts to repeat the sequence and, if this number exceeds a threshold value, stops recording the repetition attempt in question and proceeds to the next sequence to be repeated. Multiple repetition attempts or even failed repetition attempts on the part of the user are also recorded in the database.

### Mini-Mental State Exam: Folding Exercise

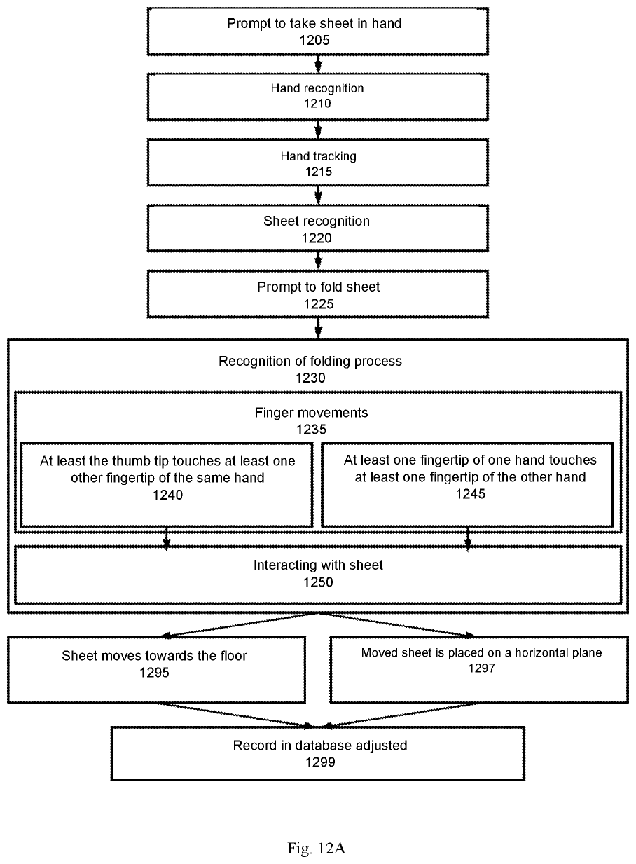

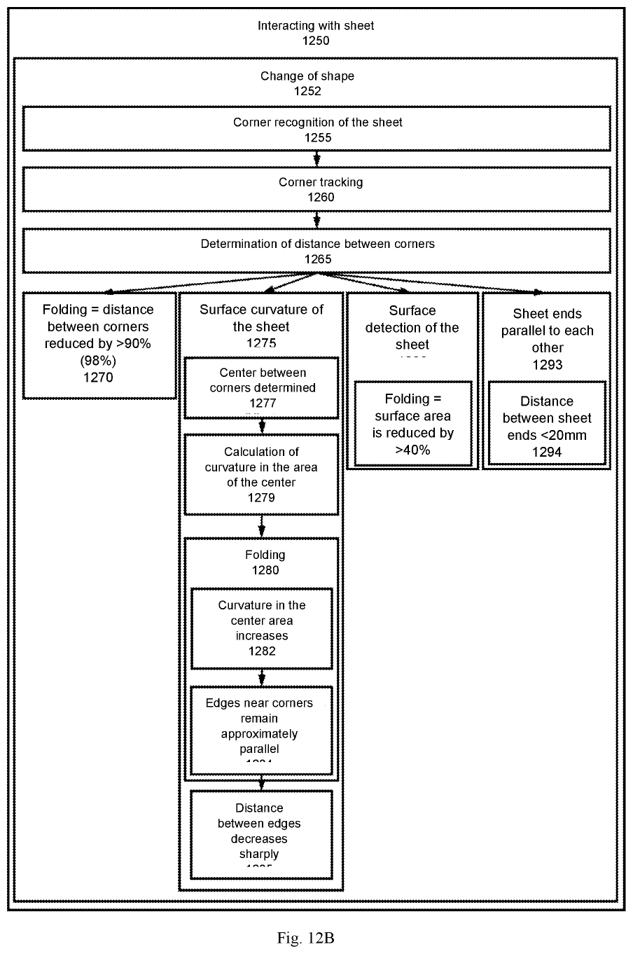

One exercise in the mini-mental state exam requires that the person being tested pick up a sheet, fold it, and lay it down or drop it, as shown in FIG. 12. For this purpose, the mobile service robot 17 has an optional device containing sheets that the person to be assessed can remove for the test, e.g. if prompted by the service robot 17. Alternatively, the mobile service robot 17 can indicate such a sheet to the person to be assessed, which is located in the premises where the test takes place. The speech output and/or the output unit of the display 2 is configured 1205 accordingly for this purpose.

The service robot 17 is configured in such a way that the sensor 3 with the design of a 3D camera, for example a time-of-flight (ToF) camera, can be used to detect and track the hands of a user, i.e. the hands are recognized in the first step 1210 and tracked in the second step 1215 when the user folds a sheet. As an alternative to a ToF camera, approaches are also possible in which hands are recognized 1210 and (hand) movements are tracked 1215 based on a single 2D camera in order to recognize corresponding gestures or the folding of a sheet 1220. The weights originate, for example, from a model that was classified using conventional machine learning methods, such as regression methods, and/or neural networks, such as convolutional neural networks. For this purpose, a large number of folding movements must be recorded in advance, labeled, and learned by the usual algorithms. Alternatively and/or additionally, skeleton models can also be created via the 2D camera on the basis of frameworks such as Open Pose or PoseNet in conjunction with Tensorflow.

The detection of the movements is performed over time, for example using the visual person tracking module 112 and/or the laser-based laser-based person tracking module 113. In the first step, the hands are recognized 1210 and segmented from the overall image. In the second step, objects 1220 located in the hands are recognized via segmentation, for example using a fault-tolerant segmentation algorithm (e.g., RANSAC framework) that allows pattern recognition. Tracking methods adequately described in the prior art allow the recording of movements over time 1215. Initially, there is no sheet in the user's hands. Then the user picks up a sheet and folds it, after which the sheet moves in negative Z-direction, i.e., it moves towards the floor. The last sheet movement does not necessarily involve one or both of the user's hands. The sheet is determined, for example, by means of sheet classification, i.e., using two- or three-dimensional data of the camera 185 created previously by recording images of the sheet and labeling the images. The term “sheet” encompasses both paper and materials that have an equivalent effect on the exercise and/or that have similar dimensions and possibly properties similar to a sheet of paper.

At the beginning of the exercise, the service robot 17 prompts the user to take a sheet 1205. A speech output via a loudspeaker 192 of the service robot 17 is used for this purpose, for example. Additionally, or alternatively, an indication on a display 2 can also be used, or a combination of both methods. From time of the prompt, object recognition for the sheet starts, for example. The service robot 17 analogously prompts the user to fold the sheet 1225, e.g., in half. Then the service robot 17 observes the folding process and, upon completion of the folding process, the service robot 17 prompts the user to lay down or drop the sheet. Alternatively or additionally, the information for folding and/or to laying down or dropping the sheet may be provided directly following a previous prompt of the same exercise.

In one aspect, a 3D camera is used, such as a Kinect or Astra Orbbec. The challenge in recognizing elements of the hand, i.e., the fingers, and finger tracking 1230 derived from this is that, from the perspective of the camera 185, individual fingers may be obscured, making direct estimates impossible. This is the case with gestures performed without an object in the hand. If, on the other hand, a sheet is folded by one or more hands, some of the fingers may also be obscured, depending on the type of folding process. The folding process can be recognized or classified as such on the basis of finger movements 1235, for example, if at least one thumb and at least one and preferably several fingers of the same hand touch each other at the level of the fingertips 1240, i.e., at least two fingers are detected and tracked, for example. Alternatively, one or more fingers of one hand may touch one or more fingers of the other hand, e.g., in the area of the fingertips 1245. In all cases, the sheet is being acted upon by at least one finger 1250. For example, the sheet is between these fingers, with the sheet being recognized as described in the following paragraph.

The system and method alternatively and/or additionally provide for the recognition of a sheet and its change of shape (step 1252), with this sheet being in contact or in interaction with at least one finger. In one aspect, the recognition targets the four corners of the sheet 1255 located in one or both hands of the user. In doing so, each corner is tracked individually over time 1260 and the distance between these corners is determined 1265. A successful folding is recognized, for example, a) if the distance between two corners in three-dimensional space is reduced by more than 90%, e.g., reduced by more than 98% 1270. Alternatively and/or additionally, the distance between two opposite edges of the sheet can also be tracked and a folding process can be recognized if the distance falls below these specified values. Additionally and/or alternatively, (b) the surface of the sheet is tracked with respect to its curvature 1275. For this purpose, the folding module determines the center between two corners 1277 and monitors (tracks) the curvature of the sheet 1279 in these areas, for example. In this case, a successful fold 1280 is recognized if the curvature increases over time in this area 1282, while the sheet edges/margins near the corners exhibit approximately parallel motion 1284 (i.e. in particular those that are folded) and the distance between the sheet edges decreases sharply 1285, e.g. to a distance of less than 2 mm, making an individual detection of the two approximately equally sized sheet sections generally no longer possible, since the depth resolution of the camera 185 cannot detect two sheets lying on top of each other due to the small thickness of the sheets. In addition and/or alternatively, (c) the area of the sheet in three-dimensional space is also detected over time, with a depth of the sheet of less than 2 mm remaining undetected or only poorly detected. A folding of the sheet is determined by the fact that the area of the sheet is reduced by more than 40% over time, e.g. by approx. 50%. This approach can also be implemented, for example, without explicitly analyzing and tracking the fingers. Alternatively and/or additionally, (d) the distance of the ends of a sheet margin parallel to each other is detected and evaluated 1293 and, if the distance of the sheet ends to each other is less than 20 mm, a folding is recognized 1294. The overall detection accuracy can be increased by combining two or more of these three detection variants. If this exercise has been successfully completed, i.e. the sheet has been folded and it subsequently moves in the direction of the center of the earth 1295, or alternatively comes to rest on a plane 1297, this is noted in a database 1299, in particular in the database in which the test results are stored.

FIG. 61 provides a summary illustration of the system, for example a service robot 17, for the recognition of a folding exercise: The system includes a processing unit 9, a memory 10, and a sensor for the contactless detection of a person's movement such as, for example, a 2D and/or 3D camera 185, a LIDAR 1, a radar sensor, and/or an ultrasonic sensor 194, and several modules in its memory 10. These include a sheet detection module 4705, a folding movement detection module 4710 for detecting a folding movement of a sheet, a skeleton creation module 5635 for creating a skeleton model of the person, a sheet distance corner edge module 4720 for detecting the distances between the edges and/or corners of a sheet, a sheet shape change module 4725 for detecting the change in shape of a sheet, a sheet curvature module 4730 for detecting the curvature of a sheet, a sheet dimension module 4740 for detecting the dimensions of a sheet, and/or a sheet margin orientation module 4745 for detecting the orientation of sheet margins. Further, the memory 10 includes a fingertip distance module 4750 for detecting the distance of fingertips from at least one hand, and a sheet detection module 4705 for detecting a sheet, for example comprising a sheet segmentation module 4755 for detecting a sheet and/or a sheet classification module 4760. Furthermore, the system includes an output device such as a loudspeaker 192 and/or a display 2 for communicating instructions and an interface 188 to a terminal 13. In one aspect, the system has a person recognition module 110, a person identification module 111, a tracking module (112, 113), a movement evaluation module 120, a skeleton creation module 5635, and/or a skeleton model-based feature extraction module 5640. The sequence includes a detection, identification, and tracking of at least one hand of a person; a detection, identification, and tracking of a sheet; and a joint classification of dimensions, shapes, and/or movements of the detected sheet and elements of a hand as a folding process. In one aspect, there is further an identification of the sheet by means of a fault-tolerant segmentation algorithm and, for example, a sheet classification and/or classification of a folding operation based on a comparison with two-dimensional or three-dimensional patterns, including shape patterns and/or movement patterns.

### Mini-Mental State Exam: Sentence Exercise

As part of the test, the service robot 17 may further prompt the user to spontaneously think of a sentence. When evaluating this sentence, spelling and grammar are not relevant, but the sentence must contain at least one subject and one predicate. For this purpose, the service robot 17 uses the communication devices (display 2; loudspeaker 192) to prompt the person to be assessed to think of a spontaneous sentence 1305 and to use his or her fingers to write it on the touchpad of the service robot 17 1320. This may be achieved using a display output 1310 or a speech output 1315. In a second aspect, a pen or pen-like object is provided by the service robot 17 for this purpose 1320. In a third aspect, a pen and a sheet of paper are provided for the person to use to write down the sentence 1325 and the service robot 17 prompts the person using the communication device to hold the written sheet in front of a camera 185 of the service robot 17 1330 so that it can be recorded and stored in the memory 10 of the service robot 17. For this purpose, the sensor system (2D, 3D camera 185) tracks the user movements 1335, e.g. by means of the visual person tracking module 112 and/or the laser-based person tracking module 113, uses the internal object recognition of a sheet (see previous approaches) and recognizes that the user is holding the sheet in front of the 2D camera of the service robot 17 1340 and the service robot 17 recognizes the sheet 1345, which the service robot 17 photographs with the 2D camera 1350.

In a subsequent process step, OCR processing is performed on the sentence contained in the photograph 1355. For this purpose, the processor of the service robot 17 makes use of corresponding established libraries for image or text processing that allow OCR processing to be performed. Depending on the aspect, such data processing may be possible in the cloud. In a further step, a natural language parser 1360 is used to determine the existence of subject and predicate in the sentence. For this purpose, the captured sentence is broken down into individual words in the first step (tokenization) 1365. Then, the stem form of the words is formed (stemming and/or lemmatization) 1370. Subsequently, the POS (part-of-speech) tagging is carried out, which classifies the words into subject, predicate, object, etc. 1375. A neural network-based approach can also be employed in this context. For this purpose, toolkits such as NLTK or SpaCy can be used. The results are stored in a memory in step 1380 and a comparison is made in the next step 1385 to establish whether a subject and a predicate occur in the sentence provided by the user. If so, the successful completion of the exercise is noted in a database (step 1390).

### Mini-Mental State Exam: Pentagon Exercise

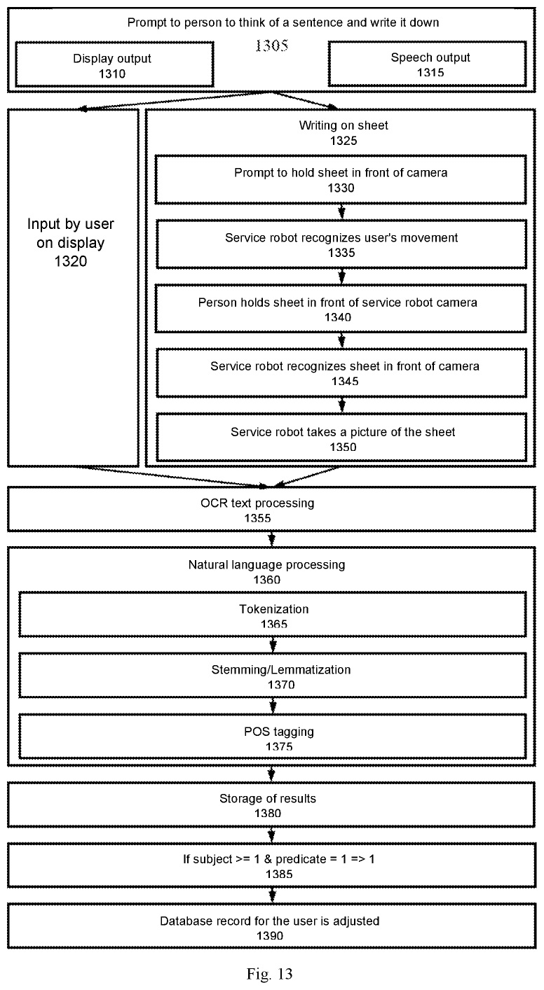

Another element of the test involves drawing two overlapping pentagons. For this test, in a first aspect, the person to be assessed is given the opportunity to produce the drawings on a display 2 located on the service robot 17. In a second aspect, the display 2 is freely movable within the room in which the service robot 17 and the user are located but is wirelessly connected to the service robot 17 via an interface 188 (such as WLAN). In this aspect, the user may complete the drawing either with his or her fingers or by means of a tablet-compatible pen. In a third aspect, the user may produce the drawing on a sheet of paper, after which he or she is prompted by the service robot 17 by means of the communication devices to hold the completed drawing in front of a camera 185 of the service robot 17. The camera 185 records the image. In this respect, these processes are analogous to those described in FIG. 13 in 1305 to 1350, except that, in this case, it is not a sentence to be written down, but rather pentagons to be drawn.

The captured images are compared by the processing unit with those stored in a database. This is achieved using a rule set 150 that compares the features of an image with features of classified images and then makes a classification based on probabilities. Methods described in the prior art are employed as classification mechanisms, which have previously been created based on automated training, in particular using methods based on neural networks. Alternatively, classification mechanisms can be employed that were created without training and whose classification features were determined in the form of defined rules based on characteristic features of a pentagon and of overlapping pentagons (such as the number of angles and lines). This also takes, for example, rounded edges, unevenly drawn lines and, if necessary, lines that do not form a closed pentagon into account. In the context of such an evaluation, smoothing approaches can be used, e.g. to simplify the classification. If a threshold value is reached in the similarity comparison (e.g. correlation) between the pattern recorded by the service robot 17 and the comparison pattern stored in the rule set 150 or the recognition rules for two overlapping pentagons, the successful completion of the exercise is noted in a database.

### Manipulation Detection

The service robot 17 includes a function for detecting manipulation by third parties while completing the exercises. For this purpose, the sensors that are also used to analyze the user and his or her activities detect the presence of other persons in the room 1405. In the process, an analysis is made of whether the person(s) (including the user) position themselves spatially during the test in such a way that they can potentially manipulate the service robot 17, i.e. whether they are at a “critical distance” away from the service robot 17 1410. Possible manipulations include, in one aspect, entering data on a display 2 of the service robot. In addition, the distance of the person from the service robot 17 is determined and then a determination is made using at least one of the following three methods of whether the person is positioned sufficiently close to the service robot 17 to be able to make inputs (in particular on the display 2), if necessary: a) a blanket distance value is assumed, for example 75 cm. If the distance falls below this value, the service robot 17 assumes that the display 2 can be used (step 1415). Alternatively and/or additionally, the distance between the person's hand and/or fingers and the service robot 17 can also be detected, whereby the distance starting from which manipulation is assumed is shorter than that of the person per se. b) The arm length of the person is determined via the skeleton model 1420, in particular by determining the distances between a shoulder skeleton point and a hand skeleton point or the finger skeleton points. If this distance is not reached, the service robot 17 assumes that operation is possible 1425. c) The height of the person, which is determined by the service robot 17 1430, is used to infer an average arm length 1435 (e.g. which is stored in the memory 10) and, if this distance is not reached, operation/manipulation is assumed to be possible 1425. In addition to these three approaches, the service robot 17 can calculate the positioning of the person in the room relative to the position of the display 2 (step 1440). If, for example, the orientation of the shoulders, hips, etc., or the frontal plane of the person derived therefrom is approximately parallel to the display 2 or at an angle of less than 45° to it, and the person is oriented in the direction of the display 2, e.g. as indicated by the primary direction of movement of the person, the posture of the arms, head, knees, feet, facial features, etc., this increases the likelihood of interaction with the display. Depending on the orientation of the sensor system of the service robot 17, this approach can also be implemented for other elements of the service robot 17 instead of a display 2, for example for a switch-off button. In such a case, instead of the plane that the display 2 forms relative to the person, a virtual plane is considered that is oriented orthogonally to the axis of symmetry of the control element 186 towards the center of the service robot 17. In a second, optional step, the sensors analyze whether the input or manipulation of the service robot 17 is performed by the user or by a third person 1450. For this purpose, the service robot 17 tracks the persons within its surroundings based on characteristic features 1445 using a method generally described in the prior art (for example, based on height, the dimensions of the limbs, gait features, color and texture of the person's surface, e.g. clothing, etc.), e.g. by means of the visual person tracking module 112 and/or the laser-based person tracking module 113. Differentiation into users and third parties is carried out through the identification made at the service robot 17, whereby it is assumed that the person identifying him- or herself is the user. This is done with respect to inputs made via the display 2 using the optical sensors of the service robot 17. In summary, a determination of an orientation of the person relative to the service robot 17 may be performed here by determining the angle between the frontal plane of the person and the axis perpendicular to the control elements 186 of the service robot 17, projected in each case in a horizontal plane, and by comparing the determined angle to a threshold value under which an increased probability of manipulation is detected. In one aspect, the person may be registered at the service robot 17 and the person's identification features may be acquired and stored, which is followed, for example, by the capture and tracking of the person, the acquisition of identification features of the person, the comparison of the acquired identification features with the identification features of the person stored during the registration procedure, and the comparison with a threshold value, in which case similarities are compared, with a threshold value implying a minimum similarity. An increased probability of manipulation is detected if the value falls below the threshold value, while a lower probability of manipulation is detected if the threshold value is exceeded. Finally, the determined manipulation probabilities can be multiplied to determine a manipulation score, which is stored together with the evaluation results, for example, when or after the robot performs evaluations with the captured person. Depending on the type of comparison, other interpretations can also be made, as was shown, for example, in the introduction.

FIG. 62 illustrates an aspect of a system for detecting manipulation. The system, for example a service robot, comprises a processing unit 9, a memory 10, and a sensor for the contactless detection of the movement of at least one person, for example a 2D and/or 3D camera 185, a LIDAR 1, a radar sensor, and/or an ultrasonic sensor 194. The system includes modules with rules in its memory 10. These modules include, for example, a manipulation attempt detection module 4770 that detects manipulation on the part of at least one person detected in the surroundings of the system, a person identification module 111, a person-robot distance determination module 4775 for determining the distance of at least one person from the service robot 17, a height-arm length-orientation module 4780 for determining the height, arm length, and/or orientation of at least one person, and/or an input registration comparison module 4785 for performing a comparison to determine whether a person identified by the system is making inputs in the system, e.g. via the control elements 186. In addition, the system includes, for example, an output device such as a loudspeaker 192, a display 2 for communicating instructions, and/or an interface 188 to a terminal 13. In one aspect, the system has a person recognition module 110, a tracking module (112, 113), a movement evaluation module 120, a skeleton creation module 5635, skeleton model-based feature extraction module 5640, and/or a movement planner 104.

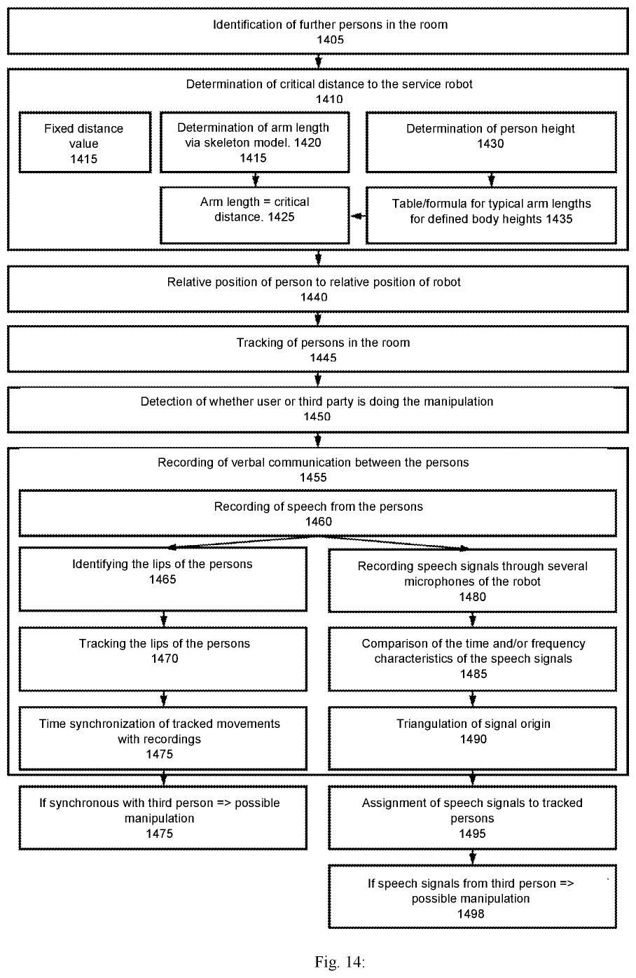

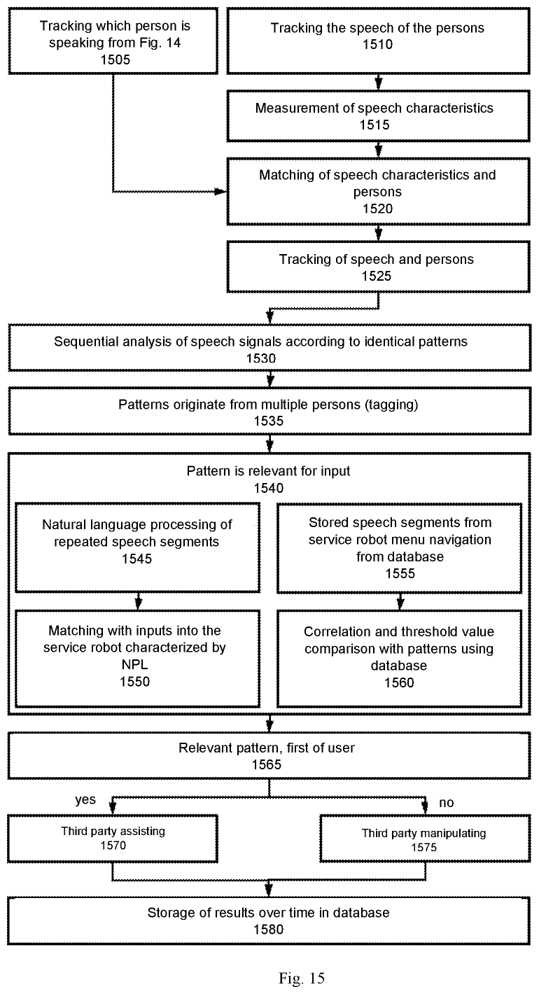

To exclude the possibility that third parties are only providing input at the instruction of the user, available microphones 193 are used to evaluate verbal communication between persons 1455 (in FIG. 14). For this purpose, speech signals are recorded 1560 within the surroundings of the service robot 17 via at least one integrated microphone 193. An identification of the speech source is implemented using two alternative or additional methods, e.g. also in the speech evaluation module 132. On the one hand, a visual evaluation of lip movements can be performed for this purpose, which are first identified 1565 and tracked 1570, then synchronized 1575 in time with the speech signals recorded by the service robot 17. Image recognition and tracking techniques in the prior art are used to recognize the speech movements of the lips. This enables the service robot 17 to identify the person from whom the registered speech originates and whether it corresponds to the user who is to perform the exercise, with the speech of the user being recorded when he or she identifies him- or herself to the service robot 17. Otherwise, manipulation may be occurring 1580. One disadvantage of this approach is that it may be difficult or even impossible to detect the lip movements of third parties whose posture is not directed towards the service robot 17. The second method, which circumvents this problem, consists of the sound analysis of several microphones 193 (step 1480) attached at different positions on the service robot 17 and recorded over multiple channels with the frequency recorded over time, with the processor of the service robot 17 performing a runtime analysis 1485 and using the time offset of arriving signals to calculate the person from whom these signals originate 1490. Alternatively and/or additionally, a microphone 193 can also be used. In this case, triangulation can be performed by changing the position of the service robot 17. For this purpose, for example, the elapsed times are correlated based on the calculated time offset via triangulation to determine the origin in the room (which can be done in two or three dimensions). This origin is then matched with the positioning of the persons in the room, which is determined by the 2D or 3D camera(s) 185 or the LIDAR 1, thereby allowing the service robot 17 to determine which person has spoken 1495. If it is the third person (and not the user) with whom the speech signals are correlated, manipulation may be occurring 1498. A value in a memory can subsequently be adjusted and, in one aspect, an instruction or an error message can be generated in the user dialog.

It may be that the third person is only assisting the user with the input, i.e. does not make any input of his or her own, but rather only inputs what is spoken, recorded, etc., into the service robot 17 by means of the display 2 or microphones 193. To test this possibility, the recorded word sequences are analyzed by correlating them to the individual persons by means of at least once of the methods presented in preceding sections, for example 1505. FIG. 15 illustrates the basic points of this procedure. Alternatively or additionally, the speech in the environment of the service robot 17 can be recorded 1510 and the speakers can be differentiated based on different speech features/characteristics 1515, including in particular the speech frequencies (especially the fundamental frequencies), varying speech intensity and/or varying speaking rate, in particular within the speech evaluation module 132. This method in combination with the methods in FIG. 14, which use speech signals from people either by lip tracking or localization based on speech signal propagation, makes it possible to correlate speech signals, once identified, with people without having to determine the lip movements and/or spatial position of the speakers each time again and, as the case may be, match these with the 2D/3D people tracking results. This matching of the persons with the speech characteristics 1520 allows speech to be recorded and simultaneously tracked based on the specific user 1525. The sequences recorded in each case and stored in the memory 10 of the service robot 17 are analyzed in terms of content by looking for “prediction behavior”, i.e. a check is made of whether the same text fragments or speech fragments/patterns occur multiple times in succession 1530 and originate from different persons 1535. This is achieved by tagging the patterns and the speech characteristics correlated with the different persons, such as the fundamental frequencies (alternatively and/or additionally, the approaches mentioned in FIG. 14 can also be used). Text fragments and speech fragments/patterns refer to identical words and/or word sequences, for example. In the respect, a relevant factor for the assessment of these sequences with respect to the assistance of the user or the manipulation of the service robot 17 is the person who names a relevant sequence for the first time. If this is the user 1565, then no manipulation should be assumed, but rather an assisting activity on the part of the third party 1570. If it comes from the third person for the first time, then manipulation should be assumed 1575. For this purpose, a check is made in the first step of whether a speech fragment was first recorded by a person who is not the user before the user repeats this speech segment. For this, correlations are made, especially in the time range, in order to search for identical words. In the process, a check is made in particular of whether more than one single word occurring within a sequence is repeated. In addition or as an alternative to the correlation analysis of the speech sequences, a lexical analysis by means of natural language processing is also possible 1545. Here, words are analyzed, e.g. using methods explained in previous paragraphs, and the sequence of the tagged words is compared based, for example, on tokenization, lemmatization, and part-of-speech tagging, e.g. using spaCy or NLTK in Python. This approach also makes it possible to verify that “prompting” is not, for example, repeated acoustically by the user for recording by the service robot 17, but rather that a corresponding input is made directly by the user into the service robot 17. This is because the only repeated speech segments/patterns that are relevant are those that are recorded and evaluated in terms of content by the service robot 17 within the scope of testing, e.g. in the form of a written form with questions for the user 1540. For this purpose, text inputs made in the service robot 17 (“free text”) as well as menu-guided inputs (selection options) are correspondingly also analyzed by means of natural language processing 1550, alternatively by stored speech signals that correspond to the menu selection 1555, and the third-party speech recordings are compared with the user inputs 1560. If the approaches to manipulation detection described here detect that a user input or recording is being made by a third party or that a third party is “prompting” the user, a note is made in the memory 10 of the service robot 17 that manipulation has occurred 1580.

In summary, the method for determining a probability of manipulation comprises detecting and tracking at least one person within the surroundings of the robot by means of a contactless sensor, determining the position of the person within the surroundings of the robot, recording and evaluating audio signals, determining the position of the source of the audio signals, comparing the determined position of the person and the position of the source of the audio signals and comparing the difference in position with a threshold value, and determining the probability of manipulation of the robot based on a comparison of the difference in position with the threshold value. In this case, the determination of the position of the source of the audio signals can be performed by detecting the direction of the audio signals by means of at least one microphone and triangulating the determined directions, for example also by changing the position of the service robot 17 or by using a second microphone. The determination of the position of the source of the audio signals includes the detection of the direction of the audio signal by means of a microphone, the determination of the position of at least one person by means of the contactless sensor, the triangulation of the direction of the audio signal and the determined position of the person. Furthermore, the evaluation of the person's face, the detection of the person's lip movements over time, a temporal comparison of the detected audio signals (e.g. by means of correlation evaluations) with the detected lip movements relative to a threshold value are performed and, if the threshold value is exceeded, the detected audio signals are correlated with the captured person. The method may also include registering the person at the robot (as a user) and acquiring and storing identification features of the person (as a user). These identification features include the frequency, intensity, and/or spectrum of the audio signals emitted from the person, e.g. further comprising a detection and tracking of the person, the acquisition of identification features of the person, a comparison of the acquired identification features with the identification features of the person stored while registering the person at the robot and a comparison with a threshold value (i.e. exhibiting a minimum similarity), the registration of inputs of the person using the control elements (186) and a classification of whether a registered person (a user) is making inputs using the control elements (186). For example, an increased probability of manipulation of the robot can be determined if a person who is not registered makes inputs using the robot control elements (186). The method may further comprise, for example: a detection of words and/or word sequences in the detected audio signals or audio sequences, an allocation of the detected words and/or word sequences to captured persons, and a determination of an increased probability of robot manipulation if a comparison of the determined word sequences results in a word and/or word sequence difference that is above a threshold value, i.e. that a minimum correlation is not reached. Further, the method may include, for example, the detection of words or word sequences entered by the person via a control element (186), the detection of words and/or word sequences in the captured audio signals, the correlation of the detected words and/or word sequences from the captured audio signals with captured persons, the acquisition of the person's identification features, the determination of an increased probability of robot manipulation if a comparison of the word sequences input via the control elements (186) with word sequences determined from the detected audio signals determines a minimum similarity of the word and/or word sequence and, at the same time, a minimum similarity of the acquired identification features of the person with the identification features acquired and stored during the registration process.

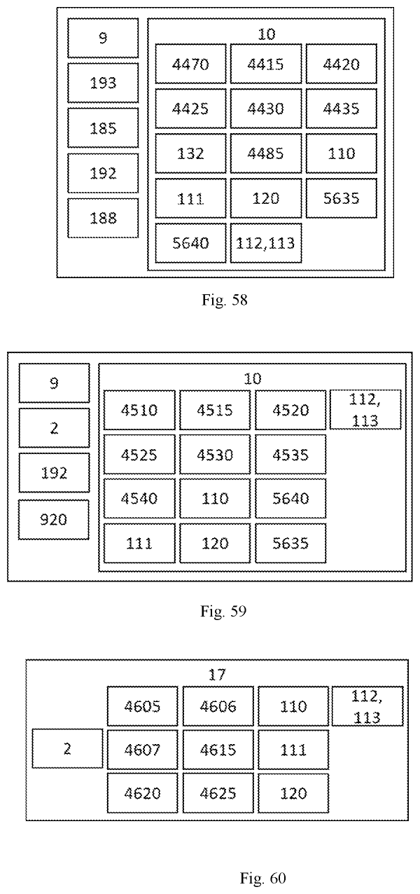

FIG. 58 shows the architectural view of the system for manipulation detection based on audio signals. This includes a processing unit 9, a memory 10 and a sensor for the contactless detection of the movement of a person detected in the surroundings of the system, at least one microphone 193, a person position determination module for determining the position of a person in the room 4415, an audio source position determination module for determining the spatial origin of an audio signal 4420, a module for correlating two audio signals 4025, an audio signal-person module 4430 for correlating audio signals with a person, and/or a speech evaluation module 132. Furthermore, an input registration comparison module 4785 is available to perform a comparison to determine whether a person identified by the system is providing input to the system. The system further includes an audio sequence input module 4435 for comparing an audio sequence (i.e. a sequence of sounds that reproduces words, for example) with a sequence of letters entered by hand. There is also, for example, an output device such as a loudspeaker 192 and/or a display 2 for transmitting instructions. A connection may be established to a terminal via an interface 188 (such as WLAN). The sensor for the contactless detection of the movement of a person is a 2D and/or 3D camera 185, a LIDAR 1, a radar, and/or an ultrasonic sensor 194. In one aspect, the system has a person recognition module 110, a person identification module 111, a tracking module (112, 113), a movement evaluation module 120, a skeleton creation module 5635, and/or a skeleton model-based feature extraction module 5640.

### User Impairment Testing

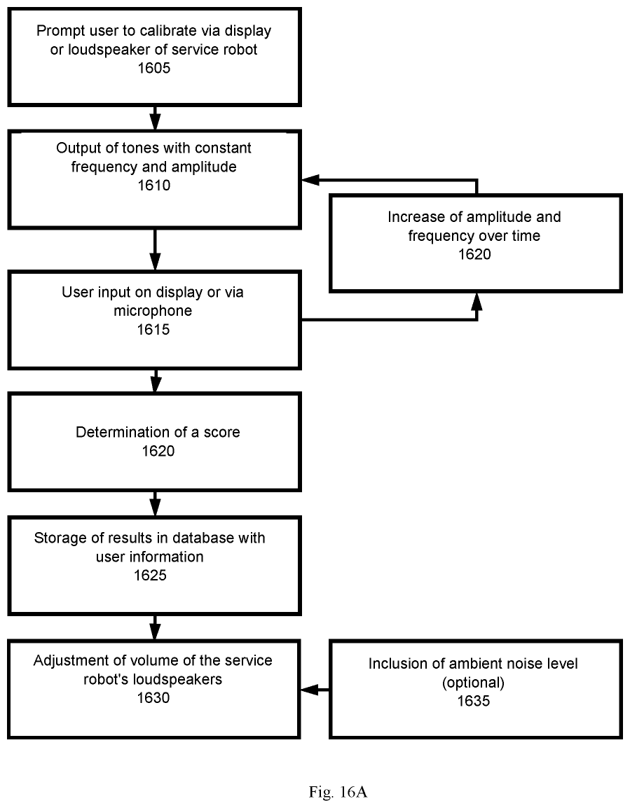

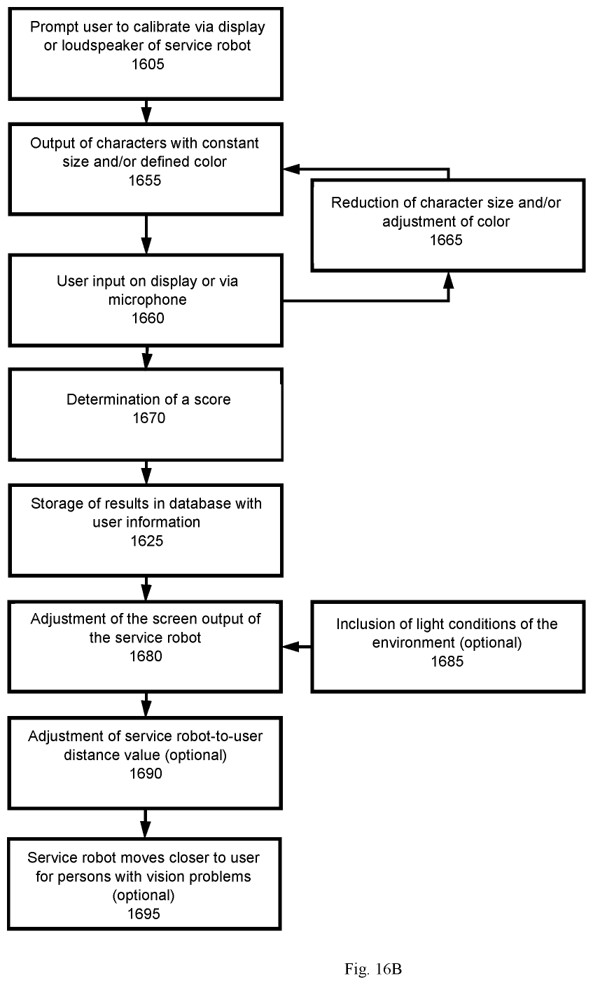

The users are primarily senior citizens who, in addition to a potential cognitive impairment to be tested by means of the procedures described in this patent application, may also suffer from hearing and visual impairments that could potentially distort the test results. In one aspect, in order to improve the accuracy of the test results, the service robot 17 is configured in such a way that the service robot 17 performs a short hearing test and additionally or alternatively a short eye test with the user before beginning the exercises. FIG. 16 provides a basic illustration of the procedural steps performed here. The service robot 17 first optionally indicates to the user via a screen output and/or acoustic output that comprehension problems may occur and that it is therefore necessary to calibrate the service robot 17 to the user. The eye and/or hearing test represents such a calibration. Then, the service robot 17 prompts the user to participate in the calibration 1605.

As part of a short listening test, the service robot 17 prompts the user to press corresponding fields in the menu on the display 2 when the user has heard certain sounds. Alternatively or additionally, speech input is also possible for the user, which in turn is evaluated using natural language processing methods as described in the prior art, for example within the speech evaluation module 132. The service robot 17 then plays a sequence of tones with different frequencies and volumes, but individually having an essentially constant frequency and volume 1610, and “inquires” each time as to whether the tone has been heard by the user. This may be achieved, for example, by the service robot 17 presenting a display 2 with input options to the user, by means of which the user may indicate the extent to which the user has heard the tone 1615. In one aspect, the sounds become lower in volume and higher in frequency 1620 over time. However, another sequence is also conceivable in this respect. The answers of the user are recorded. Subsequently, a score is determined 1625 indicating the extent to which the user has heard the tones. If, for example, the user's listening does not reach certain threshold values, i.e. if screen menu-guided or speech menu-guided positive responses provided to the service robot 17 that are evaluated accordingly fall under predefined limit values (e.g. only three of seven tones recognized), a corresponding score value can be determined on this basis. In one aspect, this score is stored in a database in the service robot 17 1630, e.g. together with user information characterizing the medical condition of the person. Alternatively or in an additional aspect, the service robot 17 may also determine whether the user needs the signals output by the service robot 17 to have a higher volume based on the volume of responses provided by the user, for example relative to the ambient noise level 1635 recorded by means of at least one additional microphone 193. In an additional and/or alternative aspect, the volume of the output of acoustic signals emitted from the service robot 17 is adjusted accordingly, for example increased if it is determined by at least one of these means described above that the user is hearing-impaired.

As part of a short eye test, the service robot 17 prompts the user to press corresponding fields in the menu on the display 2 if the user can recognize certain letters or other symbols 1650. Alternatively or additionally, speech input is also possible for the user, which in turn is evaluated using natural language processing methods as described in the prior art. The service robot 17 subsequently outputs a sequence of characters or images on the display 2 1655. In step 1660, the user also signals whether or not the user has recognized the character or which character the user has recognized. In one aspect, the characters or images become smaller over time (step 1665). However, another sequence is also conceivable in this respect. In addition and/or as a complement to this, different color patterns are also possible in order to detect possible color blindness of the user. The answers of the user are recorded. The results of the test are displayed in the form of a score 1670. If, for example, the user does not reach certain threshold values for eyesight or color blindness is detected, i.e. a certain number of objects/patterns are not recognized (such as three out of seven), this affects the score. In one aspect, this is stored in a database in the service robot 17 1675. In an additional and/or alternative aspect, the size of the letters when outputting text elements on the display 2 of the service robot 17 is adapted accordingly, as well as the menu design, if necessary, in order to be able to display required menu items with larger letters 1680. Furthermore, in an additional aspect, the color scheme of the display 2 can also be adapted to enable an improved recognition of the display menu in the event of color blindness. In an additional and/or alternative aspect, it is also possible for the service robot 17 to vary the distance to the user, for example to move closer to the user in the case of users with eyesight problems 1695. For this purpose, a parameter value is temporarily modified in the navigation module 101 that defines the usual distance between the user and the service robot 17 1690. As a final measure, the contrast and/or brightness of the display 2 of the service robot 17 can also be adapted to the environmental conditions, taking the eyesight of the user 1685 into account.

### Improvement of Signal Processing Quality Through Adaptation to Environmental Conditions

In a further aspect, independently of the eye and hearing tests, but also taking user impairments into account, the service robot 17 is capable of adapting the input and output units to the environment in such a way that operation is possible with different levels of brightness and/or background noises. For this purpose, the service robot 17 has a commercially available brightness sensor near the display to determine how much light falls on the display 2. At the same time, the brightness value of the display 2 is adapted to the environment, i.e. especially in case of an intense incidence of light, the brightness of the display 2 is increased and in case of low brightness values, the brightness of the display 2 is reduced. Alternatively or additionally, the service robot 17 is capable of detecting background noise through one or more microphones 193. In one aspect, this may result in an increase in the volume of the acoustic output of the service robot 17 when the background noise level is increased and a decrease in volume when the background noise level is low. In an additional or alternative aspect, at least one further microphone 193 records the background noise and uses noise cancellation techniques (phase shifts of the input signal around the recorded background noise) to improve the signal quality of the acoustic input signal in order to enable improved speech processing in order to prevent, for example, data capture errors, the repetition of a question or prompt by the service robot 17, etc.

Furthermore, as a measure to improve the accuracy of the test results, the service robot 17 inquires as to whether the person being evaluated is in pain, also inquiring about the intensity of the pain. For this purpose, the interaction between the service robot 17 and the person to be evaluated takes place via the communication device already described elsewhere. Such information is stored in the user's database record.

As a further measure to improve the accuracy of the test results, the service robot 17 additionally receives information from the patient administration module 160 as to when the patient was admitted to the clinic where the test is being performed and calculates the duration of the previous stay to account for the decline in cognitive performance due to extended stays at an inpatient clinic. At the same time, the patient administration module 160 records whether the patient has already received a diagnosis for a certain disease. This information is also considered when the result of the mini-mental state exam is displayed and stored in the user's database record.

The service robot 17 transmits the results of the stored test tasks described above to the patient administration module 160 via an interface 188 (such as WLAN), thereby making them available to the medical staff while also documenting the results.

### Spectrometric Measurements on the Patient

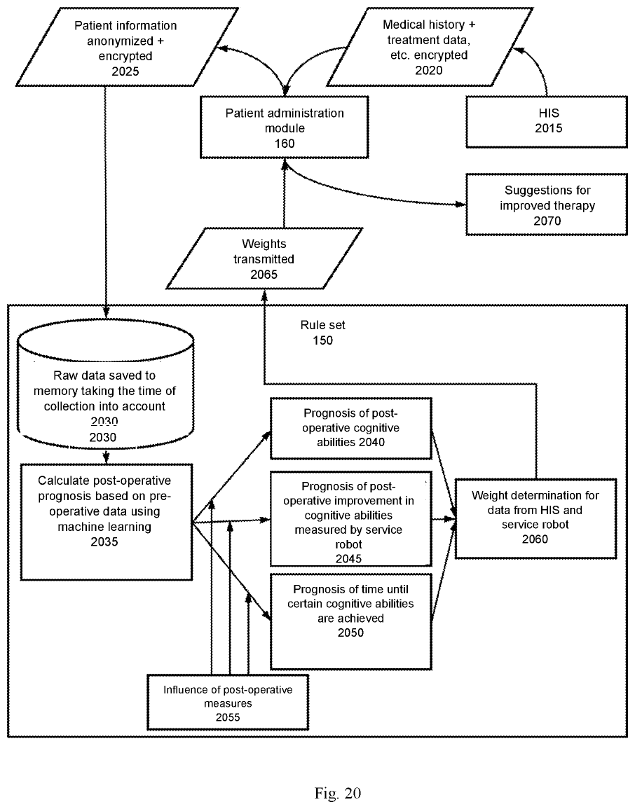

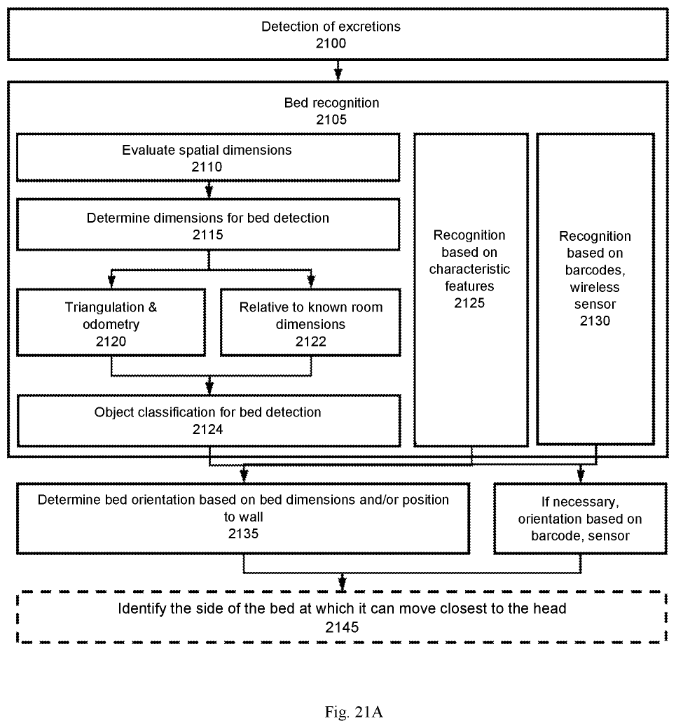

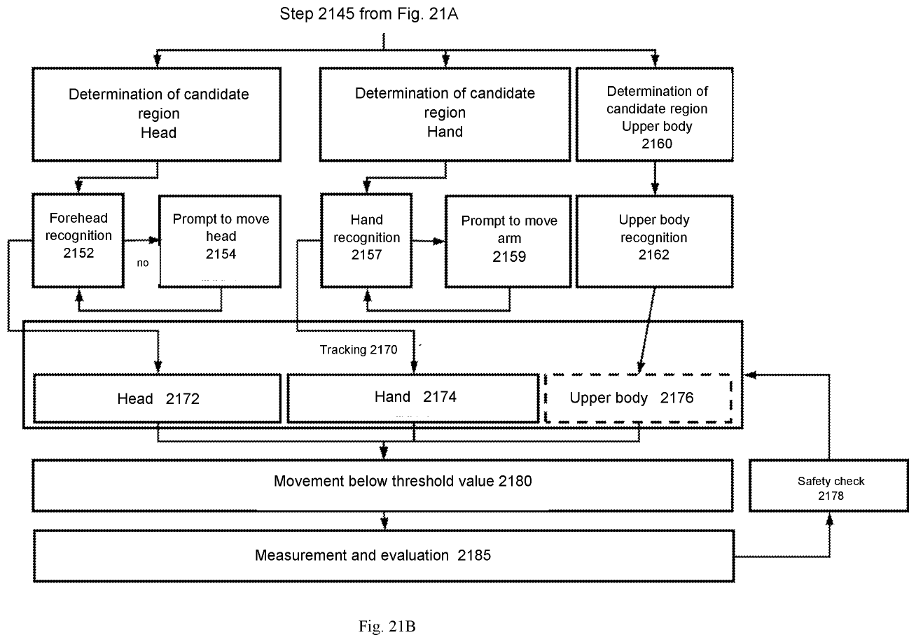

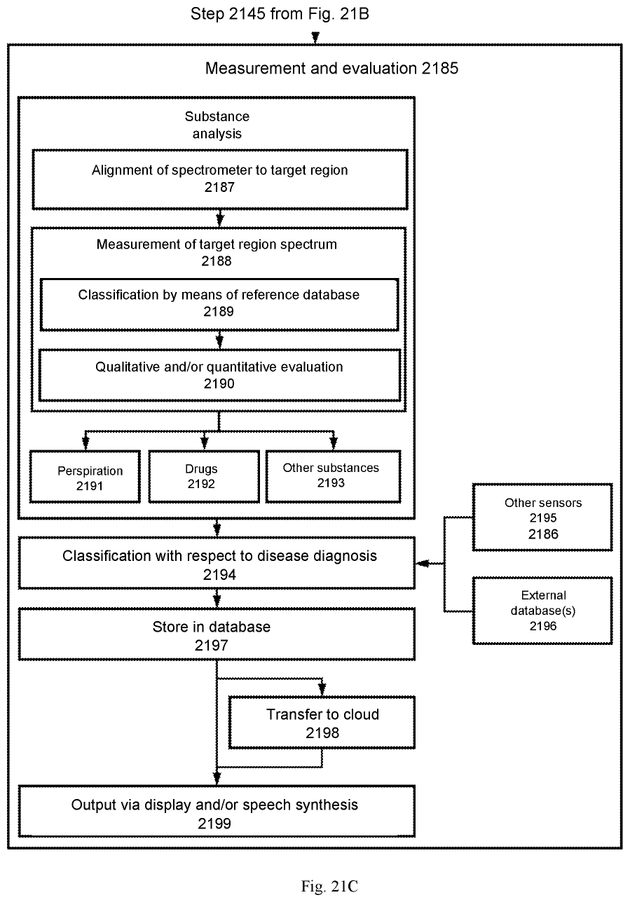

In one aspect, the service robot 17 is configured in such a way that the service robot 17 is capable of determining whether a patient exhibits certain excretions through the skin that, in one aspect, are indicative of certain diseases and can be used to diagnose them. For example, the service robot 17 can determine whether a patient perspires in bed and, if appropriate, how much. For this purpose, a spectrometer 196 may be used, for example, e.g. a near-infrared spectrometer, or in another aspect, a Raman spectrometer. The processes involved in the measurement of excretions 2100 are shown in FIG. 21. In each case, measurements can be made at different locations on the body. By way of example, the procedure is described for three locations: on the hands, on the forehead, and a measurement on the trunk, in particular the bedding. Detection of perspiration at these locations enters into the Delirium Detection Score, for example, which is another test for detecting delirium in patients.

The service robot 17 is configured such that the service robot 17 can record a patient in a bed by means of a 3D sensor, e.g. a 3D camera. For this purpose, this sensor is positioned on the service robot 17, for example, in such a way that the service robot 17 is located at a height of at least 80 cm, e.g. at a minimum height of 1.2 m, and is mounted in such a way that it can be rotated and/or tilted, for example.

The service robot 17 is capable of identifying beds 2105 based on object recognition. For this purpose, the service robot 17 can, in one aspect, use the 2D or 3D sensor, for example the LIDAR 1, to scan the room that is known a priori to have beds within it. Alternatively and/or additionally, dimensions can also be determined by means of a map stored in the memory 10, which contains, for example, spatial information such as the width and depth of a room. The room dimensions are thereby evaluated 2110. The service robot 17 can also determine the dimensions of measured objects 2115, for example by triangulation in conjunction with an implemented odometry unit 181 (step 2120), which can determine the deviations in position of the service robot 17. The dimensions of the measured objects in the room relative to the spatial information are determined 2122, for which the odometry function is not required. The determined dimensions, in particular the external dimensions of the bed, are classified based on rules stored in the memory 10 to determine whether the object is a bed 2124. In one aspect, this includes the different dimensions that a bed may assume. In additional and/or alternative aspects, a classification of objects recognized by the LIDAR 1 and/or the 2D and/or 3D camera 185 may also be based on characteristic features that uniquely identify a bed 2125. This may be the design of the wheels of the bed and/or the lifting apparatus for adjusting the height of the bed. However, classification rules created by learning typical bed features based on machine learning methods and/or neural networks can also be used. Alternatively and/or additionally, the beds may also be equipped with sensors and/or barcodes 2130 that allow bed identification, e.g. RFID or Bluetooth transmitters.

In one aspect, the positioning of the sensors on the bed can be used to determine the orientation of the bed in the room 2140, for example, by using backscatter signals that are reflected differently from the bed frame and can be used to determine the orientation of the bed in the room via the propagation time and/or phase differences. Barcodes can also be attached to the service robot 17 in such a way that reading them allows the spatial orientation of the bed to be determined. The codes stored in the sensors and/or barcodes are read out by the service robot 17 and compared with those stored in the memory 10 of the service robot 17 and assigned to beds, whereby the service robot 17 can determine that the read-out sensor and/or barcode is assigned to a bed. Alternatively and/or additionally, but particularly when a bed has been recognized as such based on its dimensions 2124, the orientation of the bed in the room may be determined via the bed dimensions, and in one aspect also by matching the position to the nearest wall 2135: That is, the service robot 17 determines the orientation of the bed, in particular the head end, based on advance information, with a priori information indicates that a bed has an essentially rectangular shape with shorter sides representing either the head end or the foot end. In this case, the shorter side is recognized as the head end, which is located, for example, closer to a wall of the room. In an alternative aspect, the service robot 17 identifies a person in the bed, in particular their head and arms, which may be evaluated, for example, in the scope of a skeleton model.

Then, in an optional step 2145, the service robot 17 determines where the service robot 17 can navigate relatively close to the patient's head. For this purpose, the service robot 17 then determines how far the service robot 17 can travel to the head end on one side of the bed. If the distance to the wall at the head end is less than 1 m on one side of the bed, the service robot 17 moves along this side of the bed. If the distance is more than 1 m, the service robot 17 determines the distance to the wall on the other side of the bed and then moves along the side of the bed where the service robot 17 can move as far as possible to the wall and continues forward as far as possible to the wall at the head end. In an alternative aspect, the service robot 17 first checks both sides for depth as described above and then travels toward the head end on the side where the service robot 17 can travel the farthest toward the wall at the head end.

Next, the service robot 17 determines a candidate region of the head 2150. For this purpose, the service robot 17 positions itself in such a way that its front faces the direction of the presumed position of the head. This can be done, for example, by rotating the service robot 17 at the position, with the service robot 17 having a rotation angle between 25° and 90° relative to the long side of the bed. The service robot 17 uses a 2D or 3D sensor to detect the surface of the bed, in particular in the area towards the head end. As an alternative and/or additional measure, the service robot 17 calculates a candidate region in which the head is usually located lying in an area at least 25 cm away from each long side of the bed and at least 10 cm away from the head end of the bed up to a distance of 60 cm away from the head end.

Alternatively and/or additionally, intervals for the width can also be stored. If the distance between long sides of the bed (relatively determined from a comparison of the bed sides and/or via predefined length intervals) is less than a defined threshold value (e.g. 20 cm), the bed is positioned longitudinally relative to the wall and the service robot 17 moves along the long side of the bed which offers adequate space. Subsequently, the service robot 17 uses the determination of the candidate region for the head already described, or alternatively, the service robot 17 scans the entire bed by means of the camera 185, the images of which are evaluated via a framework available on the market that has implemented head detection.

Based on features of the head, the service robot 17 is able to detect the forehead 2152. In one aspect, this is achieved by defining an area limited by the following facial features: about 4 cm above the line connecting the centers of the eyes lies the hairline on the sides, which is recognizable through a color contrast with the patient's skin. Alternatively and/or additionally, the shape of the head can also be used for this purpose, with the boundary of the forehead area being limited by the rounding of the head. For this purpose, approaches such as that of histograms of gradients can be used, for example, which are implemented in frameworks such as OpenCV or scikit-image. The angle whose one arm consists of a light beam from the sensor of the head and the perpendicular at the point where the light beam strikes the surface of the head can be used as a boundary here. Once the patient's forehead is identified, the service robot 17 tracks the position of the head 2172, for example by means of the visual person tracking module 112 and/or the laser-based person tracking module 113.

If the service robot 17 has difficulty identifying the patient's forehead or eyes, it may, in one aspect, switch sides of the bed to ensure that the patient has not turned the back of his or her head towards it. Alternatively and/or additionally, the service robot 17 may use its output units, such as the display 2 and/or the speech synthesis unit 133, to prompt the patient to move his or her head 2154 and, for example, to look at the service robot 17. After such a prompt, another attempt is made to identify the head or forehead.

The service robot 17 uses further classification algorithms to recognize the hands of a patient. On the one hand, this can be achieved by means of the same method applied for identifying the patient's head (i.e. two candidate regions for the hand 2157 are determined in the approximately center on the long side of the bed with a depth of approx. 30 cm parallel to the short edge of the bed). Alternatively and/or additionally, on the other hand, algorithms from the SDKs of an RGB or RGB-D camera 185 can be used to create a (proportional) skeleton model of the patient, in which case primarily the arms and hands are recognized, i.e. their skeleton points, while the connections between the skeleton points can be represented as direction vectors. If the service robot 17 does not recognize any arms or hands, the service robot 17 can use its output units, e.g. the display 2 and/or speech synthesis unit 133, to prompt the patient to move his or her hands or arms 2159, e.g. to bring them out from under the blanket. After such a prompt, another attempt is made to identify arms and/or hands. Similarly to the forehead, the service robot 17 also identifies the hand surfaces, i.e. either the back of the hand and/or the palm. Here, for improved localization, skeleton model points may alternatively and/or additionally be included, with the hand area of interest being between the wrist and finger joints. Alternatively and/or additionally, palm recognition can be performed using image classification, with the classification algorithms having been performed by training using images of palms.

Another body region of interest is the patient's upper body, which is predefined via a candidate region as extending downward from the head for a length of approx. 1.5 head heights, starting half a head height below the head, and having a width of approx. 2 head widths. Alternatively and/or additionally, the area is defined over a width of approx. 45 cm and a height of approx. 50 cm, which begins approx. 10 cm below the patient's head, alternatively positioned at approximately the center of the bed at a distance of approx. 50 cm away from the head end. There may alternatively and/or additionally be a classification based on three-dimensional shape. In one aspect, the width of the bed is scanned for the height as well as, in the area of the axis parallel to the long side, the area located in the half of the bed that is oriented towards the head end. Along the ridge line that appears in this zone, the part that is below the candidate region for the head is selected. A candidate region for the upper body 2160 can thereby be determined. A scan of the elevation relative to the mattress level detected by the 3D sensor system of the service robot 17 is then performed and, if an elevation is detected in the candidate region, this region is identified as the upper body 2162.

The service robot 17 is thereby able to detect and identify three target regions of the patient: the forehead, the palm/back of the hand, and the upper part of the trunk. These can be identified in the room by means of the sensor system, e.g. by means of the RGB-D camera 185, so that their surfaces can be displayed accordingly in a three-dimensional coordinate system. Tracking 2170 also takes place, for example, in particular for the head of the patient 2172, and in one variant also for the hand 2174 and optionally for the upper body, e.g. by means of the visual person tracking module 112 and/or the laser-based person tracking module 113. In the process, for example, the images produced by the sensors are segmented in order to define body regions by means of classification, so that the spectrometer (196) can be directed to these body regions. A corresponding classification may be stored in the memory 10. The service robot 17 may, for example, also have regions on which the measurement is to be made which are stored in the application for controlling the spectrometer.

Before the measurement, in step 2170, the service robot 17 tracks the movements of the hand or head (optionally also the upper body) over a defined period of time. If no movement is detected for a period of time exceeding a defined threshold value (e.g. 5 seconds), or if a detected movement of the hand/head does not exceed a defined threshold value (e.g. 5 mm) 2180, a measurement and evaluation of the acquired data 2185 is performed.

During the spectrometer measurement, a safety check 2178 carried out using the RGB-D camera 185 continuously tracks the patient's head or hand on which measurements are being made. If movements are detected, e.g. a rotational movement of the head or a lowering or lifting of the head, which exceeds a defined threshold value, the measurement is immediately interrupted. The service robot 17 continues to track the regions on which a measurement is to be made, and starts a new measurement attempt if the movements of the head do not reach a defined threshold value.

In addition, the service robot 17 has, for example, a near-infrared spectrometer for substance analysis 2186, which is rotatably and pivotably mounted and electronically adjustable for this purpose. The service robot 17 is able to use this mount to align the spectrometer 196 in such a way that the path of the radiation emitted by the spectrometer 196 reaches the coordinates of the target region in three-dimensional space and the reflected radiation is also detected again by the spectrometer 196 2187. Although a possible light source may be an infrared diode with focusing optics, in one aspect an infrared laser is used.

Measurement is performed, i.e. the signals from the spectrometer 196 are evaluated and classified 2189 on the basis of a reference database containing reference spectra, thereby allowing a determination of what is in or on the target region 2188, with the determination being qualitative quantitative 2190. Alternatively and/or additionally, classification rules for determining the substances from the measured spectra which work, for example, on the basis of correlation analyses, can also be stored directly. In one aspect, characteristic signals, i.e. especially spectral profiles of perspiration, can be determined 2191, which are composed of individual spectra of water, sodium, and/or chloride and occur, for example, on the patient's skin, e.g. on the forehead or hand. With regard to the target region of the trunk, the degree of dampness of the patient's blanket is recorded, i.e. the classification used here for signal evaluation considers the material of the bedding accordingly.

In addition, scanning different parts of the blanket allows, for example, the amount of water excreted as perspiration to be estimated by means of classification based on the reference database.

In another aspect, the database with reference spectra includes those that can determine the concentration of additional substances, including various drugs 2192 such as heroin, opiates (such as morphine), amphetamine, methamphetamine, cocaine (including benzoylecgonine, if applicable), 9-tetrahydrocannabinol (THC), as well as additional substances 2193, such as glucose, lactate, uric acid, urea, creatinine, cortisol, etc.

The service robot 17 has another reference database which, on the basis of the combination of various substances and/or their concentration(s), allows the classification of measured values associated with the measured spectra for various diseases 2194. Moreover, both threshold values of concentrations or of the measured substance quantity, the ratio of the substance quantities and/or concentrations relative to each other on the one hand and combinations of these on the other hand are part of this classification. An example is the combination of urea, uric acid, and creatinine, where the concentration of uric acid is greater than 0.02 mmol/l, creatinine 0.04 mmol/l (higher at lower temperatures), and urea >15 mmol/l (at low temperatures) or >100 mmol/l (at higher temperatures). In this classification, the service robot 17 accounts for the ambient temperature by means of a thermometer located in the service robot 17, the season or the outside temperature. For the latter it is equipped with an interface 188 (such as WLAN) to determine the outside temperature at its location via the cloud 18, i.e. the service robot 17 is able to collect further data for improved evaluation, either by means of additional sensors 2195 and/or via interfaces 188 (such as WLAN) to additional databases 2196.

The measurement results are stored in a database 2197 located in the service robot 17 and/or they can be transmitted to a server in the cloud 18 via an interface 188 (such as WLAN) and stored there 2198. They can then be output via a display 2 and/or a speech output 2199, for example via the service robot 17 and/or a terminal to which medical staff has access for evaluation purposes.

FIG. 63 summarizes the spectrometry system (e.g. the service robot 17): The spectrometry system includes a processing unit 9, a memory 10, and a sensor for the contactless detection of a person (e.g. a 2D and/or 3D camera 185, a LIDAR 1, a radar sensor, and/or an ultrasonic sensor 194), a spectrometer 196, and a spectrometer alignment unit 4805 for aligning the spectrometer 196 with a body region of a person, which is similar to a tilting unit. In addition, the system may include a thermometer 4850 for measuring the ambient temperature and/or an interface 188 to a terminal 13. The memory 10 includes a body region detection module 4810 for detecting body regions, a body region tracking module 4815 for tracking body regions before and/or during a spectroscopic measurement on that body region, a spectrometer measurement module 4820 for monitoring, interrupting, and/or continuing a spectrometric measurement based on movements of the body region on which the measurement is being carried out, a visual person tracking module 112, and/or a laser-based person tracking module 113. The system accesses a reference spectra database 4825 and/or a clinical picture database 4830 with stored clinical pictures and associated spectra for matching the measured spectra and determining the measured substances, which are located in the cloud 18 and/or in the memory 10. The memory 10 or cloud 18 further includes a perspiration module 4835 for determining the amount of perspired moisture, a Delirium Detection Score determination module 4840 for determining a Delirium Detection Score, and/or a cognitive ability assessment module 4845 for determining cognitive abilities. In one aspect, the system has a person recognition module 110, a person identification module 111, a tracking module (112, 113), and/or a movement evaluation module 120.

### Delirium Detection and Monitoring Based on Multiple Tests

As an alternative to the mini-mental state exam, test procedures for delirium detection have emerged in clinical diagnosis and are currently in the process of being mapped by medical staff. Delirium is an at least temporary state of mental confusion. The term primarily used to describe the test procedures is CAM-ICU, where CAM stands for “Confusion Assessment Method” and ICU for “Intense Care Unit.” The assessments made by the medical staff address, among other things, attentiveness disorders, which are assessed by means of acoustic and/or visual tests, as well as tests of disorganized thinking that require motor responses.

### Evaluation of Patient Attentiveness Disorders Based on the Recognition of a Sequence of Acoustic Signals

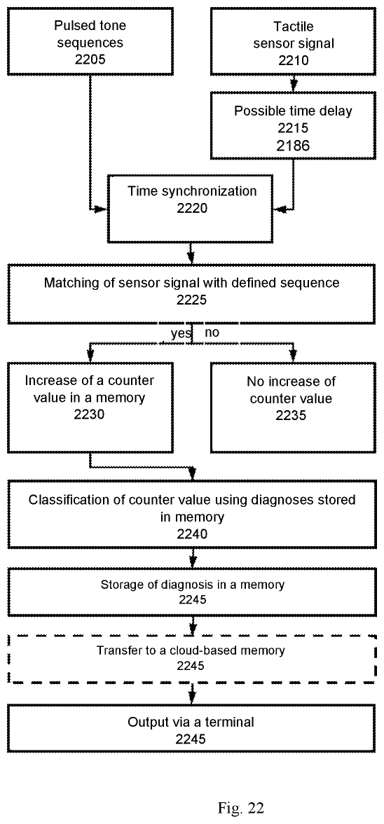

In one aspect, the service robot 17 is configured (see FIG. 22) in such a way that the service robot 17 outputs a pulsed sequence of different acoustic signals 2205 (e.g. a tone sequence) through a loudspeaker 192, e.g. at a pulse frequency of 0.3-3 Hz, e.g. approx. 1 hertz. At the same time, the service robot 17 can capture signals from at least one tactile sensor 4905 (step 2210) and synchronize 2220 them with the output signals. Each sound signal may also be assigned a value in the memory 10. There is a time delay 2215 between the output of the acoustic signals and the detection of the signals from the tactile sensor 4905, i.e. a phase shift by a maximum of half a pulse length, for example, which lags the pulsed signal. In this case, the signals of at least one tactile sensor 4905 registered with the possible phase shift are evaluated to determine whether they occur at a defined acoustic spectrum 2225 stored in the memory 10, i.e. a comparison is performed to determine whether the detected signals occur after a defined tone sequence. If this is the case, a counter in the memory 10 is increased by an integer value 2230, or alternatively no increase takes place 2235. Subsequently, a classification of the determined counter value takes place in such a way that a counter value-assigned diagnosis is assigned to the determined counter values 2240. The tones are output towards a patient to examine, for example, his or her cognitive abilities. The higher the incremented value, the less the patient's cognitive abilities are impaired. The diagnosis is stored in the memory 10 of the service robot 17 2245 or is optionally transmitted to a cloud-based memory in the cloud 18 and optionally made available to medical staff via a terminal.

The tactile sensor 4905 is a piezoelectric, piezoresistive, capacitive, or resistive sensor. However, other sensor types can be used, as described in Kappassov et al. 2015 (DOI: 10.1016/j.robot.2015.07.015). In one aspect, the tactile sensor 4905 is located on an actuator 4920 of the service robot 17 that has at least one joint and can be positioned in such a way that the service robot 17 reaches the patient's hand, i.e. the tactile sensor 4905 is positioned at a distance to the hand that is below a threshold value that, for example, is stored in a memory. In one aspect, the sensor is integrated into a robotic hand. In an alternative or additional aspect, the sensor is mounted on the surface of the service robot 17. In addition, the service robot 17 can use at least one camera 185 to identify and track the patient and determine the position of his or her hands, e.g. at that of the right hand.

### Application Example:

As part of a test to detect a patient's attention, the service robot 17 uses a loudspeaker 192 to output a sequence of letters corresponding to a word. Each letter is output at intervals of approx. one second. The patient is prompted by the service robot 17 to make a pressing movement with his or her hand upon recognizing certain letters. These pressing movements are evaluated by the described tactile sensor 4905 and then a count is made of how often certain letters were recognized. The higher the recognition rate, the lower the patient's impairment.

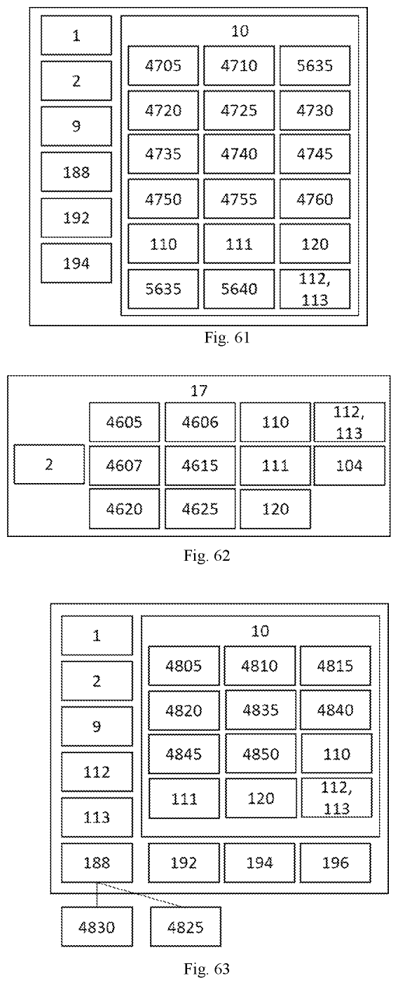

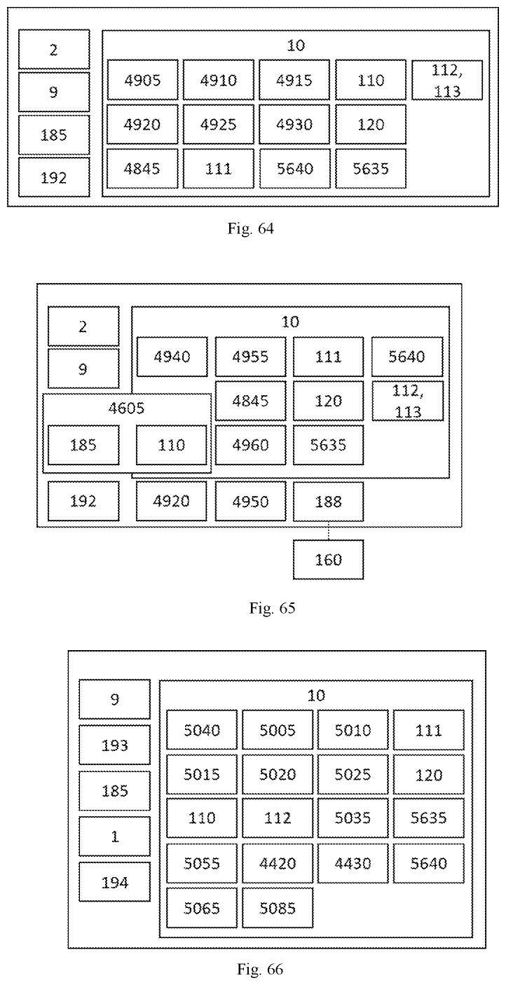

The attention analysis system is summarized as follows, as illustrated in FIG. 64: The system, e.g. a service robot 17, includes a processing unit 9, a memory 10, an output unit for acoustic signals such as a loudspeaker 192, a tactile sensor 4905, a tactile sensor evaluation unit 4910 for evaluating signals from the tactile sensor, and a tactile sensor output comparison module 4915 for performing a comparison of whether the captured signals occur after a defined output. The system may also include an actuator 4920, such as a robotic arm, and a camera 185. The tactile sensor 4905 is positioned, for example, on the actuator 4920. The memory 10 includes an actuator positioning unit 4925 that positions the tactile sensor 4905 adjacent to a person's hand by means of the actuator 4925, a person identification module 111 and/or a hand identification module 4930, and a cognitive ability assessment module 4845 for determining the cognitive abilities of the person. In one aspect, the system has a person recognition module 110, a tracking module (112, 113), a movement evaluation module 120, a skeleton creation module 5635, and/or a skeleton model-based feature extraction module 5640.

### Evaluation of the Cognitive Abilities of a Patient Based on Image Recognition

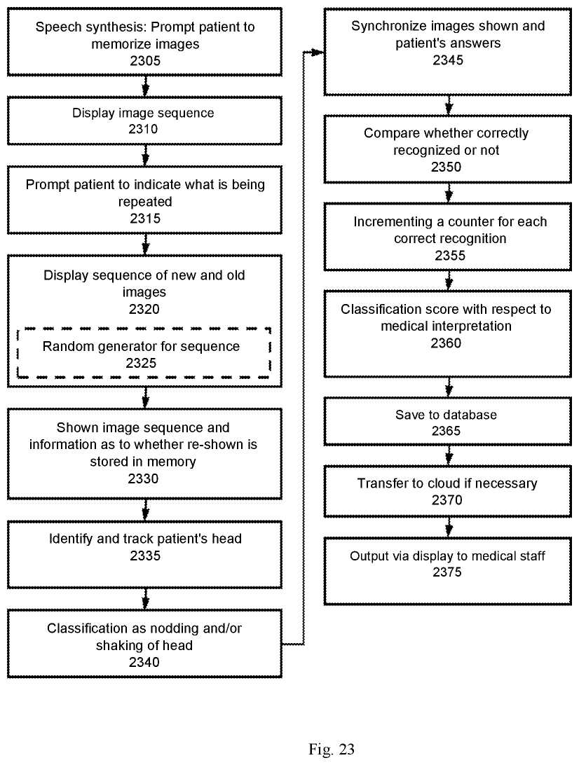

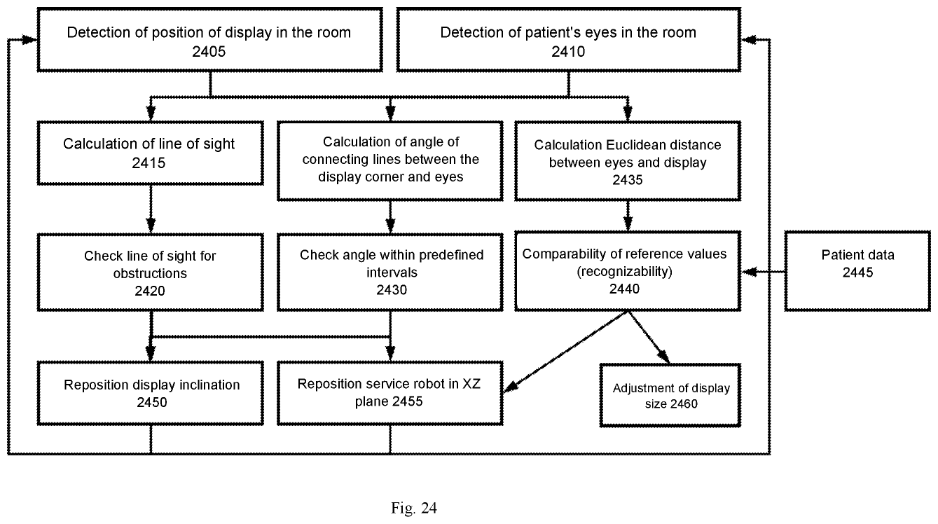

In an alternative or additional aspect, the service robot 17 is configured to evaluate and classify a patient's recognition of images in order to assess the patient's cognitive abilities, in particular his or her attention. FIG. 23 illustrates an example of the sequence used for this purpose. In the process, the service robot 17 indicates to a patient via a speech synthesis unit 133 that the service robot 17 should memorize several images 2305. Following this speech output, a sequence of images is displayed on the monitor of the service robot 17 2310, e.g. five at intervals of three seconds each. Subsequently, the patient is informed via a speech synthesis unit 133 that the service robot 17 should move its head to signal whether these subsequently shown images seem familiar to it, i.e. if the service robot 17 should carry out a classification for these in step 2315. A shake of the head is interpreted as a denial, a nod as a confirmation. Ten images are then displayed on the screen of the service robot 17 (step 2320) at intervals of three seconds. Of these, five are repeated from previous sequence of ten images, e.g. but each image only once. In one aspect, a random generator may be used to sequence the images and/or differentiate them into new image vs. previously shown image 2325. The service robot 17 stores the displayed sequence of images as well as whether or not they have already been shown 2330 and captures the patient's head movements as the images are displayed (or up to one second thereafter). For this purpose, the service robot 17 has at least one sensor, e.g. an RGB-D camera 185, that can recognize and track the head of a patient 2335, with the evaluation being performed, for example, by means of the visual person tracking module 112 and/or the laser-based person tracking module 113. This includes the rotation and/or nodding of the head. Here, the service robot 17 is able to use classification methods to detect distinctive points of the face, including the eyes, the eye sockets, the mouth, and/or the nose. For this purpose, solutions are known in the prior art (e.g. DOI: 10.1007/978-3-642-39402-7_16; 10.1007/s11263-017-0988-8) that employ, among other things, histograms of gradients. The patient's head movements are next classified for recognition of head shaking and/or nodding 2340. Frameworks in the prior art are also used for this purpose. The movements of nodding or head-shaking detected in this way are synchronized 2345 accordingly with the images displayed. Then the displayed image sequence is coded according to whether the patient has recognized an image after it being shown to him or her again or the first time 2350. The service robot 17 optionally stores the comparison of the values, e.g. with the date of execution, in a database in which the image sequences displayed are also stored, for example. For each repeated image correctly recognized by the patient, a counter is incremented 2355. The score generated through incrementation serves as a measure of whether the patient suffers from cognitive impairment. In addition, the determined score is classified and assigned a medical interpretation 2360. The score and its medical interpretation are stored in a database 2365 or optionally stored in the cloud memory in the cloud 18 2370, and are made available to medical staff for evaluation purposes via a terminal 2375.

In an optional aspect, the service robot 17 is capable of determining the position of the patient's eyes in three-dimensional space 2410 as well as the position of the display 2 (step 2405). In one aspect, the service robot 17 uses this data to check the line of sight between the eyes and the display 2 for the presence of obstacles. For example, if a patient is in bed, the guard rails may potentially represent such an obstacle. For this purpose, the service robot 17 first calculates the coordinates lying on the line of sight 2415 and checks, e.g. by means of a 3D camera, whether these coordinates of the line of sight are associated with detected obstacles 2420. If the service robot 17 identifies obstacles, the inclination angle of the display can be readjusted 2450, or alternatively and/or additionally, the service robot 17 can reposition itself in the XY plane 2455. In an alternative and/or additional aspect, the service robot 17 is configured in such a way that the service robot 17 ensures that at least one angle lies within an interval 2430 that may be device-specific by using, for example, the spatial coordinates of the display 2, e.g. the display corners, and determining the angles between the patient's eyes and the display surface (step 2425). In this way, it can be ensured, for example, that a reflective surface of the display 2 does not result in the patient's inability to recognize the display 2 sufficiently because the angle of the display evinces strong reflections from the patient's point of view. For this purpose, the service robot 17 is able to adjust the angle of the display accordingly 2450 and/or to reposition the service robot 17 in the room. Alternatively and/or additionally, the font size and/or other symbols on the display 2 can also be adjusted depending on the distance between the patient and the display 2. For this purpose, the service robot 17 first calculates the Euclidean distance between the eyes and the display 2, compares this distance with reference values stored in the memory 10 of the service robot 17 indicating whether this distance is usually acceptable for recognition, and, in an additional aspect, factors in patient data on visual ability in order to adjust the reference values as required. As a result, the service robot 17 may adjust the display size of the display 2 (i.e. the size of displayed objects and characters) and/or the service robot 17 repositions itself in the room within the XZ plane (i.e. the floor plane) such that the distances are sufficient to recognize the contents of the display.

With respect to the repositioning of the service robot 17 in the XZ plane, the angle of the display 2, and/or the resizing of the display, the service robot 17 is able, through its ability to scan its surroundings and a possibly expanded or alternative viewing clearance that is free of obstructions, the angle of the display 2, and/or the display dimensions of the display 2 to determine what would be a position in the XZ plane, a display angle, and/or a display size that would prevent the patient from encountering obstructions between his or her eyes and the display 2, that would position the display 2 spatially in such a way that it is largely free of reflections, and/or that would guarantee that the display size is sufficient for the patient's eyesight.

In an alternative and/or additional aspect, the service robot 17 has a control for the angle of the display and a dialog function in the display 2 or is configured as a speech interface. This dialog function is used to ask the patient for feedback on whether the display is sufficiently recognizable for him or her. In the event of complaints on the part of the patient, the service robot 17 can change the orientation of the display 2. In one aspect, this can be done by readjusting position of the service robot 17 relative to the patient. In another aspect, this can be done by rotating it in place, and in yet another aspect, it can be done by assuming a different position (e.g. defined over the area covered by the service robot 17 on the floor). In an alternative and/or additional aspect, the angle of the display 2 can be adjusted, whereby the tilting axes can be oriented horizontally and/or vertically.

After repositioning the display 2 and/or the service robot 17 in the XZ plane, this described process is run through again in order to verify that the patient can recognize the display 2 well.

### Robot Counts Number of Fingers on One Hand

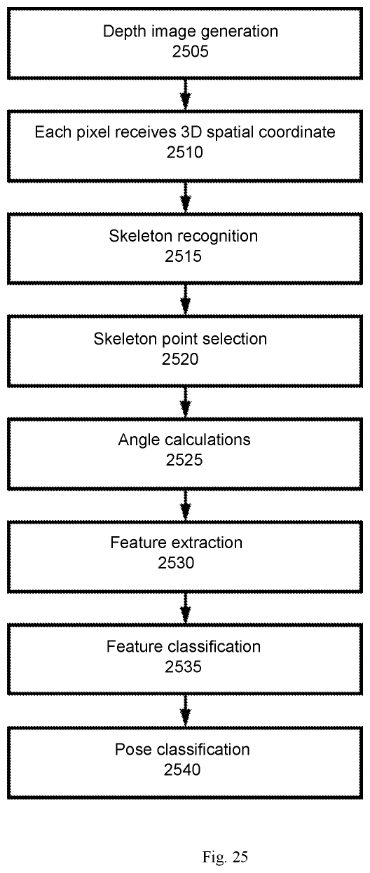

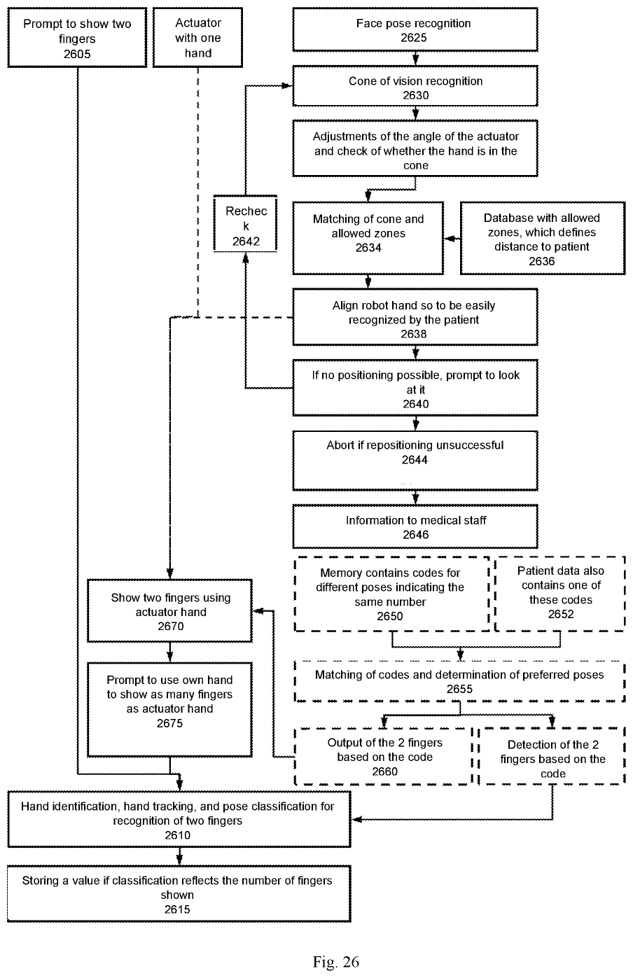

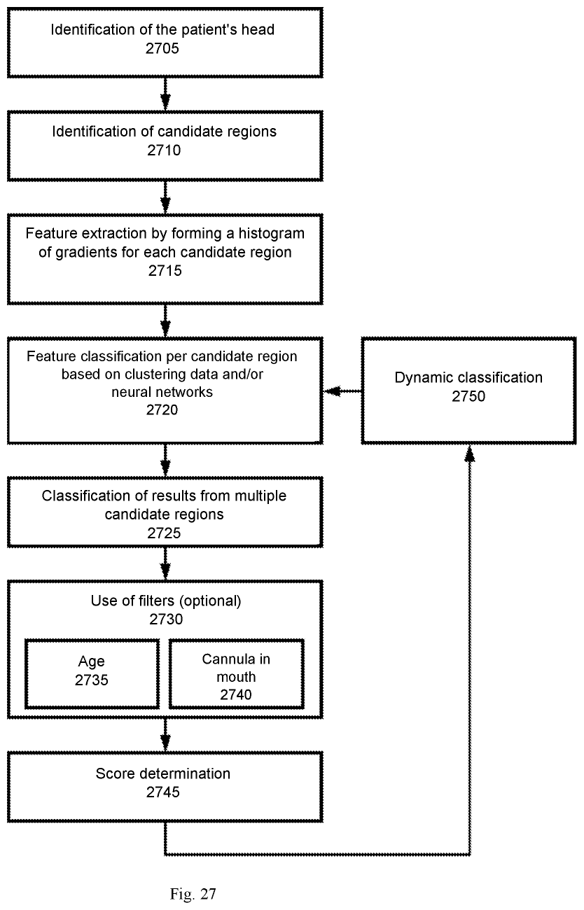

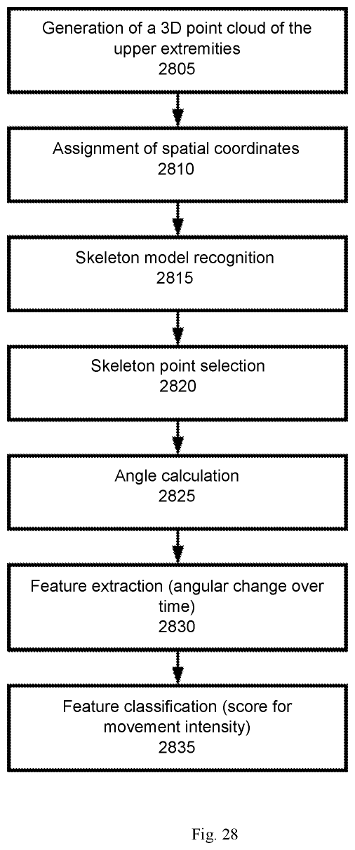

In one aspect, the service robot 17 is configured in such a way that finger identification and tracking can be performed by the camera 185, for example an RGB-D camera 185, in order to evaluate hand poses with respect to indicated numbers, for example using the visual person tracking module 112 and/or the laser-based person tracking module 113. FIG. 25 illustrates this process. For this purpose, the depth image 2505 generated by the 3D depth camera is transformed into a 3D point cloud in which each pixel of the camera is assigned a spatial coordinate 2510. On this basis, skeleton recognition 2515 is carried out using camera SDKs or third-party software, such as NUITrack. The skeleton points are recognized accordingly, including the wrist and finger joints.

Next, a joint selection 2520 takes place, i.e. only the skeleton points necessary for the calculations to be subsequently performed continue to be processed. Angle calculations 2525 are subsequently performed, for example for the angle between the third and the second phalanx, the second and the first phalanx, and the first phalanx and the metacarpal bone. (In this context, the third phalanx is generally referred to as the phalanx with the fingertip.) Since the thumb does not have a second phalanx, here it is the angle between the third and first phalanges, the first phalanx and the metacarpal bone, and, in one aspect, between the metacarpal bone and the carpal bone. Here, each phalanx or hand bone is represented as a direction vector, each from the skeleton point under consideration. Next, feature extraction 2530 is carried out, in which, for example, the angles of the above-mentioned skeleton points per finger are evaluated together. In the scope of a feature classification 2535 implemented based on defined rules, an extended index finger, for example, is defined as an angle of 180° between the first and second as well as second and third phalanges. As part of the feature classification process, threshold values can be defined that soften the 180° condition somewhat, e.g. defining an angle between 150° and 180° for the angle between the third and second phalanx, an angle between 120° and 180° for the angle between the first and second phalanx, and an angle between 90° and 180° for the angle between the metacarpal bone and first phalanx. For the thumb, it is the angle between the third and first phalanx that lies between 120° and 180°, while the angle between the first phalanx and the metacarpal bone is between 150° and 180°. In the scope of pose classification 2540, different fingers and their joint angles are considered in combination. Thus, on the one hand, manually defined values 2545 can be used to detect the value 2, which is shown using the fingers by extending the thumb and the index finger, the index finger and the middle finger, or combinations between these and/or other fingers, two of which are extended, while the other fingers, in particular between the second and third phalanx, have an angle smaller than 120°, e.g. smaller than 90°. If two other fingers are extended instead of the thumb, the angle between the third and first phalanx of the thumb is less than 120°, e.g. less than 100°, to finally be recognized as 2. Optionally, the angle between the hand bones is less than 145°, e.g. less than 120°.

Feature extraction, feature classification, and hand pose classification can be performed on the one hand by means of predefined rules, such as angle definitions of individual skeleton points and their combination, or trained by means of machine learning approaches 2550, such as support vector models, in which certain angle combinations are labeled accordingly, i.e. the combination of angles of individual phalanges to each other may indicate that, for example, two extended fingers correspond to the value 2.

In one aspect, as part of the testing of the patient's cognitive abilities, an output from the service robot 17 is initially triggered, which is output via the speech synthesis unit 133 via a loudspeaker 192 and/or via text and/or images on the screen of the service robot 17. This speech output prompts the patient to show two fingers 2605. Then the camera 185 identifies the patient's hands and the fingers thereof, and tracks the finger movements. In doing so, the service robot 17 evaluates them in the scope of pose classification to determine how many fingers are displayed 2610. In an optional aspect, as described below, consideration can be given to whether the finger pose displayed by the patient is associated with a code 2665. The service robot 17 subsequently stores a value indicating whether the patient has shown two fingers 2615, i.e. an assessment is made of the comparison of the assessed finger poses with the visually and/or acoustically output numerical values.

In an alternative and/or additional aspect, the service robot 17 has at least one actuator 4920, e.g. a robotic arm with at least one joint, which also has at least one robotic hand 2620 with at least two fingers, which are modeled on human fingers, but it may, for example, have at least five fingers, one of which corresponds to a thumb in terms of its placement and has, for example, as many phalanges as the human hand. In addition, the service robot 17 is capable of indicating numbers by means of these fingers, with extended fingers and finger poses resulting from the angles of the phalanges, which have already been classified above with respect to the recognition of phalanges by the camera 185. The service robot 17 is thereby also able to display the value 2 2670 by, for example, extending the thumb and the index finger on the robot hand, i.e. the angles between the first three phalanges are approximately 180°, for example, while the angles of the other phalanges and the phalanges and the hand bones are less than 120°. The service robot 17 is configured in such a way that the service robot 17 can use the speech output and/or display 2 to synchronize the regulation of the poses of the robotic hand such that the value 2 is displayed by the robotic hand while prompting, via display 2 and/or speech synthesis unit 133, the patient to display as many of the fingers as the robotic hand displays 2675. Hand identification, hand tracking, and pose classification are then carried out as described above in order to recognize two of the fingers 2610 on the patient in order to store a value upon determining that the patient indicated the number two 2615. In one aspect, the hand poses displayed by the patient are evaluated, for example, within time frames of 3 seconds after the service robot 17 has prompted the patient via its output units such as the loudspeaker 192 and/or display 2 to indicate a numerical value or has been shown the corresponding numbers by means of the robotic hand.

The knowledge gained in the course of this test allows an assessment of the extent of patient impairment through disorganized thinking and thereby constitutes a test procedure that can be applied for the recognition and monitoring of delirium.

In an optional alternative and/or additional aspect, the service robot 17 is configured in such a way that allows the use of fingers to indicate numbers to be based on cultural and/or national differences. In an optional alternative and/or additional aspect, a recognition of displayed numbers may be facilitated by the service robot 17 taking these differences into account when evaluating the hand poses. Possible results, for example, are that the number 2 is more likely to be displayed by the thumb and index finger among patients from Germany, while those from the USA use the index finger and middle finger to display the number 2. To implement this measure, the service robot 17 has 10 codes in its memory for different poses that indicate the same number 2650 based accordingly on country-specific/cultural differences. The patient data that the service robot 17 has in its memory 10 may also include one of these codes 2652, which indicates the national/cultural background of the patient accordingly. This means that multiple poses are stored in the memory 10 for each number, and in particular multiple finger combinations. Next, a matching of the codes takes place to determine the poses preferred by the patient 2655. Particularly regarding a possible cognitive impairment of the patient, displaying the numbers to the patient using the hand and/or finger pose familiar to him or her increases the reliability of the test. The service robot 17 is thereby able to show the patient the hand and/or finger pose, for example for the number 2, which corresponds to his or her cultural/national background 2660, which is implemented by the robot hand of the actuator 4920 2670. Alternatively and/or additionally, this information about the patient's correctly encoded cultural/national background can be used to better recognize the two fingers shown by the patient. This finger output and/or recognition in consideration of such codes is an optional implementation.

The service robot 17 is further configured to use the actuator 4920 with at least one joint to spatially orient the robotic hand in such a way that the robotic hand can be recognized by the patient in step 2638. For this purpose, the service robot 17 detects the patient's head and its orientation in the room by using established facial pose recognition methods in the prior art 2625, such as those included in the OpenPose framework. In one aspect, approaches such as that of histograms of gradients can be used, for example, which are implemented in frameworks such as OpenCV or scikit-image. Using these frameworks, the service robot 17 determines the orientation of the head in the room and calculates a field of vision for the eyes. In particular, this is understood to correspond in each case to a cone oriented perpendicular to the front of the head with an opening angle of 45°, e.g. 30° (measured from the perpendicular)—which is hereinafter referred to as “good recognizability”. The service robot 17 therefore has a cone of vision recognition function 2630. The service robot 17 also detects its position in the room and the position of the actuator 4920, in particular the position of the robot hand, and determines whether these positions are within the cone 2632. If these positions are not within the cone, the service robot 17 calculates which angular settings of the joints of the actuator 4920 are necessary to position the robot hand within the cone. For this purpose, the service robot 17 calculates a three-dimensional area within the room that has a minimum distance from the patient and that varies, for example, depending on the area of the patient's body. Such minimum distances are stored in the memory 10 of the service robot 17 in step 2636. By identifying the patient in bed, i.e. his or her head and body, an “allowed zone” is calculated in which the robot hand is allowed to move, with the distance to the patient's head being further than, for example, the distance to the trunk or arms. In one aspect, the distance to the head is 50 cm, and that to the rest of the patient's body is 30 cm. In step 2638, the service robot 17 therefore determines the part of the allowed zone where the service robot 17 can position the robot hand so that this robot hand is within the two cones. Then, in step 2638, the service robot 17 aligns the robot hand by means of the actuator 4920 so as to allow the “good recognizability” of the hand by the patient. If such positioning is not possible, the service robot 17 uses the loudspeaker 192 in step 2640 to prompt the patient to look at the service robot 17 via the output units, such as the display 2 and/or the speech synthesis unit 133. Then a new check is carried out in step 2642, i.e. steps 2630-2640 are run through. If the orientation of the determined cones does not change, the service robot 17 terminates the test in step 2644 and transmits information to medical staff in step 2646, for which purpose, for example, information is sent to the server and/or a mobile terminal via an interface 188 (such as WLAN). Alternatively and/or additionally, the service robot 17 may prompt the patient again and/or wait a little longer. If the robot hand is oriented in such a way that the patient can easily recognize it, two fingers are shown with this hand in step 2670 and the process continues as described above. This orientation of the robotic hand based on facial pose recognition and the consideration of an “allowed zone” represents an optional aspect.

The system for cognitive analysis is illustrated in FIG. 65. The system, e.g. a service robot 17, comprises a processing unit 9, a memory 10, an output unit, a numerical value output module 4940 in the memory for outputting numerical values, and a person detection and tracking unit (4605) with a camera (185) and a person recognition module (110). The output unit is a sound generator such as, for example, a loudspeaker 192, a display 2, and/or an actuator 4920, e.g. a robot arm, in one aspect including a robot hand 4950. The system memory 10 includes a hand pose detection module 4960 for detecting the hand poses of the person, a finger pose generation module 4955 for generating finger poses of the robotic hand (4950), with these finger poses representing, for example, numerical values. The system further includes a cognitive ability assessment module 4845 for assessing the cognitive abilities of the captured person. In one aspect, the system is connected to a patient administration module 160. The system has rules for determining the cognitive abilities of the captured person, which have been described elsewhere. In one aspect, the system has a person identification module 111, a tracking module (112, 113), a movement evaluation module 120, a skeleton creation module 5635, and/or a skeleton model-based feature extraction module 5640.

### Pain Status Determination

In one aspect, the service robot 17 is configured in such a way that the service robot 17 is capable of performing a test of the patient's sensation of pain. This is implemented by way of a behavioral observation of the patient by the service robot 17. The procedure for this is based on the Behavioral Pain Scale, an established scale for pain assessment used in medicine. Tests of this kind are also carried out as part of delirium monitoring. In the first step, the facial expression of the patient is analyzed while lying in a bed. Approaches were illustrated above in the description of this invention (e.g. FIG. 21 a) which enable the service robot 17 to identify and, if applicable, track the face of patients in a bed, including the required navigation of the service robot 17. In one aspect, a bed is detected by sensors and the image thereby generated is evaluated by means of pattern matching to assess whether it is indeed a bed. In one aspect, these approaches can also be used here.

### Pain Status: Emotion Recognition

In an initial part of the test, the service robot 17 evaluates the patient's emotions based on his or her facial expression. To do this, in one aspect, the service robot 17 can make use of a facial classification database that stores classification rules for classifications within a candidate region of the face and across multiple candidate regions of the face that allow inferences to be made about the patient's emotional state based on facial features, as described in more detail below. This two-step procedure deviates in this respect from the prior art, which is described, e.g. in US20170011258 or US2019012599, as a one-step procedure. As part of this implementation, histograms of gradients are used, which are implemented in frameworks such as OpenCV or scikit-image. Emotion recognition is mainly focused on emotions that can be used as a measure of the patient's tension level, which can range from a relaxed state to one of high tension, which is expressed through grimaces.

This procedure first involves identifying the patient's head in step 2705, e.g. using frameworks such as OpenPose. The necessary evaluations can be made by means of the 2D or 3D camera 185. For this purpose, candidate regions within the face are first identified 2710, for example, before the feature extraction necessary for the evaluation of the emotional state is carried out in step 2715 based on histogram-of-gradients algorithms in at least one candidate region, which allow, for example, the movements of the mouth or eyebrows to be assessed. In step 2720, based on the data collected from histograms of gradients, a feature classification is carried out using an existing feature classification, which was created by means of established clustering methods, such as k-means or support vector machines and/or based on weights collected by training a neural network, e.g. a multi-layer convolutional neural network with backpropagation with the aid of a labeling of facial expressions. In the next step, i.e. step 2725, the classifications made on the candidate region level are classified over several candidate regions. This is also achieved by means of classification based on established clustering methods of machine learning, such as k-means, support vector machines, and/or the convolutional neural networks mentioned above. For this purpose, movements of the mouth and eyebrows are evaluated together, for example.

In step 2730, the recognition algorithms can be filtered in various aspects, i.e. corrected, for example, for the age of the patient in step 2735, which can be obtained by the service robot 17 from a database via an interface 188 (such as WLAN), provided that the evaluation of emotions is carried out directly by the service robot 17. Alternatively and/or additionally, the images that the camera 185 records of the patient's head for the purpose of the recognition of emotions can also be transmitted to the cloud 18 via an interface 188 (such as WLAN) and analyzed there. In that case, any age information would be transferred from the cloud memory in the cloud 18 to the module processing the emotions. In one aspect, another filter is based on whether the patient has an endotracheal cannula (step 2740) used for the artificial ventilation of the patient through the mouth. The classification algorithms used for the described assessment of emotions have been created in one aspect, for example, based on training data with images of corresponding patients ventilated by means of an endotracheal cannula. Further details on cannula detection are provided below and can also be used, in one aspect, as part of the procedure described here.

As part of a score determination carried out in step 2745, emotions are assessed on a scale of 1-4, for which purpose detected emotions are compared to those stored in the memory 10 and assigned scale values. A value of “1” refers to a facial expression classified as normal, with the tension increasing on the scale up to a value of 4, which implies grimacing. For this purpose, a matrix is available for classification across candidate regions that assigns different facial expressions with corresponding scores.

In one aspect, the values are recorded over the course of several hours or days, which may simplify the evaluation of the patient's emotional state, for example, if a patient is in a relaxed state at the beginning of the series of emotion measurements performed by the service robot 17, which can be stored, for example, by medical staff via a terminal and a menu configuration in the memory 10 to which the service robot 17 has access. This includes information on the patient's general health, including, for example, the information that the patient is pain-free at the start of the measurement. This makes it possible to record and evaluate facial features and emotions in a pain-free state and possibly also in a state of pain, whereby the classification features of the pain-free state can be used to evaluate the state of pain and act as a filter. This dynamic classification (step 2750) of the facial expressions increases the quality of classification, as the classifications become possible based on considerations of differences in facial expression at multiple points in time. For example, a retrospective classification can also be thereby performed, where, for example, only the extracted features together with a time stamp characterizing the acquisition time are stored and reclassified. To realize this, the records of the face are stored. In summary, the resulting steps are the capture of the person, the facial recognition of the person, a selection of candidate regions within the face, a feature extraction of the surface curvatures of the candidate regions, and a classification of the surface curvatures of the candidate regions carried out individually and/or contiguously, with the classification describing a pain status.

### Pain Status: Detection of Movement of the Upper Extremities

A second part of the test focuses on movements of the upper extremities such as the upper arm, the forearm, the hand, and the fingers. For this purpose, the service robot 17 tracks these extremities over time, which the service robot 17 has recognized as described above, achieved either by means of the 2D camera and frameworks such as OpenPose or a 3D camera (possibly an RGB-D camera 185), with the evaluation being done, for example, by means of the visual person tracking module 112 and/or the laser-based person tracking module 113. In the case of the RGB-D camera 185, the sequence is such that the 3D image is converted into a point cloud in step 2805, a spatial coordinate is assigned to each point in step 2810, and skeleton model recognition is carried out in step 2015 by means of camera frameworks or other software tools in the prior art in which the skeleton points are recognized. Subsequently, a joint selection is performed in step 2820, i.e. the recognition here especially targets skeleton points such as the shoulder joint, the elbow joint, the wrist, and the finger joints. An angle calculation of these skeleton points takes place in step 2825, where the angle is defined, for example, by the direction vectors that take the skeleton point as a starting point. In a feature extraction performed in step 2830, the angles of these limbs are recorded over time. The limbs are then classified in such a way that the number of angular changes per time unit, the speed, i.e., for example, the angular velocity, etc., is used as a measure of the movement intensity. The service robot 17 classifies these movements in step 2835 using a scale of 1 to 4 and stores this value. A value of 1 means no movement within the tracked time. A value of 2 means few and/or slow movements of the arms, 3 means movements of the fingers, and 4 means a high movement intensity of the fingers, which are defined, for example, by the number of finger movements per time unit and/or their speed, which are threshold-dependent values.

### Pain Status: Pain Localization

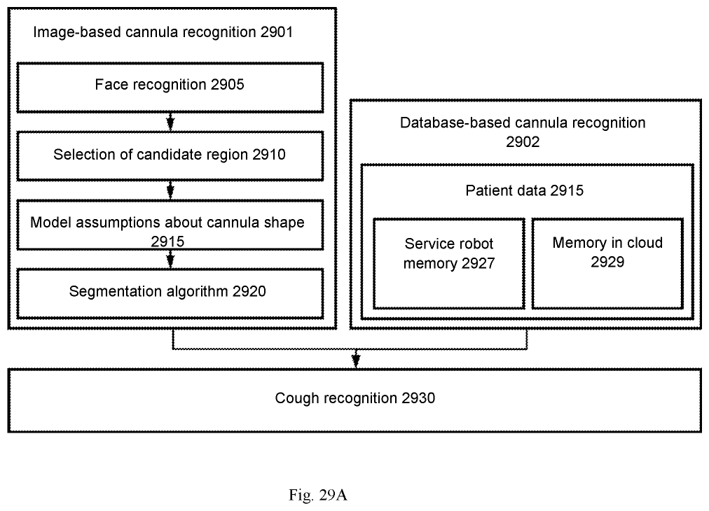

A third part of the test focuses on the patient's pain vocalization and can be carried out in two principally different sequences, which are based on two different scenarios and expressed in two assessment variants. In the first scenario, the patient is artificially ventilated, and vocalization is assessed based on coughing. In the second scenario, the patient is not ventilated, and typical pain sounds are assessed. FIG. 29 describes the procedure for this in greater detail.

### Pain Status: Pain Vocalization in Ventilated Patients

In the first scenario, the patients are ventilated. They can either have a tracheal cannula, which ensures ventilation through an opening in the throat, or an endotracheal cannula, which allows ventilation through the mouth. Image recognition algorithms allow the service robot 17 to identify such ventilated patients in step 2901. For this purpose, the head and neck regions of the patient are recorded either as a 2D or as a 3D image. These serve as candidate regions in the first step, the neck serving as a candidate region in the case of a tracheal cannula, and the mouth serving as a candidate region in the case of an endotracheal cannula. The recognition of the candidate regions is performed, for example, in conjunction with histogram-of-gradient (HoG)-based face recognition in step 2905 and the candidate regions derived from this, such as the mouth and neck, in step 2910. Both regions are evaluated accordingly. Model assumptions may be used in the process, i.e. the typical shape of a cannula (step 2915). The pixels captured by the camera 185 are then evaluated by an optionally real-time-capable and fault-tolerant segmentation algorithm 2920 to recognize such a cannula. The service robot 17 is thereby able to detect the cannula.

Alternatively and/or additionally, database-based recognition can be performed in step 2902, in which the service robot 17 queries information about the patient's ventilation in step 2925 via an interface 188 (such as WLAN) from a cloud-based database of patient information in the cloud 18 in step 2927 and/or the information is located, along with other patient data, in the memory 10 of the service robot 17 (step 2929).

For these two cases of artificial respiration, the service robot 17 determines the extent to which the patient is breathing normally or even coughing 2930. This can be determined in a number of ways. In one scenario, the service robot 17 uses data from a ventilator and/or adapter located between the cannula and the ventilator in step 2935. In one aspect, in step 2936, the service robot 17 accesses the ventilator in use via an interface 188 (such as WLAN) and acquires the evaluation curves of the ventilation cycle generated by the ventilator and captured by pressure and/or flow sensors. In step 2941, the recorded curves are compared either with threshold values that are typical for different ventilation scenarios, such as pressure- or volume-controlled ventilation, and those that occur in these ventilation scenarios in the case of coughing. Alternatively and/or additionally, labeling can also be performed in the cases, e.g. by medical staff, to recognize atypical ventilation patterns, such as coughing, which is then classified by machine learning and/or neural network algorithms as coughing in step 2942. Alternatively and/or additionally, the curves (pressure, volume, flow) can be evaluated over time and anomalies can be detected over time, i.e. deviations that occur before in the breathing cycle and no longer occur afterwards can be classified as coughing by means of machine learning and/or neural network methods. In doing so, not only can an anomaly be compared directly with the cycle before and after in step 2942, but also chains of multiple cycles can be compared to appropriately detect, for example, coughs that involve multiple cough events. In cases where the ventilator even supports coughing by adapting the ventilation, the corresponding modes of the ventilator, or alternatively the ventilation curves derived from these (pressure/flow over time) can also be recognized by the service robot 17 and taken into account accordingly in the classification of the patient's ventilation in step 2944. Alternatively and/or additionally, the service robot 17 may receive information from the ventilator that it is in cough assist mode or is triggering a cough, which would allow the system to detect a cough event. Alternatively and/or additionally, instead of accessing the evaluation of the ventilator via an interface 188 (such as WLAN), an adapter can also be accessed in step 2937 that measures the pressure and/or flow in the supply tube between the cannula and the ventilator by means of a pressure and/or flow sensor and transmits the signals, for example wirelessly, via an interface 188 to the service robot 17, which then generates corresponding evaluations of the ventilation profile, which can be evaluated as described above.

Alternatively and/or additionally, coughing can also be detected by means of at least one sensor located, for example, on the patient's body 2950. Possible sensors include, on the one hand, inertial sensors 2952 (e.g. with a magnetometer) used, for example, in the region of the chest, neck, or cheeks, strain sensors 2954 (e.g. strain gauges applied to the patient's skin at the same locations), contact microphones 193 (step 2956) (which are also applied to the patient's skin and, in one aspect, detect coughing sounds at spots where a bone is located directly under the skin surface), and a thermistor 2958 located, for example, on or in the nose. Each of these is wirelessly connected to the service robot 17 via an interface 188 (such as WLAN). This configuration allows both a direct connection to these sensors and access to the data generated by the sensors and stored in the memory 10, for example a hospital information system. In this case, this data may have already been evaluated regarding cough signals or the evaluation is carried out within the service robot 17.

Drugman et al. 2013, “Objective Study of Sensor Relevance for Automatic Cough Detection, IEEE Journal of Biomedical and Health Informatics”, Vol. 17 (3), May 2013, pages 699-707 (DOI: 10.1109/JBHI.2013.2239303) has shown that, compared to the sensors used in the previous paragraph, the detection of coughing by means of a microphone 193 works best, although the consideration limited itself to non-intubated patients, whereas in the case under consideration here, the patients have either a tracheal cannula or an endotracheal cannula. For this reason, in one aspect, at least one microphone 193 (step 2960) is used that is located on the patient and/or at another position in the room of the patient and that is directly connected to the service robot 17 (or indirectly connected to the service robot 17 with data storage (with storage taking place in one variant already in consideration of data evaluated for cough signals) in the memory 10 to which the service robot 17 has access). This is at least one microphone 193, for example, that is integrated in the service robot 17 2962, records the sounds in the environment of the patient in the memory 10 of the service robot 17, and classifies the sound signals as to whether a cough occurs. Machine learning algorithms and/or neural networks that have been trained by means of recorded coughing sounds are used for this purpose, for example.

For the creation of such a classification, a system is trained in one aspect that has at least one processor for processing the audio data and at least one audio data memory containing audio data, in one aspect also available in the form of spectral data, which is labeled accordingly.

In an alternative and/or additional aspect, the 3D sensor of the service robot 17, e.g. the 3D camera, detects movements around the mouth, but also of the chest and/or neck, i.e. a fault-tolerant segmentation algorithm 2976 is used to evaluate said candidate regions 2974. It has already been described elsewhere how the mouth can be detected. With regard to the detection of the chest and abdominal region, the candidate region 2974 is determined, for example, using the distance of the shoulder skeleton points across the skeleton model, and the same distance is determined orthogonally to this line in the direction of the feet in order to identify the trunk candidate region under the blanket, which consists of the chest and abdomen, both of which move in an evaluable way during breathing. Alternatively and/or additionally, the candidate region 2974 can also be determined, for example, as twice the head height extending from the chin downwards and approx. 2.5 times the head width. In both cases, the identification of the head can be used as initial step and thus as reference 2972 to identify the candidate regions from there. Alternatively and/or additionally, dimensions of the bed can also be used as a reference 2972 to identify these candidate regions 2974. Alternatively and/or additionally, the 3D camera may also capture the elevations on the surface of the bed, with a histogram-of-gradients assessment carried out in one aspect, with the assessment based on a classification trained by a system that includes 2D or 3D images of beds as inputs which are labeled as to whether patients are in the beds or not and which have been evaluated by means of classification methods from machine learning and/or neural networks, while the evaluation results form the classification in order to detect, in particular, the upper body of a patient.

As part of the feature extraction process carried out in step 2978, the movements of the mouth, cheeks, neck, and, upper body detected by the 3D camera are evaluated over time. Here, following Martinez et al. 2017, “Breathing Rate Monitoring during Sleep from a Depth Camera under Real-Life Conditions”, 2017 IEEE Winter Conference on Applications of Computer Vision (WACV), Mar. 24-31, 2017 (DOI: 10.1109/WACV.2017.135), interference reduction 2980 is carried out with respect to the detection of movements obscured by fabric or the blanket, i.e. primarily movements of the upper body/abdomen. Interference caused by different phases of the detected movements of the blanket, which complicates the detection of the actual respiratory movements, is eliminated by determining the power density spectrum, which facilitates the detection of the movements of the chest. In this process, the power density spectrum is determined for each pixel acquired over time in three-dimensional space using, for example, a fast Fourier transform (FFT) in step 2982, then the power density spectra for all pixels are aggregated in step 2984 and the maximum is determined via quadratic interpolation in step 2986, with the position of the maximum indicating the respiratory rate in step 2988. In step 2990, the respiratory rate is then monitored with respect to rate changes that indicate coughing. Then, in step 2990, a frequency determination of the detected body parts is performed 2990. Histogram-of-gradient calculations are used in step 2990 for this purpose, for example. The subsequent feature classification carried out in step 2992 is based on a classification generated by recording coughing movements and non-coughing movements, for which standard classification methods and/or neural networks may be used, as already described elsewhere in this document. If no cough is detected by means of one of the approaches described, this criterion is rated with a score of 1. If cough is detected, the score is rated as 2.

The sequence can be summarized as follows: detection of the person, recognition of the person's face and neck, evaluation of the face and neck region of the person for patterns describing an artificial ventilation device, and the storing of a value upon detection of a pattern describing an artificial ventilation device, with the artificial ventilation device describing a pain status.

### Pain Status: Pain Vocalization in Non-Ventilated Patients

If the service robot 17 fails to detect a cannula in the patient by means of the implemented image recognition and/or no information on artificial ventilation is stored in the database with patient information, another variant of the third part of the test is executed, with the service robot 17 analyzing sounds emitted by the patient by means of a microphone 193. These sounds are classified using algorithms that have been trained based on labeled sound data by means of machine learning algorithms and/or neural networks in such a way that the sounds allow the recognition of pain vocalization in various degrees. If no pain vocalization is detected, this criterion is given a value of 1. If a pain vocalization is detected over a duration of less than 3 seconds and with a frequency of less than three pain vocalizations per minute, this criterion is assessed as 2. A higher frequency or longer duration is assessed as 3. If, for example, verbal pain vocalizations, which in one aspect can also be determined by means of a dialog with the patient, are registered, the criterion is given a rating of 4.

In a final step, the scores over the three parts of the test are added together. The results are stored in a database, or, in one variant, transferred to a server in the cloud 18 via an interface 188 (such as WLAN) and stored there. In both variants, the medical staff has access to the evaluation, which also allows a detailed view of the test results (partial results and overall result), and a terminal can be used to visually display this data. In an alternative and/or additional aspect, the individual parts of the test can also be performed independently.

The procedure for pain status determination based on pain vocalization in non-ventilated patients can be summarized by the following steps: recording of acoustic signals, assessment of the acoustic signals via pain classification to determine whether the recorded acoustic signals represent a pain vocalization, assessment of the acoustic signals classified as a pain vocalization by means of pain intensity classification, where the pain intensity classification consists of the assignment of scale values to the recorded acoustic signals, with the scale values each representing a pain status. In one aspect, the following steps are additionally performed: a position determination of the source of acoustic signals, a position determination of the person whose pain status being determined, and a matching of the determined position by comparing it to a threshold value (i.e. with respect to a minimum similarity of the position values), and the storing of a value if the threshold value for the determined pain status is not reached.

FIG. 66 summarizes a system for determining the pain status of a person as follows: The system, for example a service robot 17, comprises a processing unit 9, a memory 10, and a sensor for the contactless detection of the person, e.g. a 2D and/or 3D camera 185, a LIDAR 1, a radar sensor, and/or an ultrasonic sensor 194. Depending on the configuration of the type of pain status determination, it may have different modules in its memory 10. In one aspect, the system has a person recognition module (110), a visual person tracking module (112), a face recognition module 5005 for recognizing the person's face, a face candidate region module 5010 for selecting candidate regions within the face, an emotion classification module 5015 for classifying the surface curvatures of the candidate regions into emotions, and an emotion assessment module for determining a scale value for the emotion 5020. The system includes, for example, a bed recognition module 5025 for recognizing a bed and/or an upper extremity evaluation module 5035 for detecting the person's upper extremities, tracking them over time, and evaluating the angles between the trunk and the upper arm, the upper arm and the forearm, and/or the phalanges and the hand bones in terms of the intensity of the angle changes, with the speed of the angular changes and/or the number of angular changes being evaluated per time unit, as well as, for example, via a pain status calculation module 5040 for determining a scale value for the pain status. In one aspect, the system includes a microphone 193 for recording acoustic signals, e.g. an audio source position determination module (4420) for evaluating the position of the source of acoustic signals and an audio signal-person module (4430) for correlating audio signals with a person. In one aspect, the system may include a pain vocalization module (5055) for classifying the intensity and frequency of the acoustic signals and determining a scale value representing a pain vocalization. In one aspect, it comprises a ventilation device recognition module 5065 for recognizing a ventilation device, i.e. for selecting candidate regions of the ventilation device, evaluating the candidate regions of the ventilation device by means of object recognition, and object classification for identifying cannulas to identify tracheal cannulas or endotracheal cannulas. In addition, the system may have a pain sensation evaluation module 5085 for evaluating sensors attached to a person, such as an inertial sensor, strain sensor, contact microphone, and/or thermistor that detect movements, air currents, and/or sounds that are classified with respect to an expression of pain. In one aspect, the system has a person identification module 111, a movement evaluation module 120, a skeleton creation module 5635, and/or a skeleton model-based feature extraction module 5640.

### Determination of Blood Pressure and Other Cardioparameters

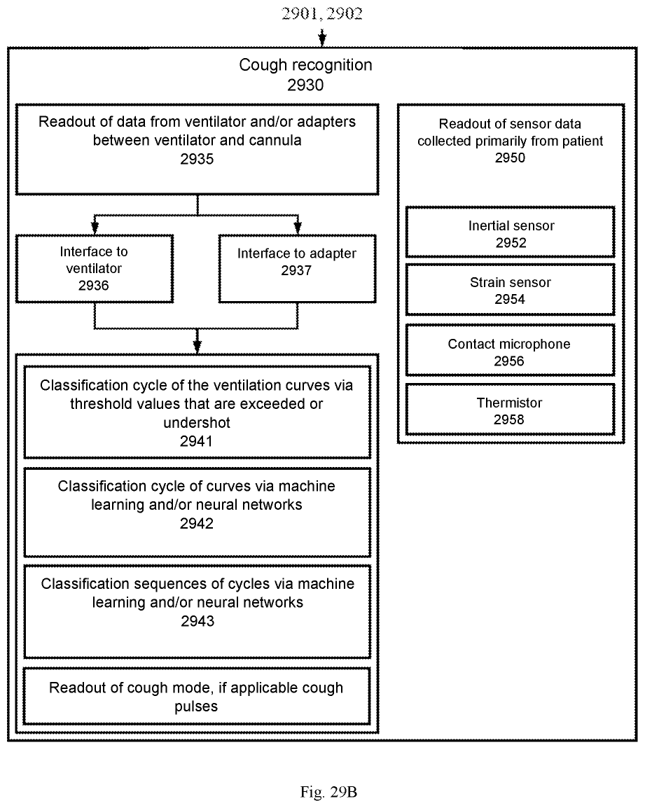

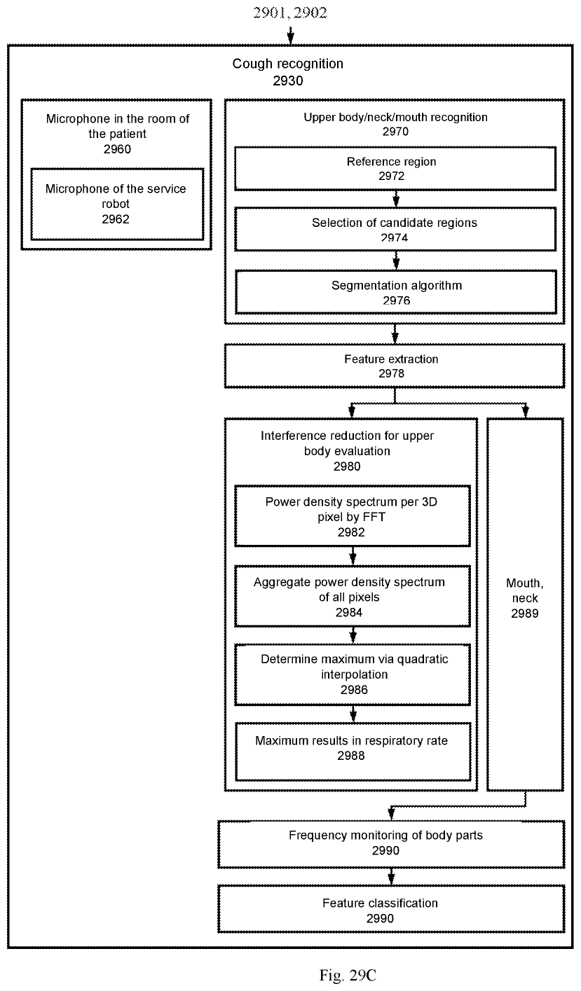

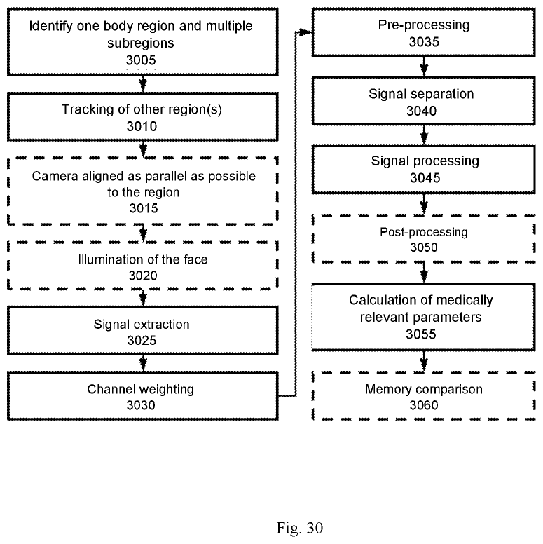

The robot 17 is further equipped with a system that detects repetitive movements of the human body that correlate with the stroke volume of the heart into the large blood vessels occurring with each heartbeat. In the process, changes are detected that result, on the one hand, from movements of the large blood vessels that propagate, for example, in the form of waves throughout the body, and/or that result from movements of the arteries in the skin. The latter are less vulnerable to variations in the illumination of the body part and/or different color tones of the skin. Alternatively and/or additionally, changes in blood volume and/or blood flow in the skin, e.g. over time, are detected, which correlate(s) with the heartbeat. FIG. 30 illustrates the process of data acquisition and analysis.

In step 3005, a body region and multiple subregions are identified. For example, the body region may be the face, with the evaluation performed using, for example, the camera 185. The system uses algorithms in the prior art to capture and track the face (alternatively other body regions), e.g. the framework OpenCV, OpenPose, or dlib, with the evaluation being performed e.g. by means of the visual person tracking module 112 and/or the laser-based person tracking module 113. For this purpose, at least the forehead, the cheeks, or the chin are captured as subregions, e.g. several body regions in combination, which are then evaluated individually and/or separately according to the steps described further below. Also, a selection of candidate regions can be made, for example, i.e. subregions of the face that are relevant for the evaluation, for which purpose segmentation methods known in the prior art (such as RANSAC) can be used. These subregions and the body region as a whole are tracked over time by the above-mentioned frameworks in step 3010.

In an optional step 3015, the camera 185 is aligned so as to be as parallel as possible to the region to be tracked. This can be achieved by minimizing the angle of coverage of the face. This angle results from an axis that is perpendicular to the camera capturing the face and an axis that is perpendicular to the sagittal plane of the face. The system determines, for example, a plane that runs through the face and that is essentially parallel to the top view of the face and that corresponds, for example, to the sagittal plane. The system has a classification based on, for example, a histograms of gradients (HoG) that describes the deviations from this top view in order to detect the inclination of the face in three-dimensional space. In one aspect, the system uses this procedure to assess a face looking into the system's camera 185 based on the extent to which the face is oriented parallel to the camera lens. In one aspect, if there are deviations, the system can adjust the inclination of the camera 185 in three-dimensional space using an appropriate mechanism such as a tilting unit 5130, e.g. by controlling two tilting axes that are orthogonal to each other and driven by servo motors. In this document, the term “tilting unit” therefore refers to a tilting unit with at least two axes which, on the one hand, enables the horizontal plane to be tilted and, on the other hand, enables rotation about the vertical axis. Alternatively and/or additionally, the wheels of the service robot 17 are controlled in such a way that the service robot 17 rotates in the direction of the person in order to reduce the determined deviation of the camera plane from the plane of the face. In an alternative and/or additional aspect, such a deviation triggers the speech output of the service robot 17 to issue instructions to the patient to align his or her face accordingly. For example, rules are stored that require alignment in the XY plane if deviation in the XY plane is detected. The adjustment of the face inclination and the actuation of the tilting mechanisms, the orientation of the service robot 17, and/or the speech output to the patient are performed, for example, until the angles between the camera plane and the facial plane have reached a minimum. Alternatively and/or additionally, the angle of coverage of the camera 185 may be minimized compared to the sagittal plane and possibly also the transverse plane, including, for example, travel maneuvers of the robot 17.

In an optional aspect, the system is configured to illuminate a body part such as the face in step 3020. This means that, during an image capture by the camera 185, the face (or other body part) is lit up by means of a light that illuminates the patient body part to be captured. At least one light is used, which is located, for example, in close proximity to the camera 185. Ideally, this light is located below and/or above the camera 185, i.e. is vertically offset relative to this camera 185. For example, the emitted light is scattered in order to ensure that the area to be captured is illuminated with the greatest possible homogeneity. Depending on the position of the face and its dimensions, a lateral arrangement of the camera 185 may possibly result in the casting of a shadow by the nose onto the cheeks, which are located to the side of the nose and whose recording provides an above-average signal-to-noise ratio. This may possibly lower the evaluation quality.

The camera 185 used provides at least one color channel for the evaluation, e.g. including at least the green color channel, since the light emitted in this case is particularly well absorbed by hemoglobin. In an additional aspect, the camera 185 also provides a color channel for the color tone orange and/or cyan. For example, the color depth per channel is at least 8 bits, and the frame rate is 30 frames per second. In one aspect, the camera 185 may also be an RGB-D camera 185 that provides depth detection in addition to color detection, e.g. based on time-of-flight sensors or speckle patterns, in order thereby to detect rhythmic vascular blood flow and rhythmic vascular expansion.

In the first step 3025, signal extraction is performed. For this purpose, the input signal is first selected on the basis of the video signals of the tracked regions, which can be either a movement caused by the pumping rhythms of the heart and/or a color change in the flow of blood, in particular hemoglobin, allowing the detection of said rhythmic vascular blood flows and/or rhythmic vascular expansions.

In the second step, a color channel evaluation is carried out on the basis of the raw data, whereby known information as to which features are shown by which color channel is included if the recording is of the blood flow. This is understood in particular to include a weighting of the channels in step 3030. In particular, the green color channel can be evaluated, or the green and red color channel (e.g. as a difference analysis of the green and red channel), or the combination of green, cyan, and orange, and so forth. As an alternative or additional step, the spatial resolution is determined in order to detect the movements. This means that the vertical and/or horizontal movements of captured features of the face are tracked, e.g. the position of the face and its subregions are captured and evaluated over time. This includes both the movements of the head and those of individual parts of the face.

The subsequent signal determination uses, for example, at least one filter in the first sub-step (pre-processing 3035). This includes a trend adjustment (e.g. with scaling and/or normalization); a moving average analysis; high-pass filtering; band-pass filtering, if applicable designed as adaptive band-pass filtering; amplitude-selective filtering; Kalman filtering; and/or continuous wavelet transform. Alternatively and/or additionally, a linear least squares polynomial approximation may be applied.

This is followed by a signal separation process 3040 in order to improve the signal-to-noise ratio and to reduce the number of feature dimensions to be considered. The process may also use principal component analysis or independent component analysis, and, in one aspect, machine learning techniques may also be employed.

Signal processing 3045 comprises the determination of the pulse rate and possibly other variables within the framework of a Fourier transform (fast, e.g. discrete Fourier transform, in particular for the determination of the maximum power spectrum density), autoregressive models (e.g. employing Burg's method), the use of band-pass filters for the detection of maxima e.g. of a peak detection, a continuous wavelet transform, and/or machine learning models, in particular non-supervised learning. Alternatively and/or additionally, a discrete cosine transform can also be used.

Further methods can be used in the scope of post-processing 3050, for example, to compensate for errors due to movements of the head, etc., for which once again Kalman filters, (adaptive) bandpass filters, outlier detection, moving averages, Bayesian fusion, and/or machine learning methods can be used.

The processing steps performed up to this point already give an account of a number of medically relevant parameters, such as pulse rate, pulse rate variability, pulse transit time, pulse waveform, etc. A further calculation of medically relevant parameters in step 3055, for example, is performed on the basis of various approaches described in the prior art, which can be used, for example, to determine the systolic and diastolic blood pressure, which allows the use of either linear or non-linear prediction methods.

The machine learning methods mentioned, e.g. neural networks such as convolutional neural networks, are able to recognize hidden and partly unknown features in the data and to take them into account during evaluation, e.g. in the scope of cluster analyses. In the process, for example, training data is used to generate weights for the classifications or for linear or non-linear prediction models, which are then used in productive operation as part of the process described.

The values determined for the pulse rate, pulse rate variability, and possibly further values, if applicable after post-processing, are compared in one aspect with values stored in the memory 10 and, based on this, the pulse rate, pulse rate variability, or further variables are determined, in particular including the systolic and diastolic blood pressure.

The filtering carried out during pre- and post-processing depends on the variables to be detected. In one aspect, a bandpass filter covering the spectrum 0-6 Hz can be used for the pulse amplitude, for example, at least 0.7-4.5 Hz. In one aspect, the signal from the pulse can also be sampled more narrowly in this frequency range, e.g. with a window of 0.1 Hz. This can be followed by smoothing by a low-pass filter. The pulse rate or heart rate, or the pulse frequency or heart frequency can be processed, for example, using a band-pass filter with width between 0.7 and 4 Hz. To determine pulse rate variability, a band-pass filter with a window between 0 and 0.4 Hz can again be used, in one aspect sampled with intervals of 0.02 Hz. The pulse transit time can be determined by comparing the values of at least two captured regions, where a bandwidth between 0.5 and, for example, 6 Hz can be evaluated, in one aspect sampled at intervals of 0.1 Hz. The pulse transit time can thereby be determined by comparing the values of multiple regions. The pulse shape results from an unsampled curve in the spectral window of approx. 0-6 Hz that is characterized, for example, by the area under the curve, the height and/or width. The pulse energy results from the first derivative of these values.

For example, the blood pressure can be determined by means of a linear model from the pulse transit time and the pulse or heart rate as well as from the preceding blood pressure value, for which linear regression models or neural networks can be used. Instead of a preceding blood pressure value, for example, the shape of the measured pulses can also be evaluated, e.g. by determining the differences between the pulse curve and the vertical line running through the maximum value.

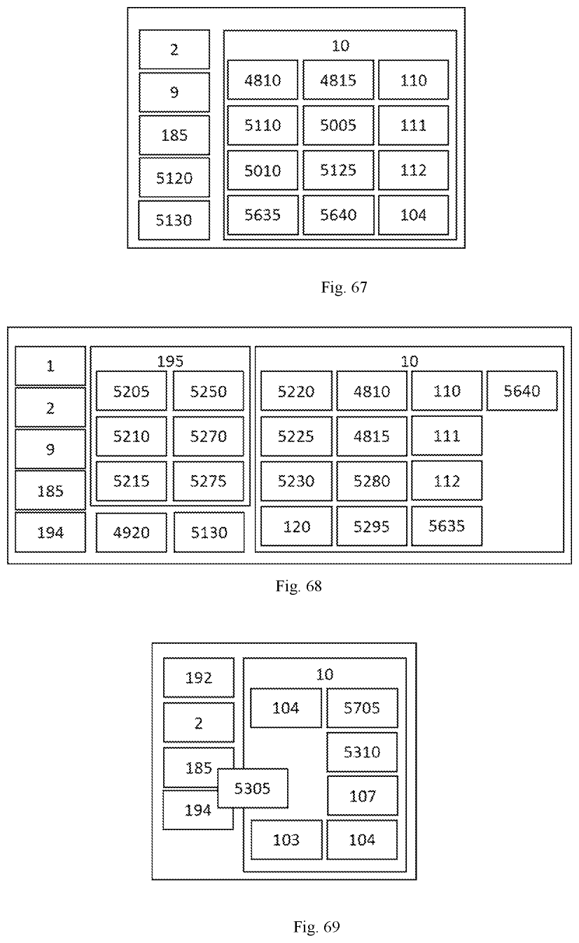

The system for determining blood pressure can be summarized as follows, as illustrated in FIG. 67: The system for determining cardiovascular parameters of a person, i.e. in one aspect a service robot 17, includes a processing unit 9, a memory 10, a camera 185 (e.g. a 2D and/or 3D camera), and further comprises a body region detection module 4810 for detecting body regions, a body region tracking module 4815, a face recognition module 5005, a face candidate region module 5010, and a cardiovascular movements module 5110 for detecting movements attributable to cardiovascular activity. The camera 185 includes at least the 8-bit green color channel. The system further includes a light 5120 in order to illuminate the face as it is recorded by the camera 185, which is located, for example, above and/or below the camera 185. The system has a blood pressure determination module 5125 for determining systolic or diastolic blood pressure 5125 and/or a tilting unit 5130 in order to minimize the angle of coverage of the camera 185 relative to the sagittal plane. For this purpose, the system has rules, e.g. for placing a vertical line between the eyes of the captured person so that the head is divided in two halves. The face is segmented, and histograms of gradients are superimposed on the individual segments. If these exhibit a (mirror-inverted) similarity that is below a threshold value, the face is regarded as having been captured vertically. The camera 185 may now be actuated via a tilting unit 5130 such that, during actuation, this comparison of the minor-inverted halves of the face is made via the histograms of gradients, with the camera positioned in such a way that the threshold values of the histograms of gradients are not reached. In one aspect, the system has a person recognition module 110, a person identification module 111, a visual person tracking module 112, a movement evaluation module 120, a skeleton creation module 5635, a skeleton model-based feature extraction module 5640, and/or a movement planner (104).

### Detection of Substances on or Below the Skin Surface

In one aspect, the service robot 17 may also be equipped with a detector 195 located, for example, on the side of the service robot 17 facing the patient. In one aspect, this detector 195 may be integrated permanently within or on the surface of the service robot 17. In an alternative and/or additional aspect, the detector 195 is mounted on an actuator 4920 such as a robotic arm and may be aligned with surfaces of the patient's body identified by the service robot 17, and, in one aspect, may contact the patient's skin, as described by way of example for aligning the spectrometer 196 with the skin of a patient. The service robot 17 may alternatively and/or additionally prompt the patient to touch the detector 195, for example with his or her finger. In one aspect, the service robot 17 is able to verify that the patient is actually touching the detector 195. In one aspect, this verification may be performed via a trial measurement, in which the acquired values are compared to those from a measurement interval stored in the memory 10 in order to assess whether the patient has actually placed a finger on the detector 195. However, this approach cannot rule out that the measurement results may be influenced by the orientation of the finger on the sensor. In an alternative and/or additional aspect, therefore, camera-based tracking of the finger is performed, with the evaluation being performed, for example, by means of the visual person tracking module 112 and/or the laser-based person tracking module 113. Such tracking has already been described elsewhere in this document. Alternatively and/or additionally, a dialog-based method may be used in which the patient is asked by the service robot 17 whether the service robot 17 has correctly placed the finger, which may be achieved by way of a display 2 and/or a speech output.

The surface of the detector 195 consists of a crystal, for example a crystal with a cubic lattice structure such as that of diamond, or a hexagonal lattice structure, or a tetragonal lattice structure. The crystal has a refractive index of 1-4, e.g. 1.3-1.4, 2.2-2.4, or for example 3.4-4.1. The spectral width of the crystal lies within an interval of 100 nm-20,000 nm, e.g. in the interval 900 nm-12,000 nm. For this purpose, the measuring procedure of the detector 195 uses deflections of an evaluation laser 5205 at the crystal surface based on the laser-induced excitation of substances, which are excited by a further laser 5210 on and/or within the skin of a patient. In this case, the area excited by the further laser 5210 interacts with, for example, the detector 195 at the location where the evaluation laser 5205 is deflected at the crystal surface. For the purpose of evaluation, feature extraction is performed in which the variations of the wavelength of the further laser 5210 and the deflection of the evaluation laser 5205 caused thereby and detected by a sensor based on the photoelectric effect are included as features. The steps shown in FIG. 30 can be used here, in particular 3025-3050, which have been described in greater detail elsewhere. The features are then classified by comparing them with feature classes stored in the memory 10. For this purpose, certain substances and their concentrations are assigned to certain wavelengths and or wavelength variations of the further laser 5210 and deflections of the evaluation laser 5205 based on these, for example. The determined classifications are subsequently stored and output via a display 2 and/or stored in the patient administration module 160.

An alternative and/or additional embodiment employs a camera-based system that is directed to the surface of a patient's skin and can take measurements. Here, in one aspect, the system can be either rigidly mounted on the service robot 17 or mounted in such a way that it can be oriented, for example, in three dimensions to allow detection of the surface of a patient's skin without the patient moving. As described for the detection of emotions, for example, the service robot 17 detects areas of the patient in which the skin surface of the patient is to be captured.

The skin surface to be captured is illuminated by the at least one camera 185, for which purpose, in one aspect LEDs, are used, which together cover a light spectrum of 550-1600 nm, e.g. at least 900-1200 nm, thereby lying in the infrared range. Here, the sensor of at least one camera 185 is an indium gallium arsenide or lead sulfide-based sensor, which in one aspect is supplemented with a silicon based sensor, which may be integrated into another camera 185. In one aspect, a laser is used instead of LEDs. The light sources may be controlled in such a way that the wavelength of the light sources varies over time. At the same time, the at least one camera 185 detects emissions excited by the light from substances located on or within the skin. Feature extraction is carried out during the measurement, in order to determine the phase and frequency emitted by substances on and within the skin, in a further aspect also taking the frequencies of the emitted light into account. In addition, different filters can be applied in pre- and/or a post-processing, e.g. band-pass and/or low-pass filters. Overall, steps 3025 to 3050 shown in FIG. 30 and described in more detail elsewhere can also be run through here. Concentrations of the substances are then determined on the basis of a feature classification.

FIG. 68 illustrates the system for substance measurement as follows: The system for measuring substances on and/or within the skin of a person, which, in one aspect, is a service robot 17, comprises a detector 195 with an evaluation laser 5205 and a further laser 5210, where the evaluation laser is deflected upon entry into a medium 5215 such as a crystal surface, and the further laser 5210 excites a substance while varying the wavelength, with the region of the excited substance interacting with the medium 5215, e.g. the crystal, at the point where the evaluation laser 5205 is deflected, and further comprising a laser variation module 5225 for feature extraction and feature classification of the wavelength variation of the further laser 5210, and a laser deflection evaluation module 5220 for evaluating the deflection of the evaluation laser. The system includes, for example, a sensor for the contactless detection of a person, a movement evaluation module (120) for evaluating detected movements of the person over time, and/or a finger positioning recognition module 5230 for the automated recognition of the positioning of a finger on the medium 5215 and the performance of the measurement after the finger is placed on the medium. In one aspect, the system for measuring substances on and/or within the skin of a person, for example, a service robot 17, comprises a detector 195 with a medium 5215 comprising a crystal with a cubic, hexagonal, or tetragonal lattice structure, a refractive index of 1-4, and a spectral width within an interval of 100 nm-20,000 nm. The system may further comprise an evaluation laser 5205 and a further laser 5210, with the evaluation laser 5205 being deflected from the crystal surface and the further laser 5210 exciting a substance while varying the wavelength, the region of the excited substance interacting with the medium 5215 at the point where the evaluation laser 5210 is deflected. The system may further comprise a laser variation module 5225 for feature extraction and feature classification of the wavelength variation of the further laser 5210, and a laser deflection evaluation module 5220 for evaluating the deflection of the evaluation laser 5205. The evaluation laser is evaluated by means of a sensor based on the photoelectric effect 5250. The system may further include an interface for the transmission of data to a patient administration system 160. The detector 195 may be positioned on an actuator 4920, and the system may include a rules module for positioning the detector 195 on a person's skin, for example, by matching the positions of the actuator 4920 with the position at which the actuator is to be positioned, and controlling the actuator in such a way that the distance between the actuator 4920 and the position at which the actuator 4920 is to be positioned is reduced to at least near zero. Furthermore, the system may include a sensor for the contactless detection of a person, e.g. a 2D or 3D camera 185, a LIDAR 1, a radar, and/or an ultrasonic sensor 194. In one aspect, the system may include a body region detection module 4810 and a body region tracking module 4815 for tracking the region of measurement. The system for measuring substances on and/or within the skin of a person is equipped, in one aspect, with a camera 185 and a tilting unit (5130) for the horizontal and/or vertical adjustment of the camera 185, a body region detection module (4810), and a body region tracking module (4815) for the identification and tracking of a person over time (identical in one aspect to the person identification module 111 and tracking modules 112 and 113), comprising at least one light source 5270 for illuminating the person's skin to be detected, with the system having a wavelength variation unit 5275 for varying the wavelength of the light emitted by at least one light source, and a wavelength variation evaluation unit 5280 for evaluating the variation of the wavelength of the captured signals. The at least one light source 5270 may be a laser (in one aspect identical to lasers 5205 and/or 5210) and/or multiple LEDs with different spectra that may be controlled accordingly. The wavelength of the emitted light is between 550 and 1600 nm, e.g. 900 and 1200 nm. The camera 185 may have, for example, a photodetector made of indium gallium arsenide or lead sulfite. In one aspect, the system includes another camera 185 for detecting light in the 400-800 nm spectrum. In one aspect, the system may have, for example, a substance classification module 5295 for the feature extraction and feature classification of acquired data and the comparison of the classified data with a substance classification, for example, an evaluation of at least the detected light is performed by comparing evaluated features to stored features. In one aspect, the system includes a person recognition module 110, a person identification module 111, a tracking module (112, 113), a movement evaluation module 120, a skeleton creation module 5635, and/or a skeleton model-based feature extraction module 5640.

### Moisture Recognition and Robot Navigation

In the environment in which the service robot 17 is moving, the floors on which the service robot 17 and a person tracked by the service robot 17 are moving may be wet, e.g. due to cleaning operations or spills. Such wet surfaces may pose a hazard associated with an increased risk of falling for persons being guided for training by the service robot 17. In one aspect, in order to reduce the risk of injury to the person, the service robot 17 includes appropriate sensor technology to detect moisture on the floor. The prior art describes various sensor technologies that may be used for this purpose:

Yamada et al 2001, “Discrimination of the Road Condition toward Understanding of Vehicle Driving Environments”, IEEE Transactions on Intelligent Transportation Systems, Vol. 2 (1), March 2001, 26-31 (DOI: 10.1109/6979.911083) describes a method for detecting moisture on the floor through the polarization of incident light. They use Brewster's angle) (53.1° as an inclination angle to set the reflection to 0 in the horizontal polarization plane, while the vertical polarization shows a strong reflection. The extent to which moisture is present on the measured surface is determined based on the ratio of the measured intensities of the horizontal and vertical polarization.

Roser and Mossmann, “Classification of Weather Situations on Single-Color Images”, 2008 IEEE Intelligent Vehicles Symposium, 2-6 Jun. 2008 (DOI: 10.1109/IVS.2008.4621205), however, proposes an approach that does not require polarizing filters and is instead based on image parameters such as contrast, brightness, sharpness, hues, and saturation, which are extracted as features from the images. Brightness is accounted for in Koschmieder's Model, which is well established in image processing. According to this model, brightness, i.e. luminance, depends primarily on the attenuation and scattering of light. Contrast, in turn, is determined by the difference between local extremes of brightness, with the brightest and darkest pixels being compared in the region under observation. With regard to sharpness, the approach is based on the Tenengrad criterion established in image processing. Hues and saturation are determined using defined pixel groups. For each detected region, a histogram with 10 areas is generated for each feature and a vector is derived from it, which contains the results of the features. These vectors can be classified using methods of machine learning/artificial intelligence, including k-NN, neural networks, decision trees, or support vector machines. Pre-labeled data is initially available to train the algorithms, and the classifications obtained in the process allow future images of the floor to be assessed in terms of the extent of moisture on the floor.

In contrast, US Patent Application No. 2015/0363651 A1 analyzes the texture of the surface captured by a camera to check for wetness, comparing two images captured at different times and performing feature extraction. The features include the spatial proximity of pixels within a region (i.e. recurring patterns are sought), the detection of edges and their spatial orientation, the similarity of gray scales among the images, the established Laws' texture energy measures, autocorrelation and power density models, and texture segmentations (both region-based and boundary-based, i.e. edges lying between pixels with different textures).

In contrast, McGunnicle 2010, “Detecting Wet Surfaces using Near-Infrared Lighting”, Journal of the Optical Society of America A, Vol. 27 (5), 1137-1144 (DOI: 10.1364/JOSAA.27.001137) uses infrared diodes with a spectrum in the vicinity of approx. 960 nm and records the light emitted from these diodes using an RGB (CCD) camera to evaluate the light spectrum accordingly. McGunnicle is able to show that wet surfaces emit a characteristic spectrum, allowing moisture to be detected on the surface.

Another approach uses radar waves instead of light in the visible or invisible range, especially ultra-wideband radar waves, which are used for substance analysis. The reflected signals can be analyzed (i.e. classified) as described in the prior art, with characteristic features recognized when moisture is measured on surfaces, which thereby allows the detection of the type of moisture.

In all cases, the sensors are arranged on the service robot 17 in such a way that the sensors capture at least the surface in front of or below the service robot 17, and in one aspect, also sideways or backwards.

In one aspect, the algorithms for moisture detection are stored in the memory 10 of the service robot 17, for example as values in a database that allow the detected light spectrum in the infrared range to be evaluated spectrally or, alternatively, the radar waves emitted by the service robot 17 and reflected from the surfaces to be evaluated. In an alternative or additional aspect, the service robot 17 has a self-learning system 3100 (see FIG. 31) to distinguish wet from dry floors. This self-learning system is particularly useful, for example, for optical methods that determine the texture and/or reflections of the surface. In this aspect, the service robot 17 traverses the surfaces 3110 over which the service robot 17 typically moves when these surfaces are in a dry state. While doing so, the service robot 17 records the surfaces by means of at least one integrated sensor 3120, and performs a feature extraction 3130, e.g. according to the approaches of Roger and Mossmann or according to the theory of US Patent Application No. 2015/0363651 A1. This is preferably done at different times of the day in order to be able to take different lighting conditions into account (daylight, artificial lighting, and/or combinations of the two). An input device, which in one aspect is connected to the service robot 17 via an interface 188 (such as WLAN), is used to assign a value to the recorded measured values that labels the recorded surfaces as dry (labeling 3140). This value, together with the recorded measured values, is stored in the memory 10 in step 3145. In a further step 3150, the service robot 17 again traverses the previously traversed surfaces at least in part, but this time the previously traversed surfaces are wet. For this purpose, an input device, which, in one aspect, is connected to the service robot 17 via an interface 188 (such as WLAN), is also used to assign a value to the recorded measured values that labels the recorded surfaces as wet (labeling 3140). The sequence of whether dry or wet surfaces are first scanned (or whether these sequences even alternate) is irrelevant with respect to the effectiveness of the method. Methods of machine learning/artificial intelligence are then used to perform a feature classification 3160 of the features recorded by the sensors, as illustrated for example in Roger and Mossmann. As a result, in step 3170, surfaces are detected as wet or dry. The results of the feature classification, i.e. whether the surfaces are wet or dry, are stored in the memory of the service robot 17 in step 3180.

During future runs of the service robot 17, the service robot 17 can access the stored classifications (e.g. for radar reflections, infrared spectral absorptions, light reflections, or textures via camera images) and use these stored classifications to evaluate measured values detected by its sensors in order to determine whether the detected surfaces are wet.

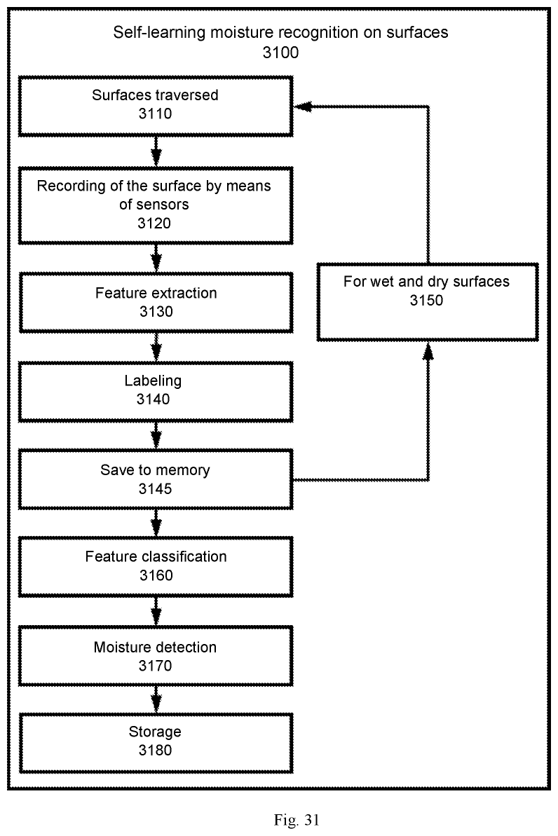

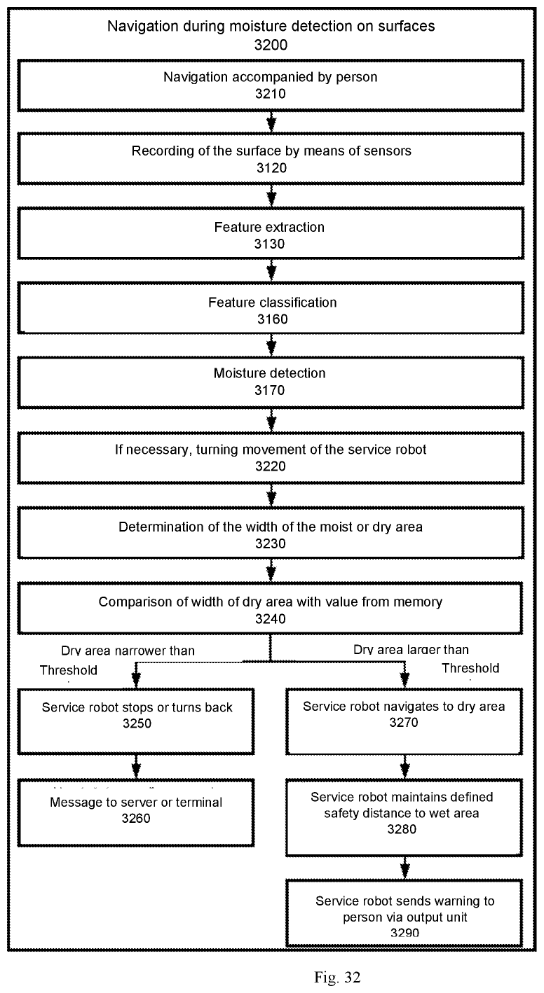

FIG. 32 illustrates the navigation of the service robot 17 while detecting moisture on surfaces 3200. If a navigating service robot 17 is accompanied by a person 3210, for example a patient during training, the service robot 17 records the surface characteristics of the floors 3120 over which the service robot 17 is moving. Feature extraction 3130, feature classification 3160, and an associated moisture detection 3170 are performed. For this purpose, in one aspect and depending on the employed sensor type or implemented evaluation algorithms, the service robot 17 may, in an optional step, detect the width of the wet or dry area 3230 by a executing a rotational movement 3220, e.g. about a vertical axis of the service robot 17. This execution may, in one aspect, be stored in the movement planner 104. Depending on the type and design of the sensor, a tilting unit 5130 may be used instead a rotational movement executed by the service robot 17, or the angle of coverage of the sensor may be sufficiently wide to detect the area in the direction of travel even without movement. The width is determined here, for example, orthogonally to the direction of travel. The width of the dry (or alternatively the wet) area is compared with a value stored in a memory 3240. In an alternative aspect, the width of the wet area is determined relative to the width of the space in which the service robot 17 is moving, for example. If the detected width of the dry area is less than the width stored in the memory, the service robot 17 does not move to the wet area, but stops and/or turns in step 3250 as stored in the movement planner 104. In an optional aspect, an output is performed via the output unit (display 2, loudspeaker 192, if applicable also a projection device 920/warning lights) indicating the surface identified as wet. In an optional aspect, the service robot 17 sends a message to a server and/or terminal via an interface 188 (such as WLAN) in step 3260. However, if the detected dry area is wider than the threshold value, the service robot 17 navigates through the dry area in step 3270 as stored in the movement planner 104. In the process, the service robot 17 maintains a minimum distance from the surface detected as wet as stored in the movement planner 104 3280. In an optional aspect, the service robot 17 may use an output unit (display 2, loudspeaker 192, if applicable also projection device 920/warning lights) to indicate the wet surface to the accompanied person 3290.

In one aspect, the classification of the moisture also includes the degree of moisture. For example, even on surfaces perceived as dry, there may be a very thin film of moisture that, however, exerts virtually no effect on the friction that an object would experience on the surface.

The steps required for the detection and assessment of moisture on surfaces can be summarized as follows: detection of a surface such as the floor, classification of the surface characteristics in order to detect moisture on the surface, segmentation of the captured surface into wet and non-wet areas, determination of the width of the captured areas, assessment of the width of the captured areas though comparison with at least one stored value.

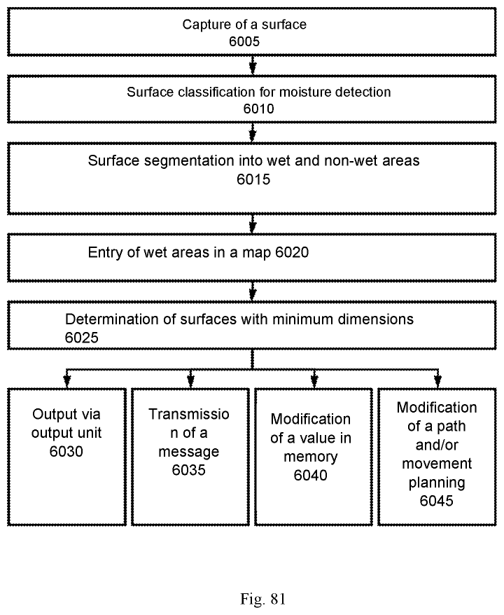

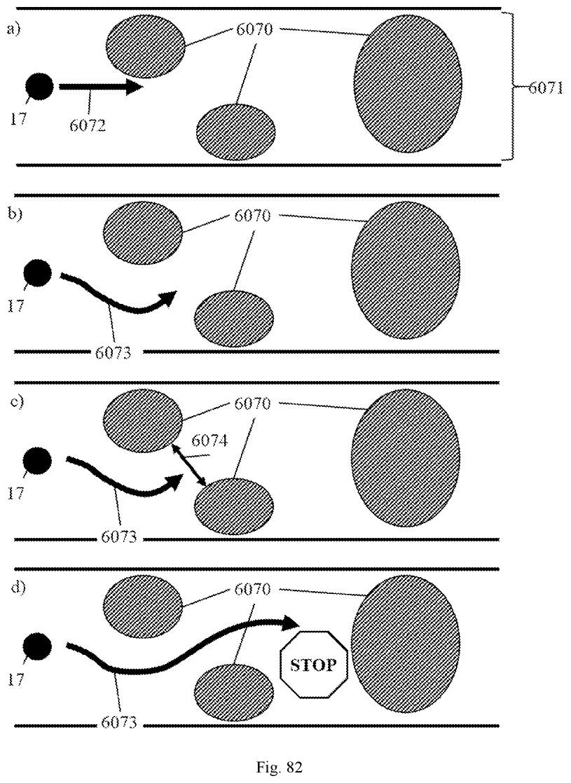

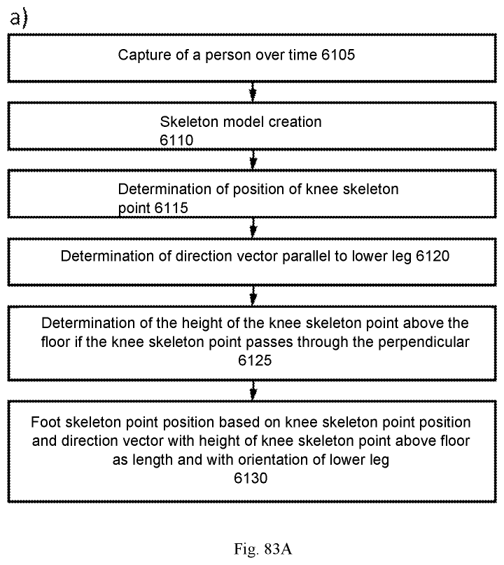

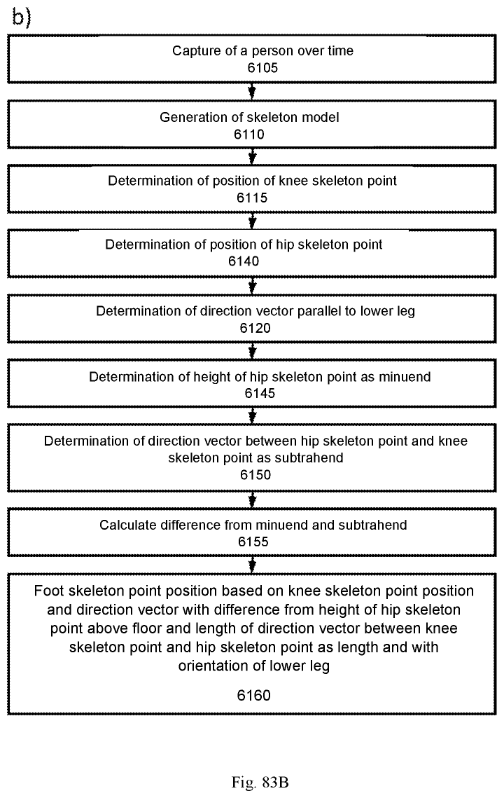

An alternative sequence can be summarized as follows, as illustrated in FIG. 81: detection of a surface 6005, surface classification for moisture detection 6010, surface segmentation into wet and non-wet areas 6015, entry of wet areas into a map 6020, determination of area with minimum dimensions 6025 and, based on this, an output via an output unit 6030, transmission of a message 6035 and/or modification of a value in the memory 10 (step 6040). Alternatively and/or additionally, path planning and/or movement planning 6045 may be modified. FIG. 82 also illustrates part of the sequence. The service robot 17 moves in a corridor 6071 along an initially planned path 6072 (see FIG. 82a), with multiple segments of moisture 6070 on the floor that are detected by the service robot. The service robot 17 plans a new path 6073 in the path planning module 103 based on the moisture representing an obstacle. The service robot 17 compares the width 6074 between surface segments stored in the memory as obstacles and determined to be wet, for example, based on a map deposited in the map module (107), maintains, for example, safety distances to these surface segments that were determined to be wet, and follows the newly calculated path (see FIG. 81 c). However, as can be seen in FIG. 81 d), an area segment detected as wet is so wide that the service robot cannot navigate around it because the width between the area classified as wet and the walls of the corridor is less than the width of the service robot 17, for which reason the service robot 17 stops in front of it.

As illustrated in FIG. 69, a system for the detection of moisture is described as follows: The system includes a sensor for the contactless detection of a surface (e.g. a camera 185 or a radar sensor 194), a segmentation module 5705 for segmenting the detected surface, a moisture detection module 5305 for classifying the segments with respect to surface moisture, and a moisture assessment module 5310 for assessing dimensions of the classified surface segments. Furthermore, it may comprise a map module 107 that includes obstacles in the surroundings of the system and the segments classified with respect to moisture. In one aspect, the system comprises a movement planner 104 and/or a path planning module 103, and, for example, an output unit (2 or 192), and outputs stored in the memory 10 for indicating the surface detected as wet. The system may be a service robot 17, for example accompanied by a person.

The system for detecting the location of moisture on a surface (e.g. a service robot 17, in one aspect accompanied by a person) includes a unit for detection (e.g. a camera 185) and a moisture detection module 5305 for classifying segments of a detected and segmented surface with respect to moisture on the surface, and a moisture assessment module 5310 for assessing dimensions of the classified surface segments. The system evaluates classified surfaces, for example, in such a way that it assesses the width of the wet area approximately perpendicular to the direction of movement of the system and defines the width of dry and/or wet areas. The system may, for example, include a movement planner 104 with rules for navigating through a dry area when the width of the area exceeds a value stored in memory 10. The movement planner 104 may include, for example, rules for determining a minimum distance to the wet area, for example, by plotting the areas classified as wet on a map and comparing its own position to the map. The system has an output unit (2 or 192) and rules stored in the memory 10 for indicating the area detected as wet and/or warnings. For example, the movement planner 104 may have rules stored for instructing the system to interrupt its movement in a predetermined target travel direction when the detected wet area width exceeds a certain threshold value or the detected dry area width falls below a certain threshold value, these rules being similar to rules used in the prior art for a mobile system to move towards an obstacle. In addition, the system may have a unit for sending a message to a server and/or terminal 13.

### Classification Method for Fallen Persons

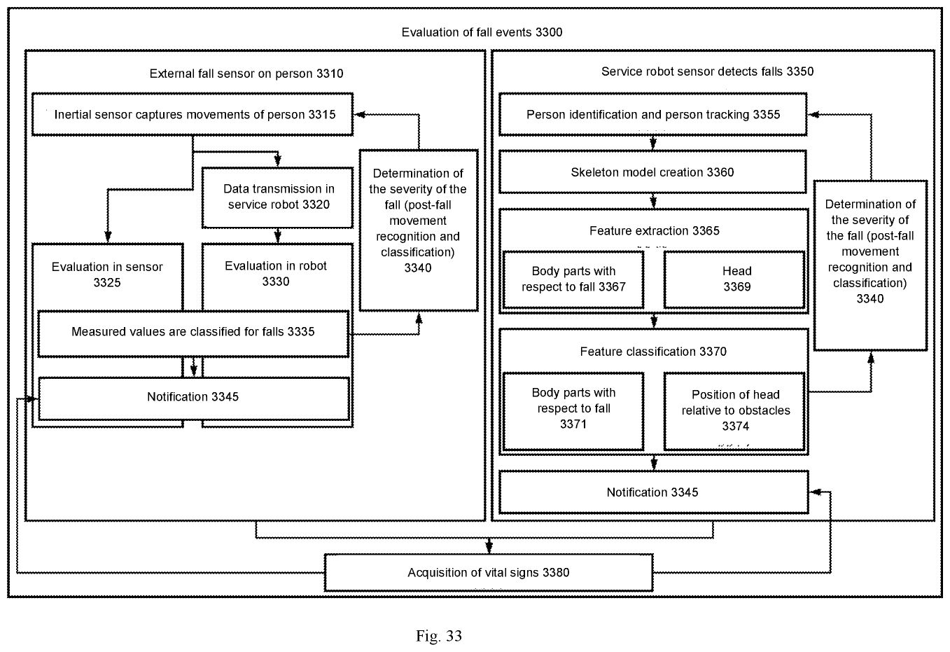

In one aspect, the service robot 17 includes fall recognition, i.e. the service robot 17 is configured such that the service robot 17 can directly or indirectly detect falls of persons. This evaluation of fall events 3300 is illustrated in FIG. 33. “Indirectly” means that the service robot 17 uses external sensors, “directly” means the service robot 17 uses its own sensors.

In one aspect, a person is equipped with a sensor unit for fall detection, i.e. the service robot 17 is connected via an interface 188 (such as WLAN) to an external fall sensor located on the person to be monitored 3310. This sensor unit includes at least one control unit, a power source, if applicable a memory, an interface 188 (such as WLAN), and at least one inertial sensor for capturing the movements of the person 3315, for example an acceleration sensor. In one aspect, the signals from the inertial sensor are evaluated within the sensor unit in step 3325, and in an alternative aspect, the signals are transmitted to the service robot 17 in step 3320, thereby allowing the evaluation of the signals in the service robot 17 3330. In step 3335, the measured values are classified as to whether the person has fallen. This classification can be made, for example, by measuring an acceleration that is above a defined threshold value. Based on a fall detection, a notification is then sent via an interface 188 (such as WLAN) in step 3345, i.e. for example, an alarm system is notified and/or an alarm is triggered (e.g. an alarm sound), etc. If the classification of the detected movements takes place within the sensor unit, the notification and/or alarm is made by the sensor unit (via an interface 188 (such as WLAN)). If the service robot 17 performs the classification of the movements, it initiates the notification and/or triggers the alarm.

In one aspect, the sensor unit is configured in such a way that the sensor unit detects the movements of the person for the purpose of detecting the severity of the fall, collects measured values for this purpose, with a classification of the measured values being performed directly within the sensor unit and/or via the service robot 17 in step 3340. Specifically, this means that this process registers the extent to which the person equipped with the acceleration sensor continues to move. Acceleration and/or orientation data of the sensor unit can also be evaluated. For this purpose, rules are stored in the memory of the sensor unit and/or the service robot 17 that trigger different notifications based on the measured movement data. For example, if, after a detected fall, the sensor unit registers movement information that is above defined threshold values and/or which is classified in such a way that the person who has fallen gets up again, the notification and/or the alarm can be modified in such a way, for example, that the priority of the notification is reduced. On the other hand, if the sensor unit detects no further movement and/or position change on the part of the fallen person following a fall event, the notification and/or the alarm can be modified, e.g. the priority of the notification can be raised. In one aspect, the notification and/or alarm occurs only after analyzing the movement behavior of the person after the fall, i.e. possibly several seconds after the actual fall, thereby possibly reducing the notifications associated with the fall event.

In one aspect, the service robot 17 is provided with a wireless sensor unit in order to capture fall events of a person in step 3350. This sensor unit may be a camera 185, e.g. a 3D camera, a radar sensor, and/or an ultrasonic sensor 194, or combinations of at least two sensors. The sensor unit is used, for example, to identify a person and/or to track the person over time in step 3355, which is implemented, for example, by means of the visual person tracking module 112 and/or the laser-based person tracking module 113. For example, the service robot 17 may be equipped with a Kinect or Astra Orbbec, i.e. an RGB-D camera 185, which is capable of creating a skeleton model of captured persons in step 3360 by means of methods described in the prior art (e.g. by means of a camera SDK, NUITrack, OpenPose, etc.), in which body joints are represented as skeleton points and the body parts connecting the skeleton points are represented, for example, as direction vectors. In addition, feature extraction 3365 is carried out in order to determine different orientations of the direction vectors as well as distances of skeleton points to the detected surface on which the captured persons are moving. By means of feature classification 3370, the service robot 17 evaluates whether the captured person is standing, walking, sitting, or has possibly fallen. In this regard, the feature classification rules may be fixed in one aspect, and in an alternative and/or additional aspect, the rules may be learned by the service robot 17 itself. In this learning process, recordings of persons who have fallen as well as recordings of persons who have not fallen are evaluated, with labeling having been carried out beforehand to define which case applies for each. Based on this, the service robot 17 can use methods of machine learning/artificial intelligence to make classifications that allow future recordings of people to be classified as to whether they have fallen or not.

Fall detection is performed here, for example, on the basis of the extraction of the following features in step 3367, whereby the body parts are evaluated, i.e. classified, with respect to the fall: Distances and distance changes in the direction of the floor or accelerations derived from the distances or distance changes (e.g. over defined minimum periods of time) of skeleton points in a direction whose vertical direction component is greater than a horizontal direction component, the vertical direction component preferably pointing towards the center of the earth. For example, a detected distance of the hip skeleton point to the floor of less than 20 cm can be classified as a fall event, or likewise a distance change of over 70 cm to under 20 cm or an acceleration of the hip skeleton point in the direction of the floor, with this acceleration falling below a defined time period, for example (e.g. 2 seconds). Alternatively and/or additionally, the orientation of at least one direction vector (as a connection between two skeleton points) in the room or the change of the direction vector in the room can be classified as a fall event. For example, an orientation of the spine and/or legs that is essentially horizontal counts as a fall event, in particular following a change of orientation (e.g. from essentially vertical to essentially horizontal), which optionally occurs within a defined time period. In an alternative and/or additional aspect, the height of the person is determined by means of the 3D camera. If the height of the person falls below a defined height, a fall event is thereby detected. As an alternative to and/or in addition to the height of the person, the area that the person occupies on the floor can also be determined. For this purpose, in one aspect, the area can be determined by means of a vertical projection of the tracked person on the floor.

After a classified fall event, the service robot 17 triggers a notification and/or alarm in step 3345, e.g. via an interface 188 (such as WLAN), in the form of an audible alarm emitted via a loudspeaker 192, etc.

In the event that the service robot 17 detects persons by means of radar and/or ultrasound 194, the external dimensions of the person are primarily detected for this purpose, including the person's height. If a height reduction is thereby detected, and possibly an acceleration of the height reduction, this is classified as a fall event, possibly accompanied by falling below threshold values. Alternatively and/or additionally, the service robot 17 may also classify the area occupied by the person (in one example: projected vertically) on the floor.

In one aspect, the service robot 17 also detects the position of the person's head in step 3369. This position is tracked with respect to the floor and/or (the position of) detected obstacles. This means, for example, that walls detected by the sensor unit (camera 185, radar sensor, and/or ultrasonic sensor 194) are captured. Alternatively and/or additionally, the position of the walls can also be determined by means of the LIDAR 1. The service robot 17 compares the horizontal position of the person's head with the (horizontal) position of walls and/or other obstacles in the room.

In an alternative and/or additional aspect, the vertical position is also considered. For example, the camera 185 may also evaluate distances of the tracked head to objects in three-dimensional space such as tables. The (two-dimensional, essentially horizontally oriented) LIDAR 1 would, for example, recognize the table legs, but not necessarily the position of a table top in the room, which the person could possibly contact with his or her head in the event of a fall. Camera-based evaluation, on the other hand, allows a three-dimensional capture of the head and other obstacles in the room and a determination of the distance between these other obstacles and the head. This determined distance is evaluated as part of the classification process carried out in step 3374. If the service robot 17 detects that the distance between the head and one of the other obstacles has fallen below a certain value, a value in the memory of the service robot 17 is modified and, if applicable, a separate notification or alarm is triggered.

In addition, the service robot 17 tracks the person after his or her fall and detects the extent to which the person straightens up or attempts to straighten up, i.e. post-fall movement recognition and classification is performed in step 3340. This means, for example, that distances in the direction of the floor, accelerations in vertical directions opposite to the floor, the orientation of body part or limb vectors, and the height and/or (projected) area of the person are evaluated. In one aspect, the degree of positional changes of skeleton points is also evaluated. Classification is performed as to the extent to which the person moves or even attempts to stand up. Values are thereby adjusted in a memory of the service robot 17 and, in one aspect, the degree of notification via interface 188 (such as WLAN) or alarm is modified.

In practical terms, this means that different alarms and/or notifications may be triggered depending on the severity of the fall. Combinations of a fall classification by means of a sensor worn on the person and a sensor data evaluation within the service robot 17 by means of a camera 185, radar, and/or ultrasound 194 are also possible. The sequence for the assessment of a fall event can be summarized as follows: capture and tracking of the movements of a person, detection of a fall event using feature extraction and classification of the orientation of limbs and/or the trunk of the person, detection and classification of the movements of the person after a fall has occurred, and assessment of the severity of the fall event.

In one aspect, the service robot 17 is also capable of recording the vital signs of the fallen person by means of its sensor system in step 3380. This may include, for example, an integrated radar sensor such as an ultra-wideband radar, as explained elsewhere. In one aspect, radar and/or camera-based methods can be used to detect parts of the person's body that are not covered by clothing, and a measurement of the person's pulse can be taken at these areas, e.g. using radar. This information can be considered in the classification of the notification and/or alarm, and, in one aspect, vital signs such as pulse can also be sent along with the notification.

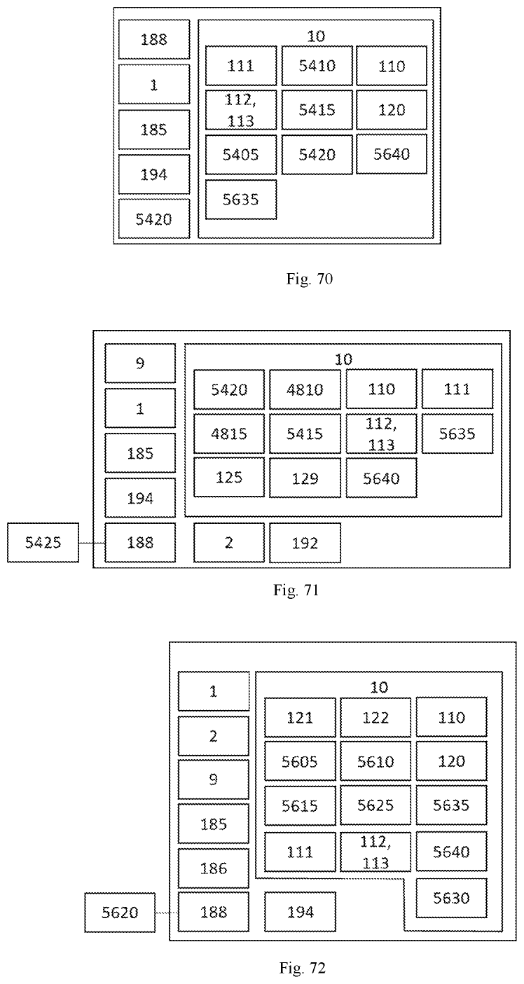

The system for fall classification is illustrated in greater detail in FIG. 70. In this figure, the system for detecting the fall of a person, e.g. a service robot 17, comprises a memory 10, at least one sensor for the contactless detection of the person's movements over time, a person identification module 111 and a person tracking module 112 or 113, a fall detection module 5405 for extracting features from the sensor data and classifying the extracted features as a fall event, a fall event assessment module 5410 for classifying the severity of the fall event. The system may further include an interface 188 to a server and/or terminal 13 for the purpose of transmitting messages. The fall detection module 5405 may, for example, have a skeleton creation module 5635 for creating a skeleton model of a person. The fall detection module 5405 may include classifications for determining distances or distance changes of skeleton points originating from the skeleton model relative to the floor; accelerations of skeleton points in the vertical direction; the orientation of direction vectors resulting from the connection of at least two skeleton points; the change in orientation of the direction vectors; the height and/or change in height of the person, e.g. by means of the person height evaluation module 5655, which determines the height of the person, e.g. by vector subtraction of two direction vectors extending from a common origin to at least one foot and at least the head of the person; the area occupied by a person projected in a vertical direction on the floor; and/or the position of the head of the person as viewed relative to the floor and/or as viewed relative to detected obstacles. The system may further comprise a vital signs recording unit 5415 for recording the vital signs of the person (e.g. a camera 185, a LIDAR 1, a radar sensor, and/or an ultrasonic sensor 194) and a vital signs evaluation module (5420) for evaluating the recorded vital signs of the person. In one aspect, the system has a person recognition module 110, a movement evaluation module 120, and/or a skeleton model-based feature extraction module 5640.

### Fall Prevention

In one aspect, the service robot 17 acquires vital signs of the person while performing a test and/or an exercise, as shown in step 3400 in FIG. 34. For this purpose, the service robot 17 identifies and tracks the person, e.g. by means of the visual person tracking module 112 and/or the laser-based person tracking module 113 in conjunction with a camera 185 or a LIDAR 1. For this purpose, person identification and person tracking are performed in step 3355, for which the person identification module 111 is used. The system is (optionally) located in front of the person (step 3420) and moves (optionally) in front of the person in step 3430. In step 3440, identification and tracking are performed for the person's body region with which the exercise and/or the test is carried out in order to record vital signs by measuring at or on this body region in step 3450. Possible body regions include the person's face, hands, chest area, etc. The procedure for detecting such a body region has been described elsewhere in this document and/or in the prior art. Examples of vital signs measured are the pulse rate, pulse rate variability, systolic and/or diastolic blood pressure, and the person's respiration (e.g. respiratory rate). The procedure for how these example vital signs can be acquired by the service robot 17, for example, has been described elsewhere in this document. However, other methods are also possible for acquiring the vital signs. The vital signs are recorded using at least one sensor, such as the camera 185 and/or the radar sensor (e.g. a microwave pulse radar, a distance control radar, a Doppler radar, a continuous wave radar, or an ultra-wideband radar) and/or combinations thereof, which detect(s) the above-mentioned body regions of the person and his or her vital signs, preferably over time.

In one aspect, the sensor used for this process detects movements on and/or under the skin and/or the clothing of the person. In an alternative and/or additional aspect, the movements of the skin surface and/or the clothing of the person are evaluated relative to the movement executed by the person towards the service robot 17, i.e. the captured body region signals are corrected for the movement of the person 3460. For this purpose, the service robot 17 captures at least one further body region in addition to the body region which is evaluated for the purpose of recording the vital signs and determines the distance of this further body region to the service robot 17. For this purpose, the process for capturing the movements of the body region for the purpose of evaluating the vital signs on the one hand and the process for capturing the body region for the purpose of determining the relative movement of the person on the other are synchronized with each other. The body region can be captured for the purpose of determining the relative movement, for example, by means of the LIDAR 1, the camera 185, e.g. the RGB-D camera 185, or the ultrasonic and/or radar sensors 194.

The measurement made by the service robot 17 may be a continuous or discontinuous measurement, e.g. at intervals of 10 seconds. The measured vital signs are stored in the memory of the service robot 17 in step 3470 and can be transmitted to other systems via an interface 188 (such as WLAN). The service robot 17 compares the determined vital signs with threshold values stored in a memory in step 3480. These values stored in the memory can be fixed and/or result dynamically from past values of the recorded vital signs, e.g. in the form of average values of previously recorded values that are evaluated over a time interval. If it is detected that the recorded vital signs exceed or fall under the threshold values in step 3490, the service robot 17 modifies a value in a memory, for example. This modification can trigger at least one of the following events: An output unit (display 2, loudspeaker 192, projection device 920, etc.) is triggered in step 3492, i.e. a speech output is initiated. In one aspect, this allows the service robot 17 to prompt the person to reduce his or her speed. In an alternative and/or additional aspect, the service robot 17 may prompt the person to sit down. In addition to or independently of this, the service robot 17 may head for a defined position 3498. This may be at least one seat that has coordinates associated with it in the map of the service robot 17. The service robot 17 may then move towards this seat. The seat may be a chair. The chair may be identified by the service robot 17 in its direct vicinity via its implemented sensors (as has been described elsewhere in this document). The service robot 17 may alternatively and/or additionally be stored on a map of the service robot 17 in the map module 107. In step 3494, the service robot 17 may trigger a notification upon detecting deviations in vital signs, sending a notification via an interface 188 (such as WLAN) and/or triggering an alarm, for example. Furthermore, in one aspect, it may reduce its speed in step 3496.

In one example of use, the service robot 17 performs a gait exercise with a person, e.g. gait training on forearm crutches. During this exercise, the service robot 17 moves in front of the person while the person follows the service robot 17. The service robot 17 captures the person by means of at least one sensor and performs feature extraction, feature classification and gait pattern classification to evaluate the person's gait pattern. During this process, the camera 185 mounted on the service robot 17 captures the person's face and determines the systolic and diastolic blood pressure over time, each of which is stored in a blood pressure memory in the service robot 17. The measured values determined for the blood pressure are evaluated over time and are optionally stored and compared with values stored in the blood pressure memory. Alternatively and/or additionally, the measured values are compared with those determined before a defined period t, e.g. t=10 seconds. If the systolic blood pressure falls by at least 20 mm Hg and/or the diastolic blood pressure falls by more than 10 mm Hg, which may indicate an increased risk of falling, for example, the blood pressure value is modified in the memory of the service robot 17. As a result of this, the service robot 17 issues a speech output prompting the person to reduce his or her walking speed. The reduced speed reduces the risk of injury in the event that the person suffers a fainting spell and falls as a result. The service robot 17 reduces its speed and sends a notification via an interface 188 (such as WLAN) to a server, which in turn alerts personnel in the vicinity of the service robot 17 and calls for assistance. The service robot 17 optionally attempts to detect a chair within its vicinity, i.e. within a defined distance away from its position. If the service robot 17 detects a chair, the service robot 17 slowly navigates to the chair and prompts the person to sit down via an output.

In a similar example, while completing a gait exercise, the service robot 17 detects the person's respiratory rate over time by evaluating the movements of the person's chest and/or abdominal region, which is achieved through the use of an ultra-wideband radar sensor mounted on the service robot 17. The measured values acquired are also stored in a memory and compared with measured values stored in a memory. Alternatively and/or additionally, the measured values are compared with those determined before a defined period t, e.g. t=10 seconds. If the respiratory rate changes by a value lying above a threshold value, the steps already described in the previous section for deviations in the measured value for blood pressure are performed.

According to FIG. 71, the system for recording vital signs can be described as follows: The system for recording the vital signs of a person, e.g. a service robot 17, comprises a processing unit 9, a memory 10 and at least one sensor for the contactless detection of the person's movements over time (e.g. a camera 185, a LIDAR 1, an ultrasonic and/or radar sensor 194), for example, a person identification module 111 and a person tracking module (112, 113) for acquiring and tracking the person, and a vital signs evaluation module 5420. It further comprises a body region detection module 4810 and a body region tracking module 4815 for tracking the region of coverage for the vital signs, and a vital signs acquisition unit 5415 for acquiring vital signs of the person, e.g. over time, using contactless and/or contact-based method. The vital signs evaluation module 5420 can, for example, perform a comparison of the acquired vital signs with at least one stored threshold value and, based on the comparison, initiate a notification of a system via an interface 188, an output via an output unit (2 or 192), a speed change of the system (e.g. a speed reduction), and/or a movement towards a target position of the system. The latter are implemented, for example, by means of a navigation module (110), e.g. by adapting path planning to a seat such as a chair located within a defined minimum distance to the system, for example. The threshold value used in the vital signs evaluation module 5420 can be dynamically determined from previously recorded vital signs, e.g. based on averaging recorded vital signs over a defined time interval. The vital signs evaluation module 5420 may further acquire body movements of the person and evaluate the acquired vital signs while comparing the acquired body movements. The acquired vital signs may include pulse rate, pulse rate variability, systolic and/or diastolic blood pressure, and/or respiratory rate. In one aspect, the system may collect data from a vital signs sensor 5425 attached to a person via an interface 188 and analyze the data in the vital signs evaluation module 5420. An application module 125 has rules for performing at least one exercise, e.g. the exercises included as examples in this document. In one aspect, the acquired and evaluated vital signs can be used to determine a risk of falling, e.g. an acute fall risk if a fall is expected to occur within a time interval of only a number of minutes. In one aspect, the system has a person recognition module 110, a person identification module 111, a tracking module (112, 113), a movement evaluation module 120, a skeleton creation module 5635, and/or a skeleton model-based feature extraction module 5640.

### Recognition of an Increased Fall Risk of a Person

Elderly people often exhibit an increased risk of falling. Various studies can be found in the prior art that have identified a large array of factors that have a major influence on fall risk. For example, the experimental work of Espy et al (2010) “Independent Influence of Gait Speed and Step Length on Stability and Fall Risk”, Gait Posture, July 2010, Vol. 32 (3), pages 278-282, (DOI: 10.1016/j.gaitpost.2010.06.013) shows that, while a slow gait results in an increased fall risk, that this risk can be reduced by shorter step lengths. Senden et al, “Accelerometry-based Gait Analysis, an additional objective approach to screen subjects at risk for falling”, Gait Posture, June 2012, Vol. 36(2), pages 296-300, (DOI: 10.1016/j.gaitpost.2012.03.015), uses acceleration sensors and other means to show that both long steps and fast walking are associated with an increased risk of falling (as determined using the standardized Tinetti test), as is the averaged square root of the vertical acceleration of the body. Prior falls were predicted on the basis of lower symmetry in the gait pattern as determined by the acceleration sensor. Van Schooten et al. (2015), “Ambulatory Fall-Risk Assessment: Amount and Quality of Daily-Life Gait Predict Falls in Older Adults, Journals of Gerontology”: Series A, Vol. 70(5), May 2015, pages 608-615 (DOI: 10.1093/gerona/glu225) also use acceleration sensors, showing that higher variance in double-step length/gait cycle in the gait direction and lower amplitude in gait in the vertical direction are associated with increased fall risk. Kasser et al (2011), “A Prospective Evaluation of Balance, Gait, and Strength to Predict Falling in Women with Multiple Sclerosis”, Archives of Physical Medicine and Rehabilitation, Vol. 92(11), pages 1840-1846, November 2011 (published Aug. 16, 2011) (DOI: 10.1016/j.apmr.2011.06.004) also reported increased asymmetry in the gait pattern as a significant predictor of fall risk.

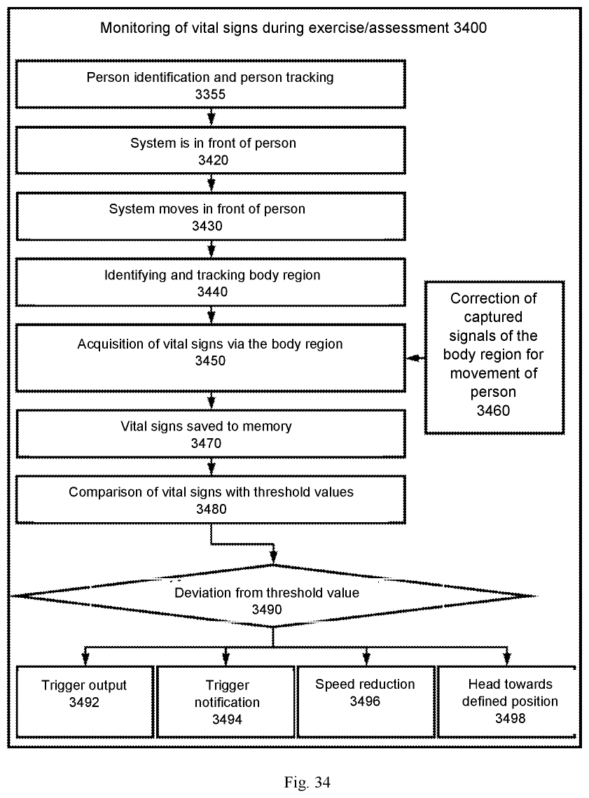

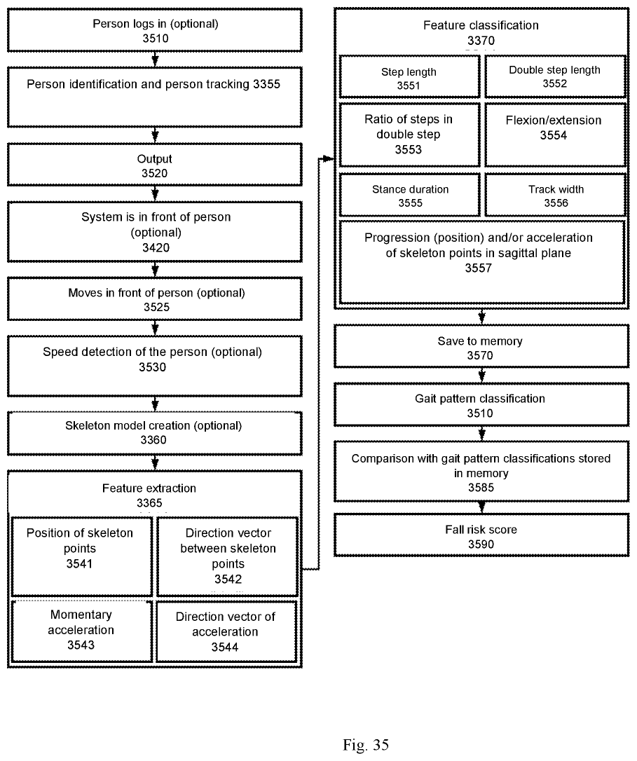

In one aspect, the service robot 17 is configured in such a way that the service robot 17 evaluates a person's gait pattern for fall risk, as explained in FIG. 35. In one (optional) aspect, the person logs in at the service robot 17 3510, which can be done using an input unit, an RFID transponder, a barcode, etc. The service robot 17 then performs person identification using its person identification module and then tracks the person 3355, e.g. using the visual person tracking module 112 and/or the laser-based person tracking module 113. A sensor is used for this tracking that enables the contactless detection of the person, e.g. a camera 185, an ultrasonic sensor, and/or a radar sensor 194. The service robot 17 uses an output 3520 of an output unit to prompt the person whose risk of falling is to be assessed to follow the service robot 17. This service robot 17 is (optionally) in front of the person in step 3420, (optionally) moves in front of the person in step 3525, and (optionally) detects the person's speed in step 3530. In one aspect, this is achieved by determining the speed of the service robot 17 while synchronously detecting the distance of the identified person, thereby determining the relative speed of the person to the service robot 17 and, based on the speed of the service robot 17 itself, the speed of the person. The speed of the service robot 17 itself is determined via its odometry unit 181 and/or by tracking obstacles stored in the stored map of the service robot 17 and the movement of the service robot relative to these obstacles.

The service robot 17 performs feature extraction in step 3365 in order to extract features from the skeleton model in step 3360, such as the positions of skeleton points 3541, direction vectors connecting skeleton points 3542, the perpendicular through a person, etc. Alternatively and/or additionally, features may be extracted from inertial sensors attached to at least one limb of the person, etc., e.g. momentary acceleration 3543, the direction of an acceleration 3544, etc. Feature extraction 3365 is followed by feature classification 3370, in which multiple features are assessed in combination. To name an example, the speed of the person may be determined as a feature from the acquired data of the service robot 17 as an alternative and/or supplement to the method described above, for which possible individual classified features may include, for example, the step length 3551 and/or double step length 3552 of the person, which the service robot 17 acquires over time, determining the speed over the step length per acquired time unit. One aspect allows the evaluation of multiple steps. In addition, in one aspect, the step length is extracted as part of feature extraction 3365 via the position of the foot skeleton points in the skeleton model, with the skeleton model being created in step 3360 through the evaluation of camera recordings of the person. In the case of the evaluation of data from the inertial sensor attached to the foot or ankle, for example, the time points and the times between the time points in which a circular motion whose radius points towards the floor starts to be detected by the sensor are acquired/extracted, i.e. the direction vectors of acceleration 3544 are evaluated for this purpose. The momentary acceleration is determined/extracted in step 3543, preferably in the sagittal plane, and the distance traveled is determined via the momentary velocities in step 3543, with the time duration between the above-mentioned time points determined in the scope of feature classification, which then represents the step length in step 3551. Each of these represent extracted features that are classified in this combination. In an alternative and/or additional aspect, the ankle is detected using a radar sensor and/or an ultrasonic sensor 194. In an alternative and/or additional aspect, the foot skeleton point is determined on the basis of the position of the knee skeleton point, a direction vector originating from the knee skeleton point with a parallel orientation to the lower leg, and the height of the knee skeleton point above the floor if the direction vector passes through the perpendicular, with the height of the knee skeleton point above the floor indicating the distance at which the foot skeleton point is located as viewed from the knee skeleton point if the direction vector passes through the perpendicular. The above-mentioned double step length 3552 is determined on the basis of the distances between the detected foot skeleton points, with the single step lengths 3551 being successively added in one aspect.

In an alternative and/or additional aspect, the service robot 17 evaluates the respective length and/or duration for the single steps within a double step for each double step and correlates the length with the duration in step 3553. The service robot 17 adds the acquired values from more than one double step in order to determine an average value over more than one double step. In one aspect, the service robot 17 evaluates flexion and/or extension 3554, i.e. the angles of the thigh relative to the perpendicular.

The service robot 17 then evaluates the person's detected speed, detected step length, double step length, and cadence. In an alternative and/or additional aspect, the stance duration in step 3555 of at least one foot, e.g. both feet and, for example, each over several steps, is also evaluated. In an alternative and/or additional aspect, the track width may also be evaluated in step 3556, whereby the distance between the ankles is evaluated. Furthermore, the service robot 17 acquires other skeleton points from the skeleton model of the person, such as the head, the shoulder skeleton points, the pelvis/hip skeleton points, etc., detects their position in the room, e.g. in three dimensions, and evaluates these parameters over time. In one aspect, this evaluation includes the height of these parameters above the floor, as well as their movement in the sagittal plane (both vertical and horizontal). In one aspect, this also includes evaluating the acceleration of at least one of the points from the skeleton model mentioned above 3557.

The service robot 17 stores the acquired values in step 3570, classifies the gait pattern in step 3580 using the classified features, and compares it to a gait pattern classification stored in its memory (or in a memory available via an interface 188 (such as WLAN)) in step 3585. For this purpose, at least one of the above-mentioned classified features, or preferably several, is/are (jointly) evaluated and compared with those from the memory. Based on the comparison of the service robots 17, the service robot 17 determines a score that reflects the fall risk in step 3590, e.g. a probability that the captured person will fall within a defined time period. In one aspect, the classification includes values for a determined speed, cadence (steps per minute), and step length as a function of such person parameters as person height. For example, persons are associated with an increased fall risk if they have a step speed of approx. 1 m/s, a cadence of less than 103 steps/min, and a step length of less than 60 cm at average height. Alternatively and/or additionally, recorded accelerations in three-dimensional space are evaluated and the harmonics are formed by means of a discrete Fourier transform. Then the ratio of the summed amplitudes of the even harmonics by the summed amplitudes of the odd harmonics is formed. Values from the vertical acceleration that are below 2.4, acceleration values in the walking direction in the sagittal plane that are below 3, and values of lateral acceleration in the frontal plane that are below 1.8 indicate an increased risk of falling. The corresponding evaluations are evaluated in the gait feature classification module 5610. In addition, multiple parameters are evaluated simultaneously, for example, such as accelerations, step length, cadence, etc.

The system, for example, for determining a score that describes the fall risk of a person, e.g. a service robot 17, can be summarized as illustrated in FIG. 72. The system for determining a score describing the fall risk of a person includes a processing unit 9, a memory 10, a sensor for detecting a person's movements over time (including a gait pattern), e.g. a camera 185, a LIDAR 1, an ultrasonic and/or radar sensor 194, a movement extraction module 121, and a movement assessment module 122, which is configured in one aspect so as to determine a fall risk score within a fall risk determination module 5430, e.g. evaluating accelerations in horizontal and/or vertical planes, step width, velocity, and/or variables derived from these, etc. The movement extraction module 121 may include a gait feature extraction module 5605 for the feature extraction of a gait pattern, while the movement assessment module 122 may include a gait feature classification module 5610 for the feature classification of a gait pattern based on the extracted features (e.g. of the skeleton points of a skeleton model of the captured person, direction vectors between the skeleton model's skeleton points, accelerations of the skeleton points or the direction vectors, the position of the skeleton points relative to each other in the room, and/or angles derived from direction vectors) and a gait pattern classification module 5615 for gait classification (comprising, for example, the step length, the double step length, the step speed, the ratio of the step lengths in the double step, the flexion and/or extension, the stance duration, the track width, and/or the progression (position) and/or the distance of skeleton points relative to one another and/or the acceleration of skeleton points), with, for example, the classification comprising a comparison of recorded gait patterns with gait patterns stored in the memory and a determination of the fall risk score. The gait pattern classification module 5615 may comprise a person speed module 5625 for determining the speed of the person, with the speed of the person being determined on the basis of the number and step width of steps covered by the person per time unit relative to the speed of a detection and evaluation unit/the system, including an odometry unit 181 and including obstacles detected in a map, and/or relative to the position of obstacles detected in a map. In addition, the system includes a person identification module 111, a person tracking module (112 or 113), and components (e.g. 2, 186) for logging the person into the system, with visual features of the person being stored and used by the person reidentification module (114), for example. The system may receive sensor data from an inertial sensor 5620 via an interface 188 and analyze this sensor data in the movement extraction module 121. The sensor may, for example, be attached to the person, e.g. on his or her lower limbs, or it may be attached to a walking aid used by the person, such as an underarm or forearm crutch, and may detect movements of the walking aid.

In one aspect, the system has a person recognition module 110, a movement evaluation module 120, a skeleton creation module 5635, and/or a skeleton model-based feature extraction module 5640. In terms of procedure, the fall risk score, which in one aspect describes latent (rather than acute) fall risk, is determined as follows: capture of the gait pattern of a person (e.g. by the above-mentioned sensor for the contactless detection of the person), extraction of features of the detected gait pattern, classification of the extracted features of the gait pattern, comparison of at least two of the classified features of the gait pattern with a gait pattern classification stored in a memory, and determination of a fall risk score.

### Mobility Test Performed by the Service Robot (Tinetti Test)

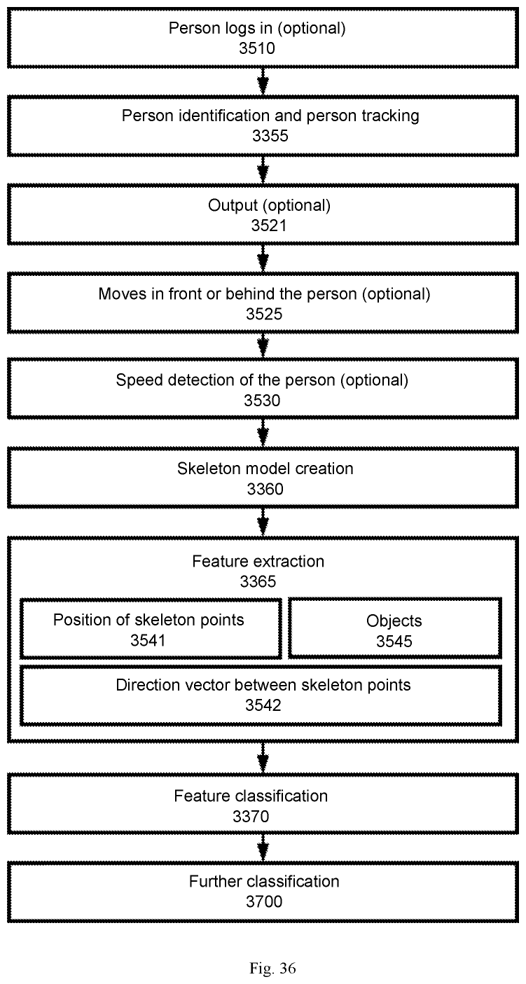

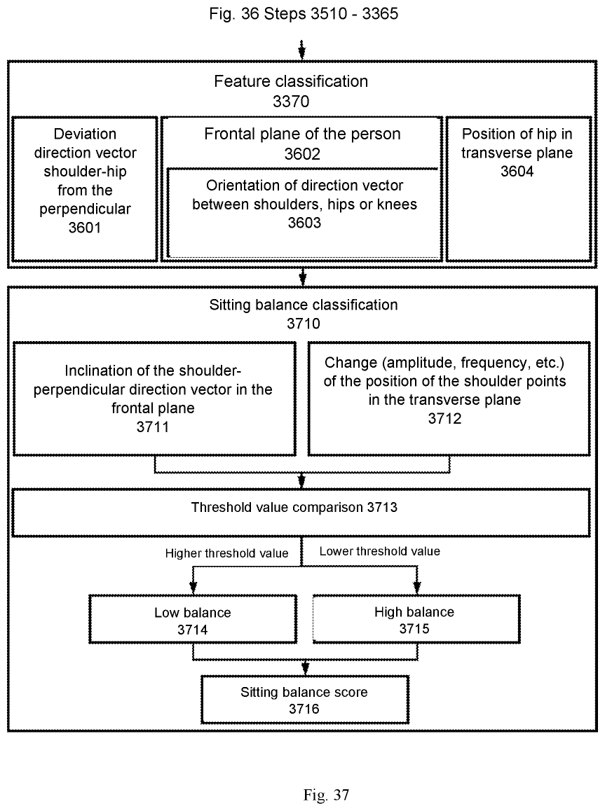

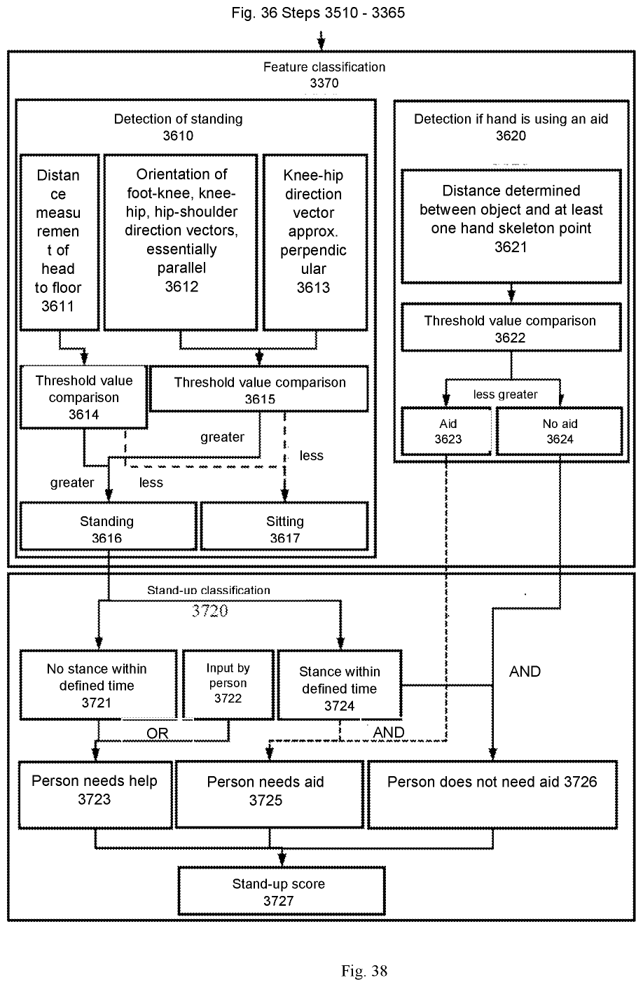

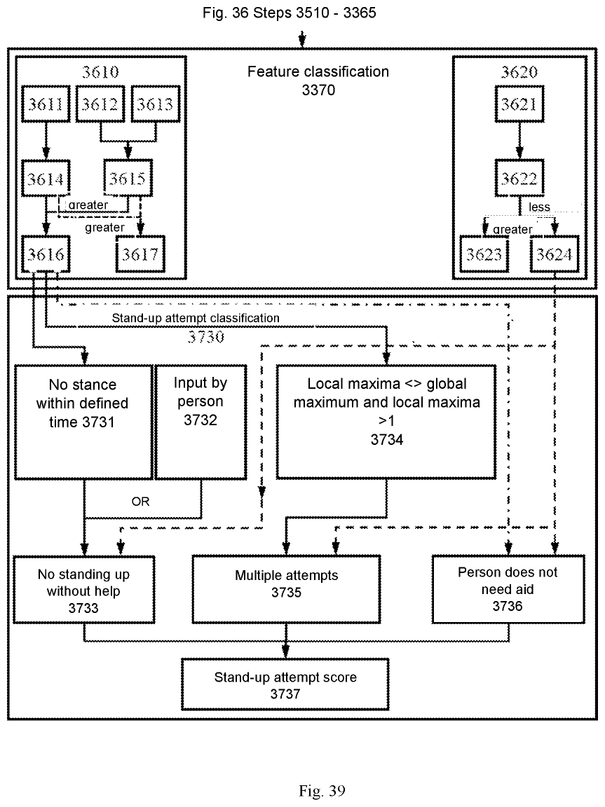

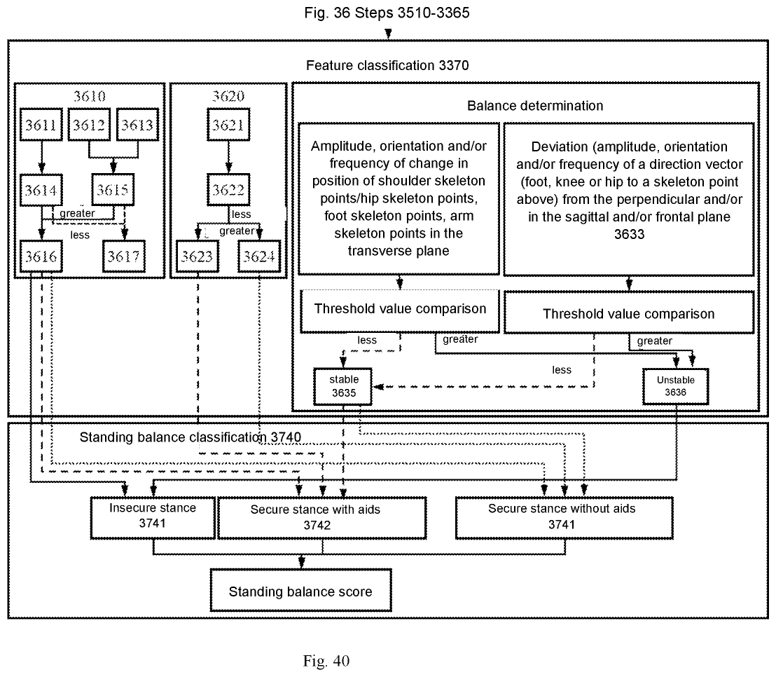

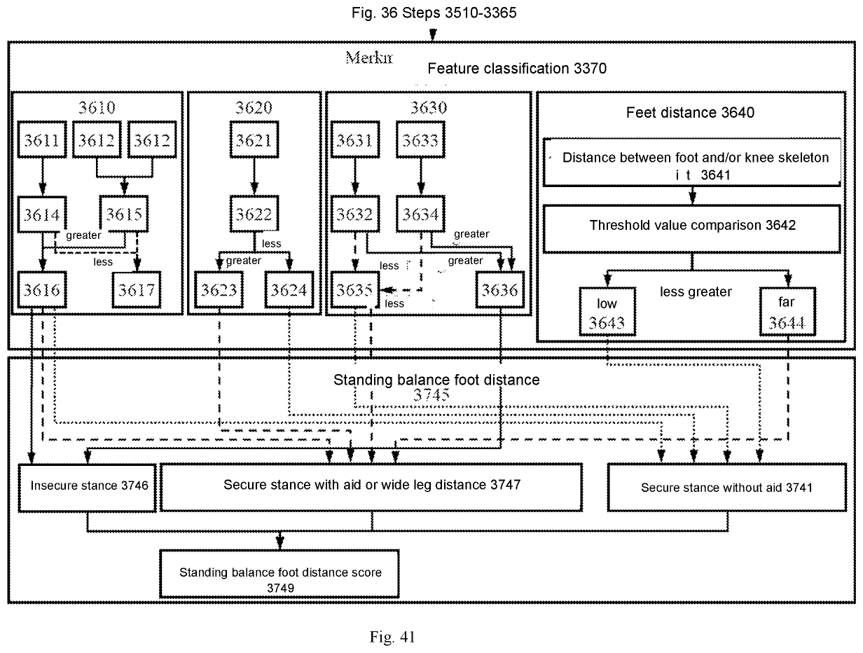

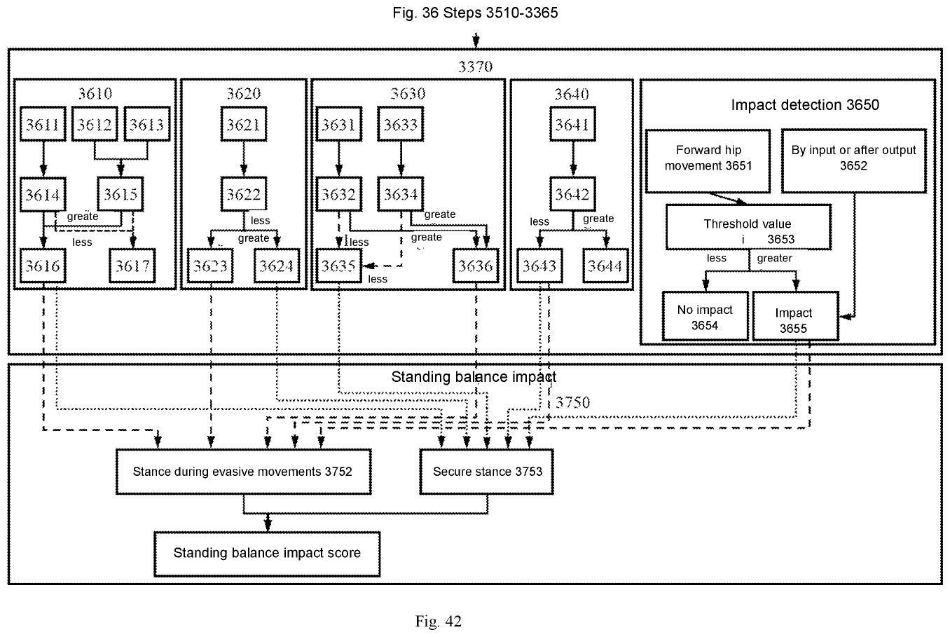

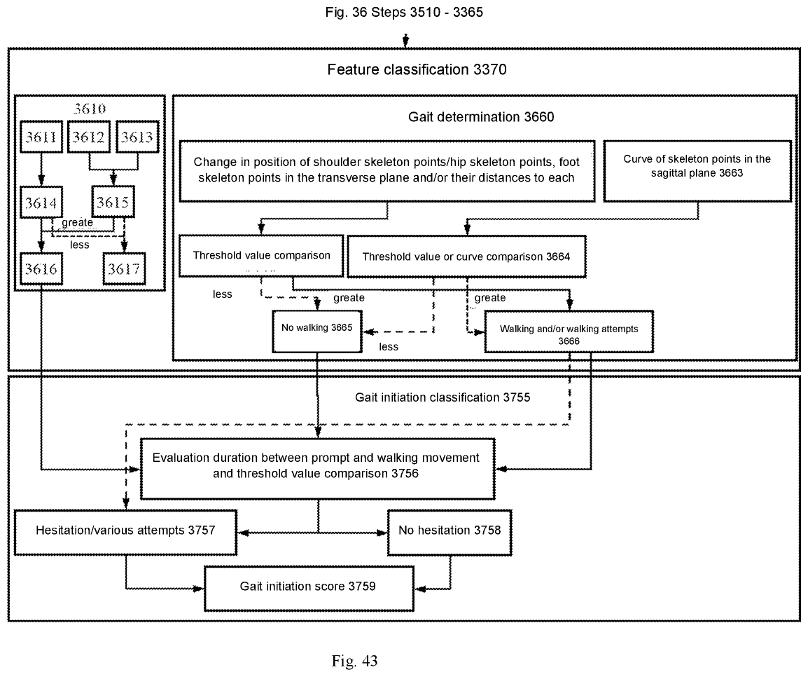

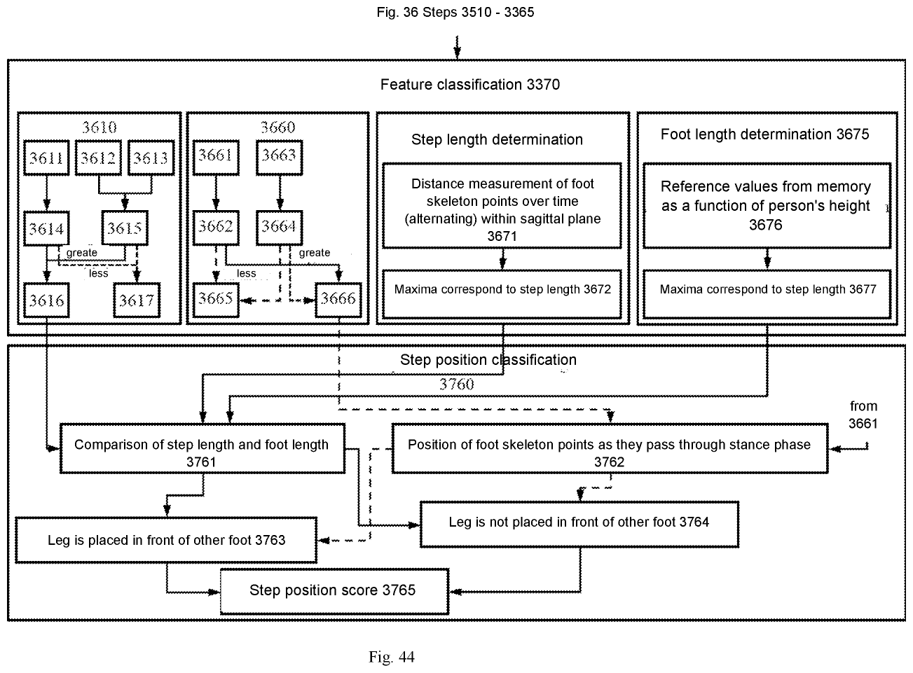

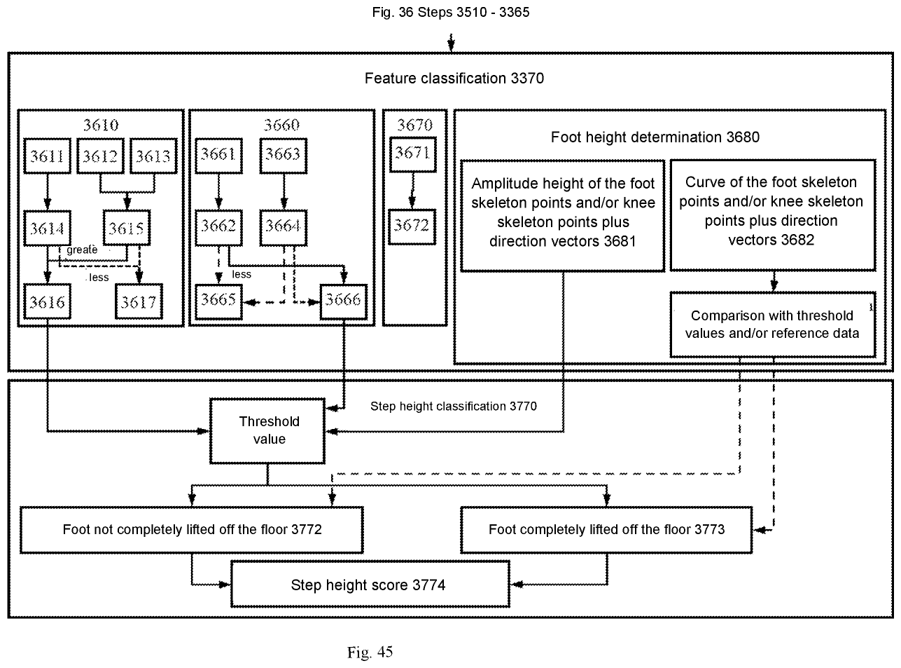

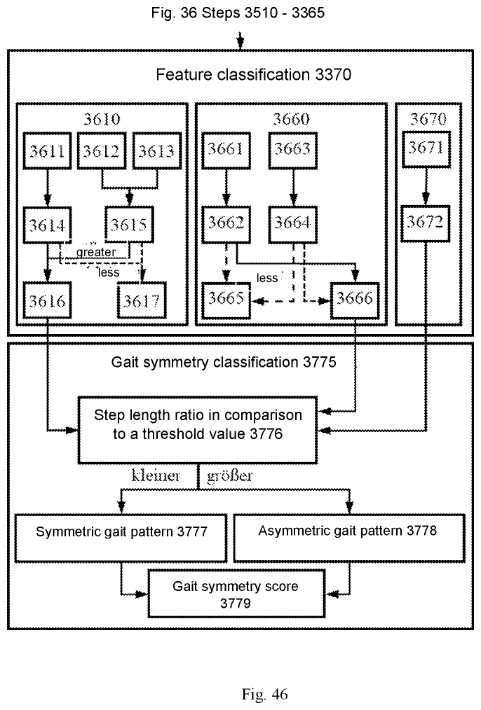

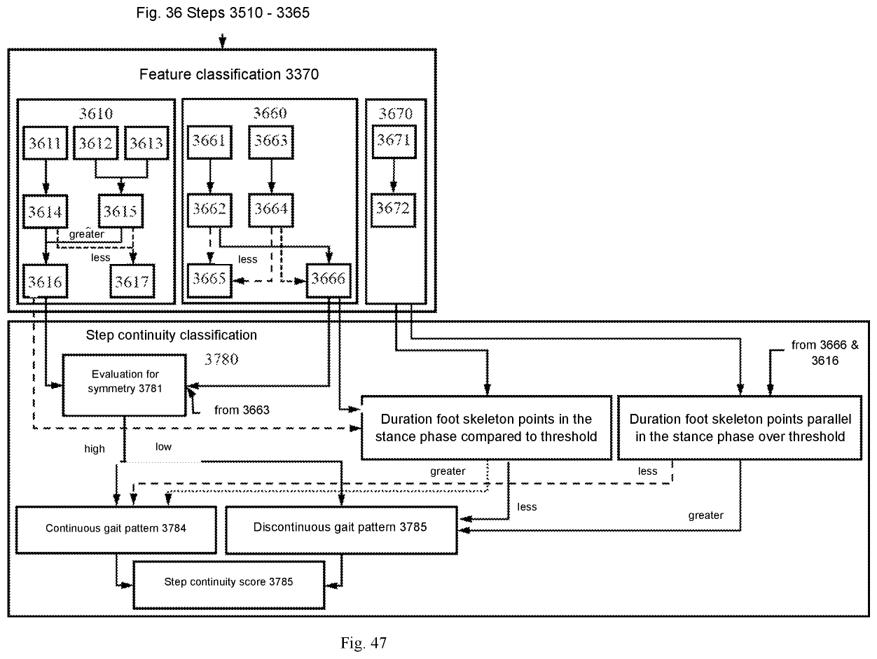

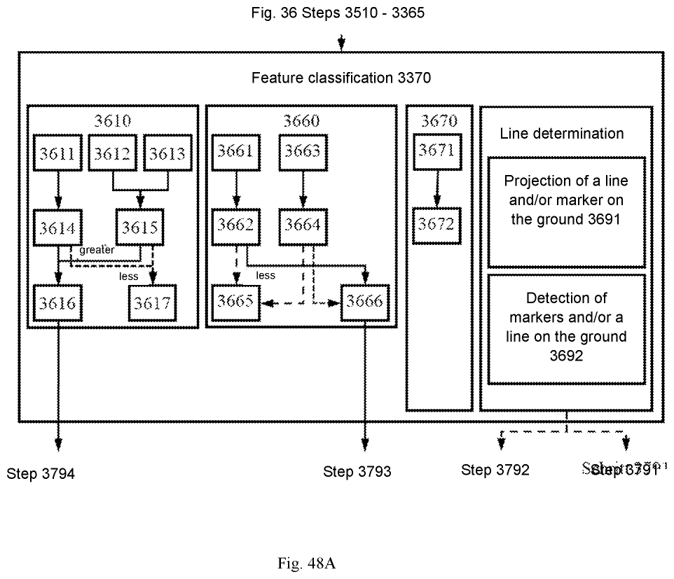

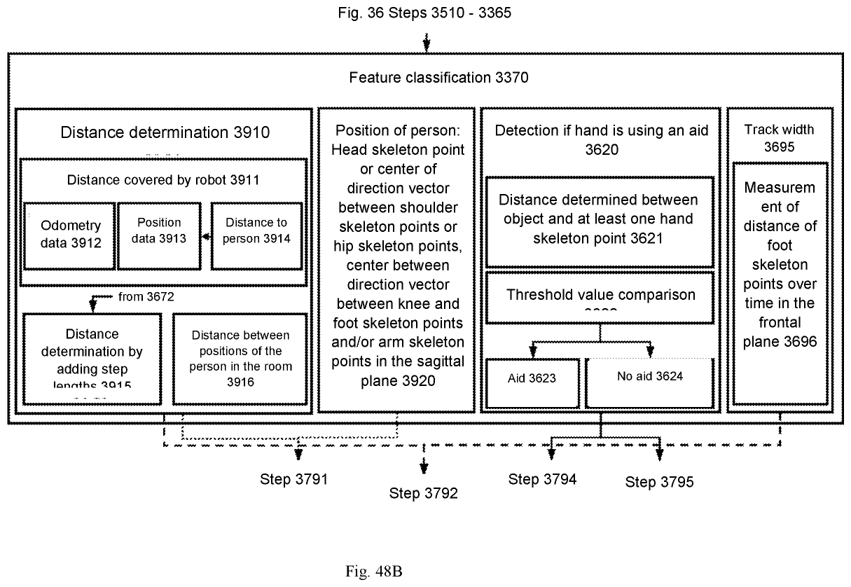

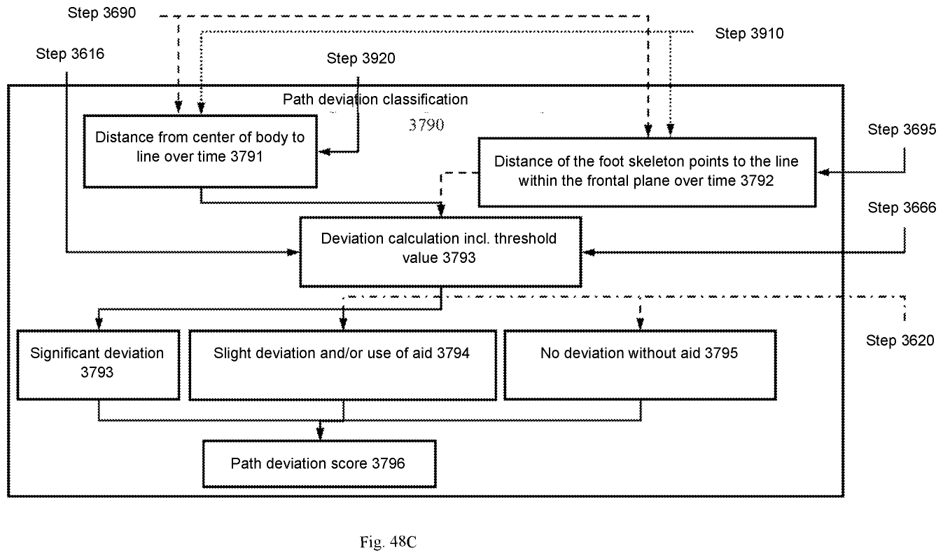

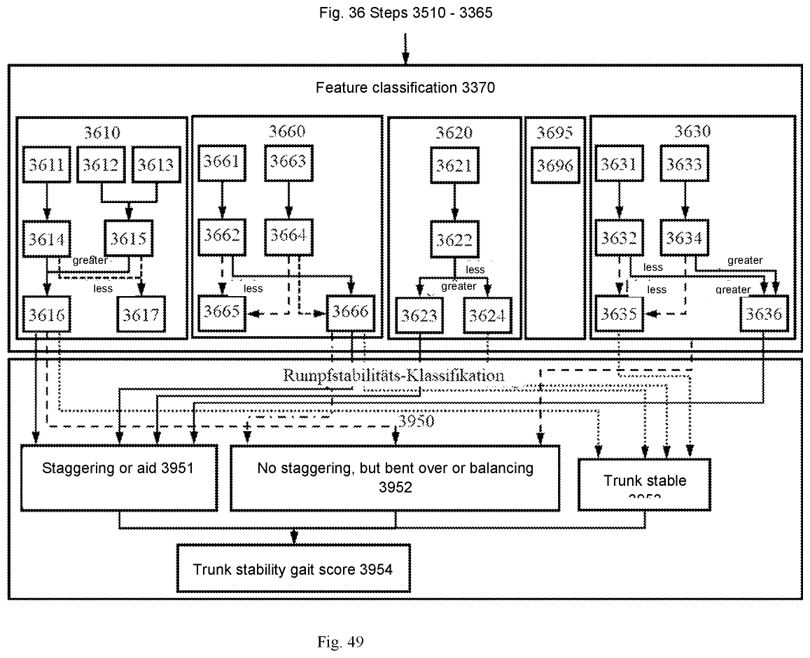

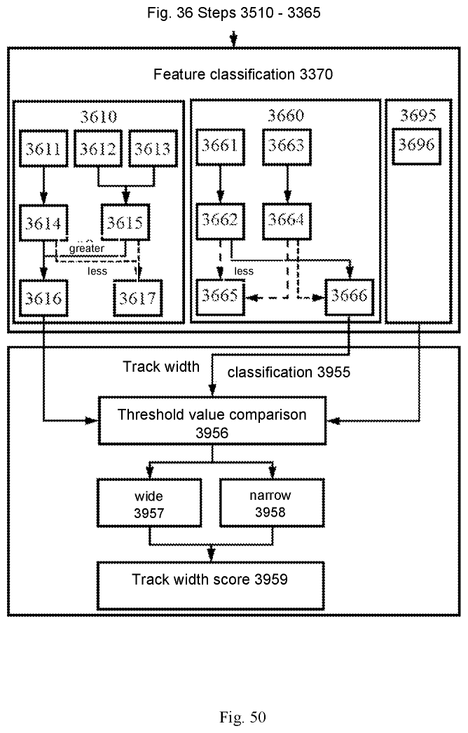

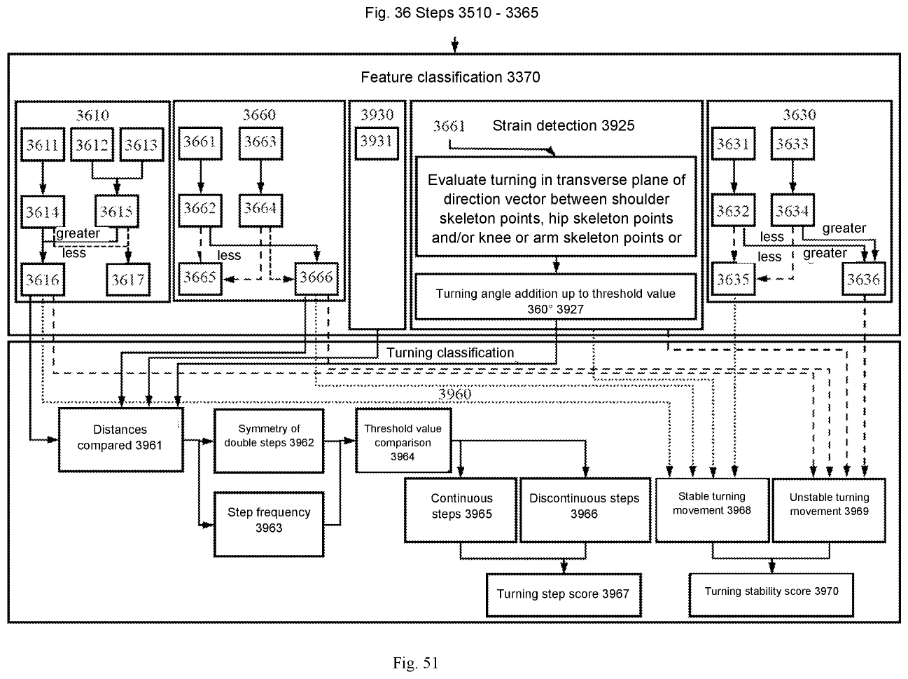

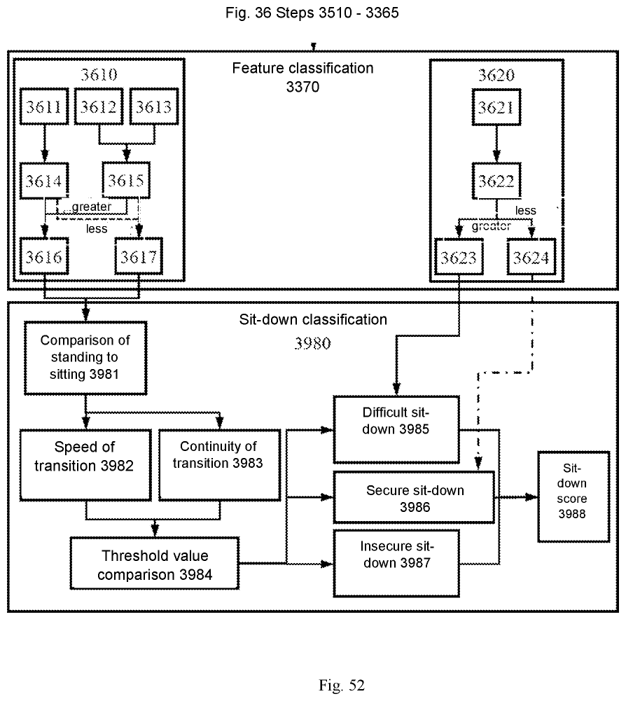

In one aspect, as shown in FIGS. 36-52, the service robot 17 is configured to evaluate various body positions and/or movements of a person while sitting, standing, and walking in order to provide a holistic view of a person's mobility. A number of the procedural steps are found in most of the steps, so they have been summarized on the basis of an example in FIG. 36. In one aspect, the service robot 17 may also move in front of the person while walking in step 3525, or in an alternative aspect, may move behind the person. To this end, as described elsewhere in this document, the person may log in at the service robot 17 in step 3510, and, in step 3355, the service robot 17 may identify and track the person, e.g. using the visual person tracking module 112 and/or the laser-based person tracking module 113 in conjunction with a LIDAR 1 and/or a camera 185. In another aspect, the service robot 17 may use an output unit to prompt the person to perform certain actions in step 3521, e.g. to stand up, walk, etc., with the outputs issued via the display 2, speech output, etc. Step 3521 is optional or dependent on the respective evaluation. In one aspect, the evaluation is preferably performed over time, for which purpose defined time intervals are used. In one aspect, the service robot 17 uses information from the skeleton model that is created by capturing the person by at least one 3D sensor and/or the camera 185 in step 3360 and that can be implemented using SDKs in the prior art. Feature extraction is performed in step 3365, including, for example, skeleton points in step 3541 and direction vectors between skeleton points in step 3542. Feature classification is subsequently carried out in step 3370, the details of which depend in particular on the respective task of the evaluation. The results of the feature classification in step 3370 are (optionally) stored and classified contiguously, which is in turn task-dependent, which is why this procedural step is referred to as “further classification” in step 3700 shown in FIG. 36. In one aspect, a threshold comparison may also be performed. A score is then determined for each task. Acquired data, such as data resulting from complete or partial evaluation and/or classification, may be stored (also on a temporary basis).

In one aspect, multiple skeleton points from the feature classification in step 3365 can be evaluated simultaneously during the feature classification carried out in step 3370 without explicitly defining angles in each case that result from the connections of the skeleton points. Instead, the position estimate in three-dimensional space (or alternatively, a position estimate for three-dimensional space based on two-dimensional data) may be carried out based on classifiers for which body poses were recorded and labeled as correct or incorrect, with the classifiers subsequently determined on this basis. Alternatively, body poses can be specified that describe a correct sequence and for which the positions and progression of skeleton points are evaluated over time. In this case, the course of skeleton points can be evaluated, for example, on the basis of a demonstration of a body pose, and a classifier can be created on this basis, which is then compared with other recorded body poses that have been specified as correct and the courses of the skeleton points in the room derived from these, after which a new classifier is created that takes all the available skeleton point progression data into account. For this purpose, for example, the Dagger algorithm can be used in Python. This way, for example, a neural network can be used to create a classifier that recognizes a correct movement and, consequently, also recognizes movements that do not proceed correctly. Body poses that are evaluated and classified are (non-exhaustively) those mentioned in the following paragraphs, including sitting balance, standing up, attempting to stand up, standing balance in different contexts, gait initiation, gait symmetry, step continuity, path deviation, trunk stability, 360° rotation, sitting down, use of forearm crutches, etc.

### Sitting Balance

As part of the evaluation, the service robot 17 detects the person and evaluates the extent to which a seated person leans to the side, slides on a chair, or sits securely or stably. In the process, features of the skeleton model are extracted, e.g. the skeleton points of the knees, pelvis, shoulder, head, etc., and direction vectors between each of the skeleton points are used to detect and evaluate the orientation of the person's body parts/limbs. In one aspect, therefore, the direction vector between at least one shoulder skeleton point and at least one hip skeleton point is evaluated (each preferably located on one half of the body; and/or parallel to the spine) and its deviation from the vertical/perpendicular 3601 in FIG. 37.

In a further aspect, the orientation of the person is evaluated, i.e. in this case, at least one direction vector between the shoulder points, between the hip points, e.g. also between the knees, etc. is detected in step 3603. Preferably, more than one direction vector is acquired. This direction vector is used, for example, to determine the frontal plane of the person in step 3602, which runs parallel to this direction vector. In another aspect, the position of the hip in the room is captured and deviations in the transverse plane over time are evaluated in step 3604. This is used to determine the extent to which the person slides back and forth in their seat, for example.

As part of the sitting balance classification in step 3710, the deviation and/or inclination of the direction vector between at least one shoulder skeleton point and at least one hip skeleton point from the vertical/perpendicular in the frontal plane is evaluated in step 3711. Furthermore, the change (amplitude, frequency, etc.) of the position of the shoulder skeleton points in the transverse plane is determined in step 3712. In step 3713, a threshold value comparison is performed via these two steps 3711 and 3712 and/or a comparison with patterns, e.g. movement patterns. If at least one of the determined values is greater than a threshold value (e.g. 1.3 m), the measurement result is classified as a low sitting balance in step 3714, otherwise as a high sitting balance in step 3715. In step 3716 score is assigned for each of these and stored in a sitting value memory.

### Standing Up

In one aspect, the service robot 17 evaluates the extent to which the person is able to stand up (see also FIG. 38). In the feature extraction process, the service robot 17 identifies objects and/or obstacles in step 3545, as described in the prior art. For this purpose, by way of example, the service robot 17 extracts the point cloud near the tracked hand skeleton points and performs a segmentation of the point cloud that allows the hands to be differentiated from the objects. This segmentation is preferably performed in real time (at 30 fps, for example). In one aspect, the captured point cloud can also be compared to point clouds stored in a memory, to which, for example, objects are assigned in order to establish an association between objects captured by sensors and their semantic meaning, which in turn allows certain objects to be classified as more relevant than others, e.g. a chair with an armrest or a walking aid as compared, for example, to a vase.

As part of the feature classification process, the standing pose is detected in step 3610. For this purpose, a distance measurement between the head and the floor is performed in step 3611, e.g. based on the position of the head skeleton point and at least one foot skeleton point. These values are compared in step 3614, if applicable with values stored in a memory and/or with a threshold value and/or a pattern. If the determined height is, for example, greater than the threshold value (e.g. 1.4 m), the person is classified as standing in step 3616, otherwise as sitting in step 3617. As an alternative to and/or in addition to the height of the person, the orientation of the direction vectors between at least one foot skeleton point and at least one knee skeleton point, at least one knee skeleton point and at least one hip skeleton point, and at least one hip skeleton point and at least one shoulder skeleton point is also evaluated in step 3612, whereby, in the case that these three direction vectors are essentially parallel to each other, as can be shown, for example, by a threshold value comparison 3615 and/or pattern matching, with the threshold value being calculated, for example, on the basis of the deviation from parallel. Alternatively and/or additionally, the orientation of a direction vector between at least one knee skeleton point and at least one hip skeleton point is evaluated with respect to the extent to which this direction vector is essentially perpendicular. If this deviation from parallel and/or from perpendicular is classified as less than the threshold value, the service robot 17 detects these features as standing in step 3616, otherwise as sitting 3 in step 617.

Furthermore, detection of whether a hand is using an aid is performed in step 3620, an aid being broadly understood here as a walking aid, an armrest of a chair, a wall, etc., i.e. anything that a person can use to help him- or herself stand up. In step 3621, the distance between at least one wrist and at least one of the extracted objects is determined. If the distances of at least one hand from the object(s) or obstacle(s) fall below a threshold value 3622 (e.g. 8 cm), this is classified as aid use in step 3623, otherwise as no aid use in step 3624. In one aspect, this assumes that there is a minimum distance to the body of the person under consideration, i.e. to the skeleton points and/or direction vectors connecting the skeleton points.

In the scope of the stand-up classification 3720, the service robot 17 makes the following classifications: if the person is standing 3721 after a defined period of time or if a person makes an input 3722, especially an input that the person is incapable of standing up (by him- or herself), then the situation is classified in step 3723 as one in which the person requires assistance. If the standing occurs within a defined time in step 3724 and the person uses aids in step 3623, then the person is classified in step 3725 as a person who requires aids to stand up 3725. The third case classified here is the case where the person does not require any aids in step 3624 and achieves a standing position in step 3724 within a defined period of time, with the person thereby able to stand up without any aids in step 3726. A stand-up score 3727 is determined based on steps 3723, 3725, and 3726.

### Attempts to Stand Up

In an alternative and/or additional variant of the preceding detection of standing up, attempts to stand up are also determined (FIG. 39). The feature classification therefore includes, in addition to the feature classification shown in FIG. 38, an evaluation of the knee-hip direction vector for its horizontal position, i.e. to establish the extent to which the service robot 17 is parallel to the transverse plane.

The following steps are performed in the stand-up test feature classification 3730: If, based on the information from the stand-up attempt feature classification 3370, no standing is detected within a defined time in step 3731, or if an input is made by the person in step 3732 and (compared to steps 3731 and 3732) no aid is detected in step 3624, the person is classified in step 3733 as a person who cannot stand up without assistance. If no aid is detected in step 3624 and the local maxima are not equal to the global maximum and the number of local maxima is greater than 1, multiple attempts to stand up are detected in step 3735. For this purpose, the progression of the skeleton points defining standing is evaluated over time and/or the angle or angular change of the direction vector between the hip and the knees with respect to the horizontal (alternatively: vertical), where the horizontal is described via the transverse plane. If, for example, it is detected 2× that the angle changes from approx. 0° (transverse plane) to approx. 30° (change in one rotational direction), but there are then changes in another rotational direction (e.g. 30° again), and only then is an angular change >>30° detected, e.g. 90°, three stand-up attempts are detected (the last of which was successful). If, on the other hand, no aids are detected in step 3624, standing occurs 3616, and the situation is classified in step 3736 as one in which the person does not require aids. An overall stand-up attempt score 3737 is assigned based on steps 3733, 3735, and 3736.

### Standing Balance

In an alternative and/or additional aspect, the service robot 17 evaluates the standing balance of a person, as shown in FIG. 40. In addition to previous evaluations, a balance determination 3630 is carried out in the feature classification 3370. For this purpose, the amplitude, orientation, and/or frequency of the change in position of at least one of the shoulder skeleton points, at least one of the hip skeleton points, or at least one of the foot skeleton points in the transverse plane 3631 is evaluated over time (for example, for 5 seconds) and a threshold value comparison is performed in step 3632 and/or a comparison with patterns, such as movement patterns. In this context, with respect to the foot skeleton points, the step length and/or existence of steps may also be evaluated in one aspect. If the amplitude, orientation, and/or frequency of the change in position are less than the threshold value 3632 (e.g. a lateral variation of 10 cm) and/or do not correspond to a pattern, stability 3635 is assumed, otherwise instability 3636 is assumed. Alternatively and/or additionally, the deviation (amplitude, orientation, and/or frequency of at least one direction vector (foot, knee or hip to at least one overlying skeleton point) from the perpendicular and/or in the sagittal and/or the frontal plane can be evaluated in steps 3633 and 3631 over time (for example, for 5 seconds). The overlying skeleton points include, for example, a head joint in addition to at least one shoulder skeleton point. Based on a threshold value comparison performed in step 3634, deviations that are below the threshold value and/or a certain pattern are labeled as stable 3635, otherwise as unstable 3636. The standing balance classification performed in step 3740 classifies the person as having an insecure stance 3741 if the person is standing 3616 but unstable 3636. The person is classified as having a “secure stance with aids” if the person is standing 3616 and using aids 3623 while maintaining a stable balance 3635. A “secure standing without aids” 3743 is assumed if the person is standing 3616, not using aids 3624, and maintaining a stable standing balance 3635. Based on this rating, a standing balance score 3744 is assigned.

Standing Balance with Feet Close Together

As an alternative to and/or in addition to a previous evaluation of the standing balance (see FIG. 41), preferably after an output 3521 by the service robot 17 prompting the person to place his or her feet closer together when standing, the foot distance 3640 is determined during feature classification 3370. For this purpose, the foot skeleton points and/or the knee skeleton points are used from the position of the extracted skeleton points 3541, as well as, in one aspect, the orientation of the direction vectors 3542 between the hip skeleton point and the knee skeleton point and/or the knee skeleton point and the foot skeleton point. Based on this data, the distance of the foot skeleton points 3641 is determined, in one aspect within the frontal plane. A threshold value comparison 3642 and/or pattern matching is then applied to classify whether the feet are far 3643 or close together (i.e. at a short distance 3643), whereby 12 cm, for example, can be used as the threshold value (from joint center to joint center).

In the standing balance foot distance classification 3745 that follows, stances are classified into three classes: The first class (insecure stance 3746) encompasses persons who stand 3616 but can only maintain an unstable balance 3636. The second class encompasses standing persons 3616 with a stable balance 3635 and who use aids 3623 or stand with a wide foot distance 3644. The third class is for persons who stand 3616, maintain a stable balance 3635, do not use aids 3624, and stand with a short foot distance 3643. This classification results in a standing balance foot distance score 3749.

In one aspect, the foot skeleton points may not be obtained directly from the SDK data that extracts the skeleton model, but rather alternatively via the knee skeleton point. Moreover, the position of the knee skeleton point, the direction vector originating from the knee skeleton point with a parallel orientation to the lower leg, and the height of the knee skeleton point above the floor if the direction vector passes through the perpendicular (between a hip skeleton point and a knee skeleton point or a knee skeleton point and the floor) are determined, with the height of the knee skeleton point above the floor indicating the distance at which the foot skeleton point is located as viewed from the knee skeleton point if the direction vector passes through the perpendicular.

### Standing Balance and Impact

As an alternative to and/or addition to an evaluation of the standing balance described above, the service robot 17 detects a person receiving at least one impact on the back (see FIG. 42). In step 3651, impact detection 3650 is performed to evaluate the forward movement of the hip, i.e. within the sagittal plane. Alternatively and/or additionally, an input at the service robot 17 or an output is evaluated in step 3652. The movements (such as an acceleration) of the hip are subjected to a threshold value comparison in step 3653 and/or a pattern matching. If the threshold value is not exceeded or the pattern is not detected, no impact is detected in step 3654, otherwise an impact is detected in step 3655. Alternatively and/or additionally, an input can also be made to the service robot 17, for example, that the patient is subsequently impacted, and/or an output can be made that represents, for example, an impact command as a result of which the person is impacted, so that the consequences of the impact on the balance of the captured person can be evaluated. The standing balance during the impact is subsequently evaluated by the standing balance impact classification 3750. At least two classes are distinguished: a) the standing balance is secure/stable 3753, which is characterized by a stance 3616, stability of balance 3635, no aids 3624, and a short foot distance 3643 after an impact 3655 has occurred; b) the person exhibits a stance while making evasive movements 3752, i.e. he or she takes evasive steps or supports him- or herself, but maintains a standing position in the process. For this purpose, the person exhibits a stance 3616, uses aids 3623, is unstable 3636 (which is made apparent by evasive movements), and exhibits a short foot distance 3643, with an impact having occurred previously 3655. This classification results in a standing balance impact score 3754.

### Standing Balance and Closed Eyes

As an alternative to and/or in addition to an evaluation of the standing balance as described above, the standing balance with closed eyes is recorded and evaluated. For this purpose, in one aspect, the service robot 17 can detect the face of the person and his or her eyes, and distinguish between closed and open eyes by changes in color, color contrast, etc., which are detected by an RGB camera. Alternatively and/or additionally, the service robot 17 issues an output, e.g. acoustically, which prompts the person to close his or her eyes. Movements detection is carried out in each case after detecting closed eyes and/or the output. The standing balance is determined analogously to FIG. 42, except that no impact is evaluated here, and a stable or unstable stance is classified in the result, which results in a standing balance eye score.

### Gait Initiation

As an alternative to and/or in addition to an evaluation described above, the service robot 17, preferably following an output that includes a prompt to walk, records the gait behavior of the tracked person and determines the time duration until gait initiation, as shown in FIG. 43. Feature classification 3370 includes a gait determination 3660. In one aspect, the change in position of the shoulder skeleton points, hip skeleton points, foot skeleton points in the transverse plane and/or the distances between the foot skeleton points 3661 are determined, in each case over time. In step 3662, a threshold value comparison and/or pattern matching is performed, and if the threshold value (e.g. 10 cm) is exceeded, walking and/or attempts to walk are assumed 3666, otherwise they are not assumed 3665. Alternatively and/or additionally, the curve of skeleton points in the sagittal plane 3663 can be evaluated, for which threshold values and/or curve comparisons 3664 and/or pattern matching can be used. Based on this, the movement is classified into walking and/or walking attempts 3666 or no walking 3665. In one aspect, walking attempts are detected if the movement is relatively slow and/or discontinuous in the sagittal or transverse plane, where “relatively slow” implies falling below a threshold value. In the scope of gait initiation classification 3755, the time duration between prompting and gait movement 3756 is evaluated. If this walking movement is, for example, above a threshold value (such as 2 seconds) and/or various walking attempts are detected in step 3666, this is classified as hesitation/various attempts in step 3757. If this walking movement takes place within a time interval that is below the threshold value, this is classified as no hesitation in step 3758. The result is assessed with a gait initiation score 3759.

### Step Position

As an alternative to and/or in addition to the above evaluations, the service robot 17 evaluates the walking movement of a person (as described, for example, in the previous section), as also shown in more detail in FIG. 44, in order to determine the step lengths of the left leg and/or the right leg.

The service robot 17 detects the distance between the foot skeleton points over time as part of feature classification 3370, with the maxima in the sagittal plane corresponding to the step length 3672. In the process, the service robot 17 alternately assesses the position of the foot skeleton points relative to each other in the sagittal plane. In one aspect, the foot length factored into the subsequent step position classification 3760, for which purpose the foot length is determined in step 3675. In one aspect, this is interpolated over the height of the person, with different foot lengths being stored in a memory for different heights of a person, i.e. reference values from the memory are used for this in step 3676.

The values determined in this way are further classified in the step position classification 3760. For this purpose, in one aspect, the step length is related to the foot length in step 3761. Alternatively and/or additionally, the position of the respective foot skeleton points in the sagittal plane is assessed when passing through the stance phase, and the position of the foot skeleton points relative to each other is compared in step 3762, with the position data originating from step 3661.

It is then assessed whether the respective leg under consideration is placed in front of the foot of the other leg 3763 or not 3764. This leg is placed in front of the foot of the other leg if the comparison of step length and foot length in step 3761 indicates that the step length is shorter than the foot length and/or if the foot skeleton point position of the leg under consideration is not placed in front of the foot of the other leg in the gait direction in the sagittal plane, as indicated by the position of the foot skeleton points when going through the stance phase 3762. Based on this classification, a step position score 3765 is assigned. In one aspect, such an evaluation may be performed separately for each leg.

Standing 3616 is to be understood here (and also in additional (e.g. subsequent) evaluations concerning walking) as meaning that the person is essentially in an upright position in which the person is located in one place (de facto standing) or can also walk at will. Otherwise, the methods described could capture any locomotion of the person that would not generally be described as walking.

In one aspect, the service robot 17 follows the person as he or she walks or moves in front of the person 3525, during which, in one aspect, the service robot 17 adjusts its speed to the speed of the person 3530, with a possibly discontinuous speed of the person being converted to a continuous speed of the service robot 17, for example by averaging the speed of the person or controlling the speed of the service robot 17 over a time interval that is adjusted to the speed of the person within the time interval.

### Step Height

As an alternative to and/or in addition to the foregoing evaluations, the service robot 17 detects the walking movement of a person as shown in FIG. 45 and classifies the extracted features in such a way that the height of the feet (above the floor) is determined 3680. For this purpose, in one aspect, the amplitude height of the foot skeleton points and/or knee skeleton points plus direction vectors is evaluated over time in step 3681 and also evaluated, for example, in the sagittal plane, with this being used with respect to the knee skeleton points for the derivation of the foot skeleton points as already described above. As an alternative to and/or in addition to this, the curve of the foot skeleton points and/or knee skeleton points plus direction vectors is evaluated in step 3682. In particular, the rises/falls of the amplitudes are evaluated, serving as a proxy for the step height, and a comparison with threshold values and/or reference data is performed in step 3683. In one aspect, the sinusoidal shape of the movements is captured here, implying a higher likelihood of the leg being lifted off the floor as far as possible, whereas a movement that is more trapezoidal in movement is more likely to imply a dragging movement in which the foot is not properly lifted off the floor. As part of step height classification 3770, the captured step heights are evaluated via a threshold value comparison 3771 and/or pattern matching. If the step heights fall below a step height threshold value (e.g. 1 cm) or prove dissimilar to a pattern, the foot is classified as not completely lifted off the floor in step 3372, otherwise as completely lifted off the floor in step 3373. In one aspect, a lifted or non-lifted foot can also be directly inferred from the evaluated curves. The results of the classification are factored into the step height score 3774. In one aspect, such an evaluation may be performed separately for each leg.

### Gait Symmetry

As an alternative to and/or in addition to the preceding evaluations, the service robot 17 evaluates the symmetry of the gait pattern when capturing the gait, as described in the preceding sections (see also FIG. 46), for example. This is performed in the scope of a gait symmetry classification 3775. In particular, this gait symmetry classification 3775 uses data from the step length determination 3760, i.e. the step lengths 3762, and, in one aspect, evaluates these when the person is standing 3616 or walking 3666. As part of this gait symmetry classification, the step length ratio is evaluated in comparison to a threshold value 3776 and/or movement patterns over time. In one aspect, the symmetry of step lengths is also evaluated per double step, where a double step is calculated by adding one step of the left leg and one step of the right leg (or vice versa). The step length ratio may be formed, in one aspect, as a ratio of the single step lengths to each other, or, in another aspect, as a ratio of a single step length to the double step length. If the respective ratio is below a threshold value or if a comparison with patterns shows, for example, a high pattern similarity, the gait pattern is classified as symmetric 3777, otherwise as asymmetric 3778. For example: a step length ratio of 1:1.1 or less (or 60:66 cm for the single step or 60:126 cm for the double step) is classified as symmetric, while larger ratios are classified as asymmetric. The classifications are then converted to a gait symmetry score 3779.

### Step Continuity

As an alternative to and/or in addition to the preceding evaluations, the service robot 17 evaluates the step continuity when capturing the gait as described in the preceding sections (see FIG. 47), for example. In one aspect, the position determination of the foot skeleton points in the stance phase 3673 can also be performed as part of the step length determination 3670.

Step continuity classification 3780 is performed, in one aspect, to evaluate the curve of the skeleton points in the sagittal plane 3663 with respect to the symmetry of the curve while standing 3616 or walking 3666 3781. High symmetry results in a classification as a continuous gait pattern 3784, otherwise the gait pattern is classified as discontinuous 3785. Alternatively and/or additionally, the step lengths 3672 are evaluated with simultaneous capture/extraction of the points at which the feet touch the floor, i.e. position detection of the foot skeleton points is performed in the stance phase 3673. If the service robot 17 detects, for example, that the distance between the foot skeleton points falls below a threshold value (e.g. 20 cm) or does not exhibit a minimum similarity in a pattern matching as determined in the process step “Distances between ankles in stance phase compared to threshold value” 3782 at the times when the left foot and the right foot (or vice versa) touch the floor, this step continuity is also classified as a discontinuous gait pattern 3785. This is the case, for example, if the person always places one foot forward and drags the second foot behind, so that both feet are approximately parallel at the moment of reaching a standing position. Alternatively and/or additionally, such a case can also be registered by the service robot 17 if both legs are parallel (in the sagittal plane) beyond a defined temporal threshold value 3783. The classifications are subsequently converted into a step continuity score 3786.

### Path Deviation

As an alternative to and/or in addition to the preceding evaluations, when capturing the gait, e.g. as described in the preceding sections, the service robot 17 evaluates the deviations of the gait from a line, as shown in FIG. 48, with the line being either virtual or real. The person is prompted via an output 3521 to move along a line that is at least 2 m long, preferably 3 m long. In one aspect, line determination 3690 is used for this purpose. In one aspect, a projection of a line and/or at least one marker on the ground 3691 may be also detected, and in an alternative and/or additional aspect, at least one marker and/or line on the ground is detected in step 3692. In an alternative and/or complementary aspect, the marker and/or line is projected onto the ground by the service robot 17. An example of how such a projection may be performed is provided earlier in this document. The line may also be virtual and comprise, for example, the person's direct connection to a marker and/or the line at which the sagittal plane of the person intersects the floor, with the line being determined at the beginning of the evaluation and/or after the output 3521 of the prompt to traverse the respective distance.

Furthermore, distance determination is performed 3910 in order to verify that the person has covered the distance along the line. In an aspect not shown in detail in FIG. 48, outputs of the service robot 17 may be issued if applicable in order to prompt the person to take more steps to reach the target distance (e.g. 3 m) or to stop when the target distance has been reached. The distance can be determined in a number of ways. In one aspect, the service robot 17 determines the distance covered by the service robot 17 3911 by means of odometry data 3912, for example, and/or using position data 3913. In the latter case, the distance is determined as the difference between at least two positions. Also, in one aspect, the distance to identified obstacles and/or objects may also be evaluated. In order to evaluate the distance covered by the person on the basis of this data, the distance to the person is evaluated over time in step 3914, and the distance covered by the person is calculated on this basis. Alternatively and/or additionally, the distance can be determined by adding the step lengths in step 3915, which were recorded in step 3672. In an alternative and/or additional aspect, the position can also be determined by evaluating the positions of the person in the room in step 3916 (see also step 3695 below), i.e. in particular by evaluating the distance between the coordinates that change when the position changes.

Furthermore, the position of the person is evaluated in step 3920 by evaluating the position of the head skeleton point, e.g. in the transverse plane, and/or the center of the direction vector between the shoulder skeleton points or the hip skeleton points and/or the center between the direction vector connecting the knee skeleton points (e.g. projected into the frontal plane), the direction vector between the ankle skeleton points (e.g. projected into the frontal plane), and/or a direction vector between at least two homogeneous arm skeleton points (e.g. projected into the frontal plane).

In a further aspect, an evaluation is made as to whether the person is using aids, as described above in step 3620.

The service robot 17 determines the distance of the center of the body from the line over time 3791 and/or the distance of the foot skeleton points from the line within the frontal plane over time 3792. A deviation calculation including a threshold value 3793 or pattern matching is then performed, i.e. the maximum of the deviation, the least squares of the individual deviations per step, etc. are calculated for the determined distances, although other approaches described in the prior art can also be used for distance evaluation.

Classification is subsequently carried out as follows: The result is classified as a significant deviation in step 3793 if the person stands 3616, walks 3666, and the value of the line deviation in the deviation calculation including threshold value 3793 is above a threshold value or has a minimum pattern similarity in a pattern matching. The result is classified as a slight deviation and/or aid use 3794 if the person stands 3616, walks 3666, and the value of the line deviation in the deviation calculation including threshold value 3793 and/or pattern matching is in an interval whose upper value is the threshold value for classification according to 3793. Furthermore, as an alternative to and/or in addition to the deviation from the line, an aid is detected in step 3620. The result is classified as no deviation without aid use 3795 if the person stands 3616, walks 3666, the value of the line deviation in the deviation calculation including threshold value 3793 is below a threshold value (or a pattern matching does not attain a pattern similarity), and no aid use is detected in step 3620. In the next step, the path deviation score is calculated in step 3796 based on this classification.

### Trunk Stability

As an alternative to and/or in addition to the previous evaluations, the service robot 17 evaluates the trunk stability during walking, which happens analogously or similarly to the standing balance determination, with the difference that the person is also walking (see FIG. 49). In the trunk stability gait classification, the result of the feature classifications 3370 of the different aspects is evaluated as follows.

The person is classified as staggering or as aid-using in step 3951 if the person stands 3616, walks 3666, uses aid 3623, and is unstable 3636. The person is classified as not staggering but bending or balancing in step 3952 if the person stands 3616, walks 3666, and either (as a partial aspect of balance determination) leans forward (e.g. evaluated via the direction vectors in 3633) or the arm skeleton points are at a distance from the body that is above a threshold value or that exhibits, for example, a pattern dissimilarity in a pattern matching, (e.g. evaluated via the periodic movements of the arm skeleton points in the transverse plane in step 3631). The person is classified as having a stable trunk 3953 if the person stands 3616, walks 3666, uses no aids 3624, and is stable 3635. In the next step, the path deviation score 3954 is calculated based on this classification.

In the case of balance determination 3630, the amplitude and/or frequency of periodic or aperiodic movements detected as substantially parallel to the frontal plane may, in one aspect, also be evaluated via threshold value comparison and/or pattern matching.

### Step Width

As an alternative to and/or in addition to the preceding evaluations, the service robot 17 evaluates the track/step width when capturing the gait, as described in the preceding sections (see FIG. 50), for example. For this purpose, the track width is evaluated 3695 as part of feature classification 3370, which is implemented, for example, as a distance measurement of the foot skeleton points over time in the frontal plane 3696. In the scope of track width classification 3955, threshold value comparison 3956 and/or pattern matching is performed using the track width data. Whether the person is standing 3616 and walking 3666 is also considered. If the track width is below the threshold value (e.g. 18 cm) or pattern matching reveals a defined minimum dissimilarity, the track width is classified as narrow 3958, otherwise as wide 3957. The result is converted into a track width score 3959. In one aspect, the track width can be corrected for the width of the hip skeleton point, which is approximated by the length of the direction vector between the hip skeleton points.

### 360° Turn

As an alternative to and/or in addition to the preceding evaluations, the service robot 17 evaluates the turning movement of the captured person when capturing the gait, as described in the previous sections (see FIG. 51), for example. This is preferably a turning movement of 360°. The step length 3930 is determined in the feature classification 3370 process. However, the process at this point is different from the one applied in step 3670, because it is not the distance in the sagittal plane that is evaluated, but rather the absolute distance, as a step position can also be oblique due to the turning movement of the person. Furthermore, data from step 3661 is used to evaluate, for example, whether and to what extent the person is rotating, i.e. a rotation detection is performed in step 3925. Also, in one aspect, the rotation executed in the transverse plane by the direction vector between the shoulder skeleton points, the hip skeleton points and/or the knee or arm skeleton points or the head is evaluated in step 3926. Here, the rotation angles are detected and added, and the added values are evaluated to determine whether the addition has reached the value of 360° (rotation angle addition to reach the threshold value 360° in step 3927 or pattern matching). In one aspect, this is done after an output issued by the service robot 17 in step 3521.

In the event of a 360° rotation (resulting from step 3925), a detected walking movement in step 3666, and standing in step 3616, an evaluation of the previously detected step lengths is performed comparing the distances between the steps in step 3961. In the process, the symmetry of the double steps 3962 is evaluated, i.e. the double step lengths and the ratio of the single step lengths to each other and/or in comparison to the double step are evaluated. Alternatively and/or additionally, the step frequency 3963 can also be evaluated, in particular with respect to its periodicity, i.e. the rise and fall of the curves and, from this, the amplitude symmetry. The periodicity and/or the symmetry of the double steps is evaluated by way of a threshold comparison performed in step 3964 and/or a pattern matching, with high symmetry resulting in steps classified as continuous 3965, otherwise discontinuous 3966. The results are converted into a turning step score 3967.

Alternatively and/or additionally, a turning stability score 3970 is also determined. For this purpose, a turning movement captured in 3925 is evaluated with respect to balance in step 3630. As a result, such turning movements in which the balance is stable 3635 and in which the person in standing 3616 and walking 3666, are classified as stable turning movements 3968. In contrast, movements in which the person is standing 3616 and walking 3666, but is unable to maintain a stable balance in step 3636, are classified as an unstable turning movement 3969. The results are carried over to the turning stability score 3970.

### Sitting Down

The service robot 17 uses, at least in part, feature classifications already described above (see FIG. 52) to detect a person's sitting down as a response to an output 3521 issued by means of at least one output device of the service robot 17. In the process, the service robot 17 performs a sit-down classification 3980 to evaluate the transition from standing 3616 to sitting 3617, with this step being described with 3981. In particular, the speed of the transition 3982 is evaluated. Furthermore, the service robot 17 determines the continuity of the transition 3983, for example by sequentially comparing the momentary velocities during the process of sitting down and/or by comparing these with values stored in a memory. Based on these two steps 3982 and 3983, a threshold value evaluation 3984 and/or a pattern matching is/are performed. Furthermore, classification results 3620 are used that evaluate whether the hand is using an aid, which includes using the hand to prop oneself up. The results are then classified as follows:

If the sit-down speed exceeds a threshold value 3984, the act of sitting down is classified as an insecure sit-down 3987. If the value of the discontinuity during the process of sitting down exceeds a threshold value 3984 and/or an aid 3623 is used, the movement is classified as a difficult sit-down 3985. If no aid is detected in step 3624 and if the speed of the act of sitting down falls below a threshold value in step 3984 and/or if the value of the discontinuity during the process of sitting down falls below a threshold value in step 3984, the act of sitting down is classified as secure in step 3986. The results of this evaluation are converted to a sit-down score 3988.

### Alternative Evaluation of the Person's Movements

In an alternative and/or additional aspect, the service robot 17 takes two- or three-dimensional images of the person and compares these images with images stored in a memory of persons who also assume the same postures or perform the same movements and which have been classified according to the extent to which the person recorded, for example, leans to the side, slips, or sits securely or stably; the extent to which this person stands up and uses aids, attempts to stand up, maintains standing balance, takes steps, exhibits gait symmetry, exhibits trunk stability, turns 360°, etc. This classification can be performed using classification methods described in the prior art, e.g. methods of machine learning/artificial intelligence. Based on this comparison, the service robot 17 classifies the recorded images of the person. The score is assigned to each exercise in an analogous manner, as described above.

In one aspect, the service robot 17 may also transmit the recorded data via an interface 188 (such as WLAN) to other systems in which the data is subsequently evaluated, e.g. using the method described for evaluation within the service robot 17.

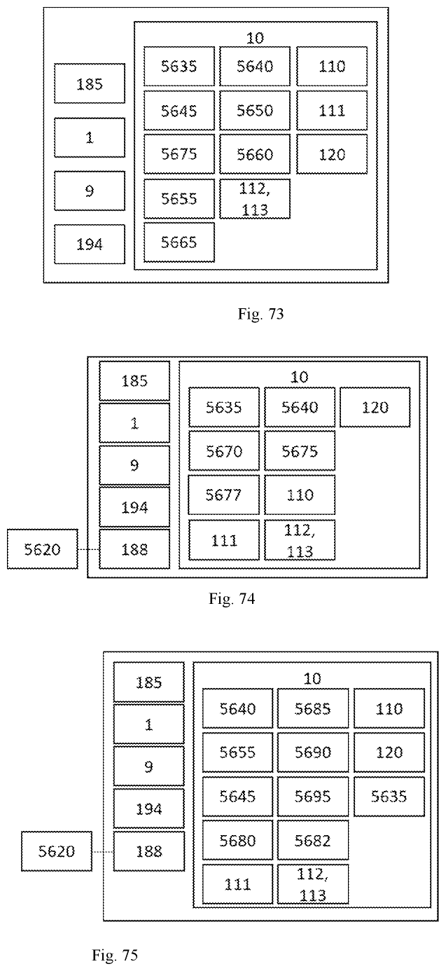

The various aspects of mobility evaluation are described below with reference to a number of figures. FIG. 73 illustrates a system, which may be a service robot, for determining the balance of a person. The system for determining the balance of a person includes a sensor for the contactless detection of a person over time, a skeleton creation module 5635 for creating a skeleton model of the person, a skeleton model-based feature extraction module 5640 for feature extraction based on skeleton points and/or direction vectors between skeleton points of the person, and a transverse skeleton point evaluation module 5645 for evaluating position changes of the skeleton points within the transverse plane with respect to amplitude, orientation, and/or frequency of the position change and for matching detected values with threshold values and/or patterns stored in the memory 10. Alternatively and/or additionally, the system for determining the balance of a person includes a sensor for the contactless detection of a person over time, a skeleton creation module 5635 for creating a skeleton model of the person, a skeleton model-based feature extraction module 5640 for feature extraction based on skeleton points and/or direction vectors between skeleton points of the person, a perpendicular skeleton point evaluation module 5650 for determining the deviation of a direction vector from the perpendicular of the person, with the direction vector connecting at least one skeleton point of the foot, knee, or hip with at least one vertically overlying skeleton point of a person standing upright. The system comprises, for example, a perpendicular skeleton point evaluation module 5650 for determining the deviation of a direction vector from the perpendicular of the person with a threshold value and/or pattern stored in the memory 10, a track width step width module 5675 for determining the track width and/or step width of a person over the distance of the foot skeleton points in the frontal plane over time when the track width has fallen below a threshold value, a person height evaluation module 5655 for evaluating the height of the person, with the height being determined, for example, by the distance between the floor and/or at least one skeleton point of the person and at least one point in the head region, e.g. through vector subtraction of two direction vectors which extend from a common origin to at least one foot and at least the head of the person. “Common origin” refers, for example, to a sensor such as a 3D camera from which depth information is acquired. The system comprises a hand distance evaluation module 5660 for determining the distance between the at least one captured hand skeleton point of the person and at least one captured object in the vicinity of the person and modifying a value in memory (10) if this distance falls below a threshold value. The system comprises a sagittal plane-based skeleton point progression evaluation module 5665 for evaluating the progression of the skeleton points within the sagittal plane and comparing the determined values to values stored in memory 10. The sensor for the contactless detection of the person may be a camera 185, a LIDAR 1, or an ultrasonic and/or radar sensor 194. In one aspect, the system includes a person recognition module 110, a person identification module 111, and/or a movement evaluation module 120.

### EXAMPLES

### Example 1: Delirium Prevention and Monitoring

The service robot 17 can be used to reduce the duration of stay of patients in hospitals if the patients are of advanced age and require an operation that is normally performed under general anesthesia. This case poses a high risk of developing dementia as a result of the anesthesia. Patients who already suffer from cognitive impairments are at high risk in this regard. The service robot 17 can be used for the automated monitoring of the cognitive abilities of patients at least once, e.g. over time, in order to provide medical staff with diagnoses that allow an improved and targeted prophylaxis and treatment of patients.

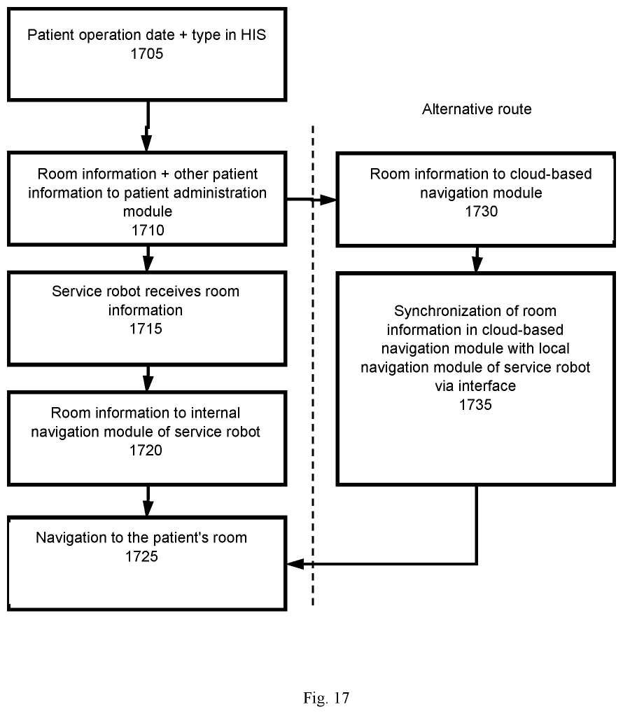

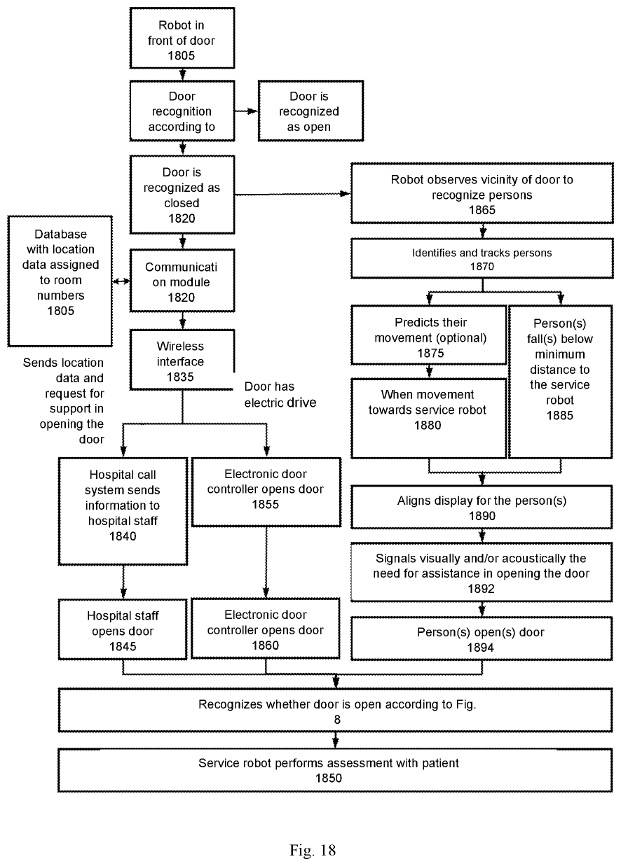

FIG. 17 shows the automated movement of the service robot 17 towards the patient for this purpose. Patient data is stored in a hospital information system (HIS) indicating that an operation is to be performed for a specific patient, including the type and date of the operation 1705. The patient data management system, which accesses the hospital information system (HIS) via an interface 188 (such as WLAN), can obtain information from the HIS as to the room where the patient is located. In addition, other information may be transmitted to the patient administration module 160 in step 1710, including the type of operation, the scheduled date, disease-related information, etc. In one aspect, the service robot 17 accesses the patient administration module 160, receives the room information in step 1715, matches the room information with information stored in its navigation module 101 in step 1720, and then moves towards the patient's room in step 1725. In another aspect, the information matching between the patient administration module 160 and the navigation module in the cloud 170 is performed in step 1730, and in step 1735, the navigation module in the cloud 170 synchronizes with the navigation module 101 of the service robot 17. The service robot 17 subsequently heads towards the patient's room 1725.

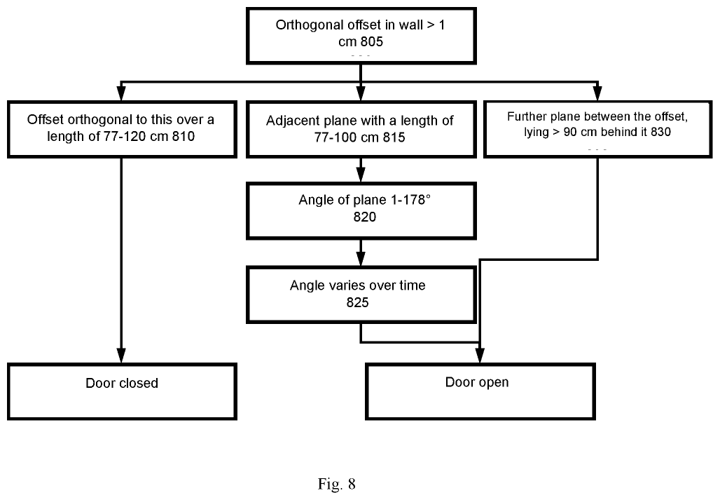

If the service robot 17 is in front of the room door of the patient 1805, the service robot 17 must pass through it in order for the patient to perform the test at the service robot 17. The service robot 17 is further configured in such a way that the service robot 17 is able to detect a door by means of its sensors, e.g. as described below or in FIG. 8 1810. If the door is open, the service robot 17 navigates directly into the patient's room in step 1815. If the door is closed 1820, the service robot 17 uses an integrated communication module 1825 connected via an interface 188 (such as WLAN) to the call system of the hospital 1840 (step 1835).

The service robot 17 uses this to transmit a signal based on its current position, which allows the medical staff to infer its position, as well as the request to please open the door to the patient's room. For this purpose, in one aspect, the service robot 17 has a database in its memory with location data assigned to room numbers 1830, which may be a part of the database with room data 109 as a component of the navigation module 101, which may be connected via an interface to a hospital information system, for example. However, this database may also be available in a cloud 18. If the room door is opened by hospital staff in step 1845, the service robot 17 enters the patient's room to perform the test with the patient in step 1850. Provided that the door to the patient's room has an electric actuator 7, the service robot 17 is configured so that it can use an interface 188 (such as WLAN) (step 1835) to directly access the door controller 1855 and send the door controller 1855 a code to open the door in step 1860.

In an alternative or additional aspect, the service robot 17 observes the surroundings 1865 in front of the door through its at least one sensor 3 and, if the service robot 17 recognizes persons, the service robot 17 tracks these recognized persons in step 1870. Moreover, in one aspect, the service robot 17 predicts the movements of the recognized persons in an optional step 1875 and, if the persons are oriented in its direction 1880, aligns itself in such a way that its display 2 is oriented to face the persons (step 1890). The tracking is performed, for example, by means of the visual person tracking module 112 and/or the laser-based person tracking module 113. In an alternative or additional aspect, the service robot 17 waits until persons are within a minimum distance of the service robot 17 1885 before the service robot 17 orients the display 2 towards these persons 1885. At the same time, the service robot 17 visually and/or acoustically signals its request for the person to open the door to the patient's room in step 1892. The person addressed opens the door in step 1894. In the process, the service robot 17 is able to detect the process of opening the door 1896 as described below or in FIG. 8. As soon as the door is open, the service robot 17 navigates into the patient's room to perform the test in step 1850. If there is no door between the service robot 17 and the patient, as is sometimes the case in intensive care units, these steps can be eliminated.

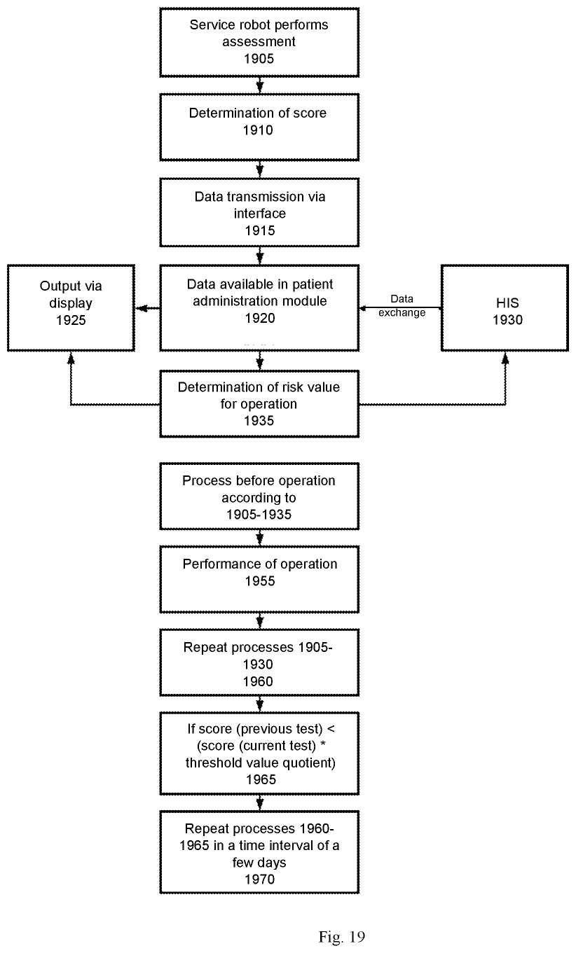

As shown in FIG. 19, the service robot 17 performs a test in step 1905, in particular the mini-mental state exam, and determines a score on this basis in step 1910, which, in the case of the mini-mental state exam, reflects the degree of cognitive impairment of the patient. Alternatively and/or additionally, other test procedures outlined in this document can be used. This data is transmitted via an interface 188 (such as WLAN) (step 1915) to the patient administration module 1920, where they are made available to the medical staff, who can access it via a display 2 (step 1925). The data may also be transferred via an interface 188 (such as WLAN) to the HIS 1930.

The patient administration module 160 is able to obtain additional data on the patient's medical history from the HIS, including, for example, medication that the patient is taking. Based on this information, the patient administration module 160 determines a risk value indicating the likelihood of a progression of the patient's dementia resulting from the planned operation 1935. This risk value may in turn be provided to the medical staff via a display 2 and/or transmitted to the HIS. Based on such information, the medical staff can initiate appropriate preventive measures to prevent or at least reduce the likelihood of possible postoperative dementia.

The service robot 17 and/or the patient administration module 160 is/are further configured in such a way that, after the operation 1955 has been completed (with this information originating from the HIS), the service robot 17 moves back to the patient 1950 as described above and again performs a test with the patient 1960, in particular a geriatric test such as the mini-mental state exam. If the score of the mini-mental state exam has worsened after the operation compared to the test result from before the operation, and if this worsening is above a certain threshold value (e.g. if the patient's score is more than 3% worse than before) (i.e. previous test score >(last test score*threshold value quotient)) 1965, this procedure is repeated after a few days in step 1970 in order to record, evaluate, and document the patient's recovery progress.

### Example 2: Experience-Based Prognosis of Postoperative Dementia

FIG. 20 shows how data is processed by the service robot 17 for therapy suggestions. The rule set 150 is connected to the patient administration module 160 in the cloud 18, which is configured in such a way that patient information can be transferred in anonymized form to the rule set 150 in step 2025. For this purpose, the patient administration module 160 may receive further relevant data beforehand from the hospital information system 2015 via an interface 188 (such as WLAN), including the type of operation, the type of anesthesia, comorbidities, medication taken, delirium prevention measures, postoperative measures to alleviate or treat delirium, etc., as well as the results of the exercises performed with the aid of the service robot 17 in step 2020. The latter may alternatively and/or additionally also originate from the patient administration module 160. This data, which is respectively available as a time series, is anonymized in the patient administration module 160, encrypted, and transmitted to the rule set 150, where it is stored 2030.

In the next step, established methods of machine learning and neural networks are used in step 2035 to develop a prognosis as to the likelihood that the patient will suffer from postoperative delirium based on the available preoperative information, such as the results of the mini-mental state exam performed with the service robot 17, patient-specific information such as age, comorbidities, type of anesthesia, type of operation, medication taken, etc. 2040. A determinant of delirium is the degree of cognitive impairment immediately after the operation, when the service robot 17 usually performs the first test, with which the parameters collected in the scope of a CAM-ICO test, the Delirium Detection Score, the Behavioral Pain Scale, the Critical Care Point Observation Tool, the Richmont Agitation Sedation Scale, the Motor Activity Assessment Scale, in a various expressions and/or data collected by the service robot as described above and (e.g. see the “Delirium detection” section), e.g. the data collected in Example 11, 12, or 17, etc. Improvements to the degree of delirium that occurs over a fixed period time over which the service robot 17 determines cognitive abilities 2045 represents a further determinant. A further alternative or additional determinant is the time required to (re)achieve a certain level of cognitive abilities 2050. This data can be used in the form of a training data set.

In an additional and/or alternative step, the effect of interventions on the determinants described in the previous paragraph is estimated using established methods of machine learning and/or neural networks. These interventions include the use of mild anesthesia and accompanying measures such as the provision of a caregiver, medicinal treatment, etc. 2055

Based on the machine learning estimates, weights are determined 2060 that are transmitted from the rule set 150 to the patient administration module 160 in step 2065 and are used to make recommendations to the medical staff on how to proceed in creating treatment plans for the particular patient in step 2070 according to specific test results determined by the service robot 17. These recommendations may be provided to the medical staff preoperatively, postoperatively, and/or over time by the patient administration module 160. The updates to these recommendations are optionally based on inputs in the HIS that are accessible to the patient administration module 160, inputs in the patient administration module 160, as well as results of the mini-mental state exam, on the basis of the test for the Delirium Detection Score with, for example, sweat detection, the Confusion Assessment Method with, for example, cognitive ability evaluation in conjunction with the detection of acoustic signal sequences, image recognition or finger recognition, and/or the determination of pain status on the basis of an evaluation of emotions, movements of the upper extremities, potential coughing and/or acoustic articulations of pain, which the service robot 17 completes/performs with or on the patient, as described above.

The system can be summarized as a system for the prognosis of postoperative dementia or delirium comprising a processing unit, a memory, and at least one interface 188 (such as WLAN) by means of which the system exchanges data with a mobile data acquisition unit, which has at least one camera 185, and, in one aspect, a spectrometer 196. The mobile data acquisition unit, which mechanically acquires and evaluates data, is a service robot 17, for example. At the same time, in one aspect, the system as such may also be mapped in the service robot 17.

The system has an interface 188 (such as WLAN), by means of which it receives data regarding a person's state of health, treatments, medication status, delirium prevention measures, measures for postoperative delirium treatment, and/or evaluations of measurements performed by the mobile data acquisition unit, e.g. the service robot 17. The system evaluates the data correlated with the persons over time. In a first step, historical data is evaluated that reflect the pre- and postoperative course of patients' illnesses and treatments. Prognoses are made, i.e. in particular the probability of occurrence postoperative data is predicted, e.g. the probability of occurrence of postoperative dementia, its course, etc., for which methods machine learning are used, for example. The prognoses also take medical interventions into account, such as the initiation of certain pre- and postoperative treatments, with their influence on the postoperative course of persons' diseases is predicted. On the basis of these evaluations with historical data, rules are determined and stored, i.e. especially weights (or regression coefficients if regression models are used). In a second step, these can then be used for prognoses based on data obtained via an interface 188 (such as WLAN), including data that has been collected and, in one aspect, processed by the mobile data acquisition unit. This data primarily includes newly acquired data about patients for whom the future course of the disease is still unclear at the time of collection or data which has not yet been exhaustively collected. This second step can be carried out in a separate system that receives the rules or weights from the above-mentioned system. The sequence for the experience-based prognosis of postoperative dementia/delirium can be summarized as follows: capture of persons over time, determination of health status data of the persons based on the capture of the person over time, receipt of preoperative data for the persons, receipt of intervention data for the persons, determination of the influence of the preoperative data and the intervention data on the health status data for the persons through the calculation of a weight estimate for parameters of preoperative data and intervention data, and, for example, prognosis of the health status of a captured person based on the weight estimate and newly collected preoperative data and intervention data for a person.

### Example 3: Sweat Detection on Patients

In contrast to the detection and evaluation of sweat on the skin of patients who are in a bed, the service robot 17 can also perform such an evaluation on patients who are not in a bed. For this purpose, the service robot 17 is able to identify the postures/poses of a patient based on skeleton recognition by means of frameworks in the prior art. The service robot 17 can use the RGB camera to record images of the surface of the patient and evaluate it to determine whether the patient is clothed. To do this, the patient accesses classification algorithms designed to allow skin to be recognized by its color. In one aspect, cross-validation may be carried out here in such a way that the color and/or texture of the target region of the skin on which the measurement is to be performed is compared to that of the person's face, which can be recognized via frameworks in the prior art, e.g. using approaches such as histograms of gradients implemented in frameworks like OpenCV or scikit-image. In one aspect, a filter can be used in the process that applies color corrections due to the fact that detected colors on the face may be darker than on the area of the skin where the measurement is to be made. Regions of the face that may be considered for the determination of the comparison value are the cheeks or the forehead (the process for identifying the latter region has already been described above). Such a correction factor may, in one aspect, also be applied on a seasonal basis. If the matching process performed here results in a similarity value that is above a defined threshold value, the captured measurement area is recognized as skin. An alternative and/or additional filter can also be used that excludes color tones that are untypical for skin (certain shades of red, blue, green, yellow), etc.

The service robot 17 detects a target region of skin for measurement and determines if this is skin or not. If it is not, the service robot 17 detects additional target regions and/or asks the patient via the speech synthesis unit 133 to expose a suitable region. For this purpose, the service robot 17 tracks the region in question, e.g. the patient's arm, and after the patient moves less than defined by a certain threshold value, for example, the evaluation of the spot starts again in order to identify whether the skin is exposed there or if it is still covered by clothing. If the service robot 17 has detected this covered skin, the service robot 17 performs the measurements described elsewhere.

### Example 4: Triggering Elements of Embodiment Based on Cardiovascular Indicators

The service robot 17 calculates the pulse rate/frequency of a person with whom the service robot 17 interacts through the use of the service robot 17 of the described system to determine the pulse rate and pulse frequency by recording and evaluating the face and the corresponding cardiovascular movements of the facial surface and/or head and/or vascular blood flows beneath the skin. In case the service robot 17 includes elements of embodiment, i.e. such elements that at least partially emulate a person, e.g. those that represent a stylized head or parts thereof, such as stylized eyes, a stylized mouth, etc., the determined pulse frequency is used for the unconscious interaction with the person effected by imitating the frequency. This interaction may include, for example, synchronizing the blinking of stylized eyes to the pulse frequency. Alternatively and/or additionally, in case the service robot 17 has a stylized and moving chest, its movement frequency can also be adapted to the pulse frequency. In an alternative and/or additional aspect, if the person has an above-average pulse frequency, indicating, for example, a high level of nervousness (alternatively, other determined parameters indicate a high level of nervousness), the service robot 17 may attempt to calm the patient by selecting a frequency of movement of, for example, the stylized eyes, chest, or other elements that is lower than the frequency identified in the patient. At the same time, the service robot 17 determines the patient's pulse frequency over time and, if necessary, reduces its movement frequency until the patient also has a normal pulse frequency. The difference between the detected pulse frequency and the movement rate of the service robot 17 can remain approximately constant. With reference to eyes, for example, “stylized” means that the eyes may be eyes implemented using hardware, e.g. spheres with circles printed on them and hemispheres that may mechanically cover the printed circles on the spheres. The eyes can also be shown on a display in the form of circles, for example. A stylized mouth may be defined similarly to a smiley, for example, e.g. through a line that can assume different orientations and/or curvatures.

Alternatively and/or additionally, the service robot 17 can identify as well as track the chest of a person by means of the camera 185, which can be achieved, for example, using a framework such as OpenPose, OpenCV, etc. with the aid of the visual person tracking module 112 and/or the laser-based person tracking module 113, for example. The camera 185 and the two person tracking modules 112 and 113, as well as other possible sensors, such as the LIDAR 1, are also referred to collectively as the person detection and tracking unit 4605. This person detection and tracking unit 4605 allows the service robot 17 to detect movements of a person over time, capturing, for example, breathing on the part of the person. If the patient is approximately facing the service robot, this detection includes movements in the horizontal direction as well as in depth. These movements can be determined, for example, using a band-pass filter with a range of 0.005 to 0.125 Hz, but at least 0.05 to 0.08 Hz, and a subsequent fast Fourier transform. This can be used to determine the respiratory rate, which can be used in place of the pulse frequency to imitate patient movements and calm the patient where necessary.

The detection of the pulse rate/frequency and/or respiration/respiratory rate is performed by means of a movement frequency detection unit 4606, consisting, for example, of the camera 185 and computer-implemented methods for determining the pulse rate/frequency and/or respiration/respiratory rate described elsewhere in this document, although other movements of the person are also conceivable. Specifically, the parameters for pulse and/or respiration are acquired and evaluated by a pulse-respiratory evaluation unit 4615. In particular, respiration is detected and evaluated by a movement signal detection and processing unit 4620, which distinguishes background signals of the body from those of the clothing. Details regarding this are provided elsewhere in this document with respect to signal processing. The stylized faces or facial elements, heads, trunks or chests mentioned above are also referred to as stylized embodiment elements 4625. These are moved by a movement unit 4607 at a certain frequency. This may be implemented in different ways depending on the type of stylized embodiment element. Eye movements on a display, for example, may be implemented purely by software, while physical embodiment elements may require, for example, actuators that move eyelids or move a stylized chest. In one aspect, the system further comprises a person recognition module 110, a person identification module 111, a tracking module (112, 113), and/or a movement evaluation module 120. FIG. 60 provides an overview of the components of the system.

The synchronization of the movements of a person with a service robot 17 is characterized by the following aspects ASBPS1 to ASBPS19:

ASBPS1. System for synchronizing the movements of a person and a system comprising a person detection and tracking unit (4605), a movement frequency detection unit (4606) for detecting the frequency of the person's movements, and a movement unit (4607) for moving stylized embodiment elements (4625) of the system at a frequency that is within a defined bandwidth around the detected frequency of the person's movements.
ASBPS2. System according to ASBPS1 further comprising a pulse-respiratory evaluation unit (4615) for measuring the pulse rate and/or respiratory rate of the person.
ASBPS3. System according to ASBPS1 further comprising a movement signal detection and processing unit (4620) for detecting and evaluating detected movements of the person by means of a band-pass filter and for the subsequent processing of the band-pass filtered signals by means of a fast Fourier transform.
ASBPS4. System according to ASBPS1, wherein the stylized embodiment elements (4625) are implemented in hardware and/or software.
ASBPS5. System according to ASBPS4, wherein stylized embodiment elements (4625) implemented in software comprise displaying at least one stylized face or facial element on a display 2.
ASBPS6. System according to ASBPS5, wherein hardware-implemented stylized embodiment elements (4625) comprise at least one of a stylized face, facial element, or a trunk or chest.
ASBPS7. System according to ASBPS5, wherein the movement of stylized embodiment elements (4625) comprises the movement of a stylized face, facial element, trunk, or chest by means of the movement unit (4607).
ASBPS5. System according to ASBPS4, wherein the stylized embodiment elements are triggered to imitate respiratory movement by the movement unit (4607).
ASBPS5. System according to ASBPS1, wherein the system is used to calm the person.
ASBPS10. Computer-implemented method for the synchronization of the movements of a person and a system comprising
the capture and tracking of the movements of a person;
the determination of the frequency of the person's movements;
the moving of stylized embodiment elements (4625) of the system at a frequency that is within a defined range of the determined frequency of the detected movements of the person.
   ASBPS11. Computer-implemented method according to ASBPS10, wherein the captured movements of the person are band-pass filtered, and Fourier transformed.
   ASBPS12. Computer-implemented method according to ASBPS10, wherein the movement of stylized embodiment elements (4625) comprises movement of a stylized face, facial element, trunk, or chest and/or imitates respiration.
   ASBPS13. Computer-implemented method according to ASBPS10, wherein the movement of the stylized embodiment elements (4625) by the movement unit (4607) is kept to a lower frequency than the detected frequency of the person's movements.
   ASBPS14. Computer-implemented method according to ASBPS10, wherein the frequency difference between the movements of the stylized embodiment elements (4625) and the person is kept approximately constant over time by the movement unit (4607).
   ASBPS15. Computer-implemented method according to ASBPS10, wherein the captured movements of the person are the pulse rate and/or respiratory rate.
   ASBPS16. Computer-implemented method according to ASBPS10, wherein the movements of the stylized embodiment elements (4625) are adjusted by the movement unit (4607) to a frequency that is lower than the frequency of the detected movements of the person.
   ASBPS17. Computer-implemented method according to ASBPS10, wherein the movements of the stylized embodiment elements (4625) are controlled by the movement unit (4607) in such a way as to slow down over time.
   ASBPS18. Computer-implemented method according to ASBPS10, wherein the frequency difference between the stylized embodiment elements (4625) and the person is kept approximately constant over time by the movement unit (4607).
   ASBPS19. Computer-implemented method according to ASBPS10, wherein the range of frequency initiated by the movement unit (4607) moves downwards and/or upwards in an interval of 50% or downwards and/or upwards in an interval of less than 15% from the captured frequency of the person.

### Example 5: Method, Device, and/or System for Performing a Get Up and go Test

The determination of a score for getting up and sitting down on a chair is characterized here by the following aspects ASASS1 to ASASS20:

ASASS1. Computer-implemented method for detecting and evaluating the coverage of a distance by a person comprising
the output of an instruction via an output unit;
the capture and tracking of a person over time over this distance, whereby
   the person covers a distance of 3 m in length between a start position and a turning position and a total distance of 6 m in length, including a chair at the start position.
   ASASS2. Computer-implemented method according to ASASS1, wherein the determination of the distance covered by the person is performed by the creation of a skeleton model;
feature extraction of skeleton points and/or direction vectors between skeleton model skeleton points;
feature classification of the skeleton points to determine distances between foot skeleton points in the sagittal plane when they reach minima above the floor, with the distances between the foot skeleton points in the sagittal plane representing the step length;
   further comprising the addition of the step lengths in order to determine the distance the person has covered.
   ASASS3. Computer-implemented method according to ASASS1, wherein the distance covered by the person is determined by tracking movements of the person between the start position and the turning position.
   ASASS4. Computer-implemented method according to ASASS1 comprising the detection of a turning movement of the person and comparison of this turning movement with patterns.
   ASASS5. Computer-implemented method according to ASASS4 comprising the detection of a turning movement of the person at the turning position.
   ASASS6. Computer-implemented method according to ASASS1, wherein a detection of the turning movement of the person is performed and the position of the turning movement defines the turning position.
   ASASS7. Computer-implemented method according to ASASS4-6, wherein the detection of the turning movement is performed by
the creation of a skeleton model;
feature extraction of skeleton points and/or direction vectors between skeleton model skeleton points;
feature classification of the skeleton points in order to determine a rotation of symmetrical skeleton points around the perpendicular passing through the person and/or
feature classification of the skeleton points to determine an angular change of over 160° of symmetrical skeleton points to the line connecting the start and turning positions.
   ASASS8. Computer-implemented method according to ASASS1, wherein the turning position is determined by a detected marker on the floor.
   ASASS9. Computer-implemented method according to ASASS1 further comprising the determination of the getting up of the person from and/or a sitting down of the person on a chair.
   ASASS10. Computer-implemented method according to ASASS9, wherein the determination of the person getting up from and/or a person sitting down on a chair is performed by evaluating the lean of the upper body over time.
   ASASS11. Computer-implemented method according to ASASS10, wherein evaluation of the lean of the upper body over time is performed by
the creation of a skeleton model;
feature extraction of skeleton points and/or direction vectors between skeleton model skeleton points;
feature classification of the skeleton points to determine the orientation of a direction vector between a hip skeleton point and a shoulder skeleton point and/or head skeleton point and a comparison of the orientation with a threshold value and/or pattern and/or
feature classification of the skeleton points to determine the angular change between direction vectors oriented from a knee skeleton point toward a hip and/or foot skeleton point and a comparison of the angular change with a threshold value and/or pattern.
   ASASS12. Computer-implemented method according to ASASS10, wherein the determination of the person getting up from and/or sitting down on a chair is performed via evaluation of the height of the person and/or a change in height of the person compared to a threshold value and/or pattern.
   ASASS13. Computer-implemented method according to ASASS10, wherein the determination of the person getting up from and/or a person sitting down on a chair is performed via the detection, tracking, and evaluation of the movements of the person's head over time and a recognized, at least partially circular movement of the head within the sagittal plane.
   ASASS14. Computer-implemented method according to ASASS1 further comprising the detection and evaluation of the time between the person getting up from and/or a person sitting down on a chair or the determination of the time for covering the distance.
   ASAS15. Computer-implemented method according to ASASS14 further comprising the generation of a score for the determined time.
   ASASS16. Computer-implemented method according to ASASS1 further comprising the performance of a test for the person's hearing ability, vision ability, and/or mental ability.
   ASASS17. Device for performing a method according to ASASS1-ASASS16.
   ASASS18. System comprising a processing unit (9), a memory (10), and at least one sensor for the contactless detection of the movement of a person, with a chair detection module (4540), an output device such as a loudspeaker (192) and/or a display (2) for transmitting instructions, a time-distance module (4510) for determining the time required to cover the distance and/or a speed-distance module (4515) for determining the speed of the captured person on a path, and a time-distance assessment module (4520) for assessing the time required to cover the distance.
   ASASS19. System according to ASASS18 further comprising a hearing test unit (4525), an eye test unit (4530), and/or a mental ability test unit (4535).
   ASASS20. System according to ASASS18 further comprising a projection device (920) for projecting a turning position onto the floor.

### Example 6: Method, Device, and System for Evaluating a Folding Exercise of a Mini-Mental State Exam

The determination of a score in the evaluation of a folding exercise is characterized here by the following aspects AMMTF1 to AMMTF25:

AMMTF1. Computer-implemented method for capturing and evaluating a folding exercise comprising
the detection, identification, and tracking of at least one hand of a person;
the detection, identification, and tracking of a sheet of paper;
the joint classification of dimensions, shapes, and/or movements of the detection sheet and elements of a hand as a folding process.
   AMMTF2. Computer-implemented method according to AMMTFG1 comprising the folding of the sheet approximately in half.
   AMMTF3. Computer-implemented method according to AMMTFG1, wherein the tracking of at least one hand of the person comprises the creation of a skeleton model of at least one hand of the person.
   AMMTF4. Computer-implemented method according to AMMTFG1 comprising the identification of the hand by means of a fault-tolerant segmentation algorithm.
   AMMTF5. Computer-implemented method according to AMMTFG4 further comprising a sheet classification and/or classification of a folding process based on comparison with two-dimensional or three-dimensional patterns.
   AMMTF6. Computer-implemented method according to AMMTFG1, wherein the classification of the folding process comprises the tips of at least one thumb and at least one additional finger touching as hand movements.
   AMMTF7. Computer-implemented method according to AMMTFG1, wherein the classification of the folding process comprises the detection of a change in shape of a sheet in interaction with at least one member of a hand.
   AMMTF8. Computer-implemented method according to AMMTFG1 comprising the identification and tracking of at least one corner and/or edge of a sheet.
   AMMTF9. Computer-implemented method according to AMMTFG8 further comprising the determination of a distance between at least two corners and/or edges of the sheet over time.
   AMMTF10. Computer-implemented method according to AMMTFG9 further comprising a classification of the folding process by comparing determined distances with a threshold value and/or pattern and a detection of a folding process if the determined distance is below the threshold value and/or a minimum pattern similarity is detected.
   AMMTF11. Computer-implemented method according to AMMTFG1, wherein the classification of the folding process comprises detecting a curvature of the sheet that is above a threshold value and/or that exhibits a minimum pattern similarity.
   AMMTF12. Computer-implemented method according to AMMTFG1, wherein the classification of the folding process comprises a distance reduction between at least two sheet edges.
   AMMTF13. Computer-implemented method according to AMMTFG1, wherein the classification of the folding process comprises an approximately parallel alignment of the ends of a sheet margin and/or a distance between the ends of a sheet edge margin that is less than 20 mm.
   AMMTF14. Computer-implemented method according to AMMTFG1, wherein the classification of the folding process comprises the reduction in the size of the detected and tracked sheet over time by more than 40%.
   AMMTF15. Computer-implemented method according to AMMTFG1 further comprising an output on a display 2 and/or a speech output for folding and laying down a sheet or folding and dropping a sheet.
   AMMTF16. Computer-implemented method according to AMMTFG15 further comprising the detection of the sheet over time and the adjustment of a value in a memory after detecting a folding process and the laying down and/or dropping of the sheet.
   AMMTF17. Device for performing a method according to AMMTFG1-AMMTFG16.
   AMMTF18. System comprising a processing unit (9), a memory (10), and a sensor for the contactless detection of the movement of a person, whose memory (10) includes a sheet detection module (4705) for detecting a sheet and a folding movement detection module (4710) for detecting a folding movement of a sheet.
   AMMTF19. System according to AMMTFG18, the system comprising a skeleton creation module (5635) for creating a skeleton model of the person or parts of the person.
   AMMTF20. System according to AMMTFG18, wherein the folding movement detection module (4710) comprises via a sheet distance corner edge module (4720) for detecting the distance of edges and/or corners of a sheet, a sheet shape change module (4725), a sheet curvature module (4730), a sheet dimension module (4740), and/or a sheet margin orientation module (4745).
   AMMTF21. System according to AMMTFG18 further comprising a fingertip distance module (4750) for detecting the distance of fingertips from at least one hand.
   AMMTF22. System according to AMMTFG18, wherein the sheet detection module (4705) includes a sheet segmentation module (4755) and/or a sheet classification module (4760).
   AMMTF23. System according to AMMTFG18 comprising an output device such as a loudspeaker (192) and/or a display (2) for transmitting instructions.
   AMMTF24. System according to AMMTFG18 comprising an interface (188) to a terminal (13).
   AMMTF25. System according to AMMTFG18, wherein at least one sensor for the contactless detection of the movement of a person is a 2D and/or 3D camera (185), a LIDAR (1), a radar sensor, and/or an ultrasonic sensor (194).

### Example 7: Manipulation Detection

Manipulation detection is characterized here by the following aspects AM1 to AM18:

AM1. Computer-implemented method for determining a probability of manipulation of a robot comprising
the detection and tracking of at least one person in the vicinity of the robot, and
determination of a probability of manipulation of the robot by that person.
   AM2. Computer-implemented method according to AM1 further comprising
the determination of the position of at least one person in the vicinity of the robot, and
the determination of the distance of at least one person to the robot.
   AM3. Computer-implemented method according to AM2 further comprising the determination of an increased probability of manipulation upon determining that the distance of at least one person to the robot has fallen below a distance threshold value.
   AM4. Computer-implemented method according to AM1 comprising a skeleton model generation of the captured and tracked person and an extraction and classification of skeleton points.
   AM5. Computer-implemented method according to AM4 further comprising a determination of the orientation of the person relative to the robot.
   AM6. Computer-implemented method according to AM5 further comprising the determination of an orientation of the person relative to the robot by determining the angle between the frontal plane of the person and the axis perpendicular to the control elements 186 of the robot, projected in each case in a horizontal plane, and by comparing the determined angle to a threshold value under which an increased probability of manipulation is detected.
   AM7. Computer-implemented method according to AM1 further comprising
the registration of the person at the robot and
the capture and storage of identification features of the person.
   AM8. Computer-implemented method according to AM7 further comprising
the capture and tracking of the person;
the capture of identification features of the person;
the comparison of the captured identification features with the identification features of the person stored according to AM7 and comparison of these with a threshold value;
detection of an increased probability of manipulation if the value falls below the threshold value and detection of a lower probability of manipulation if the threshold value is exceeded.
   AM9. Computer-implemented method according to AM3, AM6, and/or AM8 comprising a multiplication of the manipulation probabilities in order to determine a manipulation score.
   AM10. Computer-implemented method according to AM9 comprising the performance of evaluations by the robot with the person and the storing of the manipulation score together with the evaluation results.
   AM11. Device for performing a method according to AM1-AM10.
   AM12. System comprising a processing unit (9), a memory (10), and a sensor for the contactless detection of the movement of at least one person, comprising a manipulation attempt detection module (4770) for detecting a manipulation attempt on the part of at least one person.
   AM13. System according to AM12 further comprising a person identification module (111).
   AM14. System according to AM12 further comprising a person-robot distance determination module (4775) for determining the distance of at least one person to the robot.
   AM15. System according to AM14, wherein the person-robot distance determination module (4775) has a height-arm length-orientation module (4780) for estimating the height, arm length, and/or orientation of at least one person to the robot.
   AM16. System according to AM13 further comprising an input registration comparison module (4785) for matching whether a person registered with the system is detected by the system or is entering inputs into the system via the control elements (186).
   AM18. System according to AM12, wherein at least one sensor for the contactless detection of the movement of at least one person is a 2D and/or 3D camera (185), a LIDAR (1), a radar sensor, and/or an ultrasonic sensor (194).

### Example 8: Manipulation Detection 2

Manipulation detection is characterized here by the following aspects AMM1 to AMM17:

AMM1. Computer-implemented method for determining a probability of manipulation of a robot comprising
the detection and tracking of at least one person in the vicinity of the robot by means of a contactless sensor;
the determination of the position of the person in the vicinity of the robot;
the recording and evaluation of audio signals;
the determination of the position of the source of the audio signals;
the comparison of the determined position of the person and the position of the source of the audio signals and the comparison of the difference in position with a threshold value and
the determination of a probability of manipulation of the robot based on the comparison of the difference in position with the threshold value.
   AMM2. Computer-implemented method according to AMM1, wherein the determination of the position of the source of the audio signals is performed by detecting the direction of the audio signals by at least one microphone and triangulating the determined directions.
   AMM3. Computer-implemented method according to AMM1, wherein the determination of the position of the source of the audio signals comprises
the detection of the direction of the audio signal by means of a microphone;
the detection of the position of at least one person by the contactless sensor;
the triangulation of the direction of the audio signal and the determined position of the person.
   AMM4. Computer-implemented method according to AMM1 further comprising
the evaluation of the person's face;
the detection of lip movements over time;
a temporal comparison of detected audio signals with the detected lip movements relative to a threshold value and
depending on the threshold value, correlation of the detected audio signals with the captured person.
   AMM5. Computer-implemented method according to AM1 further comprising
the registration of the person at the robot and
the detecting and storing identification characteristics of the person, wherein identification characteristics include frequency, intensity, and/or spectrum of the audio signals from the person.
   AMM6. Computer-implemented method according to AM5 further comprising
the capture and tracking of the movements of a person;
the capture of identification features of the person;
the comparison of the captured identification features with the identification features of the person stored according to AM5 and comparison of these with a threshold value;
the registration of inputs of the person at the control elements (186) and
a classification of whether a registered person makes inputs at the control elements (186).
   AMM7. Computer-implemented method according to AM5 further comprising a determination of an increased probability of manipulation of the robot if a person who is not registered makes inputs using the robot control elements (186).
   AMM8. Computer-implemented method according to AMM1 further comprising
the detection of words and/or word sequences in the captured audio signals;
the correlation of the detected words and/or word sequences with captured persons;
the determination of a probability of manipulation of the robot by comparing the detected words and/or word sequences and assessing them relative to a threshold value.
   AMM9. Computer-implemented method according to AMM1 comprising
the detection of words or word sequences input by the person by means of a control element (186);
the detection of words and/or word sequences in the captured audio signals;
the correlation of the detected words and/or word sequences from the detected audio signals to captured persons;
the capture of identification features of the person;
the determination of an increased probability of manipulation of the robot if a comparison of the word sequences input via the control elements (186) with word sequences detected from the captured audio signals results in a match that is above a threshold value and, at the same time, there is a match in the comparison of captured identification features of the person with identification features captured and stored during registration that is above a threshold value.
   AMM10. Computer-implemented method according to AMM1, wherein the determination of the position of the source of the audio signals is performed by repositioning the microphone and acquiring the audio signals from two microphone positions with subsequent triangulation.
   AMM11. Computer-implemented method according to AMM1 comprising the determination of a manipulation score by multiplying determined manipulation probabilities.
   AMM12. Device for performing a method according to AMM1-AMM11.
   AMM13. System for manipulation evaluation based on audio signals comprising a processing unit (9), a memory (10), operating elements (186), a sensor for the contactless detection of the movement of a person, a manipulation attempt detection module (4770), at least one microphone (193), a person position determination module (4415) for detecting the position of a person, an audio source position determination module (4420) for determining the spatial origin of an audio signal, an audio signal comparison module for comparing two audio signals (4425), and an audio signal-person module (4430) for assigning audio signals to a person.
   AMM14. System according to AMM13 comprising a speech evaluation module (132).
   AMM15. System according to AMM13 comprising an input registration comparison module (4785) to perform a comparison to determine whether a person identified by the system is providing input to the system.
   AMM16. System according to AMM13, wherein the sensor for the contactless detection of the movement of a person is a 2D and/or 3D camera (185), a LIDAR (1), a radar sensor, and/or an ultrasonic sensor (194).
   AMM17. System according to AMM13 comprising an audio sequence input module (4435) for comparing an audio sequence with a sequence of letters entered by touch.

### Example 9: Spectrometry

Spectroscopy is characterized here by the following aspects ASP1 to ASP20:

ASP1. Computer-implemented method for spectrometric analysis of at least one region of a person's body comprising
the capture, tracking, and generation of an image of a person;
the segmentation of the generated image of the person into body regions;
the definition of the body regions by means of classification;
the alignment of a spectrometer (196) with a previously stored body region.
   ASP2. Computer-implemented method according to ASP1, wherein the body regions are the person's forehead, hand surface, and/or upper body.
   ASP3. Computer-implemented method according to ASP1 further comprising
the capture of movements of a particular body region over time;
the comparison of the captured movements of the body region with a threshold value and/or pattern;
a measurement of the captured body region with the spectrometer (196) depending on the threshold value comparison and/or pattern matching.
   ASP4. Computer-implemented method according to ASP3 further comprising
the monitoring of the movements of the body region during measurement and the performance of threshold value comparison and/or pattern matching;
the interruption of the measurement depending on the threshold value comparison and/or pattern matching.
   ASP5. Computer-implemented method according to ASP1 comprising
the classification of measured spectra by comparison with reference spectra and
the quantitative and/or qualitative determination of at least one measured substance based on the classification.
   ASP6. Computer-implemented method according to ASP5 comprising
the comparison of the quantity and/or quality of determined substances with stored data and
the preparation of a diagnosis of a disease.
   ASP7. Computer-implemented method according to ASP1 comprising the evaluation of the ambient temperature.
   ASP8. Computer-implemented method according to ASP5 comprising the quantitative evaluation of the perspiration of the person.
   ASP9. Computer-implemented method according to ASP1 comprising the determination of a Delirium Detection Score.
   ASP10. Computer-implemented method according to ASP1 comprising the determination of cognitive abilities of the person.
   ASP11. Device for performing a method according to ASP1-ASP10.
   ASP12. System comprising a processing unit (9), a memory (10), and a sensor for the contactless detection of a person, further comprising a spectrometer (196), a visual person tracking module (112), a body region detection module (4810) for detecting body regions, a spectrometer alignment unit (4805) for aligning the spectrometer (196) with a body region of a person, and having access to a reference spectra database (4825) containing reference spectra for matching measured spectra to determine measured substances.
   ASP13. System according to ASP12 comprising a spectrometer measurement module (4820) for monitoring a measurement process of the spectrometer (196).
   ASP14. System according to ASP12, wherein the visual person tracking module (112) includes a body region tracking module (4815).
   ASP15. System according to ASP12 comprising access to a clinical picture database (4830) with stored clinical pictures.
   ASP16. System according to ASP12 comprising a perspiration module (4835) for the quantitative determination of a person's perspiration.
   ASP17. System according to ASP12 comprising a Delirium Detection Score determination module (4840) for determining a Delirium Detection Score.
   ASP18. System according to ASP12 comprising a cognitive ability assessment module (4845) for assessing cognitive abilities of the person.
   ASP19. System according to ASP12 comprising a thermometer (4850).
   ASP20. System according to ASP12, wherein at least one sensor for the contactless detection of the movement of a person is a 2D and/or 3D camera (185), a LIDAR (1), a radar sensor, and/or an ultrasonic sensor (194).

### Example 10: Attention Analysis

Attention analysis is characterized here by the following aspects AAA1 to AAA18:

AAA1. Computer-implemented method for matching captured signals from a tactile sensor (4905) with a sequence of output acoustic signals comprising
the output of a sequence of pulsed acoustic signals;
the detection of signals by a tactile sensor (4905);
the comparison of the output sequence of acoustic signals with the signals captured by the tactile sensor (4905).
   AAA2. Computer-implemented method according to AAA1 with a pulse frequency of pulsed signals between approx. 0.3 and 3 Hz.
   AAA3. Computer-implemented method according to AAA1 with a delay or phase shift between the output pulsed acoustic signals and the signals captured by the tactile sensor (4905).
   AAA4. Computer-implemented method according to AAA3, wherein the delay or phase shift is approx. half a pulse length.
   AAA5. Computer-implemented method according to AAA3, wherein the captured signal of the tactile sensor (4905) tracks the pulsed tone sequence.
   AAA6. Computer-implemented method according to AAA1 comprising the assignment of a value to each output acoustic signal.
   AAA7. Computer-implemented method according to AAA6 further comprising the adjustment of a value upon detection of a signal according to a defined value.
   AAA8. Computer-implemented method according to AAA7, wherein the value adjustment is an incrementation of the value.
   AAA9. Computer-implemented method according to AAA7 further comprising the preparation of a diagnosis based on the adjusted value.
   AAA10. Computer-implemented method according to AAA9, wherein the diagnosis is an assessment of cognitive abilities.
   AAA11. Computer-implemented method according to AAA1 comprising the detection of a person and the detection and position determination of a hand of the person.
   AAA12. Computer-implemented method according to AAA11 comprising the positioning the tactile sensor (4905) at a distance from the hand that is below a threshold value.
   AAA13. Device for performing a method according to AAA1-AAA12.
   AAA14. System comprising a processing unit (9), a memory (10), an acoustic signal output unit (192), a tactile sensor (4905), a tactile sensor evaluation unit (4910) for evaluating signals from the tactile sensor (4905), and a tactile sensor output comparison module (4915) for performing a comparison to establish whether the captured signals occur after an output of acoustic signals.
   AAA15. System according to AAA14 comprising an actuator (4920) on which the tactile sensor (4905) is positioned.
   AAA16. System according to AAA14 comprising an actuator positioning unit (4925) for positioning the tactile sensor (4905) within a defined distance to the hand.
   AAA17. System according to AAA14 comprising a camera (185), a person identification module (111), and a hand identification module (4930).
   AAA18. System according to AAA14 comprising a cognitive ability assessment module (4845) for assessing cognitive abilities of the person.

### Example 11: Cognitive Analysis

Cognitive analysis is characterized here by the following aspects AKA1 to AKA16:

AKA1. Computer-implemented method for matching finger poses of a person detected on the basis of video signals with optically and/or acoustically outputted numerical values, comprising
the optical and/or acoustic output of numerical values;
the capture and tracking of fingers of a person;
the detection of finger poses;
the assessment of the finger poses and
the comparison of the assessed finger poses with the optically and/or acoustically outputted numerical values.
   AKA2. Computer-implemented method according to AKA1, wherein the finger poses represent numerical values.
   AKA3. Computer-implemented method according to AKA2, wherein a numerical value may represent multiple finger poses.
   AKA4. Computer-implemented method according to AKA1, wherein the optical output of numerical values represents an output of finger poses by an actuator (4920).
   AKA5. Computer-implemented method according to AKA1 further comprising the detection and tracking of the head of the person and the determination of the field of vision of the person.
   AKA6. Computer-implemented method according to AKA5 further comprising the positioning of the actuator (4920) and/or a display (2) in the person's field of vision.
   AKA7. Computer-implemented method according to AKA1 further comprising the determination of cognitive abilities of the person through an assessment of the comparison of the assessed finger poses with the visually and/or acoustically outputted numerical values.
   AKA8. Device for performing a method according to AKA1-AKA7.
   AKA9. System comprising a processing unit (9), a memory (10), an output unit and a numerical value output module (4940) for outputting numerical values, and a person detection and tracking unit (4605) comprising a camera (185) and a person recognition module (110).
   AKA10. System according to AKA9, wherein the output unit is a sound generator such as a loudspeaker (192), a display (2), or an actuator (4920).
   AKA1 1. System according to AKA10, wherein the actuator (4920) is a robot arm.
   AKA12. System according to AKA10, wherein the actuator (4920) has a robot hand (4950).
   AKA13. System according to AKA9 further comprising a hand pose detection module (4960) for detecting hand poses of the person.
   AKA14. System according to AKA12 further comprising a finger pose generation module (4955) for generating finger poses of the robotic hand (4950).
   AKA15. System according to AKA9, wherein the system is coupled to a patient administration module (160).
   AKA16. System according to AKA9 further comprising a cognitive ability assessment module (4845) for assessing cognitive abilities of the captured person.

### Example 12: Pain Status Determination

Pain status determination is characterized here by the following aspects ASB1 to ASB23:

ASB1. Computer-implemented method for determining the pain status of a person comprising
the capture of the person,
the facial recognition of the person,
the selection of candidate regions within the face;
feature extraction of the surface curvatures of the candidate regions;
the classification of the surface curvatures of the candidate regions individually and/or contiguously, with the classification describing a pain status.
   ASB2. Computer-implemented method according to ASB1, wherein the single and/or contiguous classification of the surface curvatures of the candidate regions represent/s a determination of emotion.
   ASB3. Computer-implemented method according to ASB2 further comprising the assignment of scale values to emotions and the rating of emotions on a scale.
   ASB4. Computer-implemented method according to ASB2 further comprising the evaluation of emotions over time.
   ASB5. Computer-implemented method according to ASB1 further comprising
the detection of a bed and the generation of images of the bed;
the classification of the images of the bed by comparison with patterns to detect a person to be captured.
   ASB6. Computer-implemented method for determining the pain status of a person comprising
the capture and tracking of a person's upper extremities over time;
the evaluation of angles between the trunk and the upper arm, the upper arm and the forearm and/or the phalanges and hand bones, with the evaluation of the angles describing a pain status.
   ASB7. Computer-implemented method according to ASB6 further comprising the evaluation of
the intensity of angular changes;
the speed of the angular changes and/or
the number of angular changes per time unit.
   ASB8. Computer-implemented method according to ASB7 further comprising the assignment of scale values to evaluate the angular changes.
   ASB9. Computer-implemented method for determining the pain status of a person comprising
the recording of acoustic signals;
the evaluation of the acoustic signals by means of a pain classification in order to determine whether the recorded acoustic signals represent a pain vocalization;
the assessment of the acoustic signals classified as a pain vocalization by means of a pain intensity classification, wherein
the pain intensity classification comprises assigning scale values to the recorded acoustic signals, with the scale values respectively representing a pain status.
   ASB10. Computer-implemented method according to ASB9 comprising
the determination of the position of the source of acoustic signals;
the determination of the position of the person whose pain status is being determined;
the adjustment of the determined position through comparison with a threshold value;
the storage of a value depending on the threshold comparison with respect to the determined pain status.
   ASB11. Computer-implemented method for determining the pain status of a person comprising
the capture of the person,
the recognition of the person's face and neck,
the evaluation of the person's face and neck area for patterns describing an artificial ventilation device;
the storage of a value upon detection of a pattern describing an artificial ventilation device, wherein the artificial ventilation device describes a pain status.
   ASB12. Computer-implemented method according to ASB1, ASB6, ASB9, or ASB11, wherein at least two of the methods are performed in parallel or sequentially.
   ASB13. Computer-implemented method according to ASB1, ASB6, ASB9, or ASB11 further comprising the evaluation of the determined scale values or stored values as part of a delirium detection.
   ASB14. Device for performing a method according to ASB1-ASB13.
   ASB15. System for determining the pain status of a person comprising a processing unit (9), a memory (10), a sensor for the contactless detection of the person, and a pain status calculation module (5040).
   ASB16. System according to ASB15 comprising a face recognition module (5005) for recognizing the person's face, a face candidate region module (5010) for selecting candidate regions within the face, an emotion classification module (5015) for classifying the surface curvatures of the candidate regions of the face into emotions, and an emotion assessment module (5020) for determining a scale value for the emotion.
   ASB17. System according to ASB15 comprising a bed recognition module (5025) for recognizing a bed.
   ASB18. System according to ASB15 comprising a person recognition module (110), a visual person tracking module (112), an upper extremity evaluation module (5035) for detecting and tracking the upper extremities of the person and evaluating the angles of the upper extremities.
   ASB19. System according to ASB15 comprising a microphone (193) for recording acoustic signals, a pain vocalization module (5055) for classifying the intensity and frequency of the acoustic signals and the determination of a scale value representing a pain vocalization.
   ASB20. System according to ASB15 further comprising an audio source position determination module (4420) for evaluating the position of the source of acoustic signals and an audio signal-person module (4430) for correlating audio signals with a person.
   ASB21. System according to ASB15 comprising a ventilation device recognition module (5065) for recognizing a ventilation device.
   ASB22. System according to ASB15 comprising a pain sensation evaluation module (5085) for evaluating sensors attached to a person.
   ASB23. System according to ASB15, wherein the sensor for the contactless detection of the person is a 2D and/or 3D camera (185), a LIDAR (1), a radar sensor, and/or an ultrasonic sensor (194).

### Example 13: Blood Pressure

The determination of blood pressure is characterized here by the following aspects AB1 to AB16:

AB1. Computer-implemented method for determining cardiovascular parameters of a person comprising
the capture and tracking of the face of a person;
the selection of candidate regions within the face;
the capture and analysis of movements within candidate regions of the face attributable to cardiovascular activity.
   AB2. Computer-implemented method according to AB1, wherein the movements comprise blood flow in veins.
   AB3. Computer-implemented method according to AB1, wherein the movements comprise movements of the facial surface and/or the head.
   AB4. Computer-implemented method according to AB1 comprising a two-dimensional and/or three-dimensional capture of the movements.
   AB5. Computer-implemented method according to AB1 comprising a single and/or contiguous evaluation of the candidate regions.
   AB6. Computer-implemented method according to AB1 further comprising
illumination of the face and
frontal detection of the face.
   AB7. Computer-implemented method according to AB1, wherein the evaluation of the movements includes the classification of the movements to determine systolic or diastolic blood pressure.
   AB8. Computer-implemented method according to AB1 further comprising
the determination of the orientation of the face in the room
the minimization of the angle of coverage of the face resulting from an axis that is perpendicular to a sensor for capturing the face and an axis that is perpendicular to the sagittal plane of the face.
   AB9. Device for performing a method according to AB1-AB8.
   AB10. System for the detection of cardiovascular parameters of a person comprising a processing unit (9), a memory (10), and a camera (185), further comprising a body region detection module (4810) for detecting body regions, a body region tracking module (4815), and a cardiovascular movements module (5110) for detecting movements attributable to cardiovascular activity.
   AB11. System according to AB10 further comprising a face recognition module (5005) and a face candidate region module (5010).
   AB12. System according to AB10, wherein the camera (185) provides at least the 8-bit green color channel.
   AB13. System according to AB10 further comprising a light (5120) for illuminating the face during recording by the camera (185).
   AB14. System according to AB13, wherein the light is positioned above and/or below the camera (185).
   AB15. System according to AB10 comprising a blood pressure determination module (5125) for determining systolic or diastolic blood pressure.
   AB16. System according to AB10 further comprising a tilting unit (5130) to minimize the angle of coverage of the camera (185) relative to the sagittal plane and/or a movement planner (104) to reposition the camera (185) relative to the captured face.

### Example 14: Substance Measurement

The measurement of substances under the skin, such as glucose, is characterized here by the following aspects AG1 to AG20:

AG1. System for measuring substances on and/or within the skin of a person comprising a detector (195) with an evaluation laser (5205) and a further laser (5210), where the evaluation laser (5205) is deflected upon entry into a medium (5215), such as a crystal surface, and the further laser (5210) excites a substance while varying the wavelength, with the region of the excited substance interacting with the medium (5215) at the point where the evaluation laser (5205) is deflected, and further comprising a laser variation module (5225) for feature extraction and feature classification of the wavelength variation of the further laser, and a laser deflection evaluation module (5220) for evaluating the deflection of the evaluation laser.
AG2. System for measuring substances on and/or within the skin of a person comprising a detector (195) with a medium (5215) comprising a crystal with a cubic, hexagonal, or tetragonal lattice structure, a refractive index of 1-4, and a spectral width within an interval between 100 nm-20,000 nm.
AG3. System according to AG1 and AG2 further comprising a sensor for the contactless detection of a person, a movement evaluation module (120) for evaluating detected movements of the person, and a finger positioning recognition module (5230) for the automated recognition of the positioning of a finger on the medium (5215) and the start of the measurement.
AG4. The system according to AG1 and AG2 further comprising an evaluation laser (5205) and a further laser (5210), with the evaluation laser (5205) being deflected from the crystal surface and the further laser (5210) exciting a substance while varying the wavelength, the region of the excited substance interacting with the medium (5215) at the point where the evaluation laser (5210) is deflected.
AG5. System according to AG1 and AG2 further comprising a laser variation module (5225) for feature extraction and feature classification of the wavelength variation of the further laser (5210), and a laser deflection evaluation module (5220) for evaluating the deflection of the evaluation laser (5205).
AG6. System according to AG1 and AG2, wherein the evaluation laser (5205) is evaluated by means of a sensor based on the photoelectric effect (5250).
AG7. System according to AG1 and AG2 further comprising an interface for transferring data to a patient administration system (160).
AG8. System according to AG1 or AG2, wherein the detector (195) is positioned on an actuator (4920).
AG9. System according to AG1 or AG2 further comprising a sensor for the contactless detection of a person, such as a 2D or 3D camera (185), a LIDAR (1), a radar sensor, and/or an ultrasonic sensor (194).
AG10. System according to AG9 further comprising a body region detection module (4810) and a body region tracking module (4815) for tracking the measurement region.
AG11. System for measuring substances on and/or within the skin of a person comprising a camera (185) and a tilting unit (5130) for orienting the camera (185), a body region detection module (4810) a body region tracking module (4815), at least one light source (5270) for illuminating the person's skin to be detected, a wavelength variation unit (5275) for varying the wavelength of the light emitted by at least one light source, and a wavelength variation evaluation unit (5280) for evaluating the variation of the wavelength of the captured signals.
AG12. The system according to AG11, wherein at least one light source (5270) is a laser and/or multiple LEDs with different spectra that may be controlled accordingly.
AG13. System according to AG11, wherein the wavelength of the emitted light is between 550 and 1600 nm.
AG14. System according to AG11, wherein the wavelength of the emitted light is between 900 and 1200 nm.
AG15. System according to AG11, wherein the camera (185) has a photodetector made of indium gallium arsenide or lead sulfite.
AG16. System according to AG11 comprising another camera (185) for detecting light in the 400-800 nm spectrum.
AG17. System according to AG1, AG2, and AG11, wherein the system includes a substance classification module (5295) for feature extraction and feature classification of detected light signals and the comparison of the classified light signals to substance data stored in a memory.
AG18. Computer-implemented method for measuring substances on and/or within a person's skin comprising
the alignment of a camera (185) towards the surface of a person's skin;
the capture and tracking of the surface of the person over time;
the illumination of the person using a light source (5270);
the variation of the wavelength of light emitted by at least one light source (5270);
the detection of the light reflected on the skin surface and/or within the skin;
the evaluation of at least the detected light by comparing evaluated features with stored features.
   AG19. Computer-implemented method according to AG18 further comprising the determination of the concentration of substances located on the skin surface and/or within the skin.
   AG20. Device for performing a method according to AG19-AG20.

### Example 15: Experience-Based Prognosis of Postoperative Dementia and/or Delirium

The experience-based prognosis of postoperative dementia and/or delirium is characterized here by the following aspects AEPPD1 to AEPPD8:

AEPPD1. Computer-implemented method for the prognosis of postoperative dementia and/or delirium comprising
the capture of persons over time;
the determination of the health status data of the persons based on the capture of the persons over time;
the receipt of preoperative data for the persons;
the receipt of intervention data for the persons;
the determination of the influence of the preoperative data and the intervention data on the health status data of the persons by calculating a weight estimate for parameters of the preoperative data and the intervention data.
   AEPPD2. Computer-implemented method according to AEPPD1 further comprising a prognosis of the health status of a captured person based on the weight estimate and newly collected preoperative data and intervention data for the person.
   AEPPD3. Computer-implemented method according to AEPPD2, wherein the capture of the person is partially automated.
   AEPPD4. Computer-implemented method according to AEPPD2, wherein the capture of the person is performed by a service robot (17).
   AEPPD5. Computer-implemented method according to AEPPD1, wherein machine learning methods are used for weight estimation.
   AEPPD6. Computer-implemented method according to AEPPD2, wherein the prognosis of the health status is a prediction of the probability of occurrence of postoperative dementia/delirium.
   AEPPD7. Computer-implemented method according to AEPPD1 comprising the transmission of the weight estimation to a service robot (17).
   AEPPD8. Device for performing a method according to AEPPD1-AEPPD7.

### Example 16: Detection of Moisture on Surfaces

The assessment of moisture on surfaces is characterized here by the following aspects ADFO1 to ADFO18:

ADFO1. Computer-implemented method for the assessment of the location of moisture on a surface comprising
the capture of a surface;
the classification of the surface characteristics to detect moisture on the captured surface;
the segmentation of the captured surface into wet and non-wet areas;
the determination of the width of the captured areas;
the assessment of the width of the captured areas by way of comparison with at least one stored value.
   ADFO2. Computer-implemented method according to ADFO1, wherein the determination of the width is performed perpendicular to the direction of movement of a system.
   ADFO3. Computer-implemented method according to ADFO2 comprising a movement of the system through an area assessed as dry if its determined width exceeds a threshold value.
   ADFO4. Computer-implemented method according to ADFO1 comprising an output via an output unit indicating the area assessed as wet.
   ADFO5. Computer-implemented method according to ADFO2 comprising the interruption of the movement of the system when the determined width of the area assessed as wet exceeds a threshold value and/or the width of the area assessed as dry falls below a threshold value.
   ADFO6. Computer-implemented method according to ADFO2 comprising the modification of a value in a memory and/or the transmission of a message if it is determined that the width of an area assessed as wet exceeds a threshold value.
   ADFO7. Computer-implemented method according to ADFO1 comprising the modification of a value in a memory and/or the transmission of a message if it is determined that the width of an area assessed as dry falls below a threshold value.
   ADFO8. Computer-implemented method for the assessment of the location of moisture on a surface comprising
the capture of a surface;
the classification of the surface characteristics to detect moisture on the surface;
the segmentation of the captured surface into wet and non-wet areas;
the entry of wet areas as obstacles in a map.
   ADFO9. Computer-implemented method according to ADFO8, wherein the map includes different types of obstacles.
   ADFO10. Computer-implemented method according to ADFO8 comprising
the determination of a surface classified as wet that exhibits minimum dimensions;
output via an output unit and/or
the transmission of a message and/or
the modification of a value in memory (10).
   ADFO11. Computer-implemented method according to ADFO1 or ADFO8 comprising a modification of a path planning upon detection of a surface detected as wet that has a width above a threshold value.
   ADFO12. Device for performing a method according to ADFO1-ADFO11.
   ADFO13. System for assessing moisture on a surface comprising a sensor for the contactless detection of a surface, a segmentation module (5705) for segmenting the detected surface, a moisture detection module (5305) for classifying the segments with respect to surface moisture, and a moisture assessment module (5310) for assessing dimensions of the classified surface segments.
   ADFO14. System according to ADFO13 further comprising a map module (107) that includes obstacles in the surroundings of the system and the segments classified with respect to moisture.
   ADFO15. System according to ADFO13 further comprising a movement planner (104) and/or a path planning module (103).
   ADFO16. System according to ADFO13 further comprising an output unit (2 or 192) and outputs stored in the memory (10) for indicating the area detected as wet.
   ADFO17. System according to ADFO13, wherein the sensor for the contactless detection of a surface is a camera (185) or a radar sensor (194).
   ADFO18. System according to ADFO13, wherein the system is a service robot (17).

### Example 17: Method for Fall Detection

Fall detection is characterized here by the following aspects ASE1 to ASE17:

ASE1. Computer-implemented method for detecting the fall of a person comprising
the capture and tracking of the movements of the person;
the detection of a fall event on the basis of feature extraction and the classification of the orientation of the limbs, trunk, and/or height of the person;
the detection and classification of the movements of the person after a fall has occurred; and
the assessment of the severity of the fall event.
   ASE2. Computer-implemented method according to ASE1 further comprising the transmission of a notification via an interface (188) after assessing the severity of the fall event.
   ASE3. Computer-implemented method according to ASE1 further comprising the detection at least one vital sign of the person.
   ASE4. Computer-implemented method according to ASE1, wherein the feature extraction comprises evaluating skeleton points of a skeleton model of the person.
   ASE5. Computer-implemented method according to ASE1, wherein the detection of a fall event is performed by determining distances or distance changes of extracted skeleton model skeleton points in the direction of the floor.
   ASE6. Computer-implemented method according to ASE1, wherein the detection of a fall event is performed by determining the orientation and/or orientation change of direction vectors between skeleton model points.
   ASE7. Computer-implemented method according to ASE1, wherein the detection of a fall event is performed by determining accelerations of skeleton points in the vertical direction.
   ASE8. Computer-implemented method according to ASE1, wherein the detection of a fall event is performed by determining the height and/or change in height of the person.
   ASE9. Computer-implemented method according to ASE1, wherein the detection of a fall event is performed by determining the area occupied by a person projected in the vertical direction on the floor.
   ASE10. Computer-implemented method according to ASE1 further comprising the detection of the position of the person's head and/or obstacles in the vicinity of the person.
   ASE11. Computer-implemented method according to ASE10 further comprising the evaluation of the position of the person's head relative to the floor and/or detected obstacles.
   ASE12. Device for performing a method according to ASE1-ASE11.
   ASE13. System for detecting the fall of a person comprising a memory (10), at least one sensor for detecting the movements of the person over time, a person identification module (111), a person tracking module (112 or 113), a fall detection module (5405) for extracting features from the sensor data and classifying the extracted features as a fall event, and a fall event assessment module (5410) for classifying the severity of the fall event.
   ASE14. System according to ASE13 comprising an interface (188) to a server and/or terminal (13) for the purpose of transmitting messages.
   ASE15. System according to ASE13 comprising a vital signs acquisition unit (5415) for acquiring vital signs of the person and a vital signs evaluation module (5420) for evaluating acquired vital signs of the person.
   ASE16. System according to ASE13, wherein the fall detection module (5405) includes a skeleton creation module (5635) for creating a skeleton model of a person.
   ASE17. System according to ASE13 and ASE15, wherein the sensor for detecting the movements of the person and/or the vital signs acquisition unit (5415) is a camera (185), a LIDAR (1), a radar and/or an ultrasonic sensor (194), and/or an inertial sensor (5620).

### Example 18: Method for the Contactless Acquisition of Vital Signs of a Person

The contactless acquisition of vital signs is characterized here by the following aspects ABEV1 to ABEV20:

ABEV1. Computer-implemented method for acquiring vital signs of a person comprising
the capture and tracking of the person;
the capture and tracking of a body region of the person on or through which the vital signs are acquired;
the acquisition of vital signs;
the comparison of the acquired vital signs to at least one stored threshold value and
the triggering of an event if the threshold value is exceeded or fails to be reached.
   ABEV2. Computer-implemented method according to ABEV1, wherein the event comprises a speed reduction.
   ABEV3. Computer-implemented method according to ABEV1, wherein the event comprises heading to a target position.
   ABEV4. Computer-implemented method according to ABEV3, wherein the target position is a seat.
   ABEV5. Computer-implemented method according to ABEV1, wherein at least one threshold value is dynamically determined from previously recorded vital signs.
   ABEV6. Computer-implemented method according to ABEV5, wherein the dynamically determined threshold value is based on an averaging of recorded vital signs over a defined time interval.
   ABEV7. Computer-implemented method according to ABEV1, wherein the acquisition of vital signs is performed contactlessly.
   ABEV8. Computer-implemented method according to ABEV1, wherein the acquisition of vital signs is performed by a vital signs sensor (5425) attached to the person.
   ABEV9. Computer-implemented method according to ABEV1 further comprising the capture of body movements of the person and an evaluation of the acquired vital signs with simultaneous matching of the captured body movements.
   ABEV10. Computer-implemented method according to ABEV1, wherein the acquisition of vital signs is performed depending on the captured movements of the person.
   ABEV11. Computer-implemented method according to ABEV1, wherein the vital signs acquired include pulse rate, pulse rate variability, systolic and/or diastolic blood pressure, and/or respiratory rate.
   ABEV12. Computer-implemented method according to ABEV1 further comprising the determination of the fall risk based on the acquired vital signs.
   ABEV13. Computer-implemented method according to ABEV12, wherein the fall risk is an acute fall risk.
   ABEV14. Computer-implemented method according to ABEV1, wherein the acquisition of vital signs occurs during the performance of a test and/or an exercise carried out with the person.
   ABEV15. Device for performing a method according to ABEV1-ABEV14.
   ABEV15. System for acquiring vital signs of a person comprising a processing unit (9), a memory (10), at least one sensor for the contactless detection of the person's movements over time, and a vital signs evaluation module (5420).
   ABEV17. System according to ABEV15 further comprising a body region detection module (4810) and a body region tracking module (4815) for tracking the detection region of vital signs, and a vital signs acquisition unit (5415) for acquiring vital signs of the person.
   ABEV17. System according to ABEV15, wherein the sensor for detecting the movements of the person is a camera (185), a LIDAR (1), an ultrasonic sensor, and/or a radar sensor (194).
   ABEV18. System according to ABEV15, wherein the vital signs evaluation module (5420) initiates a notification of a system via an interface (188), an output via an output unit (2 or 192), a change in velocity of the system, and/or the system's heading towards a target position.
   ABEV20. System according to ABEV15 comprising an application module (125) with rules for performing at least one exercise.
   ABEV20. System according to ABEV15 comprising an interface (188) and a vital signs sensor (5425) attached to a person.

### Example 19: Method for Determining a Score that Describes a Person's Fall Risk

The determination of a fall risk score is characterized here by the following aspects AESS1 to AESS25:

AESS1. Computer-implemented method for determining a score that describes a person's fall risk comprising
the capture of a person's gait pattern;
the extraction of features of the captured gait pattern;
the classification of the extracted features of the gait pattern;
the comparison of at least two of the classified gait pattern features to a gait pattern classification stored in a memory; and
the determination of a fall risk score.
   AESS2. Computer-implemented method according to AESS1 further comprising the determination of the speed of the person.
   AESS3. Computer-implemented method according to AESS2 further comprising the determination of the person's speed based on the number and step width of the person's steps covered per time unit.
   AESS4. Computer-implemented method according to AESS2, wherein the determination of the speed of the person is performed relative to the speed of a detection and evaluation unit that captures and evaluates the gait pattern of the person.
   AESS5. Computer-implemented method according to AESS4, wherein the speed of the detection and evaluation unit is performed with the inclusion of an odometry unit (181) in the detection and evaluation unit.
   AESS6. Computer-implemented method according to AESS4, wherein the speed of the detection and evaluation unit is performed with the inclusion of obstacles entered in a map.
   AESS7. Computer-implemented method according to AESS2, wherein the speed of the person is detected relative to the position of obstacles entered in a map.
   AESS8. Computer-implemented method according to AESS1, wherein the person's speed, step length, cadence, and/or acceleration in the horizontal and/or vertical plane are jointly evaluated.
   AESS9. Computer-implemented method according to AESS1, wherein the extracted features of the gait pattern are skeleton points of a skeleton model of the captured person, direction vectors between the skeleton model's skeleton points, accelerations of the skeleton points or the direction vectors, the positions of the skeleton points relative to each other in the room and/or angles derived from direction vectors, and wherein the classified features of the gait pattern are the step length, the length of the double step, the step speed, the ratio of the step lengths in the double step, the flexion and/or extension, the stance duration, the track width, and/or the progression (position) and/or the distance of skeleton points to each other and/or the acceleration of skeleton points.
   AESS10. Computer-implemented method according to AESS1 further comprising
the logging in of the person at the detection and evaluation unit, which captures and evaluates the gait pattern of the person;
the identification of the person by means of an optical sensor;
the storage of identification features of the person; and
the tracking of the person over time.
   AESS11. Computer-implemented method according to AESS9 comprising the determination of the position of a foot skeleton point of the person determined via
the position of the corresponding knee or hip skeleton point;
a direction vector originating from the knee skeleton point with a parallel orientation to the lower leg; and
the height of the knee skeleton point and/or hip skeleton point above the floor if the direction vector passes through the perpendicular.
   AESS12. Computer-implemented method according to AESS1, wherein the extraction of features of the gait pattern comprises the inclusion of data from an inertial sensor.
   AESS13. Device for performing a method according to AESS1-AESS12.
   AESS14. System for determining a score that describes a person's fall risk comprising a processing unit (9), a memory (10), a sensor for detecting a person's movements over time, a movement extraction module (121), and a movement assessment module (122) that includes a fall risk determination module (5430) for determining a fall risk score.
   AESS15. System according to AESS14 comprising a person identification module (111) and a person tracking module (112 or 113) and having components (e.g. 2, 186) for logging the person into the system.
   AESS16. System according to AESS14, wherein the system receives sensor data from an inertial sensor (5620) via an interface (188) and the sensor data is analyzed in the movement extraction module (121).
   AESS17. System according to AESS14, wherein the movement assessment module (122) comprises a person speed module (5625) for determining the speed of the person.
   AESS18. System according to AESS14, wherein the sensor for detecting the movements of a person over time is a camera (185), a LIDAR (1), an ultrasonic sensor, and/or a radar sensor (194).

### Example 20: Determination of the Balance of a Person

The determination of the balance of a person is characterized here by the following aspects ABEP1 to ABEP21:

ABEP1. Computer-implemented method for determining the balance of a person comprising
the contactless capture of the person over time;
the creation of a skeleton model of the captured person;
feature extraction of the skeleton model's skeleton points and/or direction vectors lying between the skeleton points;
the evaluation of the amplitude, orientation, and/or frequency of the change in position of the skeleton points within the transverse plane.
   ABEP2. Computer-implemented method according to ABEP1 further comprising
the comparison of the evaluation with a threshold value and/or pattern, and
the determination of a balance based on the threshold deviation and/or pattern deviation.
   ABEP3. Computer-implemented method for determining the balance of a person comprising
the contactless capture of the person over time;
the creation of a skeleton model of the captured person;
feature extraction of the skeleton model's skeleton points and/or direction vectors lying between the skeleton points;
the determination of a deviation of a direction vector, which is formed as a connection of at least one skeleton point of foot, knee, or hip with at least one vertically overlying skeleton point of a person standing upright, from the perpendicular of the person.
   ABEP4. Computer-implemented method according to ABEP3 further comprising
the comparison of the determined deviation with a threshold value and/or pattern, and
the determination of a balance based on the threshold deviation and/or pattern deviation.
   ABEP5. Computer-implemented method according to ABEP1 or ABEP2 further comprising the determination of the track width of the captured person via the distance of the ankles in the frontal plane over time.
   ABEP6. Computer-implemented method according to ABEP5, wherein balance is determined if the track width has fallen below the threshold value.
   ABEP7. Computer-implemented method according to ABEP1 or ABEP2 further comprising
the evaluation of the height of the person through
   the determination of the difference between the floor or at least one ankle on the one hand and at least one skeleton point in the head area on the other hand, or
   the vector subtraction of two direction vectors extending from a common origin to at least one foot and at least the head of the person
and the derivation of whether the person is sitting or standing from the height of the person and/or the distance.
   ABEP8. Computer-implemented method according to ABEP1 or ABEP2 comprising the evaluation of the orientation of at least one direction vector between at least one knee point and at least one hip point with respect to the deviation from the perpendicular.
   ABEP9. Computer-implemented method according to ABEP1 or ABEP2 comprising
the detection of objects in the vicinity of the person;
the detection of the position of the person and/or the position of at least one hand skeleton point of the person;
the determination of the distance between at least one hand skeleton point and at least one object in the vicinity of the person;
the modification of a value in the memory (10) if the distance falls below a threshold value.
   ABEP10. Computer-implemented method according to ABEP1 or ABEP2 comprising the evaluation of the progression of the skeleton points within the sagittal plane and the comparison of the progression with values stored in the memory (10).
   ABEP11. Computer-implemented method according to ABEP1 or ABEP2, wherein the balance determination comprises a standing, sitting, or walking person.
   ABEP12. Device for performing the method according to ABEP1-ABEP11.
   ABEP13. System for determining the balance of a person with a sensor for the contactless detection of a person over time, a skeleton creation module (5635) for creating a skeleton model of the person, a skeleton model-based feature extraction module (5640) for feature extraction based on skeleton points and/or direction vectors between skeleton points of the person, and a transverse skeleton point evaluation module (5645) for evaluating position changes of the skeleton points within the transverse plane with respect to amplitude, orientation, and/or frequency of the position change and for matching detected values with threshold values and/or patterns stored in the memory (10).
   ABEP14. System for determining the balance of a person with a sensor for the contactless detection of a person over time, a skeleton creation module (5635) for creating a skeleton model of the person, a skeleton model-based feature extraction module (5640) for feature extraction based on skeleton points and/or direction vectors between skeleton points of the person, a perpendicular skeleton point evaluation module (5650) for determining the deviation of a direction vector from the perpendicular of the person, with the direction vector connecting at least one skeleton point of the foot, knee, or hip with at least one vertically overlying skeleton point of a person standing upright.
   ABEP15. System according to ABEP13 or ABEP14 comprising a perpendicular skeleton point evaluation module (5650) for determining the deviation of a direction vector from the perpendicular of the person with a threshold value and/or pattern stored in memory (10).
   ABEP16. System according to ABEP13 or ABEP14 comprising a track width step width module (5675) for determining the track width and/or step width of a person over the distance of the foot skeleton points in the frontal plane over time when the track width has fallen below a threshold value.
   ABEP17. System according to ABEP13 or ABEP14 comprising a person height evaluation module (5655) for evaluating the height of the person.
   ABEP18. System according to ABEP17, wherein the height is determined
via the distance between a floor or at least one foot skeleton point on the one hand and at least one skeleton point in the head area on the other hand, or
by vector subtraction of two direction vectors extending from a common origin to at least one foot and at least the head of the person
   ABEP19. System according to ABEP13 or ABEP14 comprising a hand distance evaluation module (5660) for evaluating the distance between a hand skeleton point and further objects in the vicinity of the person and further comprising rules for a threshold value comparison of the determined distance with distance threshold values stored in the memory.
   ABEP20. System according to ABEP13 or ABEP14 comprising a sagittal plane-based skeleton point progression evaluation module (5665) for evaluating the progression of the skeleton points within the sagittal plane and comparing the determined values to values stored in memory (10).
   ABEP21. System according to ABEP13 or ABEP14, wherein the sensor for the contactless detection of the person is a camera (185), a LIDAR (1), an ultrasonic sensor, and/or a radar sensor (194).

### Example 21: Determination of the Position of an Ankle

Instead of accessing the values from a skeleton model, the ankle position can be determined on the basis of an estimate as follows: For example, the height of a knee skeleton point above the floor is determined, for example, if the vector connecting a knee skeleton point and a hip skeleton point is perpendicular. Alternatively and/or additionally, the distance between the floor and the hip skeleton point can be determined when the lower leg and the thigh are approximately perpendicular. The distance between the knee skeleton point and the hip skeleton point can be determined and subtracted from the distance between the hip skeleton point and the floor to obtain the distance between the knee skeleton point and the floor. Furthermore, a determination of a direction vector originating from the knee skeleton point with a parallel orientation to the lower leg is performed by segmenting the point cloud of the lower leg and/or an evaluation of patterns in two-dimensional space and determining the orientation of the direction vector by centering an axis through the point cloud or the patterns, e.g. using the RANSAC framework. In one aspect, an additional Bayes estimation can be made here that takes the angle of the thigh into account. This is done, for example, as the connection of the hip skeleton point with the knee skeleton point as one leg and the perpendicular, alternatively the other thigh, or the orientation of the trunk (for example, with skeleton points along the spine). In turn, probabilities may be stored in the memory 10 that describe the orientation of the lower leg represented by the point cloud and/or pattern, or by the direction vector derived from this, depending on the orientation of the thigh and determined, for example, by means of a ground truth. The position of the knee skeleton point and the direction of the lower leg are then used to determine the foot skeleton point insofar as the previously determined height of the knee skeleton point above the floor defines the length of the direction vector that starts at the knee skeleton point and the end point of the direction vector represents the foot skeleton point.

The system for determining the position of an ankle skeleton point of a captured person is illustrated in FIG. 74. The system, for example a service robot 17, comprises a processing unit 9, a memory 10, and at least one sensor for capturing movements of a person over time, e.g. an inertial sensor 5620, a camera 185, a LIDAR 1, an ultrasonic sensor, and/or a radar sensor 194. It further comprises, for example, a skeleton creation module 5635 for creating a skeleton model of the person, a skeleton model-based feature extraction module 5640 for feature extraction based on skeleton points and/or direction vectors between skeleton points of the person, and a foot skeleton point classification module 5670 for the feature classification of a foot skeleton point that determines the position of a foot skeleton point via the orientation of a direction vector that represents the lower leg that begins at the position of the corresponding knee skeleton point and has a length determined on the basis of the height of at least the corresponding knee skeleton point or hip skeleton point above the floor. In one aspect, the system may include a track width step width module 5675 for determining the track width and/or step width of a person and/or a foot skeleton point-walking aid position module (5677) for determining the position of at least one foot skeleton point relative to a position from the end point of at least one forearm crutch or underarm crutch when the forearm crutch or underarm crutch contacts the floor. In one aspect, the system includes a person recognition module 110 and/or a movement evaluation module 120. The sequence itself may be summarized as follows according to FIG. 83 a): capture of a person over time (step 6105), creation of at least a portion of a skeleton model of the captured person (step 6110), determination of the position of a knee skeleton point for the leg for which the foot skeleton point is to be determined (step 6115), determination of a direction vector originating from the knee skeleton point with a parallel orientation to the lower leg (step 6120), determination of the height of the knee skeleton point above the floor if the knee skeleton point passes through the perpendicular (step 6125), determination of the position of the foot skeleton point by forming a direction vector from the determined knee skeleton point, with the direction vector to be formed having the same orientation as the determined direction vector, whereby the direction vector to be formed has a length corresponding to the height of the knee skeleton point above the floor if the knee skeleton point passes through the perpendicular (step 6130). Alternatively and/or additionally, the sequence according to FIG. 83 b) can also be designed as follows: capture of a person over time (step 6105), creation of at least part of a skeleton model of the captured person (step 6110), determination of the position of a knee skeleton point for the leg for which the foot skeleton point is to be determined (step 6115), determination of the position of a hip skeleton point for the leg for which the foot skeleton point is to be determined (step 6140), determination of a direction vector originating from the knee skeleton point with a parallel orientation to the lower leg (step 6120), determination of the height of the hip skeleton point above the floor as a minuend (step 6145), determination of the length of the direction vector connecting the hip skeleton point and the knee skeleton point as a subtrahend (step 6150), determination of the difference of the minuend and the subtrahend (step 6155), determination of the position of the foot skeleton point (step 6160) by forming a direction vector from the determined knee skeleton point which has the same orientation as the determined direction vector and which is oriented parallel to the lower leg from the knee skeleton point, wherein the direction vector to be formed has a length corresponding to the determined difference.

The determination of the position of an ankle is characterized here by the following aspects AEPF1 to AEPF11:

AEPF1. Computer-implemented method for determining the position of a foot skeleton point of a skeleton model of a captured person comprising
the capture of a person over time;
the creation of at least part of a skeleton model of the captured person;
the determination of the position of a knee skeleton point for the leg for which the foot skeleton point is to be determined;
the determination of a direction vector originating from the knee skeleton point with a parallel orientation to the lower leg;
the determination of the height of the knee skeleton point above the floor if the knee skeleton point passes through the perpendicular;
the determination of the position of the foot skeleton point by forming a direction vector from the determined knee skeleton point, with the direction vector to be formed having the same orientation as the determined direction vector and the direction vector to be formed having a length corresponding to the height of the knee skeleton point above the floor if the knee skeleton point passes through the perpendicular.
   AEPF2. Computer-implemented method for determining the position of a foot skeleton point of a skeleton model of a captured person comprising
the capture of a person over time;
the creation of at least part of a skeleton model of the captured person;
the determination of the position of a knee skeleton point for the leg for which the foot skeleton point is to be determined;
the determination of the position of a hip skeleton point for the leg for which the foot skeleton point is to be determined;
the determination of a direction vector originating from the knee skeleton point with a parallel orientation to the lower leg;
the determination of the height of the hip skeleton point above the floor as a minuend;
the determination of the length of the direction vector connecting the hip skeleton point and the knee skeleton point as a subtrahend;
the determination of the difference between the minuend and the subtrahend;
the determination of the position of the foot skeleton point by forming a direction vector from the determined knee skeleton point that has the same orientation as the determined direction vector and that is oriented parallel to the lower leg from the knee skeleton point, the direction vector to be formed having a length equal to the determined difference.
   AEPF3. Computer-implemented method according to AEPF1 and AEPF2, wherein the position of the foot skeleton point is used to determine the track width and/or step width of a person.
   AEPF4. Computer-implemented method according to AEPF1 and AEFP2, wherein the position of the determined foot skeleton point is evaluated relative to the position of at least one end point of a forearm crutch.
   AEPF5. Computer-implemented method according to AEPF1 and AEFP2, wherein the position of the foot skeleton point is evaluated relative to the foot length, wherein the foot length is determined based on an estimate of the height of the person and foot lengths corresponding to the height.
   AEPF6. Computer-implemented method according to AEPF9, wherein the height of the person is determined through
the vector subtraction of two direction vectors extending from a common origin to at least one foot and at least the head of the person, or
via the distance between a floor or at least one foot skeleton point on the one hand and at least one skeleton point in the head area on the other hand
   AEPF7. Computer-implemented method according to AEPF1 and AEFP2, wherein a direction vector originating from the knee skeleton point with a parallel orientation to the lower leg is determined by
the segmentation of the point cloud of the lower leg and/or an evaluation of patterns in two-dimensional space
the determination of the orientation of the direction vector by the centering of an axis through the point cloud or the patterns.
   AEPF8. Device for performing a method according to AEPF1-AEPF7.
   AEPF9. System for determining the position of an point of a captured person comprising a processing unit (9), a memory (10), at least one sensor for capturing movements of a person over time, a skeleton creation module (5635) for creating a skeleton model of the person, a skeleton model-based feature extraction module (5640) for feature extraction based on skeleton points and/or direction vectors between skeleton points of the person, and a foot skeleton point classification module (5670) for the feature classification of a foot skeleton point that determines the position of a foot skeleton point via the orientation of a direction vector that represents the lower leg that begins at the position of the corresponding knee skeleton point and has a length determined on the basis of the height of at least the corresponding knee skeleton point or hip skeleton point above the floor.
   AEPF9. System according to AEPF17 comprising a track width step width module (5675) for determining the track width and/or step width of a person.
   AEPF10. System according to AEPF17 comprising a foot skeleton point-walking aid position module (5677) for determining the position of at least one foot skeleton point relative to the position from the end point of at least one forearm crutch or underarm crutch when the forearm crutch or underarm crutch contacts the floor.
   AEPF11. The system of AEPF17, wherein the sensor for detecting the movements of the person over time is an inertial sensor (5620), a camera (185), a LIDAR (1), an ultrasonic sensor, and/or a radar sensor (194).

### Example 22: Classification of a Turning Movement of a Person

The system for classifying a turning movement is illustrated on the basis of FIG. 75. The system for classifying the turning movement of a person, e.g. a service robot 17, comprises a processing unit 9, a memory 10, and at least one sensor for detecting the movements of the person over time, a skeleton model-based feature extraction module 5640 for feature extraction based on skeleton points and/or direction vectors between the skeleton points of the person, and a turning movement feature classification module 5680 for feature classification of a turning movement, wherein, in one aspect, the turning movement is determined via the angular change of at least one direction vector projected in the transverse plane between two skeleton points over time, and, in an alternative and/or additional aspect, the turning movement is determined by way of the angular change of at least one direction vector connecting the shoulder skeleton points, hip skeleton points, knee skeleton points, foot skeleton points, arm skeleton points and/or captured skeleton points of the head to each other, respectively. The turning movement feature classification module (5680) may further include an angle evaluation module (5682) for adding up detected angles and/or angular changes. In one aspect, the system includes a person recognition module 110, a movement evaluation module 120, and/or a skeleton creation module 5635.

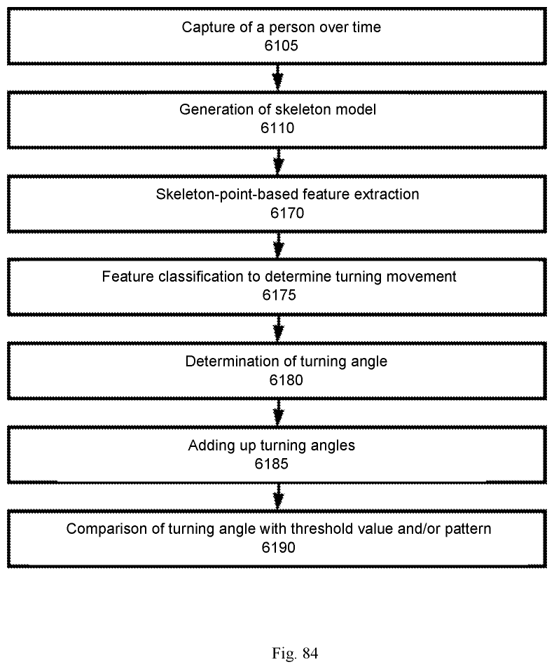

The system may further comprise a foot skeleton point distance determination module 5685 for determining the absolute distance of the foot skeleton points, a person height evaluation module 5655 for evaluating the height of the person, a hip-knee orientation module 5690, for example, for evaluating the orientation of at least one direction vector between at least one knee skeleton point and at least one hip skeleton point with respect to deviation from perpendicular, and/or a transverse skeleton point evaluation module 5645 e.g. for evaluating position changes of the skeleton points within the transverse plane that, for example, evaluates the amplitude, orientation, and/or frequency of the position change of the skeleton points within the transverse plane and/or determines a deviation of a direction vector, which is formed as a connection of at least one skeleton point of the foot, knee, or hip with at least one vertically overlying skeleton point of a person standing upright from the perpendicular of the person and compares it with a threshold value stored in the memory 10. The system may further have a turning movement-height-balance-step length classification module 5695 for classifying the person's turning movement, height, balance, and/or step length. The sensor for detecting the movements of a person over time may be a camera 185, a LIDAR 1, a radar sensor, an ultrasonic sensor 194, and/or at least one inertial sensor 5620. The turning movement detection method is summarized, for example, in FIG. 84 with the following steps: capture of the person over time 6105, creation of a skeleton model of the captured person 6110, feature extraction of skeleton points 6170 from the skeleton model and/or direction vectors between the skeleton points of the person, feature classification comprising the determination of a turning movement 6175 of a direction vector over time, in one aspect the additional determination of turning angles 6180, for example, from at least one turning movement of a direction vector, the adding up of the angles and/or angular changes 6185, and the comparison the sum of the added turning angles with a threshold value and/or a pattern 6190.

The classification of the turning movement of a person is characterized here by the following aspects AKDP1 to AKDP22:

AKDP1. Computer-implemented method for classifying the turning movement of a person comprising
the capture of the person over time;
the creation of a skeleton model of the captured person;
feature extraction of skeleton points from the skeleton model and/or direction vectors between the person's skeleton points;
feature classification comprising the determination of a turning movement of a direction vector over time.
   AKDP2. Computer-implemented method according to AKDP1 further comprising
the determination of turning angles from at least one turning movement of a direction vector;
the adding up of the angles and/or angular changes; and
the comparison of the sum a threshold value and/or a pattern.
   AKDP3. Computer-implemented method according to AKDP1, wherein the turning movement is determined via an angular change of at least one direction vector projected into the transverse plane between two skeleton points over time.
   AKDP4. Computer-implemented method according to AKDP1, wherein the turning movement is determined via the angular change of at least one direction vector connecting the shoulder skeleton points, hip skeleton points, knee skeleton points, foot skeleton points, and arm skeleton points, respectively.
   AKDP5. Computer-implemented method according to AKDP1 further comprising the determination of the distance of the foot skeleton points.
   AKDP6. Computer-implemented method according to AKDP1 further comprising the determination of the height of the person.
   AKDP7. Computer-implemented method according to AKDP6, wherein the height of the person is determined via
the distance between a floor or at least one ankle on the one hand and at least one skeleton point in the head area on the other hand and/or
by vector subtraction of two direction vectors extending from a common origin to at least one foot and at least the head of the person.
   AKDP8. Computer-implemented method according to AKDP1, wherein the evaluation of the orientation of at least one direction vector between at least one knee point and at least one hip point with respect to the deviation from the perpendicular.
   AKDP9. Computer-implemented method according to AKDP1, wherein the balance of the person is evaluated.
   AKDP10. Computer-implemented method according to AKDP9, wherein the balance is determined through an evaluation of the amplitude, orientation, and/or frequency of the change in position of the skeleton points within the transverse plane and comparison with threshold values and/or patterns stored in the memory (10).
   AKDP11. Computer-implemented method according to AKDP9, wherein the balance is determined based on a determination of the deviation of a direction vector from the perpendicular of the person.
   AKDP12. Computer-implemented method according to AKDP11, wherein the direction vector is formed as a connection of at least one skeleton point from the foot, knee, or hip with at least one vertically overlying skeleton point of a person standing upright.
   AKDP13. Computer-implemented method according to AKDP1, wherein the classification of a turning movement includes the person's height, balance, and/or step length.
   AKDP14. Device for performing a method according to AKDP1-AKDP13.
   AKDP15. System for classifying a turning movement of a person comprising a processing unit (9), a memory (10), and at least one sensor for capturing movements of a person over time, a skeleton model-based feature extraction module for feature extraction (5640) based on skeleton points and/or direction vectors between skeleton points of the person and/or direction vectors between at least two skeleton points, and a turning movement feature classification module (5680) for feature classification of a turning movement.
   AKDP16. System according to AKDP15, wherein the turning movement feature classification module (5680) comprises an angle evaluation module (5682), e.g. for adding up determined angles and/or angular changes.
   AKDP17. System according to AKDP15 further comprising a foot skeleton point distance determination module (5685) for determining the distance of the foot skeleton points.
   AKDP18. System according to AKDP15 further comprising a person height evaluation module (5655) for evaluating the height of the person.
   AKDP19. System according to AKDP15 further comprising a hip-knee orientation module (5690) for evaluating the orientation of at least one direction vector between at least one knee skeleton point and at least one hip skeleton point with respect to deviation from perpendicular.
   AKDP20. System according to AKDP15 comprising a transverse skeleton point evaluation module (5645) for evaluating position changes of the skeleton points within the transverse plane.
   AKDP21. System according to AKDP15 comprising a turning movement-height-balance-step length classification module (5695) for classifying the person's turning movement, height, balance, and/or step length.
   AKDP22. System according to AKDP15, wherein the sensor for capturing the movements of a person over time is a camera (185), a LIDAR (1), a radar sensor, an ultrasonic sensor (194), and/or at least one inertial sensor (5620).

### Example 23: Classification of a Person's Gait

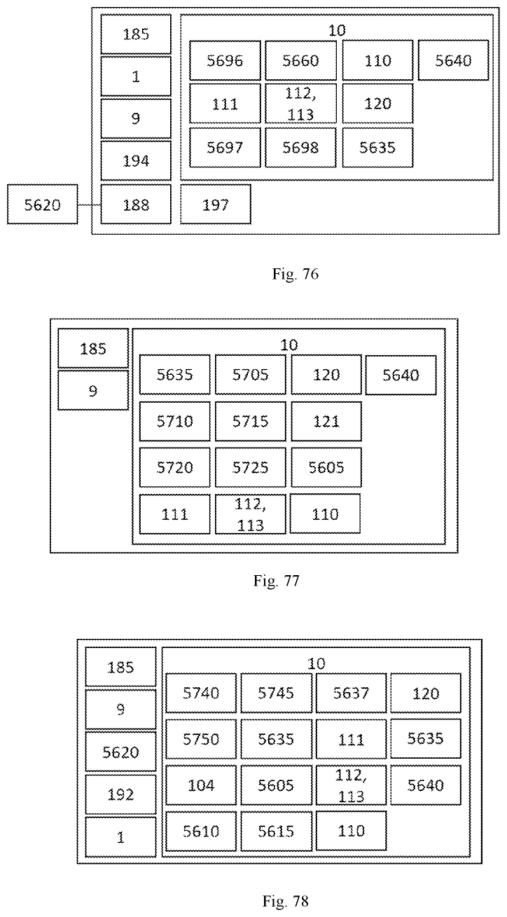

The gait classification system is set up as illustrated in FIG. 76: The system for classifying the gait of a person, e.g. a service robot 17, comprises a processing unit 9, a memory 10, at least one sensor for capturing a person over time (e.g. a camera 185, a LIDAR 1, a radar sensor, an ultrasonic sensor 194, or an inertial sensor 5620), and a position determination line module for determining the position of the person over time relative to a straight line 5696. On the one hand, the line may be determined, for example, by means of at least one sensor for capturing a person, or it can be determined on the other hand, for example, by evaluating the direction of movement of the person movement direction module 5698, thereby eliminating the need for markers on the floor. The position determination line module 5696 for determining the position of the person over time relative to the straight line can, for example, determine the distance of the person to the straight line as the distance of the person's center of gravity or head projected into the transverse plane, as the distance of the foot skeleton points of the person to the straight line determined by creating a skeleton model (e.g. by the skeleton creation module 5635), and/or as the maximum or average value of the distance covered to the straight line, and determine it in relation to a distance threshold value. The distance itself is determined by means of a distance module (5696), e.g. by determining the step lengths of the person and adding these step lengths, with the step length being determined by determining the distance between two foot skeleton points within the sagittal plane; by evaluating the change in position of the person within a coordinate system and determining the distance between two points within the coordinate system; and/or by evaluating odometry data acquired by the odometry unit 181 of the service robot 17, which indicates the position of the service robot 17. Alternatively, this can also be determined through navigation functions that, in addition to odometry, can also be also used for position determination by way of matching of the captured surroundings of the service robot 17 with an environment stored in a map. Accordingly, determining the position of the person relative to the service robot 17, for which the distance covered can be determined by means of the navigation functions, also allows the determination of the position of the person. In one aspect, the system includes a hand distance point evaluation module 5660 for evaluating the distance between a hand skeleton point and other objects in the vicinity of the person in order to detect whether the person is holding onto objects, which has an influence on the person's gait. This can be noted by adjusting a value in the memory. This is then used to classify the person's gait based on the deviation from the straight line and the distance from the hand skeleton points to detected objects/obstacles. In one aspect, the system may include a projection device 197 for projecting a straight line onto the floor. In one aspect, the system includes a person recognition module 110, a movement evaluation module 120, a skeleton creation module 5635, and/or a skeleton model-based feature extraction module 5640.

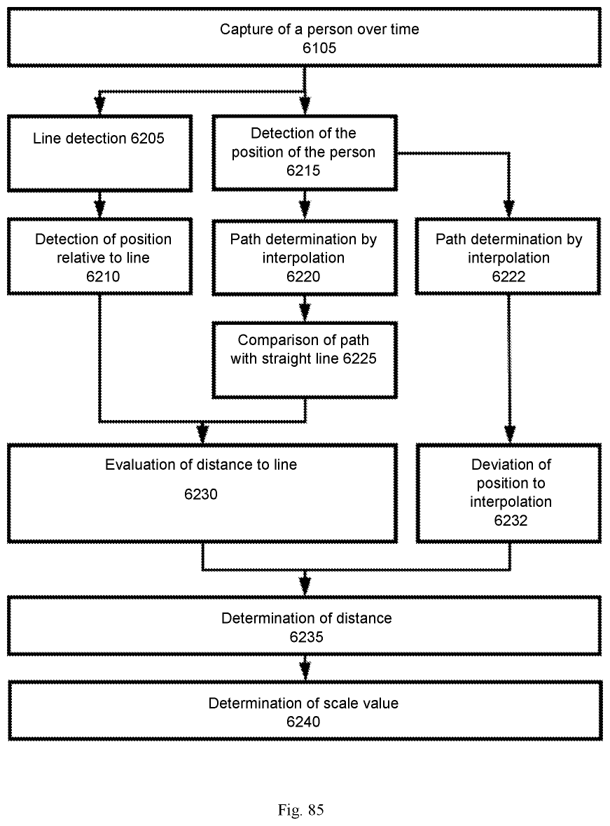

The process can be summarized according to the steps illustrated in FIG. 85: the capture of a person over time 6105, e.g. by means of the sensor for capturing a person. In one aspect, the process includes the detection of a straight imaginary line or straight line on the floor 6205, the detection of the position of the person over time relative to the straight line 6210, with the person essentially following the line, and the evaluation of the distance of the person to the line over a defined distance 6230 (using the steps 6210 and 6230 in the position determination line module 5696), followed by the determination of the distance 6235 of the distance covered by the person (by the distance determination line module 5696) and the determination of a scale value 6240 as the value of a gait (pattern) classification. Alternatively and/or additionally, capturing a person over time 6105 can be followed by the capture of the position of the person over time 6215, the determination of a path resulting from the position of the person captured over time 6220 (in the movement direction module 5698, e.g. by interpolating the touchdown points of foot skeleton points of a skeleton model), comparing the path with a straight line 6225 that is approximately parallel to the path of the person, and evaluating the distance of the person to the line over a defined distance 6230 (using steps 6225 and 6230 in the position determination line module 5696), followed by the determination of the distance 6235 covered by the person, and the determination of a scale value 6240 as the value of a gait (pattern) classification. An alternative sequence can be summarized as follows: capture of a person 6105, detection of the positions of the person over time 6215, determination of a path resulting from the positions of the person detected over time 6222 (e.g. by interpolating the position data of the person or touchdown points of foot skeleton points of a skeleton model), evaluation of the positions of the person relative to the path over a defined distance 6232, determination of a distance covered 6235, and determination of a scale value 6240. Steps 6220 and 6222 may differ in the type of interpolation, with 6222 being primarily by linear interpolation, while nonlinear interpolation methods may also be considered for step 6220 in addition to linear interpolation.

The classification of a person's gait is characterized here by the following aspects AKGP1 to AGDP24:

AGDP1. Computer-implemented method for classifying a person's gait comprising
the capture of a person;
the detection of a straight imaginary line or straight real line on the floor;
the detection of the positions of the person over time relative to the straight line, wherein the person essentially follows the line; and
the evaluation of the distance of the person to the straight line over a defined distance.
   AGDP2. Computer-implemented method for classifying a person's gait comprising
the capture of a person;
the capture of the positions of the person over time;
the determination of a path resulting from the person's positions captured over time;
the comparison of the path with a straight line;
the evaluation of the positions of the person relative to the straight line over a defined distance.
   AGDP3. Computer-implemented method for classifying a person's gait comprising the capture of a person;
the capture of the positions of the person over time;
the determination of a path resulting from the person's positions captured over time;
the evaluation of the positions of the person relative to the path over a defined distance.
   AGDP4. Computer-implemented method according to AGDP1-3 further comprising the determination of a scale value based on the evaluation.
   AGDP5. Computer-implemented method according to AGDP1-3, wherein the straight line results from the starting position of a person and the movement of a person.
   AGDP6. Computer-implemented method according to AGDP1, wherein the straight line interacts with the floor.
   AGDP7. Computer-implemented method according to AGDP1, wherein the straight line is projected onto the floor.
   AGDP8. Computer-implemented method according to AGDP1-3 comprising the determination of the distance covered by the person along the straight line by determining the step lengths of the person and adding these step lengths, wherein the step length is determined by determining the distance between two foot skeleton points within the sagittal plane.
   AGDP9. Computer-implemented method according to AGDP1-3 comprising the determination of the distance covered by the person along the straight line by evaluating the change in position of the person within a coordinate system and determining distance between two points within the coordinate system.
   AGDP10. Computer-implemented method according to AGDP1-3 comprising the determination of the distance covered by the person along the straight line by evaluating odometry data.
   AGDP11. Computer-implemented method according to AGDP1-3 comprising the capture and evaluation of the position of the person relative to a system whose position is determined over time using navigation functions.
   AGDP12. Computer-implemented method according to AGDP1-3 comprising the determination of the distance of the person to the straight line as the distance of the person's center of gravity or head projected into the transverse plane.
   AGDP13. Computer-implemented method according to AGDP1-3 comprising the determination of the distance of the person to the straight line as the distance of the foot skeleton points of the person to the straight line determined by creating a skeleton model.
   AGDP14. Computer-implemented method according to AGDP1-3, wherein the distance of the person to the straight line is determined as a maximum or average value over the distance covered.
   AGDP15. Computer-implemented method according to AGDP1-3 comprising the determination of the distance of at least one hand skeleton point of the person to detected objects and/or obstacles.
   AGDP16. Computer-implemented method according to AGDP15, wherein the gait of the person is classified based on the deviation from the straight line and the distance of the hand skeleton points to detected objects/obstacles.
   AGDP17. Device for performing a method according to AGDP1-AGDP16.
   AGDP18. System for classifying a person's gait comprising a processing unit (9), a memory (10), at least one sensor for the capture of a person over time, and a position determination line module (5696) for determining the person's position over time relative to a line.
   AGDP19. System according to AGDP18, wherein at least one sensor for the capture of a person over time also detects a line on the floor.
   AGDP20. System according to AGDP18 comprising a projection device (197) for projecting a line onto the floor.
   AGDP21. System according to AGDP18, comprising a distance module (5697) for determining the distance covered by the person.
   AGDP22. System according to AGDP18 comprising a hand distance evaluation module (5660) for evaluating the distance between a hand skeleton point and further objects in the vicinity of the person.
   AGDP23. System according to AGDP18, wherein the sensor for the capture of a person is a camera (185), a LIDAR (1), a radar sensor, an ultrasonic sensor (194), or an inertial sensor (5620).
   AGDP24. System according to AGDP18 comprising a movement direction module (5698) for determining the direction of movement of a person.

### Example 24: Modification of Signals of an Optical Sensor

When capturing movements of a person as part of the evaluation of a skeleton model, clothing provides for a potential inaccuracy in the detection of the movements, because a portion of the movements of the person's body, and in particular the kinetic energy, is absorbed by the clothing and also converted into movements that are not necessarily synchronous with the person's movement. This decreases the capture quality of the movements. For this reason, as described below, correction calculations are implemented in order to reduce the effects of the movement of the clothing on the detection of the movements of the skeleton points, thereby improving the signal-to-noise ratio.

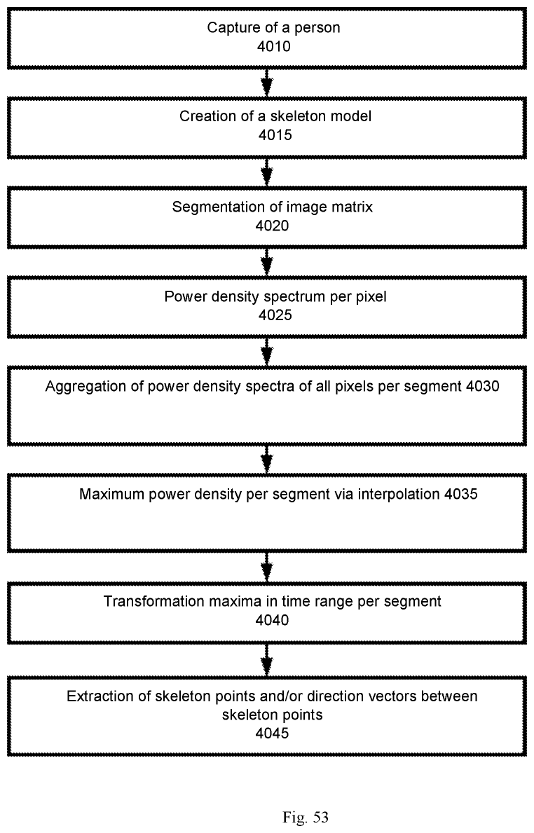

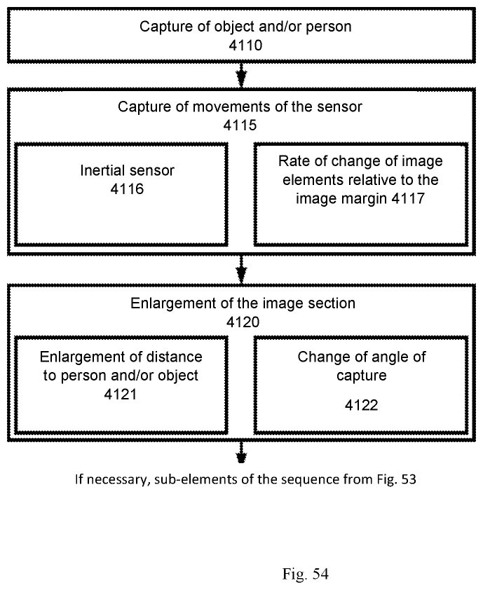

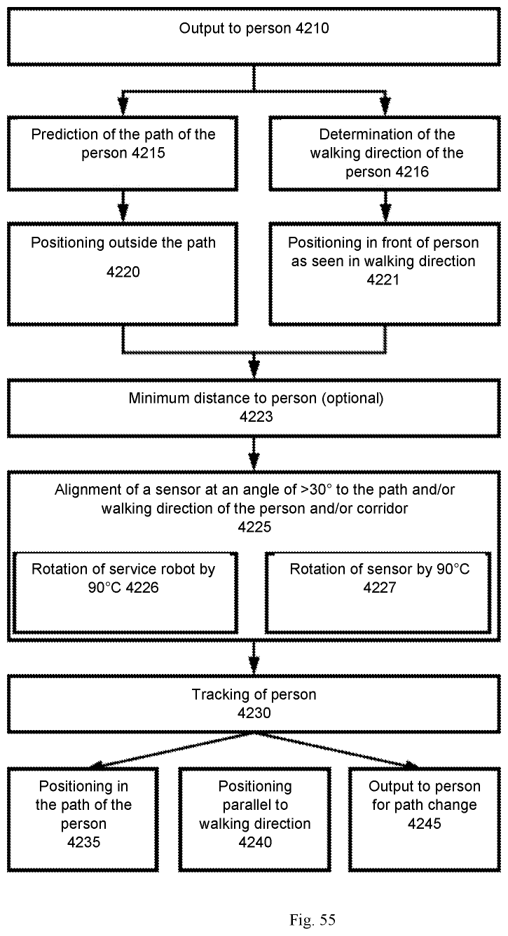

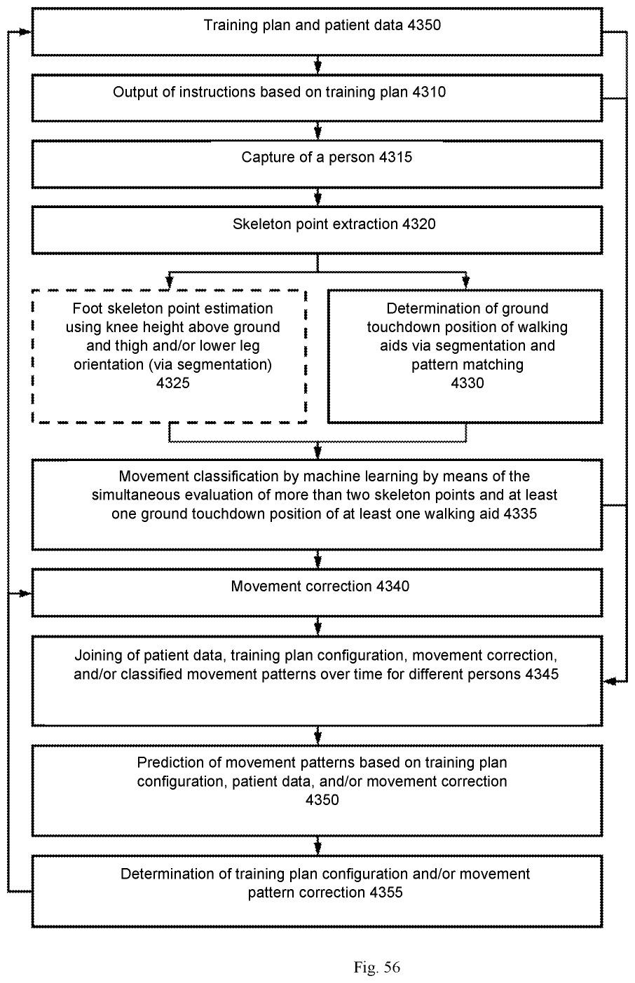

As shown in FIG. 53, the person is detected in step 4010, e.g. by the camera 185 of a service robot or else by another stationary or mobile camera. A skeleton model 4015 is created based on the captured data, e.g. using a camera SDK, OpenPose, etc. The matrix of the image is segmented into regions of interest/segments 4020, and in one aspect, into the regions in which the skeleton points are detected. Here, a region of interest/segment comprises, for example, a roughly circular area (in a 2D view) that extends around the skeleton points. For each region of interest/segment, the power density spectrum per pixel is calculated 4025 (e.g. by means of a fast Fourier transform) and aggregated 4030 over all pixels (element of an image matrix) of the region of interest/segment. Quadratic interpolation, for example, is applied to determine the maximum of the power density per region of interest/segment 4035. The maxima are transformed into the time range 4040, with each region of interest/segment then representing a corrected position and/or movement of the skeleton point. In a further step, the skeleton points and/or direction vectors between the skeleton points can be extracted 4045.

The system for modifying the signals of an optical sensor or for evaluating sensor data of a person, for example by reducing the signal-to-noise ratio, is illustrated in FIG. 77 as follows: The system comprises a processing unit 9, a memory 10, an image matrix segmentation module 5705 for segmenting the image matrix into regions of interest, and a power density module 5710 for power density determination and processing. In one aspect, the system further comprises a skeleton creation module 5635 for creating a skeleton model of the person, a skeleton point selection module 5720 for selecting skeleton points of the skeleton model (e.g. as part of the image matrix segmentation module 5705), and a skeleton model correction module 5715 for determining new positions of identified skeleton points. In one aspect, the system also includes a movement evaluation module 120 with a movement extraction module (121) comprising a gait feature extraction module 5605 and additional modules for classifying extracted features of movements. The system may be a service robot (17). In one aspect, the system has a person recognition module 110 and/or a skeleton model-based feature extraction module 5640.

The modification of signals of an optical sensor is characterized here by the following aspects AMSS1 to AMSS18:

AMSS1. Computer-implemented method for detecting a person and/or an object comprising
the resolution of the object and/or person as an image matrix;
the segmentation of the image matrix into regions of interest;
the determination of the power density spectrum for each element of the image matrix within a region of interest;
the aggregation of the power density spectra across all elements of the region of interest; and
the determination of the maximum of the power density through interpolation.
   AMSS2. Computer-implemented method according to AMSS1, wherein the segmentation of the image matrix is based on a skeleton model and the segments comprise the regions around the skeleton points.
   AMSS3. Computer-implemented method for modifying the position of skeleton points of a skeleton model comprising
the capture of a person over time;
the resolution of the person as an image matrix;
the segmentation of the image matrix into regions of interest;
the determination of the power density spectrum for each element of the image matrix within a region of interest;
the aggregation of the power density spectra across all elements of the region of interest; and
the determination of the maximum of the power density through interpolation.
   AMSS4. Computer-implemented method for improving the signal-to-noise ratio in the creation of a skeleton model of a person comprising
the capture of a person over time;
the determination of a skeleton model of the person;
the segmentation of elements of the skeleton model, wherein the power density spectrum is determined for each pixel of a segment;
the aggregation of the power density spectra per segment;
the determination of the maximum of the power density;
the transformation of the maxima into the time range and
the further processing of the obtained values in the scope of a classification depending on location and/or time progression.
   AMSS5. Computer-implemented method according to AMSS1, AMSS3 or AMSS4, wherein the power density spectra are aggregated by means of a fast Fourier transform.
   AMSS6. Computer-implemented method according to AMSS1, AMSS3, or AMSS4, wherein the interpolation is a quadratic interpolation.
   AMSS7. Computer-implemented method according to AMSS1 or AMSS3 further comprising a transformation of the maxima into the time range.
   AMSS8. Computer-implemented method according to AMSS4 or AMSS7, wherein the transformation of the maxima into the time range is carried out by means of an inverse fast Fourier transform.
   AMSS9. Computer-implemented method according to AMSS1, AMSS3 or AMSS4 comprising the capture and evaluation of movement parameters and/or movement patterns of a person.
   AMSS10. Computer-implemented method according to AMSS1, AMSS3, or AMSS4, wherein the movement parameters and/or movement patterns of a person are gait parameters of the person that are captured and evaluated in a spatially and temporally resolved way.
   AMSS11. Computer-implemented method according to AMSS1, AMSS3, or AMSS4 further comprising the determination of new skeleton point positions for a skeleton model of a captured person.
   AMSS12. Computer-implemented method according to AMSS11, wherein the positions of the skeleton points are corrected positions.
   AMSS13. Computer-implemented method according to AMSS1, AMSS3, or AMSS4, wherein the person is a clothed person, or the captured area of a person is a clothed area.
   AMSS14. Computer-implemented method according to AMSS1 and AMSS3, wherein the regions of interest represent skeleton points of the person based on the creation of a skeleton model.
   AMSS15. Device for performing a method according to AMSS1-AMSS14.
   AMSS16. System for evaluating sensor data of a person comprising a processing unit (9), a memory (10), an image matrix segmentation module (5705) for segmenting the image matrix into regions of interest, and a power density module (5710) for power density determination and processing.
   AMSS17. System according to AMSS16 further comprising a skeleton creation module (5635) for creating a skeleton model of the person, a skeleton point selection module (5720) for selecting skeleton points of the skeleton model, and a skeleton model correction module (5715) for determining new positions of identified skeleton points.
   AMSS18. System according to AMSS16 further comprising a movement evaluation module (120) with a movement extraction module (121) comprising a gait feature extraction module (5605).

### Example 25: Image Correction

In one aspect, the service robot 17 includes an image correction mechanism (see. FIG. 54) designed for navigating uneven surfaces, with the service robot 17 being capable of capturing objects and/or persons by means of a sensor 4110. The service robot 17 may, in one aspect, move outside of buildings. In this regard, in one aspect, the surfaces over which the service robot 17 moves may be uneven and may result in a “jerking” movement that moves the area covered by the camera 185 and may cause a person and/or object captured by the camera 185 that is/are completely captured on level ground to not be completely captured, at least temporarily. For this reason, the service robot includes a detection of the movements of the sensor 4115.

In one aspect, the jerking is detected by the service robot 17. For this purpose, in one aspect, an inertial sensor 4116 can be used to detect the movements of the sensor, and in an alternative and/or additional aspect, the image of a camera 185 is directly evaluated with respect to artifacts, objects, persons, markers, skeleton points of a skeleton model, etc. that are present in the image and their distance from the margin of the captured image area, whereby individual elements of an image can be tracked. For this purpose, the service robot detects the rate of change of these image elements relative to the margin of the image 4117, i.e. if the distance of these artifacts, objects, persons, markers, skeleton model points, etc. changes at a rate that is below a threshold value, this is classified as jerking. Alternatively and/or additionally, the distance of these to the image margin is evaluated. Alternatively and/or additionally for the case that a person is captured for whom a skeleton model is created, the system detects whether the skeleton model is completely determined. Subsequently, the image section is enlarged 4020, which, in one aspect, is realized by extending the distance of the sensor to object and/or the person 4021. Alternatively and/or additionally, a zoom function is used for this purpose and/or the angle of coverage of the sensor is expanded 4022, for example by increasing the distance to the captured person. This ensures that the object and/or the person is/are still within the image section and can be tracked, despite any jerking movements. In an alternative and/or additional aspect, an interpolation of the movements of the skeleton model is carried out as shown in the previous examples (see “determination of the power density spectrum”).

The system for adjusting an image section is illustrated in FIG. 78: The system is described as a system for optically capturing a person by means of a sensor with a processing unit 9, a memory 10, and an image section adjustment module 5740 for enlarging the image section, e.g. based on the movements of the sensor. The system may further comprise a person tracking module (112 or 113). In order to determine whether it is necessary to adjust the image section based on a movement, the system may employ multiple alternative and/or additional modules: a) an image section rate of change module 5745 for evaluating the rate of change of the person's position within the image section; b) an image section distance module 5750 for evaluating the distance of the person to the margin of the image section includes; c) an inertial sensor 5620; and d) a skeleton creation module 5635 for creating a skeleton model of the person, and a skeleton point image section module (5760) for determining the number of skeleton points in the image section, whose variation is used to determine movement. The system includes, for example, an image section enlargement unit 5755 for enlarging the image section by increasing the distance of the system to the captured person. The image section enlargement unit 5755 includes, for example, a movement planner 104, a motor controller 192 and/or a zoom function. The sensor is, for example, a camera 185 or a LIDAR 1. The system may include, for example, a gait feature extraction module 5605 for feature extraction of a gait pattern, a gait feature classification module 5610 for feature classification of a gait pattern, and/or a gait pattern classification module 5615 for gait pattern classification. In one aspect, the system is a service robot 17. In one aspect, the system includes a person recognition module 110, a movement evaluation module 120, a skeleton creation module 5635, and/or a skeleton model-based feature extraction module 5640. The sequence can be summarized as follows: the system captures and tracks a person within an image section, detects at least one movement, and enlarges the image section, e.g. by increasing the distance to the captured person, via a speed reduction and/or by increasing the angle of capture by means of a zoom function, either using the lens with a view of the real image section and/or using a software-based solution that evaluates what functions as image section, whereby in the latter case the image section processed in software is smaller than the image section captured by the sensor.

Image correction is characterized here by the following aspects ABK1 to ABK23:

ABK1. Computer-implemented method for movement correction for object capture comprising
the capture and tracking of a person within an image section;
the detection of at least one movement; and
the enlargement of the image section.
   ABK2. Computer-implemented method according to ABK1, wherein the detection of at least one movement comprises the rate of change of the position of the person and/or parts of the person within the image section.
   ABK3. Computer-implemented method according to ABK1, wherein the detection of at least one movement comprises the evaluation of the distance of the person and/or parts of the person in the image section to the margin of the image section.
   ABK4. Computer-implemented method according to ABK1, wherein the detection of at least one movement is performed by an inertial sensor (5620).
   ABK5. Computer-implemented method according to ABK1 further comprising the creation of a skeleton model of the person and a location- and time-dependent evaluation of extracted skeleton points after capturing the person.
   ABK6. Computer-implemented method according to ABK5, wherein at least one movement is detected by changing the number of detected skeleton points within a skeleton model located within the image section.
   ABK7. Computer-implemented method according to ABK1, wherein the image section is enlarged by increasing the distance to the captured person.
   ABK8. Computer-implemented method according to ABK7, wherein the image section is enlarged by means of a speed reduction.
   ABK9. Computer-implemented method according to ABK1, wherein the image section is enlarged by increasing the angle of coverage by means of a zoom function.
   ABK10. Computer-implemented method according to ABK1, wherein the detecting movements originate from unevenness of the ground.
   ABK11. Computer-implemented method according to ABK1, wherein the detecting movements originate from movements of the sensor.
   ABK12. Device for performing a method according to ABK1-ABK11.
   ABK13. System for movement correction for object capture comprising a processing unit (9), a memory (10) and a sensor for capturing an object and/or a person over time, and an image section adjustment module (5740) for adjusting an image section containing the object and/or the person.
   ABK14. System according to ABK13 further comprising a person tracking module (112 or 113).
   ABK15. System according to ABK13 further comprising an image section rate of change module (5745) for evaluating the rate of change of the position of the person and/or the object within the image section.
   ABK16. System according to ABK13 further comprising an image section distance module (5750) for evaluating the distance of the person and/or the object to the margin of the image section.
   ABK17. System according to ABK13 comprising an inertial sensor (5620) for evaluating movements of the sensor for capturing an object and/or a person.
   ABK18. System according to ABK13 comprising a skeleton creation module (5635) for creating a skeleton model of the person and a skeleton point image section module (5760) for determining the number of skeleton points within the image section, for example.
   ABK19. System according to ABK13 comprising an image section enlargement unit (5755) for enlarging the image section by increasing the distance of the system to the captured person and/or object.
   ABK20. System according to ABK19, wherein the image section enlargement unit (5755) includes a movement planner (104) and a motor controller (192).
   ABK21. System according to ABK19, wherein the image section enlargement unit (5755) includes a zoom function.
   ABK22. System according to ABK13 further comprising a gait feature extraction module (5605) for feature extraction of a gait pattern, a gait feature classification module (5610) for feature classification of a gait pattern, and/or a gait pattern classification module (5615) for gait pattern classification.
   ABK23. System according to ABK13, wherein the sensor is a camera (185) or a LIDAR (1).

### Example 26: Navigation of the Service Robot for the Purpose of Capturing Lateral Recordings of a Person

The service robot 17 identifies and tracks persons over time. While doing so, the service robot 17 tracks the person not only approximately parallel to the path that the person is covering, but also at an angle greater than 30°, preferably greater than 45°, in an aspect approx. 90° to this path. Rules are stored in the service robot for this purpose, as shown in FIG. 55: By means of the output unit, the service robot uses an output 4210 to instruct a person to walk essentially straight ahead and/or to follow a certain path. The service robot 17 predicts, for example, the path 4215 that the person is to travel, for example, by means of a path planning module 103, positions itself outside the predicted path of the person 4220, possibly at a minimum distance to the predicted path 4223, and positions itself such that the service robot 17 can track the person at an angle greater than 30°, preferably greater than 45°, in an aspect approx. 90° to the predicted path 4225. Alternatively and/or additionally to path prediction, the service robot 4226 can determine the walking direction of the person 4216 while positioning itself in front of the person 4221 as seen in the walking direction, wherein in one aspect a minimum distance 4223 to the person is maintained (in the walking direction and/or perpendicular to the walking direction), and the positioning is performed such that it's at least one sensor for capturing the person captures the person at an angle of greater than 30°, preferably greater than 45°, in an aspect approx. 90° to the tracked walking direction of the person. For example, in the case of a rigidly mounted sensor, this can mean that the service robot 4226 rotates greater than 30°, preferably greater than 45°, in an aspect approx. 90° towards this path, and/or the service robot aligns at least one potentially movable sensor at an angle of greater than 30°, preferably greater than 45°, in an aspect approx. 90° 4227 towards this path and/or the walking direction of the person. In one aspect, instead of the walking direction and/or path of the person, an obstacle, e.g. an object such as a wall, a line on the floor, etc. may also serve as a reference for the angle of alignment. In one aspect, the angle of positioning relative to the person is derived from an analysis of the skeleton model and/or the gait pattern of the person. In an alternative and/or additional aspect, the service robot 17 (or at least one sensor that captures the person) essentially rotates in place. The person 4230 is tracked. In an alternative and/or additional aspect, the service robot moves alongside the person for a defined time and/or a defined distance while capturing the person essentially from the side. The service robot 17 then navigates back into the path of the person 4235, in one aspect in front of the person, in an alternative aspect behind the person, such that tracking again occurs essentially parallel to the path of the person. Alternatively and/or additionally, the service robot 17 positions itself parallel to the walking direction of the person 4240 ahead of or behind the person while assuming approximately the same speed as the person. Alternatively and/or additionally, the service robot 17 may also instruct the person to change his or her path via an output 4245 of the output unit.

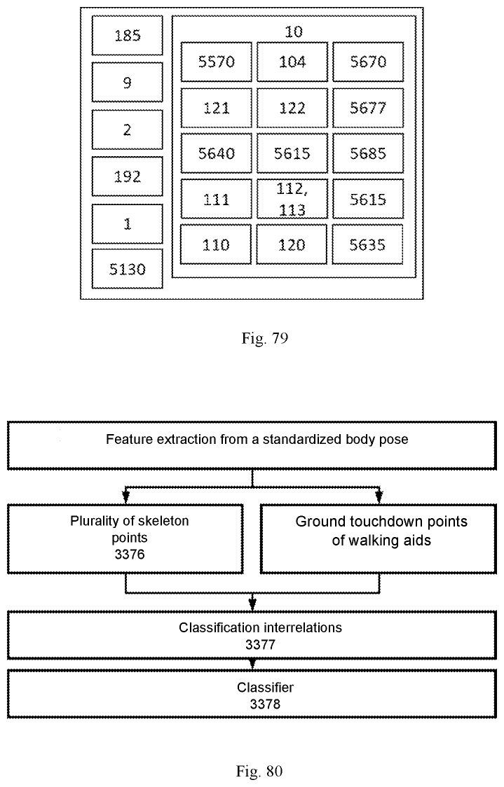

The system for navigating the service robot for the purpose of capturing lateral recordings of a person can be summarized as follows, as illustrated in FIG. 79: The system for positioning a detection and/or evaluation unit at an angle greater than 30° to the walking direction of a person (e.g. a service robot 17) includes a processing unit 9, a memory 10, and at least one sensor for capturing a person over time, further comprising a person tracking module (112 or 113) and a positioning module 5570 for initiating and monitoring the positioning. In one aspect, the system includes an output unit such as a display 2 and/or a loudspeaker 192. The system further comprises a movement planner 104, e.g. for making a prediction of the path to be covered by the person, moving the detection and evaluation unit adjacent to the person, maintaining an approximately constant distance to the person, assuming a defined angle, and/or rotating the detection and evaluation unit. The system includes, in one aspect, a tilting unit 5130 that allows the sensor to be oriented while the orientation of the detection and evaluation unit is fixed, and further includes, for example, a camera 185 and/or a LIDAR 1. The system may include, for example, a movement extraction module 121 for feature extraction of a movement pattern of the person, and a movement assessment module 120, wherein the movement extraction module 121 may be, for example, a skeleton model-based feature extraction module 5640, and the movement assessment module 122 may include a gait feature classification module 5610 for feature classification of the gait pattern of the person, and/or a gait pattern classification module 5615 for gait pattern classification of the person. However, the movement extraction module 121 may relate to a movement pattern other than gait-related movements described in this document, as may the movement assessment module 122. In one aspect, the system includes a person recognition module 110, a movement evaluation module 120, and/or a skeleton creation module 5635. The sequence can be summarized as follows: capture and tracking the person by at least one sensor, determination of the walking direction of the person, and repositioning of the detection and/or evaluation unit, wherein the repositioning of the detection and/or evaluation unit enables, for example, an essentially lateral capture of the person or the capture of the person in the sagittal plane. In one aspect, an instruction to the person to walk essentially straight ahead is outputted. The sequence further comprises a prediction of a path to be covered by the person based on the person's walking direction, for example with subsequent repositioning of the detection and/or evaluation unit to the path at the angle of coverage or at the angle of coverage to a wall. The angle of coverage results, for example, from a mid-centered axis of the sensor and a wall, on the one hand, and the walking direction and/or the predicted path on the other hand, each projected onto a horizontal plane. In a further step, a continuous recalculation of the angle of capture and the positioning of the detection and/or evaluation unit can be performed so that the angle of capture is kept approximately constant. Further, for example, a continuous calculation of the distance between the detection and/or evaluation unit and the person may be performed, as well as a positioning of the detection and/or evaluation unit in such a way that a minimum value for the distance between the detection and/or evaluation unit and the person is maintained. Additionally, the repositioning of the detection and/or evaluation unit may be performed after a defined time and/or distance so that the angle of capture thereafter is essentially smaller than 30°, as well as the repositioning of the detection and/or evaluation unit after a defined time and/or distance so that the angle of capture thereafter is essentially smaller than 30°. In an additional aspect, for example, in the course of the capture and tracking of the person, an output can be made with an indication of the direction of movement of the person and/or that of the detection and/or evaluation unit, and/or an evaluation of the movement pattern can be made taking the walking direction of the person into account. In one aspect, the evaluation of the movement pattern includes the detection of the ground touchdown points of walking aids, which are evaluated together with the position of the feet of the captured person, for which purpose, for example, the foot skeleton point classification module 5670, the foot skeleton point-walking aid position module 5677 and/or the foot skeleton point distance determination module 5685, which have already been described elsewhere, can be used.

The navigation of the service robot for the purpose of capturing lateral recordings of a person is characterized here by the following aspects NSRSA1-NSRSA18:

NSRSA1. Computer-implemented method for positioning a detection and/or evaluation unit at an angle of capture greater than 30° relative to the walking direction of a person comprising
the detection and tracking of the person by at least one sensor,
the determination of the walking direction of the person, and
the repositioning of the detection and/or evaluation unit.
   NSRSA2. Computer-implemented method according to NSRSA1 comprising outputting an instruction to the person to walk essentially straight ahead.
   NSRSA3. Computer-implemented method according to NSRSA1, wherein the repositioning of the detection and/or evaluation unit enables an essentially lateral capture of the person or the capture of the person in the sagittal plane.
   NSRSA4. Computer-implemented method according to NSRSA1 comprising the prediction of a path to be covered by the person based on the walking direction of the person.
   NSRSA5. Computer-implemented method according to NSRSA4, wherein the detection and/or evaluation unit is repositioned in its angle of capture towards the path.
   NSA6. Computer-implemented method according to NSRSA4, wherein the detection and/or evaluation unit is repositioned in its angle of capture towards an object.
   NSRSA7. Computer-implemented method according to NSRSA4, wherein the angle of capture results from a mid-centered axis of the sensor and an object on the one hand, and the walking direction and/or the predicted path on the other hand, each projected onto a horizontal plane.
   NSRSA8. Computer-implemented method according to NSRSA1 further comprising a continuous recalculation of the angle of capture and the positioning of the detection and/or evaluation unit in such a way that the angle of capture is kept approximately constant.
   NSRSA9. Computer-implemented method according to NSRSA1 further comprising
a continuous calculation of the distance between the detection and/or evaluation unit and the person; and
the positioning of the detection and/or evaluation unit so as to maintain a minimum value for the distance between the detection and/or evaluation unit and the person.
   NSRSA10. Computer-implemented method according to NSRSA1 further comprising the repositioning of the detection and/or evaluation unit after a defined time and/or distance so that the angle of capture thereafter is essentially smaller than 30°.
   NSRSA11. Computer-implemented method according to NSRSA1 further comprising the repositioning of the detection and/or evaluation unit after a defined time and/or distance so that the angle of capture thereafter is essentially smaller than 30°.
   NSRSA12. Computer-implemented method according to NSRSA1 further comprising, in the course of the capture and tracking of the person, an output with an indication of the direction of movement of the person and/or that of the detection and/or evaluation unit.
   NSRSA13. Computer-implemented method according to NSRSA1 further comprising an evaluation of the movement pattern taking the direction of movement of the person into account.
   NSRSA14. Device for performing the computer-implemented method according to NSRSA1-13.
   NSRSA15. System for positioning a detection and/or evaluation unit at an angle greater than 30° to the walking direction of a person, comprising a processing unit (9), a memory (10), at least one sensor for capturing the person over time, a tracking module (112, 113) for tracking the person, and a positioning module (5570) for initiating and monitoring the positioning.
   NSRSA16. System according to NSRSA15 further comprising a movement planner (104) for making a prediction of the path to be covered by the person, moving the detection and/or evaluation unit adjacent to the person, for maintaining an approximately constant distance between the detection and/or evaluation unit and the person, assuming a defined angle of capture, and/or for rotating the detection and/or evaluation unit.
   NSRSA17. System according to NSRSA15 comprising a tilting unit (5130), with which the sensor can be aligned while the orientation of the detection and/or evaluation unit remains fixed.
   NSRS18. System according to NSRSA15 comprising a movement extraction module (121) for feature extraction of a movement pattern of the person and a movement assessment module (122).

### Example 27: Movement Pattern Prediction

In one aspect, the service robot 17 communicates with a system that follows, for example, the following sequence: A training plan and patient data are stored in a memory 10, e.g. in the service robot 17 and/or in the cloud 18, to which the service robot 17 is connected via an interface 188 (step 4305). The system, for example the service robot 17, issues instructions based on the training plan (step 4310), which are stored in the memory 10, whereby these can be output, for example, via a display 2 and/or a loudspeaker 192. Furthermore, a person such as a patient (step 4315) is captured over time, e.g. by means of the visual or laser-based person tracking module 112, 113. For this purpose, a 2D and/or 3D camera 185 such as an RGB-D camera is used. Furthermore, a skeleton point extraction 4320 is performed, with the skeleton points originating from a skeleton model. This can be achieved, for example, using the SDK for a Microsoft Kinect, or OpenPose.

In an optional aspect, a foot skeleton point is not obtained via the SDK, but by means of a separate estimation described in step 4325. This is done using an approach as explained in Example 21.

The ground touchdown position of walking aids such as forearm crutches or underarm crutches is determined in step 4330. This is done using a segmentation algorithm, e.g. RANSAC, and pattern matching, where the patterns may originate from 2D and/or 3D data and describe the shape of the walking aid. Also, coordinates are evaluated in two-dimensional or three-dimensional space.

The next step is a movement classification by machine learning by means of the simultaneous evaluation of more than two skeleton points and at least one ground touchdown position of at least one walking aid (step 4335). In the process, the skeleton points and the ground touchdown point are evaluated in relation to each other. At least one foot skeleton point is included for this process. The classifier used for this is, in one aspect, created using a neural network based on supervised learning, with the captured body positions of the person having been assessed. In one aspect, a filter can be used when creating a classifier in order to reduce the information used for classification. The next step is, for example, a reduction of the extracted features, e.g. a dimension reduction. Maxima can be processed on the one hand, or average values of the extracted features on the other. Later on, cost functions can be applied, for example. PyTorch, for example, can be used as a software tool.

In one aspect, body poses of a gait pattern using walking aids such as forearm or underarm crutches are recorded, these body poses describing a correct gait pattern. For these body poses, the positions and the progression of at least two skeleton points and at least one end point of a forearm or underarm crutch are recorded over time and contiguously evaluated. In the process, the progression of skeleton points and/or touchdown points of the forearm or underarm crutches can be evaluated in each case, for example, on the basis of a demonstration of a body pose, and a classifier can be created on the basis of this, which is then compared with additionally recorded body poses that are predefined as correct and the courses of the skeleton points and touchdown points of the forearm or underarm crutches in the room which are derived from this, with a classifier then being created again that takes all the progression data of the skeleton points and underarm/forearm crutch positions into account. This narrows down the at least one classifier. The DAgger algorithm in Python can be used for this purpose, for example. This way, for example, a neural network can be used to create a classifier that recognizes a correct movement and, consequently, also recognizes movements that do not proceed correctly. FIG. 80 illustrates this method, in which feature extraction is first performed from a standardized body pose, e.g. a gait pattern 3375. In a next step, multiple skeleton points 3376 and the ground touchdown points of a walking aid such as the forearm or underarm crutches 3377 are captured and classified contiguously, thereby generating a classifier 3378. This process can be applied iteratively with a variety of body poses, which are standardized or correspond to a correct sequence.

In the next step 4440, a movement correction is performed based on rules stored in memory 10. Outputs via a loudspeaker 192 and/or a display 2 are associated with this. The data may be stored, for example, in the form of a matrix combining recognized movement patterns with associated correction outputs. The correction outputs are prioritized in such a way that only a defined number of correction outputs occur within a defined time frame, e.g. a maximum number, which in turn depends on the length of the outputs and/or movements of the system such as the service robot.

In step 4345, the patient data (mostly time-invariant data such as age, weight, height, type of operation, operated side, etc.), the training plan configurations (which may be time-variant, such as 5 min training on the first day, 10 min training on the second day, distance to be covered of 50 m, etc.), the outputted movement corrections (e.g. straightening the upper body, placing the forearm crutches differently), and/or the classified movement patterns over time (such as the angles between limbs, step length, track width, etc.) for the captured persons are all joined together with the aim of evaluating the data. For this purpose, common join commands for a database can be used, for example, provided that the data is stored in a database in the memory 10. The joining can be performed, for example, for each recorded exercise. This data is stored in the memory 10, which is located either in the service robot 17 or in the cloud 18. Based on the acquired data, a prediction of movement patterns based on the training plan configuration, patient data, and/or movement correction is made (step 4350). This way, it is possible to determine, for example, which parameters for a training plan configuration can be achieved for certain patient types (age, etc.) and which movement corrections can be used to achieve a movement pattern for a patient that meets certain requirements (e.g. that is especially fluid, especially close to producing the normal gait pattern, etc.). The movement patterns can be classified, in one aspect, e.g. as a “normal” movement pattern vs. a disease-related movement pattern. The prediction of movement patterns is achieved by machine learning algorithms. For example, a structural equation model can be used, e.g. from the semopy toolkit for Python or a regression model based on scikit-learn for Python. In one aspect, neural networks can also be used here. Based on this evaluation, a determination is made of which training plan configuration and/or which movement correction brings about which result. For this purpose, a prediction is made to determine which combination of which influencing factors, such as training plan configurations, personal data, and movement corrections, leads to which movement patterns. Based on this, a training plan configuration and/or the classification of the movement correction is adapted (step 4355), i.e., the training plan configurations and/or movement corrections that lead to defined movement patterns are transmitted. The transmission is made to a system for collecting the movement data based on outputs of a training plan, e.g., a service robot 17, a cell phone or a computer, which is either stationary or mobile, e.g. a tablet. The system may be, for example, a service robot 17 or a stationary system. In one aspect, the transmission may also be to a rule set 150 from which rules are distributed to more than one other device.

The sequence can be summarized as follows: the joining of personal data, training plan configurations, movement corrections, and classified movement patterns over time for different persons in at least one memory (10); the prediction of movement patterns based on the training plan configurations, personal data, and/or the movement corrections; the determination of training plan configurations and/or movement corrections that lead to defined movement patterns; the transmission of the training plan configurations and/or movement corrections to a system for capturing movement patterns. Furthermore: the capture of a person, the creation of a skeleton model of the person, extraction of skeleton points of the person over a movement pattern, movement classification of the extracted skeleton points for assessing movement patterns, and the determination of a movement correction.

The recognition and evaluation of gait parameters is characterized here by the following aspects AEAG1 to AEAG14):

AEAG1. Computer-implemented method for predicting movement patterns comprising
the joining of personal data, training plan configurations, movement corrections, and classified movement patterns over time for different persons in at least one memory (10);
the prediction of movement patterns based on the training plan configurations, personal data, and/or the movement corrections;
the determination of training plan configurations and/or movement corrections that lead to defined movement patterns;
the transmission of the training plan configurations and/or movement corrections to a system for capturing movement patterns.
   AEAG2. Computer-implemented method according to AEAG1 comprising the output of instructions based on a training plan.
   AEAG3. Computer-implemented method according to AEAG1 comprising the capture of a person, the creation of a skeleton model of the person, extraction of skeleton points of the person over a movement pattern, movement classification of the extracted skeleton points in order to assess movement patterns, and the determination of a movement correction.
   AEAG4. Computer-implemented method according to AEAG3, further comprising the estimation of the position of the associated foot skeleton point for one leg by way of a determination of the distance between the associated knee skeleton point and the floor, the determination of the orientation of the associated lower leg as a direction vector whose length is derived from the distance between the associated knee skeleton point and the floor.
   AEAG5. Computer-implemented method according to AEAG4, wherein the distance between the knee skeleton point and the floor is determined when the direction vector between the knee skeleton point and the associated hip skeleton point is approximately perpendicular.
   AEAG6. Computer-implemented method according to AEAG4, wherein the distance between the knee skeleton point and the floor is determined by the difference between the distance between the hip skeleton point and the floor and the hip skeleton point and the knee skeleton point.
   AEAG7. Computer-implemented method according to AEAG3 comprising the determination of the ground touchdown position of the walking aid used by the captured person.
   AEAG8. Computer-implemented method according to AEAG3 and AEAG7, wherein the movement classification is performed by simultaneously evaluating more than two skeleton points and at least one ground touchdown position of the at least one walking aid.
   AEAG9. Computer-implemented method according to AEAG3, wherein the movement correction comprises an output via a loudspeaker (192) and/or a display (2).
   AEAG10. Computer-implemented method according to AEAG8, wherein the evaluated skeleton points are at least one foot skeleton point.
   AEAG11. Computer-implemented method according to AEAG1, wherein the system is a service robot 17.
   AEAG12. Device for performing a method according to AEAG1-AEAG11.
   AEAG13. Device for performing a method according to AEAG11, wherein the system for capturing movement patterns is a service robot 17.
   AEAG14. Device for performing method according to AEAG11, wherein the system for capturing movement patterns is a cell phone, a tablet computer, or a stationary computer.

### REFERENCE TERMS

1 LIDAR
2 Display
3 Sensor for the contactless detection of a person
4 Pressure-sensitive bumper
5 Support wheel
6 Drive wheel
7 Drive unit
8 Energy source
9 Processing unit
10 Memory
17 Service robot
13 Terminal
18 Cloud
100 Software level
101 Navigation module
102 2D or 3D environment detection module
103 Path planning module
104 Movement planner
105 Self-localization module
106 Mapping module
107 Map module
108 Loading module
110 Person recognition module
111 Person identification module
112 Visual person tracking module
113 Laser-based person tracking module
114 Person reidentification module
115 Seat recognition module
120 Movement evaluation module
121 Movement extraction module
122 Movement assessment module
130 Human/robot interaction module
131 Graphic user interface
132 Speech evaluation module
133 Speech synthesis unit
150 Rule set
151 Rule set processing unit
152 Rule set memory
160 Patient administration module
161 Patient administration module processing unit
162 Patient administration module memory
170 Navigation module in the cloud
171 Navigation processing unit
172 Navigation memory
180 Hardware level
181 Odometry unit
183 RFID
185 Camera
186 Control elements
188 Interface
190 Charge control
191 Motor controller
192 Loudspeaker
193 Microphone
194 Radar sensor and/or ultrasonic sensor
195 Detector
196 Spectrometer
197 Projection device
905 Chair
910 Person
915 Projected marker
920 Projection device
925, 930, 935, 940 Various lines
3610 Determination of standing
3611 Distance measurement of head to floor
3612 Orientation of foot-knee, knee-hip, hip-shoulder direction vectors, essentially parallel
3613 Knee-hip direction vector approx. perpendicular
3614 Threshold value comparison
3615 Threshold value comparison
3616 Standing
3617 Sitting
3620 Detection if hand is using an aid
3621 Distance determined between object and at least one hand skeleton point
3622 Threshold value comparison
3623 Aid
3624 No aid
3630 Balance determination
3631 Amplitude, orientation and/or frequency of change in position of shoulder skeleton points/hip skeleton points, foot skeleton points, arm skeleton points in the transverse plane
3632 Threshold value comparison
3633 Deviation (amplitude, orientation and/or frequency of a direction vector (foot, knee or hip to a skeleton point above) from the perpendicular and/or in the sagittal and/or frontal plane
3634 Threshold value comparison
3635 stable
3636 unstable
3640 Foot distance
3641 Distance between foot and/or knee skeleton points
3642 Threshold value comparison
3643 short
3644 long
3660 Gait determination
3661 Change in position of shoulder skeleton points/hip skeleton points, foot skeleton points in the transverse plane and/or their distances to each other
3662 Threshold value comparison
3663 Curve of the skeleton points in the sagittal plane
3664 Threshold value or curve comparison
3665 No walking
3666 Walking and/or Walking attempts
3670 Step length determination
3671 Distance measurement of foot skeleton points over time (alternating) within sagittal plane
3672 Maxima correspond to step length
3695 Track width
3696 Distance measurement of foot skeleton points over time in the frontal plane
3931 Step length
3932 Distance of foot skeleton points detected
4415 Person position determination module
4420 Audio source position determination module
4425 Audio signal comparison module
4430 Audio signal-person module
4435 Audio sequence input module
4510 Time-distance module
4515 Speed-distance module
4520 Time-distance assessment module
4525 Hearing test unit
4530 Eye test unit
4535 Mental ability test unit
4540 Chair detection module
4605 Person detection and tracking unit
4606 Movement frequency detection unit
4607 Movement unit
4615 Pulse-respiratory evaluation unit
4620 Movement signal detection and processing unit
4625 Stylized embodiment elements
4705 Sheet detection module
4710 Folding motion detection module
4720 Sheet distance corner edge module
4725 Sheet shape change module
4730 Sheet curvature module
4740 Sheet dimension module
4745 Sheet margin orientation module
4750 Fingertip distance module
4755 Sheet segmentation module
4760 Sheet classification module
4770 Manipulation attempt detection module
4775 Person-robot distance determination module
4780 Height-arm length-orientation module
4785 Input registration comparison module
4805 Spectrometer alignment unit
4810 Body region detection module
4815 Body region tracking module
4820 Spectrometer measurement module
4825 Reference spectra database
4830 Clinical picture database
4835 Perspiration module
4840 Delirium Detection Score determination module
4845 Cognitive ability assessment module
4850 Thermometer
4905 Tactile sensor
4910 Tactile sensor evaluation unit
4915 Tactile sensor output comparison module
4920 Actuator
4925 Actuator positioning unit
4930 Hand identification module
4940 Numerical value output module
4950 Robot hand
4955 Robot hand finger pose generation module
4960 Hand pose detection module
5005 Face recognition module
5010 Face candidate region module
5015 Emotion classification module
5020 Emotion assessment module
5025 Bed recognition module
5035 Upper extremity evaluation module
5040 Pain status calculation module
5055 Pain vocalization module
5065 Ventilation device recognition module
5085 Pain sensation evaluation module
5110 Cardiovascular movements module
5120 Light
5125 Blood pressure determination module
5130 Tilting unit
5205 Evaluation laser
5210 Further laser
5215 Medium
5220 Laser deflection evaluation module
5225 Laser variation module
5230 Finger positioning recognition module
5250 Sensor based on the photoelectric effect
5270 Light source
5275 Wavelength variation unit
5280 Wavelength variation evaluation unit
5295 Substance classification module
5305 Moisture detection module
5310 Moisture assessment module
5405 Fall detection module
5410 Fall event assessment module
5415 Vital signs acquisition unit
5420 Vital signs evaluation module
5425 Vital signs sensor
5430 Fall risk determination module
5605 Gait feature extraction module
5610 Gait feature classification module
5615 Gait pattern classification module
5620 Inertial sensor
5625 Person speed module
5635 Skeleton creation module
5640 Skeleton model-based feature extraction module
5645 Transverse skeleton point evaluation module
5650 Perpendicular skeleton point evaluation module
5655 Person height evaluation module
5660 Hand distance evaluation module
5665 Sagittal plane-based skeleton point progression evaluation module
5670 Foot skeleton point classification module
5675 Track width step width module
5677 Foot skeleton point-walking aid position module
5680 Turning movement feature classification module
5682 Angle evaluation module
5685 Foot skeleton point distance determination module
5690 Hip-knee orientation module
5695 Turning movement-height-balance-step length classification module
5696 Position determination line module
5697 Distance module
5698 Movement direction module
5705 Segmentation module
5710 Power density module
5715 Skeleton model correction module
5720 Skeleton point selection module
5740 Image section adjustment module
5745 Image section rate of change module
5750 Image section distance module
5755 Image section enlargement unit
5760 Skeleton point image section module
5570 Positioning module
6070 Detected moisture on the floor
6071 Corridor
6072 Initially determined path
6073 Newly calculated path based on moisture as obstacle
6074 Determined distance between two wet surface segments

1. A computer-implemented method for positioning a detection and/or evaluation unit at an angle of capture greater than 30° relative to the walking direction of a person, comprising
detection and tracking of the person by at least one sensor,
determination of the walking direction of the person, and
repositioning of the detection and/or evaluation unit.

2. The computer-implemented method according to claim 1, comprising outputting an instruction to the person to walk essentially straight ahead.

3. The computer-implemented method according to claim 1, wherein the repositioning of the detection and/or evaluation unit enables an essentially lateral detection of the person.

4. The computer-implemented method according to claim 1, comprising a prediction of a path to be covered by the person based on the walking direction of the person.

5. The computer-implemented method according to claim 4, wherein the repositioning of the detection and/or evaluation unit is done in the angle of capture towards the path.

6. The computer-implemented method according to claim 4, wherein the repositioning of the detection and/or evaluation unit is done in the angle of capture towards an object.

7. The computer-implemented method according to claim 4, wherein the angle of capture results from a mid-centered axis of the sensor on the one hand and an object, the walking direction, and/or the predicted path on the other hand, each projected onto a horizontal plane.

8. The computer-implemented method according to claim 1, further comprising a continuous recalculation of the angle of capture; and
the positioning of the detection and/or evaluation unit such that the angle of capture is kept approximately constant.

9. The computer-implemented method according to claim 1, further comprising a continuous calculation of the distance between the detection and/or evaluation unit and the person; and
positioning of the detection and/or evaluation unit such that a minimum value for the distance between the detection and/or evaluation unit and the person is maintained.

10. The computer-implemented method according to claim 1, further comprising the repositioning of the detection and/or evaluation unit after a defined time and/or distance such that the angle of capture thereafter is essentially smaller than 30°.

11. The computer-implemented method according to claim 1, further comprising the repositioning of the detection and/or evaluation unit after a defined time and/or distance such that the angle of capture thereafter is essentially smaller than 30°.

12. The computer-implemented method according to claim 1, further comprising, in the course of the detection and tracking of the person, an output with an indication of the direction of movement of the person and/or that of the detection and/or evaluation unit.

13. The computer-implemented method according to claim 1, further comprising an evaluation of the movement sequence taking into account the direction of movement of the person.

14. A device for performing the computer-implemented method according to claim 1.

15. A system for positioning a detection and/or evaluation unit at an angle greater than 30° to the walking direction of a person, comprising a processing unit, a memory, and at least one sensor for detection of the person over time, a tracking module for tracking the person, and a positioning module for initiating and monitoring the positioning.

16. The system according to claim 15, further comprising a movement planner for creating a prediction of the path to be covered by the person, for moving the detection and/or evaluation unit adjacent to the person, for maintaining an approximately constant distance between the detection and/or evaluation unit and the person, for taking a defined angle of capture, and/or for rotating the detection and/or evaluation unit.

17. The system according to claim 15, comprising a tilting unit, which allows the alignment of the sensor while the orientation of the detection and/or evaluation unit remains fixed.

18. The system according to claim 15, comprising a movement sequence extraction module for feature extraction of a movement sequence of the person and a movement sequence assessment module.
